(19) United States
(12) Patent Application Publication
(10) Pub. No.: US 2026/0085300 A1
(43) Pub. Date: Mar. 26, 2026

(54) SYSTEMS AND COMPOSITIONS FOR FUSION POLYPEPTIDES AND METHODS OF USE THEREOF

(71) Applicant: Epicrispr Biotechnologies, Inc., South San Francisco, CA (US)

(72) Inventors: THOMAS BLAIR GAINOUS, South San Francisco, CA (US); XIAO YANG, South San Francisco, CA (US); GIOVANNI CAROSSO, South San Francisco, CA (US); LEI S. QI, South San Francisco, CA (US); DANIEL O. HART, II, South San Francisco, CA (US)

(21) Appl. No.: 19/123,019

(22) PCT Filed: Oct. 27, 2023
(86) PCT No.: PCT/US2023/078022

§ 371 Date: Apr. 21, 2025

Related U.S. Application Data
(60) Provisional application No. 63/381,169, filed on Oct. 27, 2022.

(51) Int. Cl. C12N 9/22; C07K 14/47

(52) U.S. Cl. CPC C12N 9/226; C07K 14/4703; C07K 2319/00

(57) ABSTRACT

The present disclosure provides polypeptide molecules comprising one or more polypeptide chains and systems, compositions, and methods of use thereof, wherein the polypeptide molecule comprising one or more polypeptide chains (or engineered polypeptide chain) can be used to effect regulation of a target gene in a cell (e.g., an endogenous target gene in a cell).

### CROSS REFERENCE

This application claims the benefit of U.S. Provisional Application No. 63/381,169, filed Oct. 27, 2022, which is entirely incorporated herein by reference.

### BACKGROUND

Gene expression modulation is a critical aspect of functional genomics and therapeutic development, allowing for the precise control of gene activity levels. For example, various gene modulators (e.g., transcriptional regulators) can be utilized to regulate expression or activity of a target gene in the cell. These effectors can be transcriptional activators, repressors, or epigenetic modifiers.

### SUMMARY OF THE INVENTION

Provided herein is a polypeptide molecule comprising a polypeptide chain exhibiting at least about 73% identity to the polypeptide sequence of SEQ ID NO: 1. In some embodiments, the polypeptide molecule further comprises a first polypeptide domain and a second polypeptide domain that are coupled to one another via the polypeptide chain, wherein at least one of the first polypeptide domain and the second polypeptide domain is (i) at least a portion of an endonuclease or (ii) a gene modulator configured to modulate a target gene in a cell. In some embodiments, the at least one of the first polypeptide domain and the second polypeptide domain is the at least the portion of the endonuclease. In some embodiments, the at least the portion of the endonuclease is disposed N-terminal to the polypeptide chain. In some embodiments, the second polypeptide domain is the gene modulator. In some embodiments, the at least one of the first polypeptide domain and the second polypeptide domain is the gene modulator. In some embodiments, the first polypeptide domain is the gene modulator, and the second polypeptide domain is an additional gene modulator. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the at least the portion of the endonuclease is a Cas protein. In some embodiments, the Cas protein is a deactivated Cas (dCas) protein that exhibits reduced nuclease activity. In some embodiments, the at least the portion of the endonuclease has a size of less than about 800 amino acid residues. In some embodiments, the gene modulator is a transcriptional activator. In some embodiments, the gene modulator is a transcriptional repressor. In some embodiments, the transcriptional repressor comprises one or more members selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator and the additional gene modulator are substantially the same. In some embodiments, the gene modulator and the additional gene modulator are substantially different. In some embodiments, the additional gene modulator is selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator is KRAB, or a modification thereof, and wherein the additional gene modulator is DNMT, or a modification thereof. In some embodiments, the gene modulator is EZH2, or a modification thereof, and the additional gene modulator is DNMT, or a modification thereof. In some embodiments, the DNMT comprises DNMT3A, DNMT3L, a modification thereof, or a combination thereof.

Also provided herein is a polypeptide molecule comprising a gene modulator coupled to a polypeptide chain exhibiting at least about 50% identity to the polypeptide sequence of SEQ ID NO: 1, wherein, upon formation of a complex comprising the polypeptide molecule and a target polynucleotide sequence operatively coupled to a target gene of a cell, the complex is configured to effect a greater change in expression level of the target gene, as compared to a control complex comprising the target polynucleotide sequence and the gene modulator in absence of the polypeptide chain. In some embodiments, the polypeptide molecule further comprises at least a portion of an endonuclease. In some embodiments, the greater change in expression level of the target gene is characterized by reduced expression of the target gene by at least about 15%, at least about 20%, at least about 30%, at least about 40%, at least about 50%, at least about 60%, at least about 70%, or at least about 80% compared to the control complex. In some embodiments, the greater change in expression level of the target gene persists for at least about 10 days, at least 20 days, at least 30 days, or at least 40 days post transfection. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the at least the portion of the endonuclease is a Cas protein. In some embodiments, the Cas protein is a deactivated Cas (dCas) protein that exhibits reduced nuclease activity. In some embodiments, the at least the portion of the endonuclease has a size of less than about 800 amino acid residues. In some embodiments, the gene modulator is a transcriptional activator. In some embodiments, the gene modulator is a transcriptional repressor. In some embodiments, the transcriptional repressor comprises one or more members selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator and the additional gene modulator are substantially the same. In some embodiments, the gene modulator and the additional gene modulator are substantially different. In some embodiments, the additional gene modulator is selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator is KRAB, or a modification thereof, and wherein the additional gene modulator is DNMT, or a modification thereof. In some embodiments, the gene modulator is EZH2, or a modification thereof, and the additional gene modulator is DNMT, or a modification thereof. In some embodiments, the DNMT comprises DNMT3A, DNMT3L, a modification thereof, or a combination thereof.

Provided herein is a polypeptide molecule comprising a first polypeptide domain coupled to a second polypeptide domain via a polypeptide chain, wherein the polypeptide chain is greater than 18 amino acids, wherein the first polypeptide domain is a gene modulator, wherein the second polypeptide domain is an additional gene modulator or at least a portion of an endonuclease, and wherein, upon formation of a complex comprising the polypeptide molecule and a target polynucleotide sequence operatively coupled to a target gene of a cell, the complex is configured to effect a greater change in expression level of the target gene, as compared to a control complex that comprises the target polynucleotide sequence, the first polypeptide domain, and the second polypeptide domain in absence of the polypeptide chain, wherein the greater change in expression level persists greater than 30 days. In some embodiments, the polypeptide chain exhibits at least about 50% identity to the polypeptide sequence of SEQ ID NO: 1. In some embodiments, the second polypeptide domain is the at least a portion of an endonuclease. In some embodiments, the second polypeptide domain is the additional gene modulator. In some embodiments, the greater change in expression level of the target gene is characterized by a reduced expression of the target gene by at least about 15%, at least about 20%, at least about 30%, at least about 40%, at least about 50%, at least about 60%, at least about 70%, or at least about 80% compared to the control complex. In some embodiments, the greater change in expression level of the target gene persists for at least about 40 days post transfection. In some embodiments, a first polypeptide sequence encoding the first polypeptide domain or a second polypeptide sequence encoding the second polypeptide domain is non-natural polypeptide sequence. In some embodiments, the polypeptide chain exhibits at least about 74%, at least about 75%, at least about 80%, at least about 85%, at least about 90%, at least about 95%, at least about 99%, or substantially 100% sequence identity to the polypeptide sequence of SEQ ID NO: 1. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the at least the portion of the endonuclease is a Cas protein. In some embodiments, the Cas protein is a deactivated Cas (dCas) protein that exhibits reduced nuclease activity. In some embodiments, the at least the portion of the endonuclease has a size of less than about 800 amino acid residues. In some embodiments, the gene modulator is a transcriptional activator. In some embodiments, the gene modulator is a transcriptional repressor. In some embodiments, the transcriptional repressor comprises one or more members selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator and the additional gene modulator are substantially the same. In some embodiments, the gene modulator and the additional gene modulator are substantially different. In some embodiments, the additional gene modulator is selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator is KRAB, or a modification thereof, and wherein the additional gene modulator is DNMT, or a modification thereof. In some embodiments, the gene modulator is EZH2, or a modification thereof, and the additional gene modulator is DNMT, or a modification thereof. In some embodiments, the DNMT comprises DNMT3A, DNMT3L, a modification thereof, or a combination thereof.

In some aspects, a polypeptide molecule comprising a first polypeptide domain comprising at least a portion of an endonuclease, a second polypeptide domain encoding a first gene modulator; and a polypeptide chain linking the first polypeptide domain and the second polypeptide domain, wherein the polypeptide chain exhibits at least about 50% identity to the polypeptide sequence of SEQ ID NO: 1, wherein the first polypeptide domain is disposed N-terminal to the polypeptide chain, and wherein the second polypeptide domain is disposed C-terminal to the polypeptide chain is provided. In some embodiments, the endonuclease is a Cas protein. In some embodiments, the Cas protein is a deactivated Cas (dCas) protein that exhibits reduced nuclease activity. In some embodiments, the at least the portion of the endonuclease has a size of less than about 800 amino acid residues. In some embodiments, the polypeptide molecule further comprising a third polypeptide domain encoding a second gene modulator. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the first gene modulator and the second gene modulator are transcriptional repressors. In some embodiments, the first gene modulator and the second gene modulator are substantially the same. In some embodiments, the gene modulator and the additional gene modulator are substantially different. In some embodiments, the first gene modulator or the second gene modulator is selected from the group consisting of KRAB, DNMT, EZH2, ZNF689, and a modification thereof. In some embodiments, the DNMT comprises DNMT3A, DNMT3, a modification thereof, or a combination thereof. In some embodiments, the gene modulator is KRAB, or a modification thereof, and the additional gene modulator is DNMT, or a modification thereof. In some embodiments, the gene modulator is EZH2, or a modification thereof, and the additional gene modulator is DNMT, or a modification thereof. In some embodiments, the DNMT comprises DNMT3A, DNMT3, a modification thereof, or a combination thereof.

In some aspects, a polypeptide molecule comprising a first polypeptide domain comprising a first gene modulator, a second polypeptide domain comprising a second gene modulator, and a polypeptide chain linking the first polypeptide domain and the second polypeptide domain, wherein the polypeptide chain exhibits at least about 50% identity to the polypeptide sequence of SEQ ID NO: 1, wherein the first polypeptide domain is disposed N-terminal to the polypeptide chain, and wherein the second polypeptide domain is disposed C-terminal to the polypeptide chain is provided. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the first gene modulator and the second gene modulator are transcriptional repressors. In some embodiments, the first gene modulator and the second gene modulator are substantially the same. In some embodiments, the gene modulator and the additional gene modulator are substantially different. In some embodiments, the first gene modulator or the second gene modulator is selected from the group consisting of KRAB, DNMT, EZH2, ZNF689, and a modification thereof. In some embodiments, the DNMT comprises DNMT3A, DNMT3, a modification thereof, or a combination thereof. In some embodiments, the gene modulator is KRAB, or a modification thereof, and the additional gene modulator is DNMT, or a modification thereof. In some embodiments, the gene modulator is EZH2, or a modification thereof, and the additional gene modulator is DNMT, or a modification thereof. In some embodiments, the DNMT comprises DNMT3A, DNMT3, a modification thereof, or a combination thereof.

Provided herein is a system comprising the polypeptide molecule described herein. In some embodiments, the system further comprises a Cas protein. In some embodiments, the system further comprises a guide nucleic acid capable of forming a complex with the polypeptide molecule, wherein the complex exhibits specific binding to a target gene. In some embodiments, a first polypeptide sequence encoding the first polypeptide domain or a second polypeptide sequence encoding the second polypeptide domain is non-natural polypeptide sequence. In some embodiments, the polypeptide chain exhibits at least about 74%, at least about 75%, at least about 80%, at least about 85%, at least about 90%, at least about 95%, at least about 99%, or substantially 100% sequence identity to the polypeptide sequence of SEQ ID NO: 1. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the at least the portion of the endonuclease is a Cas protein. In some embodiments, the Cas protein is a deactivated Cas (dCas) protein that exhibits reduced nuclease activity. In some embodiments, the at least the portion of the endonuclease has a size of less than about 800 amino acid residues. In some embodiments, the gene modulator is a transcriptional activator. In some embodiments, the gene modulator is a transcriptional repressor. In some embodiments, the transcriptional repressor comprises one or more members selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator and the additional gene modulator are substantially the same. In some embodiments, the gene modulator and the additional gene modulator are substantially different. In some embodiments, the additional gene modulator is selected from the group consisting of KRAB, DNMT, EZH2, ZNF689, and a modification thereof. In some embodiments, the DNMT comprises DNMT3A, DNMT3L or a combination thereof. In some embodiments, the gene modulator is KRAB, or a modification thereof, and wherein the additional gene modulator is DNMT, or a modification thereof. In some embodiments, the gene modulator is EZH2, or a modification thereof, and the additional gene modulator is DNMT, or a modification thereof. In some embodiments, the DNMT comprises DNMT3A, DNMT3L, a modification thereof, or a combination thereof.

In one aspect, a method, comprising, contacting a cell with a system comprising a polypeptide molecule comprising a polypeptide chain exhibiting at least about 73% identity to the polypeptide sequence of SEQ ID NO: 1 is provided. In some embodiments, the polypeptide further comprises a first polypeptide domain and a second polypeptide domain that are coupled to one another via the polypeptide chain, wherein at least one of the first polypeptide domain and the second polypeptide domain is (i) at least a portion of an endonuclease or (ii) a gene modulator sequence for modulating a target gene in a cell. In some embodiments, the at least one of the first polypeptide domain and the second polypeptide domain is at least the portion of the endonuclease. In some embodiments, the at least the portion of the endonuclease is disposed N-terminal to the polypeptide chain. In some embodiments, the second polypeptide domain is the gene modulator. In some embodiments, the at least one of the first polypeptide domain and the second polypeptide domain is the gene modulator. In some embodiments, first polypeptide domain is the gene modulator, and the second polypeptide domain is an additional gene modulator. In some embodiments, a first polypeptide sequence encoding the first polypeptide domain or a second polypeptide sequence encoding the second polypeptide domain is non-natural polypeptide sequence. In some embodiments, the polypeptide chain exhibits at least about 74%, at least about 75%, at least about 80%, at least about 85%, at least about 90%, at least about 95%, at least about 99%, or substantially 100% sequence identity to the polypeptide sequence of SEQ ID NO: 1. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the at least the portion of the endonuclease is a Cas protein. In some embodiments, the Cas protein is a deactivated Cas (dCas) protein that exhibits reduced nuclease activity. In some embodiments, the at least the portion of the endonuclease has a size of less than about 800 amino acid residues. In some embodiments, the gene modulator is a transcriptional activator. In some embodiments, the gene modulator is a transcriptional repressor. In some embodiments, the transcriptional repressor comprises one or more members selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator and the additional gene modulator are substantially the same. In some embodiments, the gene modulator and the additional gene modulator are substantially different. In some embodiments, the additional gene modulator is selected from the group consisting of KRAB, DNMT, EZH2, ZNF689, and a modification thereof. In some embodiments, the DNMT comprises DNMT3A, DNMT3L or a combination thereof. In some embodiments, the gene modulator is KRAB, or a modification thereof, and wherein the additional gene modulator is DNMT, or a modification thereof. In some embodiments, the gene modulator is EZH2, or a modification thereof, and the additional gene modulator is DNMT, or a modification thereof. In some embodiments, the DNMT comprises DNMT3A, DNMT3L, a modification thereof, or a combination thereof.

In another aspect, a method, comprising contacting a cell with a system comprising a polypeptide molecule comprising a gene modulator coupled to a polypeptide chain exhibiting at least about 50% identity to the polypeptide sequence of SEQ ID NO: 1, wherein, upon formation of a complex comprising the polypeptide molecule and a target polynucleotide sequence operatively coupled to a target gene of the cell, the complex effects a greater change in expression level of the target gene, as compared to a control complex comprising the target polynucleotide sequence, the gene modulator in absence of the polypeptide chain is provided. In some embodiments, the polypeptide molecule further comprises at least a portion of an endonuclease. In some embodiments, the greater change in expression level of the target gene is characterized by reduced expression of the target gene by at least about 15%, at least about 20%, at least about 30%, at least about 40%, at least about 50%, at least about 60%, at least about 70%, or at least about 80% compared to the control complex. In some embodiments, the greater change in expression level of the target gene persists for at least about 10 days, at least 20 days, at least 30 days, or at least 40 days post transfection. In some embodiments, a first polypeptide sequence encoding the first polypeptide domain or a second polypeptide sequence encoding the second polypeptide domain is non-natural polypeptide sequence. In some embodiments, the polypeptide chain exhibits at least about 74%, at least about 75%, at least about 80%, at least about 85%, at least about 90%, at least about 95%, at least about 99%, or substantially 100% sequence identity to the polypeptide sequence of SEQ ID NO: 1. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the at least the portion of the endonuclease is a Cas protein. In some embodiments, the Cas protein is a deactivated Cas (dCas) protein that exhibits reduced nuclease activity. In some embodiments, the at least the portion of the endonuclease has a size of less than about 800 amino acid residues. In some embodiments, the gene modulator is a transcriptional activator. In some embodiments, the gene modulator is a transcriptional repressor. In some embodiments, the transcriptional repressor comprises one or more members selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator and the additional gene modulator are substantially the same. In some embodiments, the gene modulator and the additional gene modulator are substantially different. In some embodiments, the additional gene modulator is selected from the group consisting of KRAB, DNMT, EZH2, ZNF689, and a modification thereof. In some embodiments, the DNMT comprises DNMT3A, DNMT3L or a combination thereof. In some embodiments, the gene modulator is KRAB, or a modification thereof, and wherein the additional gene modulator is DNMT, or a modification thereof. In some embodiments, the gene modulator is EZH2, or a modification thereof, and the additional gene modulator is DNMT, or a modification thereof. In some embodiments, the DNMT comprises DNMT3A, DNMT3L, a modification thereof, or a combination thereof.

In another aspect, a method, comprising contacting a cell with a system comprising a polypeptide molecule comprising a first polypeptide domain coupled to a second polypeptide domain via a polypeptide chain, wherein the polypeptide chain is greater than 18 amino acids, wherein the first polypeptide domain is a gene modulator, and the second polypeptide domain is an additional gene modulator or at least a portion of an endonuclease, wherein, upon formation of a complex comprising the polypeptide molecule and a target polynucleotide sequence operatively coupled to a target gene of the cell, the complex effects a greater change in expression level of the target gene, as compared to a control complex comprising the polynucleotide sequence, the first polypeptide domain, and the second polypeptide domain in absence of the polypeptide chain, wherein the greater change in expression level persists greater than 30 days is provided. In some embodiments, the polypeptide chain exhibits at least about 50% identity to the polypeptide sequence of SEQ ID NO: 1. In some embodiments, the second polypeptide domain is the at least a portion of an endonuclease. In some embodiments, the second polypeptide domain is the additional gene modulator. In some embodiments, the greater change in expression level of the target gene is characterized by a reduced expression of the target gene by at least about 15%, at least about 20%, at least about 30%, at least about 40%, at least about 50%, at least about 60%, at least about 70%, or at least about 80%. In some embodiments, the greater change in expression level of the target gene persists for at least about at least 40 days post transfection. In some embodiments, a first polypeptide sequence encoding the first polypeptide domain or a second polypeptide sequence encoding the second polypeptide domain is non-natural polypeptide sequence. In some embodiments, the polypeptide chain exhibits at least about 74%, at least about 75%, at least about 80%, at least about 85%, at least about 90%, at least about 95%, at least about 99%, or substantially 100% sequence identity to the polypeptide sequence of SEQ ID NO: 1. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the at least the portion of the endonuclease is a Cas protein. In some embodiments, the Cas protein is a deactivated Cas (dCas) protein that exhibits reduced nuclease activity. In some embodiments, the at least the portion of the endonuclease has a size of less than about 800 amino acid residues. In some embodiments, the gene modulator is a transcriptional activator. In some embodiments, the gene modulator is a transcriptional repressor. In some embodiments, the transcriptional repressor comprises one or more members selected from the group consisting of KRAB, DNMT, EZH2, ZNF689, and a modification thereof. In some embodiments, the DNMT comprises DNMT3A, DNMT3L, a modification thereof, or a combination thereof. In some embodiments, the gene modulator and the additional gene modulator are substantially the same. In some embodiments, the gene modulator and the additional gene modulator are substantially different. In some embodiments, the additional gene modulator is selected from the group consisting of KRAB, DNMT, EZH2, ZNF689, and a modification thereof. In some embodiments, the DNMT comprises DNMT3A, DNMT3L or a combination thereof. In some embodiments, the gene modulator is KRAB, or a modification thereof, and wherein the additional gene modulator is DNMT, or a modification thereof. In some embodiments, the gene modulator is EZH2, or a modification thereof, and the additional gene modulator is DNMT, or a modification thereof. In some embodiments, the DNMT comprises DNMT3A, DNMT3L, a modification thereof, or a combination thereof.

In another aspect, a method for modulating a target gene in a cell, comprising (a) contacting the target cell with a complex comprising (i) a first polypeptide domain comprising at least a portion of an endonuclease, a second polypeptide domain encoding a gene modulator, and a chain sequence linking the first polypeptide domain and the second polypeptide domain, wherein the polypeptide chain exhibits at least about 50% identity to the polypeptide sequence of SEQ ID NO: 1, wherein the first polypeptide domain is disposed N-terminal to the polypeptide chain, and wherein the second polypeptide domain is disposed C-terminal to the polypeptide chain, (ii) a guide nucleic acid molecule exhibiting specific binding to a target polynucleic acid; and (b) upon the contacting, binding the target gene with the complex to effect a greater change in expression level of the target gene, as compared to a control complex that comprises the guide nucleic acid molecule, the first polypeptide domain, the second polypeptide domain in absence of the polypeptide chain is provided. In some embodiments, the endonuclease is a Cas protein. In some embodiments, the Cas protein is a deactivated Cas (dCas) protein that exhibits reduced nuclease activity. In some embodiments, the at least the portion of the endonuclease has a size of less than about 800 amino acid residues. In some embodiments, the polypeptide molecule further comprising a third polypeptide domain encoding a second gene modulator. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the first gene modulator and the second gene modulator are transcriptional repressors. In some embodiments, the first gene modulator and the second gene modulator are substantially the same. In some embodiments, the gene modulator and the additional gene modulator are substantially different. In some embodiments, the first gene modulator or the second gene modulator is selected from the group consisting of KRAB, DNMT, EZH2, ZNF689, and a modification thereof. In some embodiments, the DNMT comprises DNMT3A, DNMT3, a modification thereof or a combination thereof. In some embodiments, the gene modulator is KRAB, or a modification thereof, and the additional gene modulator is DNMT, or a modification thereof. In some embodiments, the gene modulator is EZH2, or a modification thereof, and the additional gene modulator is DNMT or a modification thereof. In some embodiments, the DNMT comprises DNMT3A, DNMT3, a modification thereof or a combination thereof.

In another aspect, a method for modulating a target gene in a cell, comprising (a) contacting the target cell with a complex comprising (i) a first polypeptide domain comprising a first gene modulator; a second polypeptide domain comprising a second gene modulator; and a polypeptide chain linking the first polypeptide domain and the second polypeptide domain, wherein the polypeptide chain exhibits at least about 50% identity to the polypeptide sequence of SEQ ID NO: 1, wherein the first polypeptide domain is disposed N-terminal to the polypeptide chain, and wherein the second polypeptide domain is disposed C-terminal to the polypeptide chain, (ii) a guide nucleic acid molecule exhibiting specific binding to a target polynucleic acid; and (b) upon the contacting, binding the target gene with the complex to effect a greater change in expression level of the target gene, as compared to a control complex that comprises the guide nucleic acid molecule, the first polypeptide domain, and the second polypeptide domain in absences of the polypeptide chain. In some embodiments, a length of the polypeptide chain is greater than 18 amino acid residues. In some embodiments, a length of the polypeptide chain is greater than 20 amino acid residues. In some embodiments, the polypeptide chain does not comprise the polypeptide sequence of GGGGS (SEQ ID NO: 38) or GGGS (SEQ ID NO: 39). In some embodiments, the polypeptide chain comprises at most 2 repeats of the polypeptide sequence of GGS. In some embodiments, the polypeptide molecule comprises a plurality of the polypeptide chain. In some embodiments, the first gene modulator and the second gene modulator are transcriptional repressors. In some embodiments, the first gene modulator and the second gene modulator are substantially the same. In some embodiments, the gene modulator and the additional gene modulator are substantially different. In some embodiments, the first gene modulator or the second gene modulator is selected from the group consisting of KRAB, DNMT, EZH2, ZNF689, and a modification thereof. In some embodiments, the DNMT comprises DNMT3A, DNMT3, a modification thereof or a combination thereof. In some embodiments, the gene modulator is KRAB, or a modification thereof, and the additional gene modulator is DNMT, or a modification thereof. In some embodiments, the gene modulator is EZH2, or a modification thereof, and the additional gene modulator is DNMT or a modification thereof. In some embodiments, the DNMT comprises DNMT3A, DNMT3, a modification thereof or a combination thereof.

Provided herein is a polypeptide chain linking two heterologous polypeptide domains, wherein a number of G residue in the polypeptide chain is between about 22% and about 65%, wherein a length of the polypeptide chain is between 13 amino acid residues and 53 amino acid residues. In some embodiments, the polypeptide chain (i) does not comprise the polypeptide sequence of GGGGS (SEQ ID NO: 38) or GGGS (SEQ ID NO: 39) or (ii) comprises at most 2 repeats of the polypeptide sequence of GGS.

Also provided herein is a polypeptide molecule comprising a first polypeptide domain linked to a second polypeptide domain via a polypeptide chain, wherein at least one of the first polypeptide domain and the second polypeptide domain is a gene modulator configured to modulate a target gene in a cell, wherein the polypeptide molecule is configured to effect a greater change in expression level of the target gene in the cell, as compared that by a control polypeptide molecule comprising the first polypeptide domain linked to the second polypeptide domain via a control linker having the polypeptide sequence of any one of SEQ ID NOs: 37-50. In some embodiments, the at least one of the first polypeptide domain and the second polypeptide domain is the at least the portion of the endonuclease. In some embodiments, the second polypeptide domain is the gene modulator. In some embodiments, the at least one of the first polypeptide domain and the second polypeptide domain is the gene modulator. In some embodiments, the first polypeptide domain is the gene modulator, and the second polypeptide domain is an additional gene modulator. In some embodiments, the polypeptide chain exhibits at least about 50% identity to the polypeptide sequence of SEQ ID NO: 1. In some embodiments, a number of G residue in the polypeptide chain is less than about 65%. In some embodiments, a length of the polypeptide chain is greater than 13 amino acid residues. In some embodiments, the length of the polypeptide chain is less than 50 amino acid residues. In some embodiments, the greater change in expression level of the target gene is characterized by reduced expression of the target gene by at least about 15%, at least about 20%, at least about 30%, at least about 40%, at least about 50%, at least about 60%, at least about 70%, or at least about 80% compared to the control polypeptide molecule. In some embodiments, the greater change in expression level of the target gene persists for at least about 10 days, at least 20 days, at least 30 days, or at least 40 days post transfection.

Provided herein is a method for modulating a target gene in a cell, comprising: (a) contacting the target cell with a complex comprising (i) a first polypeptide domain linked to a second polypeptide domain via a polypeptide chain, (ii) a guide nucleic acid molecule exhibiting specific binding to a target polynucleic acid; and (b) upon the contacting, binding the target gene with the complex to effect a greater change in expression level of the target gene in the cell, as compared that by a control polypeptide molecule comprising the guide nucleic acid molecule and the first polypeptide domain linked to the second polypeptide domain via a control linker having the polypeptide sequence of any one of SEQ ID NOs: 37-50. In some embodiments, the at least one of the first polypeptide domain and the second polypeptide domain is the at least the portion of the endonuclease. In some embodiments, the second polypeptide domain is the gene modulator. In some embodiments, the at least one of the first polypeptide domain and the second polypeptide domain is the gene modulator. In some embodiments, the first polypeptide domain is the gene modulator, and the second polypeptide domain is an additional gene modulator. In some embodiments, the polypeptide chain exhibits at least about 50% identity to the polypeptide sequence of SEQ ID NO: 1. In some embodiments, a number of G residue in the polypeptide chain is less than about 65%. In some embodiments, a length of the polypeptide chain is greater than 13 amino acid residues. In some embodiments, the length of the polypeptide chain is less than 50 amino acid residues. In some embodiments, the greater change in expression level of the target gene is characterized by reduced expression of the target gene by at least about 15%, at least about 20%, at least about 30%, at least about 40%, at least about 50%, at least about 60%, at least about 70%, or at least about 80% compared to the control polypeptide molecule. In some embodiments, the greater change in expression level of the target gene persists for at least about 10 days, at least 20 days, at least 30 days, or at least 40 days post transfection.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows gene expression profile (30 days post transfection) of a heterologous target gene by polypeptide molecules.

FIG. 2 shows gene expression profile (44 days post transfection) of a heterologous target gene by polypeptide molecules.

FIG. 3 shows gene expression profile of a heterologous target gene at different time points by polypeptide molecules.

FIG. 4 shows suppression of an endogenous gene encoding CXCR4 by polypeptide molecules.

### DETAILED DESCRIPTION

### Definitions

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” or “approximately” generally means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

The term “heterologous,” when used herein with reference to a polypeptide sequence or a nucleic acid sequence, indicates that the polypeptide sequence or the nucleic acid sequence is (1) disposed (e.g., in an environment, such as a cell, a virus, or a fusion polypeptide molecule or a fusion polynucleotide molecule) where it is not normally found (e.g., not normally found in nature); or (2) comprises two or more subsequences that are not found in the same relationship to each other as normally found in nature. For example, a polypeptide can comprise a first polypeptide sequence and a second polypeptide sequence that are not found together in a single polypeptide in nature, and thus the first polypeptide sequence and the second polypeptide sequence can be heterologous to each other. In another example, a polynucleotide can comprise a first polynucleotide sequence and a second polynucleotide sequence that are not found together in a single polynucleotide in nature, and thus the first polynucleotide sequence and the second polynucleotide sequence can be heterologous to each other.

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g., altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

It is understood that, in certain instances, thymidine (T) and uracil (U) may be used interchangeably to represent the respective complementary base in a DNA or RNA polynucleotide. The interchangeability is intended to encompass the functional equivalence of thymine (T) in DNA and uracil (U) in RNA, as both bases can form complementary base pairs with adenine (A) through Watson-Crick base pairing. Any reference to a polynucleotide sequence herein that includes thymine (T), or uracil (U) should be construed as referring to the corresponding DNA or RNA sequences with the appropriate base.

The term “sequence identity” generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin and Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 50% to 100% and integer values therebetween. In general, this disclosure encompasses sequences with at least 50%, at least 55%, at least 60%, at least 65%, at least 70%, at least 75%, at least 80%, at least 85%, at least 90%, at least 95%, or at least 98% sequence identity with any sequence provided herein.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some embodiments, genes do not encode a polypeptide, for example, ribosomal RNA genes (rRNA) and transfer RNA (tRNA) genes. In some embodiments, the term “gene” includes not only the transcribed sequences, but in addition, also includes non-transcribed regions including upstream and downstream regulatory regions, enhancers, and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “expression profile” generally refers to quantitative (e.g., abundance) and qualitative expression of one or more genes in a sample (e.g., a cell). The one or more genes can be expressed and ascertained in the form of a nucleic acid molecule (e.g., an mRNA or other RNA transcript). Alternatively, or in addition to, the one or more genes can be expressed and ascertained in the form of a polypeptide (e.g., a protein measured via Western blot). An expression profile of a gene may be defined as a shape of an expression level of the gene over a time period (e.g., at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 11 hours, at least or up to about 12 hours, at least or up to about 16 hours, at least or up to about 18 hours, at least or up to about 24 hours, at least or up to about 36 hours, at least or up to about 48 hours, at least up to about 3 days, at least up to about 4 days, at least up to about 5 days, at least up to about 6 days, at least up to about 7 days, at least up to about 8 days, at least up to about 9 days, at least up to about 10 days, at least up to about 11 days, at least up to about 12 days, at least up to about 13 days, at least up to about 14 days, etc.). Alternatively, an expression profile of a gene may be defined as an expression level of the gene at a time point of interest (e.g., the expression level of the gene measured at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 11 hours, at least or up to about 12 hours, at least or up to about 16 hours, at least or up to about 18 hours, at least or up to about 24 hours, at least or up to about 36 hours, at least or up to about 48 hours, at least up to about 3 days, at least up to about 4 days, at least up to about 5 days, at least up to about 6 days, at least up to about 7 days, at least up to about 8 days, at least up to about 9 days, at least up to about 10 days, at least up to about 11 days, at least up to about 12 days, at least up to about 13 days, or at least up to about 14 days after treating a cell to induce such expression level).

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some embodiments, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants, and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In some embodiments, an engineered or recombinant polynucleotide (e.g., a genomic DNA sequence) can be modified or altered by a gene editing moiety. For example, at least the portion of the endonuclease (e.g., an engineered Cas protein) as disclosed herein is not a naturally occurring nuclease (e.g., not a naturally occurring Cas protein). In another example, an engineered gene effector as disclosed herein is not a naturally occurring gene effector.

The terms “engineered” and “modified” are used interchangeably herein. The terms “engineering” and “modifying” are used interchangeably herein. The terms “engineered cell” or “modified cell” are used interchangeably herein. The terms “engineered characteristic” and “modified characteristic” are used interchangeably herein.

The term “enhanced expression,” “increased expression,” or “upregulated expression” generally refers to production of a moiety of interest (e.g., a polynucleotide or a polypeptide) to a level that is above a normal level of expression of the moiety of interest in a host strain (e.g., a host cell). The normal level of expression can be substantially zero (or null) or higher than zero. The moiety of interest can comprise an endogenous gene or polypeptide construct of the host strain. The moiety of interest can comprise a heterologous gene or polypeptide construct that is introduced to or into the host strain. For example, a heterologous gene encoding a polypeptide of interest can be knocked-in (KI) to a genome of the host strain for enhanced expression of the polypeptide of interest in the host strain.

The term “enhanced activity,” “increased activity,” or “upregulated activity” generally refers to activity of a moiety of interest (e.g., a polynucleotide or a polypeptide) that is modified to a level that is above a normal level of activity of the moiety of interest in a host strain (e.g., a host cell). The normal level of activity can be substantially zero (or null) or higher than zero. The moiety of interest can comprise a polypeptide construct of the host strain. The moiety of interest can comprise a heterologous polypeptide construct that is introduced to or into the host strain. For example, a heterologous gene encoding a polypeptide of interest can be knocked-in (KI) to a genome of the host strain for enhanced activity of the polypeptide of interest in the host strain.

The term “reduced expression,” “decreased expression,” or “downregulated expression” generally refers to a production of a moiety of interest (e.g., a polynucleotide or a polypeptide) to a level that is below a normal level of expression of the moiety of interest in a host strain (e.g., a host cell). The normal level of expression is higher than zero. The moiety of interest can comprise an endogenous gene or polypeptide construct of the host strain. In some embodiments, the moiety of interest can be knocked-out or knocked-down in the host strain. In some examples, reduced expression of the moiety of interest can include a complete inhibition of such expression in the host strain.

The term “reduced activity,” “decreased activity,” or “downregulated activity” generally refers to activity of a moiety of interest (e.g., a polynucleotide or a polypeptide) that is modified to a level that is below a normal level of activity of the moiety of interest in a host strain (e.g., a host cell). The normal level of activity is higher than zero. The moiety of interest can comprise an endogenous gene or polypeptide construct of the host strain. In some embodiments, the moiety of interest can be knocked-out or knocked-down in the host strain. In some examples, reduced activity of the moiety of interest can include a complete inhibition of such activity in the host strain.

The term “subject,” “individual,” or “patient,” as used interchangeably herein, generally refers to a vertebrate, preferably a mammal such as a human. Mammals include, but are not limited to, murines, simians, humans, farm animals, sport animals, and pets. Tissues, cells, and their progeny of a biological entity obtained in vivo or cultured in vitro are also encompassed.

The term “treatment” or “treating” generally refers to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” generally refers to the quantity of a composition, for example a composition comprising heterologous polypeptides, heterologous polynucleotides, and/or modified cells (e.g., modified stem cells), that is sufficient to result in a desired activity upon administration to a subject in need thereof. Within the context of the present disclosure, the term “therapeutically effective” generally refers to that quantity of a composition that is sufficient to delay the manifestation, arrest the progression, relieve, or alleviate at least one symptom of a disorder treated by the methods of the present disclosure. Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

### Systems, Compositions, and Methods of Use Thereof

In some aspects, the present disclosure provides a polypeptide molecule (or engineered polypeptide molecules) comprising a polypeptide chain (e.g., comprising the polypeptide sequence of SEQ ID NO: 1) capable of modulating (e.g., editing, activating or reducing) expression or activity level of a target gene in a cell (e.g., an endogenous target gene, a heterologous target gene, etc.), compositions thereof, and methods of use thereof.

In some embodiments, the systems, compositions, and methods described herein comprise a polypeptide molecule comprising a polypeptide domain (e.g., at least a portion of an endonuclease or a gene modulator) operatively coupled to the polypeptide chain disclosed herein. In some embodiments, the systems, compositions, and methods described herein modulate (e.g., edit, activate, suppress) expression or activity level of a target gene (e.g., heterologous gene, endogenous gene) in a cell. In some embodiments, the systems, compositions, and methods described herein comprise a polypeptide molecule comprising a first polypeptide domain and a second polypeptide domain that are coupled to one another via the polypeptide chain disclosed herein. For example, a polypeptide molecule can comprise at least a portion of an endonuclease (e.g., engineered CRISPR/CAS nuclease, or a deactivated variant thereof) and a gene modulator (e.g., a transcriptional activator or transcriptional repressor) that are coupled to one another via the polypeptide chain disclosed herein. In another example, a polypeptide molecule can comprise a gene modulator (e.g., a transcriptional activator or transcriptional repressor) and an additional gene modulator (e.g., a transcriptional activator or transcriptional repressor that is substantially the same or different from the gene modulator) that are operatively coupled to one another via the polypeptide chain disclosed herein. In some aspects, the present disclosure provides one or more polynucleotides encoding the polypeptide molecule disclosed herein. In some embodiments, the systems, compositions, and methods described herein further comprise a guide nucleic acid. In some embodiments, a guide nucleic acid may be capable of forming a complex with the polypeptide molecule disclosed herein, wherein the complex can be operatively coupled to a target gene to modulate (e.g., edit, activate, suppress) expression of the target gene.

### Polypeptide Molecule

In some embodiments, the systems, compositions, and methods described herein comprise a polypeptide molecule comprising a polypeptide chain (e.g., comprising the polypeptide sequence of SEQ ID NO: 1). In some embodiments, a polypeptide molecule can comprise a plurality (e.g., at least or up to one, at least or up to two, at least or up to three, at least or up to four, at least or up to five, at least or up to six, at least or up to seven, at least or up to eight, at least or up to nine, at least or up to ten) of the polypeptide chains disclosed herein. In some embodiments, a polypeptide molecule can comprise a polypeptide domain operatively coupled to the polypeptide chain disclosed herein. In some embodiments, a polypeptide molecule can comprise a plurality of polypeptide domains (e.g., at least or up to one, at least or up to two, at least or up to three, at least or up to four, at least or up to five, at least or up to six, at least or up to seven, at least or up to eight, at least or up to nine, at least or up to ten) that are coupled to one another via one or more polypeptide chains disclosed herein.

In some embodiments, at least one polypeptide domain of a plurality of polypeptide domains can be coupled to the polypeptide chain disclosed herein. In some embodiments, at least two polypeptide domains of a plurality of polypeptide domains can be coupled to each other via one or more polypeptide chains disclosed herein. In some embodiments, at least three polypeptide domains of a plurality of polypeptide domains are coupled to each other via or more polypeptide chains disclosed herein. In some embodiments, at least four polypeptide domains of a plurality of polypeptide domains are coupled to each other via one or more polypeptide chains disclosed herein. In some embodiments, at least five polypeptide domains of a plurality of polypeptide domains are coupled to each other via one or more polypeptide chains disclosed herein. In some embodiments, at least six polypeptide domains of a plurality of polypeptide domains are coupled to each other via one or more polypeptide chains disclosed herein. In some embodiments, at least seven polypeptide domains of a plurality of polypeptide domains are coupled to each other via one or more polypeptide chains disclosed herein. In some embodiments, at least eight polypeptide domains of a plurality of polypeptide domains are coupled to each other via one or more polypeptide chains disclosed herein. In some embodiments, at least nine polypeptide domains of a plurality of polypeptide domains are coupled to each other via one or more polypeptide chains disclosed herein. In some embodiments, at least ten polypeptide domains of a plurality of polypeptide domains are coupled to each other via one or more polypeptide chains disclosed herein.

In some embodiments, at least one of the first polypeptide domain and the second polypeptide domain of the polypeptide molecule described herein can be (i) at least a portion of an endonuclease (e.g., an CRISPR/CAS nuclease, an engineered or a deactivated variant thereof) or (ii) a gene modulator (e.g., a transcriptional activator or transcriptional repressor), wherein the at least the first polypeptide domain and the second polypeptide domain are operatively coupled to one another via the polypeptide chain.

In some embodiments, at least one of the first polypeptide domain and the second polypeptide domain of the polypeptide molecule can be at least the portion of the endonuclease (e.g., an CRISPR/CAS nuclease, an engineered or a deactivated variant thereof). In some embodiments, the at least the portion of the endonuclease can be disposed N- or C-terminal to the polypeptide chain. In some embodiments, the at least the portion of the endonuclease can be disposed N-terminal to the polypeptide chain. In some embodiments, the at least the portion of the endonuclease can be disposed C-terminal to the polypeptide chain. In some embodiments, a polypeptide molecule can comprise at least a portion of the endonuclease, and the second polypeptide domain (e.g., a gene modulator, a gene editing moiety, or an effector protein), wherein the at least the portion of the endonuclease and the second polypeptide domain are operatively coupled to one another via the polypeptide chain disclosed herein. In some embodiments, at least the portion of the endonuclease can be disposed N-terminal to the polypeptide chain, and the second polypeptide domain can be disposed C-terminal to the polypeptide chain. In some embodiments, at least the portion of the endonuclease can be disposed C-terminal to the polypeptide chain, and the second polypeptide domain can be disposed N-terminal to the polypeptide chain. For example, the first polypeptide domain can be at least a portion of an endonuclease (e.g., an CRISPR/CAS nuclease, an engineered or a deactivated variant thereof), and the second polypeptide domain can be a gene modulator (e.g., a transcriptional activator or transcriptional repressor). In some embodiments, the at least the portion of the endonuclease and the gene modulator can be coupled to one another via the polypeptide chain disclosed herein (e.g., a polypeptide molecule comprising, in an N>C terminal direction, a contiguous polypeptide that comprises at least a portion of an endonuclease, a polypeptide chain, and a gene modulator or a gene modulator, a polypeptide chain, and at least a portion of an endonuclease).

In some embodiments, at least one of the first polypeptide domain and the second polypeptide domain of the polypeptide molecule can be a gene modulator (e.g., a transcriptional activator or transcriptional repressor). In some embodiments, a gene modulator can be disposed N- or C-terminal to the polypeptide chain. In some embodiments, a gene modulator can be disposed N-terminal to the polypeptide chain. In some embodiments, a gene modulator can be disposed C-terminal to the polypeptide chain. In some embodiments, a gene modulator and the second polypeptide domain (e.g., at least a portion of an endonuclease, an additional gene modulator, a gene editing moiety, or an effector protein) can be coupled to one another via the polypeptide chain disclosed herein. In some embodiments, a gene modulator can be disposed N-terminal to the polypeptide chain, and the second polypeptide domain can be disposed C-terminal to the polypeptide chain. In some embodiments, a gene modulator can be disposed C-terminal to the polypeptide chain, and the second polypeptide domain can be disposed N-terminal to the polypeptide chain. For example, the first polypeptide domain can be a gene modulator (e.g., a transcriptional activator or transcriptional repressor), and the second polypeptide domain can be an additional gene modulator (e.g., a transcriptional activator or transcriptional repressor). In some embodiments, a polypeptide molecule can comprise a gene modulator and an additional gene modulator that are operatively coupled to one another via the polypeptide chain disclosed herein (e.g., a polypeptide molecule comprising, in an N>C terminal direction, a contiguous polypeptide that comprises a gene modulator, a polypeptide chain, and an additional gene modulator). In some embodiments, the gene modulator and the additional gene modulator can be substantially the same. In some embodiments, the gene modulator and the additional gene modulator can be substantially different.

In some embodiments, a polypeptide molecule can comprise at least two polypeptide domains that are operatively coupled to one or more polypeptide chains (e.g., comprising the polypeptide sequence of SEQ ID NO: 1) or one or more linkers (e.g., comprising the polypeptide sequence selected from SEQ ID NOs: 37-50). In some embodiments, the first polypeptide domain (e.g., at least a portion of an endonuclease) and the second polypeptide domain (e.g., a gene modulator) can be operatively coupled to one another via the polypeptide chain (e.g., a polypeptide molecule comprising, in an N>C terminal direction, a contiguous polypeptide that comprises [Polypeptide Domain 1]-[Polypeptide Chain]-[Polypeptide Domain 2]). In some embodiments, the first polypeptide domain and the second polypeptide domain can be operatively coupled to one another via the polypeptide chain, wherein the second polypeptide domain may be further coupled to another polypeptide chain (e.g., a polypeptide molecule comprising, in an N>C terminal direction, a contiguous polypeptide that comprises [Polypeptide Domain 1]-[Polypeptide Chain]-[Polypeptide Domain 2]-[Polypeptide Chain]). In some embodiments, the first polypeptide domain and the second polypeptide domain can be coupled to one another via the polypeptide chain, wherein the second polypeptide domain can be further coupled to a linker (e.g., comprising the polypeptide sequence selected from SEQ ID NOs: 37-50) (e.g., a polypeptide molecule comprising, in an N>C terminal direction, a contiguous polypeptide that comprises [Polypeptide Domain 1]-[Polypeptide Chain]-[Polypeptide Domain 2]-[Linker]). In some embodiments, the first polypeptide domain and the second polypeptide domain can be coupled to one another via a linker (e.g., comprising the polypeptide sequence selected from SEQ ID NOs: 37-50), wherein the second polypeptide domain can be further coupled to the polypeptide chain (e.g., a polypeptide molecule comprising, in an N>C terminal direction, a contiguous polypeptide that comprises [Polypeptide Domain 1]-[Linker]-[Polypeptide Domain 2]-[Polypeptide Chain]). For example, the first polypeptide domain can be at least a portion of an endonuclease (e.g., comprising the polypeptide sequence selected from SEQ ID NOs: 3-5 or 51-250), and the second polypeptide domain can be a gene modulator (e.g., comprising the polypeptide sequence selected from SEQ ID NOs: 6-17). In another example, the first polypeptide domain can be a gene modulator (e.g., comprising the polypeptide sequence selected from SEQ ID NOs: 6-17), and the second polypeptide domain can be an additional gene modulator (e.g., comprising the polypeptide sequence selected from SEQ ID NOs: 6-17). In some embodiments, the first polypeptide domain can be a gene modulator (e.g., comprising the polypeptide sequence selected from SEQ ID NOs: 6-17), and the second polypeptide domain can be at least a portion of an endonuclease (e.g., comprising the polypeptide sequence selected from SEQ ID NOs: 3-5 or 51-250).

In some embodiments, a polypeptide molecule described herein can comprise at least three polypeptide domains (e.g., at least a portion of an endonuclease, a first gene modulator, and an additional gene modulator) that are operatively coupled to one another via one or more polypeptide chains (e.g., comprising the polypeptide sequence of SEQ ID NO: 1) or one or more linkers (e.g., comprising the polypeptide sequence selected from SEQ ID NOs: 37-50). In some embodiments, the first polypeptide domain (e.g., at least a portion of an endonuclease) and the second polypeptide domain (e.g., a first gene modulator) can be coupled to one another via the polypeptide chain, or a linker. In some embodiments, the second polypeptide domain (e.g., a first gene modulator) and the third polypeptide domain (e.g., a second gene modulator) can be coupled to one another via the polypeptide chain, or a linker. In some embodiments, the first polypeptide domain and the second polypeptide domain can be coupled to one another via a, and the second polypeptide domain and the third polypeptide domain can be coupled to one another via the polypeptide chain (e.g., a polypeptide molecule comprising, in an N>C terminal direction, a contiguous polypeptide that comprises [Polypeptide Domain 1]-[Linker]-[Polypeptide Domain 2]-[Polypeptide Chain]-[Polypeptide Domain 3]). In some embodiments, the first polypeptide domain and the second polypeptide domain can be coupled to one another via the polypeptide chain, and the second polypeptide domain and the third polypeptide domain can be coupled to one another via another polypeptide chain (e.g., a polypeptide molecule comprising, in an N>C terminal direction, a contiguous polypeptide that comprises [Polypeptide Domain 1]-[Polypeptide Chain]-[Polypeptide Domain 2]-[Polypeptide Chain]-[Polypeptide Domain 3]). In some embodiments, the first polypeptide domain and the second polypeptide domain can be coupled to one another via the polypeptide chain, and the second polypeptide domain and the third polypeptide domain can be coupled to one another via a linker (e.g., a polypeptide molecule comprising, in an N>C terminal direction, a contiguous polypeptide that comprises [Polypeptide Domain 1]-[Polypeptide Chain]-[Polypeptide Domain 2]-[Linker]-[Polypeptide Domain 3]). In some embodiments, the third polypeptide domain can be further coupled to a polypeptide chain (e.g., comprising the polypeptide sequence of SEQ ID NO: 1) or a linker (e.g., comprising the polypeptide sequence selected from SEQ ID NOs: 37-50), wherein the third polypeptide domain is disposed N-terminal to the polypeptide chain or the linker. In some embodiments, the first polypeptide domain can be at least a portion of an endonuclease (e.g., comprising the polypeptide sequence selected from SEQ ID NOs: 3-5 or 51-250), and the second polypeptide domain and the third polypeptide domain can be gene modulators (e.g., comprising the polypeptide sequence selected from SEQ ID NOs: 6-17). In another example, the first polypeptide domain, the second polypeptide domain, and the third polypeptide domain can be gene modulators (e.g., comprising the polypeptide sequence selected from SEQ ID NOs: 6-17). In some embodiments, the first polypeptide domain and the third polypeptide domain can be gene modulators (e.g., comprising the polypeptide sequence selected from SEQ ID NOs: 6-17), and the second poly peptide domain can be at least a portion of an endonuclease (e.g., comprising the polypeptide sequence selected from SEQ ID NOs: 3-5 or 51-250).

In some embodiments, the polypeptide molecule described herein can comprise at least four polypeptide domains (e.g., at least a portion of an endonuclease, a first gene modulator, a second gene modulator, and a third gene modulator) that are operatively coupled to one another via one or more polypeptide chains (e.g., comprising the polypeptide sequence of SEQ ID NO: 1), or one or more linkers (e.g., comprising the polypeptide sequence selected from SEQ ID NOs: 37-50). In some embodiments, the first polypeptide domain (e.g., at least a portion of an endonuclease) and the second polypeptide domain (e.g., a first gene modulator) can be coupled to one another via a polypeptide chain or a linker. In some embodiments, the second polypeptide domain (e.g., a first gene modulator) and the third polypeptide domain (e.g., a second gene modulator) can be coupled to one another via a polypeptide chain or a linker. In some embodiments, the third polypeptide domain (e.g., a second gene modulator) and the fourth polypeptide domain (e.g., a third gene modulator) can be coupled to one another via a polypeptide chain or a linker. In some embodiments, the first polypeptide domain and the second polypeptide domain can be coupled to one another via a linker, while the second polypeptide domain and the third polypeptide domain can be coupled to one another via the polypeptide chain, and the third polypeptide domain and the fourth polypeptide domain can be coupled to one another via a linker (e.g., a polypeptide molecule comprising, in an N>C terminal direction, a contiguous polypeptide that comprises [Polypeptide Domain 1]-[Linker]-[Polypeptide Domain 2]-[Polypeptide chain]-[Polypeptide Domain 3]-[Linker]-[Polypeptide Domain 4]). In some embodiments, the first polypeptide domain and the second polypeptide domain can be coupled to one another via a linker, while the second polypeptide domain and the third polypeptide domain can be coupled to one another via a linker, and the third polypeptide domain and the fourth polypeptide domain can be coupled to one another via the polypeptide chain (e.g., a polypeptide molecule comprising, in an N>C terminal direction, a contiguous polypeptide that comprises [Polypeptide Domain 1]-[Linker]-[Polypeptide Domain 2]-[Linker]-[Polypeptide Domain 3]-[Polypeptide chain]-[Polypeptide Domain 4]). In some embodiments, the first polypeptide domain and the second polypeptide domain can be coupled to one another via a linker, while the second polypeptide domain and the third polypeptide domain can be coupled to one another via the polypeptide chain, and the third polypeptide domain and the fourth polypeptide domain can be coupled to one another via the polypeptide chain (e.g., a polypeptide molecule comprising, in an N>C terminal direction, a contiguous polypeptide that comprises [Polypeptide Domain 1]-[Linker]-[Polypeptide Domain 2]-[Polypeptide chain]-[Polypeptide Domain 3]-[Polypeptide Chain]-[Polypeptide Domain 4]). In some embodiments, the first polypeptide domain and the second polypeptide domain can be coupled to one another via the polypeptide chain, while the second polypeptide domain and the third polypeptide domain can be coupled to one another via a linker, and the third polypeptide domain and the fourth polypeptide domain can be coupled to one another via a linker (e.g., a polypeptide molecule comprising, in an N>C terminal direction, a contiguous polypeptide that comprise: [Polypeptide Domain 1]-[Polypeptide chain]-[Polypeptide Domain 2]-[Linker]-[Polypeptide Domain 3]-[Linker]-[Polypeptide Domain 4]). In some embodiments, the first polypeptide domain and the second polypeptide domain can be coupled to one another via the polypeptide linker, while the second polypeptide domain and the third polypeptide domain can be coupled to one another via a linker, and the third polypeptide domain and the fourth polypeptide domain can be coupled to one another via the polypeptide chain (e.g., a polypeptide molecule comprising, in an N>C terminal direction, a contiguous polypeptide that comprises [Polypeptide Domain 1]-[Polypeptide chain]-[Polypeptide Domain 2]-[Linker]-[Polypeptide Domain 3]-[Polypeptide Chain]-[Polypeptide Domain 4]). In some embodiments, the first polypeptide domain and the second polypeptide domain can be coupled to one another via a linker, while the second polypeptide domain and the third polypeptide domain can be coupled to one another via a linker, and the third polypeptide domain and the fourth polypeptide domain can be coupled to one another via the polypeptide chain (e.g., a polypeptide molecule comprising, in an N>C terminal direction, a contiguous polypeptide that comprises: [Polypeptide Domain 1]-[Linker]-[Polypeptide Domain 2]-[Linker]-[Polypeptide Domain 3]-[Polypeptide chain]-[Polypeptide Domain 4]). In some embodiments, the first polypeptide domain and the second polypeptide domain can be coupled to one another via the polypeptide chain, while the second polypeptide domain and the third polypeptide domain can be coupled to one another via the polypeptide chain, and the third polypeptide domain and the fourth polypeptide domain can be coupled to one another via a linker (e.g., a polypeptide molecule comprising, in an N>C terminal direction, a contiguous polypeptide that comprises [Polypeptide Domain 1]-[Polypeptide chain]-[Polypeptide Domain 2]-[Polypeptide chain]-[Polypeptide Domain 3]-[Linker]-[Polypeptide Domain 4]). In some embodiments, the first polypeptide domain and the second polypeptide domain can be coupled to one another via the polypeptide chain, while the second polypeptide domain and the third polypeptide domain can be coupled to one another via the polypeptide chain, and the third polypeptide domain and the fourth polypeptide domain can be coupled to one another via the polypeptide chain (e.g., a polypeptide molecule comprising, in an N>C terminal, direction a contiguous polypeptide that comprises [Polypeptide Domain 1]-[Polypeptide chain]-[Polypeptide Domain 2]-[Polypeptide chain]-[Polypeptide Domain 3]-[Polypeptide chain]-[Polypeptide Domain 4]). In some embodiments, the fourth polypeptide domain can be further coupled to another polypeptide chain (e.g., comprising the polypeptide sequence of SEQ ID NO: 1) or a linker (e.g., comprising the polypeptide sequence selected from SEQ ID NOs: 37-50 wherein the fourth polypeptide domain is disposed N-terminal to the polypeptide chain or a linker. In some embodiments, the first polypeptide domain can be at least a portion of an endonuclease (e.g., comprising the polypeptide sequence selected from SEQ ID NOs: 3-5 or 51-250), and the second polypeptide domain, the third polypeptide domain, and the fourth polypeptide domain can be gene modulators (e.g., comprising the polypeptide sequence selected from SEQ ID NOs: 6-17). In another example, the first polypeptide domain, the second polypeptide domain, the third polypeptide, and the fourth polypeptide domain can be gene modulators (e.g., comprising the polypeptide sequence selected from SEQ ID NOs: 6-17). In some embodiments, the first polypeptide domain, the third polypeptide domain, and the fourth polypeptide domain can be gene modulators (e.g., comprising the polypeptide sequence selected from SEQ ID NOs: 6-17), and the second polypeptide domain can be at least a portion of an endonuclease (e.g., comprising the polypeptide sequence selected from SEQ ID NOs: 3-5 or 51-250).

In some embodiments, the polypeptide molecule disclosed herein can further comprise one or more heterologous nuclear export signal(s) (NES(s)) or nuclear localization signal(s) (NLS(s)). In some embodiments, the NLS is positioned at the N-terminus of the polypeptide molecule. In some embodiments, the NLS is positioned at the C-terminus of the polypeptide molecule. Non-limiting examples of NLSs include an NLS sequence derived from: the NLS of the SV40 virus large T-antigen, having the amino acid sequence PKKKRKV (SEQ ID NO: 18), PKKKRKVEAS (SEQ ID NO: 19), or PKKKRKVGSGS (SEQ ID NO: 20; SV40 with linker sequences underlined); the c-myc NLS having the amino acid sequence PAAKRVKLD (SEQ ID NO: 21), RQRRNELKRSP (SEQ ID NO: 22), PAAKRVKLDGSGS (SEQ ID NO: 23; c-myc with linker sequences underlined); the NLS from nucleoplasmin (e.g., the nucleoplasmin bipartite NLS with the sequence KRPAATKKAGQAKKKK (SEQ ID NO: 24)); the hRNPA1 M9 NLS having the sequence NQSSNFGPMKGGNFGGRSSGPYGGGGQYFAKPRNQGGY (SEQ ID NO: 25); the sequence RMRIZFKNKGKDTAELRRRRVEVSVELRKAKKDEQILKRRNV (SEQ ID NO: 26) of the IBB domain from importin-alpha; the sequences VSRKRPRP (SEQ ID NO: 27) and PPKKARED (SEQ ID NO: 28) of the myoma T protein; the sequence PQPKKKPL (SEQ ID NO: 29) of human p53; the sequence SALIKKKKKMAP (SEQ ID NO:30) of mouse c-abl IV; the sequences DRLRR (SEQ ID NO: 31) and PKQKKRK (SEQ ID NO: 32) of the influenza virus NS1; the sequence RKLKKKIKKL (SEQ ID NO: 33) of the Hepatitis virus delta antigen; the sequence REKKKFLKRR (SEQ ID NO: 34) of the mouse Mx 1 protein; the sequence KRKGDEVDGVDEVAKKKSKK (SEQ ID NO: 35) of the human poly(ADP-ribose) polymerase; and the sequence RKCLQAGMNLEARKTKK (SEQ ID NO: 36) of the steroid hormone receptors (human) glucocorticoid. In some embodiments, the one or more NLSs are of sufficient strength to drive accumulation of the DNA-targeting the polypeptide molecule disclosed herein in a detectable amount in the nucleus of a eukaryotic cell.

In some embodiments, the polypeptide molecule disclosed herein can comprise at least 1 or more (e.g., at least 2, at least 3, at least 4, at least 5, at least 6, at least 7, at least 8, at least 9, at least 10, or more) NLSs. In some embodiments, the polypeptide molecule can comprise about or more than about 1, 2, 3, 4, 5, 6, 7, 8, 9, 10, or more NLSs at or near the N-terminus, about or more than about 1, 2, 3, 4, 5, 6, 7, 8, 9, 10, or more NLSs at or near the C-terminus, or a combination thereof (e.g., zero or at least one or more NLS at the N-terminus and zero or at one or more NLS at the C-terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the polypeptide molecule, an NLS can be coupled to the C-terminal of the polypeptide molecule.

Sequences

SEQ ID NO: 1 (Polypeptide Chain)

| | |
|---|---|
| 1 | GKESGSVGGS GGSSEQLAQF RSLDG |

SEQ ID NO: 2 (Unmodified Polypeptide Chain)

| | |
|---|---|
| 1 | KESGSVSSEQ LAQFRSLD |

SEQ ID NO: 3 (Un1Cas12fl or “CasMINI”)

| | |
|---|---|
| 1 | MAKNTITKTL KLRIVRPYNS AEVEKIVADE KNNREKIALE KNKDKVKEAC |
| 51 | SKHLKVAAYC TTQVERNACL FCKARKLDDK FYQKLRGQFP DAVFWQEISE |
| 101 | IFROLQKQAA EIYNQSLIEL YYEIFIKGKG IANASSVEHY LSDVCYTRAA |
| 151 | ELFKNAAIAS GLRSKIKSNF RLKELKNMKS GLPTTKSDNF PIPLVKQKGG |
| 201 | QYTGFEISNH NSDFIIKIPF GRWQVKKEID KYRPWEKFDF EQVQKSPKPI |
| 251 | SLLLSTQRRK RNKGWSKDEG TEAEIKKVMN GDYQTSYIEV KRGSKIGEKS |
| 301 | AWMLNLSIDV PKIDKGVDPS IIGGIDVGVK SPLVCAINNA FSRYSISDND |
| 351 | LFHENKKMFA RRRILLKKNR HKRAGHGAKN KLKPITILTE KSERFRKKLI |
| 401 | ERWACEIADF FIKNKVGTVQ MENLESMKRK EDSYENIRLR GFWPYAEMON |
| 451 | KIEFKLKQYG IEIRKVAPNN TSKTCSKCGH LNNYENFEYR KKNKFPHFKC |
| 501 | EKCNFKENAD YNAALNISNP KLKSTKEEP |

SEQ ID NO: 4 (deactivated nuclease variant of Un1Cas12fl
or “dCasMINI”)

| | |
|---|---|
| 1 | MAKNTITKTL KLRIVRPYNS AEVEKIVADE KNNREKIALE KNKDKVKEAC |
| 51 | SKHLKVAAYC TTQVERNACL FCKARKLDDK FYQKLRGQFP DAVEWQEISE |
| 101 | IFRQLQKQAA EIYNQSLIEL YYEIFIKGKG IANASSVEHY LSRVCYRRAA |
| 151 | ELFKNAAIAS GLRSKIKSNF RLKELKNMKS GLPTTKSDNF PIPLVKQKGG |
| 201 | QYTGFEISNH NSDFIIKIPF GRWQVKKEID KYRPWEKFDF EQVQKSPKPI |
| 251 | SLLLSTQRRK RNKGWSKDEG TEAEIKKVMN GDYQTSYIEV KRGSKICEKS |
| 301 | AWMLNLSIDV PKIDKGVDPS IIGGIAVGVR SPLVCAINNA FSRYSISDND |
| 351 | LFHENKKMFA RRRILLKKNR HKRAGHGAKN KLKPITILTE KSERFRKKLI |
| 401 | ERWACEIADE FIKNKVGTVQ MENLESMKRK EDSYENIRLR GFWPYAEMQN |
| 451 | KIEFKLKQYG IEIRKVAPNN TSKTCSKCGH LNNYFNFEYR KKNKFPHFKC |
| 501 | EKCNFKENAA YNAALNISNP KLKSTKERP |

SEQ ID NO: 5 (deactivated nuclease variant of Cas9 or
“dCas9”)

| | |
|---|---|
| 1 | MDKKYSIGLA IGTNSVGWAV ITDEYKVPSK KFKVLGNTDR HSIKKNLIGA |
| 51 | LLFDSGETAE ATRLKRTARR RYTRRKNRIC YLQEIFSNEM AKVDDSFFHR |
| 101 | LEESELVEED KKHERHPIFG NIVDEVAYHE KYPTIYHLRK KLVDSTDKAD |
| 151 | LRLIYLALAH MIKERGHFLI EGDLNPDNSD VDKLFIQLVQ TYNQLFEENP |
| 201 | INASGVDAKA ILSARLSKSR RLENLIAQLP GEKKNGLEGN LIALSLGLTP |
| 251 | NFKSNEDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI |
| 351 | FFDQSKNGYA GYIDGGASQE EFYKFIKPIL EKMDGTEELL VKLNREDLLR |
| 401 | KQRTEDNGSI PHQIHLGELH AILRRQEDFY PELKDNREKI EKILTFRIPY |
| 451 | YVGPLARGNS RFAWMTRKSE ETITPWNFEE VVDKGASAQS FIERMTNEDK |
| 501 | NLPNEKVLPK HSLLYEYFTV YNELTKVKYV TEGMRKPAFL SGEQKKAIVD |
| 551 | LLFKTNRKVT VKQLKEDYFK KIECFDSVEI SGVEDRENAS LGTYHDLLKI |
| 601 | IKDKDELDNE ENEDILEDIV LTLTLFEDRE MIEERLKTYA HLEDDKVMKQ |
| 651 | LKRRRYTGWG RLSRKLINGI RDKQSGKTIL DELKSDGFAN RNEMQLIHDD |
| 701 | SLTFKEDIQK AQVSGQGDSL HEHIANLAGS PAIKKGILQT VKVVDELVKV |
| 751 | MGRHKPENIV IEMARENQTT QKGQKNSRER MKRIEEGIKE LGSQILKEHP |
| 801 | VENTQLQNEK LYLYYLQNGR DMYVDQELDI NRLSDYDVDA IVPQSFLKDD |
| 851 | SIDNKVLTRS DKNRGKSDNV PSEEVVKKMK NYWRQLLNAK LITQRKEDNL |
| 901 | TKAERGGLSE LDKAGFIKRQ LVETRQITKH VAQILDSRMN TKYDENDKLI |
| 951 | REVKVITLKS KLVSDERKDF QFYKVREINN YHHAHDAYLN AVVGTALIKK |
| 1001 | YPKLESEFVY GDYKVYDVRK MIAKSEQEIG KATAKYFFYS NIMNFFKTEI |
| 1051 | TLANGEIRKR PLIETNGETG EIVWDKGRDF ATVRKVLSMP QVNIVKKTEV |
| 1101 | QTGGFSKESI LPKRNSDKLI ARKKDWDPKK YGGFDSPTVA YSVLVVAKVE |
| 1151 | KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE |
| 1251 | DNEQKQLFVE QHKHYLDEII EQISEFSKRV ILADANLDKV LSAYNKHRDK |
| 1301 | PIREQAENII HLFTLINLGA PAAFKYEDTT IDRKRYTSTK EVLDATLIHQ |
| 1351 | SITGLYETRI DLSQLGGD |

SEQ ID NO: 6 (KRAB - 1)

| | |
|---|---|
| 1 | DAKSLTAWSR TLVTFKDVFV DFTREEWKLL DTAQQIVYRN VMLENYKNLV |
| 51 | SLGYQLTKPD VILRLEKGEE P |

SEQ ID NO: 7 (KRAB - 2)

| | |
|---|---|
| 1 | DAKSLTAWSR TLVTFKDVFV DFTREEWKLL DTAQQILYRN VMLENYKNLV |
| 51 | SLGYQLTKPD VILRLEKGEE PWLVEREIHQ ETHPDSETAF EIKSSV |

SEQ ID NO: 8 (DNMT3A)

| | |
|---|---|
| 1 | TYGLLRRRED WPSRLQMFFA NNHDQEFDPP KVYPPVPAEK RKPIRVLSLE |
| 51 | DGIATGLLVL KDLGIQVDRY IASEVCEDSI TVGMVRHQGK IMYVGDVRSV |
| 101 | TQKHIQEWGP FDLVIGGSPC NDLSIVNPAR KGLYEGTGRL FFEFYRLLHA |
| 151 | DRPKEGDDRP FFWLFENVVA MGVSDKRDIS RFLESNPVMI DAKEVSAAHR |
| 201 | ARYFWGNLPG MNRPLASTVN DKLELQECLE HGRIAKESKV RTITTRSNSI |
| 251 | KQGKDQHFPV FMNEKEDILW CTEMERVEGF PVHYTDVSNM SRLARQRLLG |
| 301 | RSWSVPVIRH LFAPLKEYFA CV |

SEQ ID NO: 9 (DNMT3L)

| | |
|---|---|
| 1 | MAAIPALDPE AEPSMDVILV GSSELSSSVS PGTGRDLIAY EVKANQRNIE |
| 51 | DICICCGSLQ VHTQHPLFEG GICAPCKDKF LDALFLYDDD GYQSYCSICC |
| 101 | SGETLLICGN PDCTRCYCFE CVDSLVGPGT SGKVHAMSNW VCYLCLPSSR |
| 151 | SGLLQRRRKW RSQLKAFYDR ESENPLEMFE TVPVWRRQPV RVLSLFEDIK |
| 201 | KELTSLGFLE SGSDPGQLKH VVDVTDTVRK DVEEWGPEDL VYGATPPLGH |
| 251 | TCDRPPSWYL FQFHRLLQYA RPKPGSPRPF FWMFVDNLVL NKEDLDVASR |
| 301 | FEMEPVTIPD VHGGSLQNAV RVWSNIPAIR SRHWALVSEL EELSLLAQNK |
| 351 | QSSKLAAKWP TKLVKNCELP LREYFKYFEST |

SEQ ID NO: 10 (EZH3)

| | |
|---|---|
| 1 | MGQTGKKSEK GPVCWRKRVK SEYMRLRQLK RFRRADEVKT MESSNRQKIL |
| 51 | ERTETLNQEW KQRRIQPVHI MTSVSSLRGT RECSVTSDLD FPAQVIPLKT |
| 101 | LNAVASVPIM YSWSPLQQNF MVEDETVLHN IPYMGDEVLD QDGTFIEELI |
| 151 | KNYDGKVHGD RECGFINDEI FVELVNALGQ YNDDDDDDDG DDPDEREEKQ |
| 201 | KDLEDNRDDK ETCPPRKFPA DKIFEAISSM FPDKGTAEEL KEKYKELTEQ |
| 251 | QLPGALPPEC TPNIDGPNAK SVQREQSLHS FHTLFCRRCF KYDCFLHPFH |
| 301 | ATPNTYKRKN TETALDNKPC GPQCYQHLEG AKEFAAALTA ERIKTPPKRP |
| 351 | GGRRRGRLPN NSSRPSTPTI SVLESKDTDS DREAGTETGG ENNDKEEEEK |
| 401 | KDETSSSSEA NSRCQTPIKM KPNIEPPENV EWSGAEASMF RVLIGTYYDN |
| 451 | FCAIARLIGT KTCRQVYEFR VKESSIIAPV PTEDVDTPPR KKKRKHRLWA |
| 501 | AHCRKIQLKK DGSSNHVYNY QPCDHPRQPC DSSCPCVIAQ NFCEKFCQCS |
| 551 | SECQNRFPGC RCKAQCNTKQ CPCYLAVREC DPDLCLTCGA ADHWDSKNVS |
| 601 | CKNCSIQRGS KKHLLLAPSD VAGWGIFIKD PVQKNEFISE YCGEIISQDE |
| 651 | ADRRGKVYDK YMCSELENLN NDFVVDATRK GNKIRFANHS VNPNCYAKVM |
| 701 | MVNGDHRIGI FAKRAIQTGE ELFFDYRYSQ ADALKYVGIE REMEIP |

SEQ ID NO: 11 (ZNF689)

| | |
|---|---|
| 1 | APPSAPLPAQ GPGKARPSRK RGRRPRALKF VDVAVYFSPE EWGCLRPAQR |
| 51 | ALYRDVMRET YGHLGALGCA GPKPALISWL ERNTD |

SEQ ID NO: 12 (DNMT3A/DNMT3L with linker sequences
underlined)

| | |
|---|---|
| 1 | MAAIPALDPE AEPSMDVILV GSSELSSSVS PGTGRDLIAY EVKANQRNIE |
| 51 | DICICCGSLQ VHTQHPLFEG GICAPCKDKF LDALFLYDDD GYQSYCSICC |
| 101 | SGETLLICGN PDCTRCYCFE CVDSLVGPGT SGKVHAMSNW VCYLCLPSSR |
| 151 | SGLLQRRRKW RSQLKAFYDR ESENPLEMFE TVPVWRRQPV RVLSLFEDIK |
| 201 | KELTSLGFLE SGSDPGQLKH VVDVTDTVRK DVEEWGPEDL VYGATPPLGH |
| 251 | TCDRPPSWYL FQFHRLLQYA RPKPGSPRPF FWMFVDNLVL NKEDLDVASR |
| 301 | FEMEPVTIPD VHGGSLQNAV RVWSNIPAIR SRHWALVSEL EELSLLAQNK |
| 351 | QSSKLAAKWP TKLVKNCFLP LREYFKYFST GSGGSGGSGG SGTYGLLRRR |
| 401 | EDWPSRLQMF FANNHDQEFD PPKVYPPVPA EKRKPIRVLS LFDGIATGLL |
| 451 | VLKDLGIQVD RYIASEVCED SITVGMVRHQ GKIMYVGDVR SVTQKHIQEW |
| 101 | GPFDLVIGGS PCNDLSIVNP ARKGLYEGTG RLFFEFYRLL HADRPKEGDD |

SEQ ID NO: 11 (ZNF689)

| | |
|---|---|
| 1 | APPSAPLPAQ GPGKARPSRK RGRRPRALKF VDVAVYFSPE EWGCLRPAQR |
| 51 | ALYRDVMRET YGHLGALGCA GPKPALISWL ERNTD |

SEQ ID NO: 12 (DNMT3A/DNMT3L with linker sequences
underlined)

| | |
|---|---|
| 1 | MAAIPALDPE AEPSMDVILV GSSELSSSVS PGTGRDLIAY EVKANQRNIE |
| 51 | DICICCGSLQ VHTQHPLFEG GICAPCKDKF LDALFLYDDD GYQSYCSICC |
| 101 | SGETLLICGN PDCTRCYCFE CVDSLVGPGT SGKVHAMSNW VCYLCLPSSR |
| 151 | SGLLQRRRKW RSQLKAFYDR ESENPLEMFE TVPVWRRQPV RVLSLFEDIK |
| 201 | KELTSLGFLE SGSDPGQLKH VVDVTDTVRK DVEEWGPEDL VYGATPPLGH |
| 251 | TCDRPPSWYL FQFHRLLQYA RPKPGSPRPF FWMFVDNLVL NKEDLDVASR |
| 301 | FEMEPVTIPD VHGGSLQNAV RVWSNIPAIR SRHWALVSEL EELSLLAQNK |
| 351 | QSSKLAAKWP TKLVKNCFLP LREYFKYFST GSGGSGGSGG SGTYGLLRRR |
| 401 | EDWPSRLQMF FANNHDQEFD PPKVYPPVPA EKRKPIRVLS LFDGIATGLL |
| 451 | VLKDLGIQVD RYIASEVCED SITVGMVRHQ GKIMYVGDVR SVTQKHIQEW |
| 101 | GPFDLVIGGS PCNDLSIVNP ARKGLYEGTG RLFFEFYRLL HADRPKEGDD |
| 151 | RPFFWLFENV VAMGVSDKRD ISRFLESNPV MIDAKEVSAA HRARYFWGNL |
| 201 | PGMNRPLAST VNDKLELQEC LEHGRIAKFS KVRTITTRSN SIKQGKDQHE |
| 251 | PVEMNEKEDI LWCTEMERVE GFPVHYTDVS NMSRLARQRL LGRSWSVPVI |
| 301 | RHLFAPLKEY FACV |

SEQ ID NO: 13 (VP16)

| | |
|---|---|
| 1 | DALDDFDLDM L |

SEQ ID NO: 14 (VP64)

| | |
|---|---|
| 1 | DALDDEDLDM LGSDALDDED LDMLGSDALD DEDLDMLGSD ALDDEDLDML |

SEQ ID NO: 15 (p65)

| | |
|---|---|
| 1 | QYLPDTDDRH RIEEKRKRTY ETFKSIMKKS PESGPTDPRP PPRRIAVPSR |
| 51 | SSASVPKPAP QPYPFTSSLS TINYDEFPTM VEPSGQISQA SALAPAPPQV |
| 101 | LPQAPAPAPA PAMVSALAQA PAPVPVLAPG PPQAVAPPAP KPTQAGEGTL |
| 151 | SEALLQLQFD DEDLGALLGN STDPAVFTDL ASVDNSEFQQ LLNQGIPVAP |
| 201 | HTTEPMLMEY PEAITRLVTG AQRPPDPAPA PLGAPGLPNG LLSGDEDESS |
| 251 | IADMDESALL SQISS |

SEQ ID NO: 16 (RTA)

| | |
|---|---|
| 1 | RDSREGMELP KPEAGSAISD VFEGREVCQP KRIRPFHPPG SPWANRPLPA |
| 51 | SLAPTPTGPV HEPVGSLTPA PVPQPLDPAP AVTPEASHLL EDPDEETSQA |
| 101 | VKALREMADT VIPQKEEAAI CGQMDLSHPP PRGHLDELTT TLESMTEDLN |
| 151 | LDSPLTPELN EILDTELNDE CLLHAMHIST GLSIFDTSLE |

SEQ ID NO: 17 (VP64-p65-Rta or “VPR” with linker sequences
underlined)

| | |
|---|---|
| 1 | DALDDEDLDM LGSDALDDED LDMLGSDALD DEDLDMLGSD ALDDEDLDML |
| 51 | GSGGSGSQYL PDTDDRHRIE EKRKRTYETF KSIMKKSPFS GPTDPRPPPR |
| 101 | RIAVPSRSSA SVPKPAPQPY PFTSSLSTIN YDEFPTMVFP SGQISQASAL |
| 151 | APAPPQVLPQ APAPAPAPAM VSALAQAPAP VPVLAPGPPQ AVAPPAPKPT |
| 201 | QAGEGTLSEA LLQLQFDDED LGALLGNSTD PAVFTDLASV DNSEFQQLLN |
| 251 | QGIPVAPHTT EPMLMEYPEA ITRLVTGAQR PPDPAPAPLG APGLPNGLLS |
| 301 | GDEDESSIAD MDESALLSQI SSGSGSGSRD SREGMELPKP EAGSAISDVE |
| 351 | EGREVCQPKR IRPFHPPGSP WANRPLPASL APTPTGPVHE PVGSLTPAPV |
| 401 | PQPLDPAPAV TPEASHLLED PDEETSQAVK ALREMADTVI PQKEEAAICG |
| 451 | QMDLSHPPPR GHLDELTTTL |

In some embodiments, the polypeptide molecule as disclosed herein can be configured to modulate (e.g., edit, activate, suppress) expression level of a target gene, upon formation of a complex comprising the polypeptide molecule and a target polynucleotide sequence operatively coupled to a target gene of a cell. In some embodiments, the polypeptide molecule disclosed herein (e.g., a polypeptide molecule comprising a gene modulator coupled to the polypeptide chain) can be configured to effect a greater change in expression level of the target gene, as compared to a control complex (e.g., a polypeptide molecule comprising a gene modulator in absence of the polypeptide chain). The control complex can be with a polypeptide molecule comprising at least a portion of an endonuclease (e.g., dCas) alone without any gene modulators operatively coupled to the at least the portion of the endonuclease via the polypeptide chain. The control complex can be with a polypeptide molecule comprising a gene modulators alone without any polypeptide chain operatively coupled to the gene modulators.

In some embodiments, a polypeptide molecule can comprise a first polypeptide domain coupled to a second polypeptide domain via the polypeptide chain disclosed herein, wherein the first polypeptide domain can be a gene modulator and the second polypeptide domain can be an additional gene modulator or at least a portion of an endonuclease, that can effect (e.g., in conjunction with a guide nucleic acid) a greater change in expression level of the target gene, as compared to a control complex (e.g., a polypeptide molecule comprising a first polypeptide domain and a second polypeptide domain in absence of the polypeptide chain).

In some embodiments, a polypeptide molecule comprising a first polypeptide domain coupled to a second polypeptide domain via the polypeptide chain disclosed herein, wherein the first polypeptide domain is a gene modulator and the second polypeptide domain is an additional gene modulator, can effect (e.g., either alone or in conjunction with at least a portion of an endonuclease, such as Cas or dCas protein, and a guide nucleic acid) a greater change in expression level of the target gene, as compared to a control complex (e.g., a polypeptide molecule comprising a gene modulator and an additional gene modulator in absence of the polypeptide chain). In some embodiments, a polypeptide molecule comprising a first polypeptide domain coupled to a second polypeptide domain via the polypeptide chain disclosed herein, wherein the first polypeptide domain is a gene modulator and the second polypeptide domain is at least a portion of an endonuclease, can effect a greater change in expression level of the target gene, as compared to a control complex (e.g., a polypeptide molecule comprising at least a portion of an endonuclease and a gene modulator in absence of the polypeptide chain). The control complex can be a polypeptide molecule comprising at least a portion of endonuclease and/or one or more gene modulators in absence of the polypeptide chain.

In some embodiments, the expression level of the target gene that is reduced via the polypeptide molecule as disclosed herein can be at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%, as compared to the expression level of the target gene of the control complex (e.g., a polypeptide molecule in absence of a polypeptide chain).

In some embodiments, the expression level of the target gene that is reduced via the polypeptide molecule as disclosed herein can be less than the expression level of the target gene in the cell that is reduced by a control complex (e.g., a polypeptide molecule in absence of a polypeptide chain), by at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 15%, at least or up to about 20%, at least or up to about 25%, at least or up to about 30%, at least or up to about 40%, at least or up to about 50%, at least or up to about 60%, at least or up to about 70%, at least or up to about 80%, at least or up to about 90%, at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, or at least or up to about 10-fold.

In some embodiments, the expression level of the target gene that is activated via the polypeptide molecule as disclosed herein can be at least or up to about 50%, at least or up to about 60%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, at least or up to about 100%, at least or up to about 1010%, at least or up to about 102%, at least or up to about 103%, at least or up to about 104%, at least or up to about 105%, at least or up to about 106%, at least or up to about 107%, at least or up to about 108%, at least or up to about 109%, at least or up to about 110%, at least or up to about 115%, at least or up to about 120%, at least or up to about 125%, at least or up to about 130%, at least or up to about 140%, at least or up to about 150%, at least or up to about 160%, at least or up to about 170%, at least or up to about 180%, at least or up to about 190%, at least or up to about 200%, or more, as compared to the expression level of the target gene in the cell activated by a control complex (e.g., a polypeptide molecule in absence of a polypeptide chain).

In some embodiments, the expression level of the target gene that is activated via the polypeptide molecule as disclosed herein can be at least or up to about 1%, at least or up to about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least or up to about 15%, at least or up to about 16%, at least or up to about 17%, at least or up to about 18%, at least or up to about 19%, at least or up to about 20%, at least or up to about 25%, at least or up to about 30%, at least or up to about 40%, at least or up to about 50%, at least or up to about 60%, at least or up to about 70%, at least or up to about 80%, at least or up to about 90%, at least or up to about 100%, at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.1-fold, at least or up to about 1.2-fold, at least or up to about 1.3-fold, at least or up to about 1.4-fold, at least or up to about 1.5-fold, at least or up to about 1.6-fold, at least or up to about 1.7-fold, at least or up to about 1.8-fold, at least or up to about 1.9-fold, at least or up to about 2-fold, at least or up to about 2.5-fold, at least or up to about 3-fold, at least or up to about 3.5-fold, at least or up to about 4-fold, at least or up to about 4.5-fold, at least or up to about 5-fold, at least or up to about 6-fold, at least or up to about 7-fold, at least or up to about 8-fold, at least or up to about 9-fold, at least or up to about 10-fold, at least or up to about 15-fold, at least or up to about 20-fold, at least or up to about 30-fold, at least or up to about 40-fold, at least or up to about 50-fold, or more greater than the expression level of the target gene in the cell activated by a control complex (e.g., a polypeptide molecule in absence of a polypeptide chain).

In some embodiments, the changes in the expression level of the target gene via the polypeptide molecule as disclosed herein can be substantially sustained (e.g., persisting or maintaining at least about 50%, at least about 60%, at least about 70%, at least about 80%, at least about 90%, at least about 95%, at least about 99%, or about 100% of a peak value or that of the modulated level) for at least or up to about 1 hour, at least or up to about 2 hours, at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 8 days, at least or up to about 9 days, at least or up to about 10 days, at least or up to about 11 days, at least or up to about 12 days, at least or up to about 13 days, at least or up to about 14 days, at least or up to about 3 weeks, at least or up to about 4 weeks, at least or up to about 2 months, at least or up to about 4 months, or at least or up to about 6 months.

### Polypeptide Chain

In some embodiments, the polypeptide chain disclosed herein can have a size about 5 amino acid residues to about 50 amino acid residues. In some embodiments, the polypeptide as disclosed herein can have a size about 5 amino acid residues to about 10 amino acid residues, about 5 amino acid residues to about 15 amino acid residues, about 5 amino acid residues to about 18 amino acid residues, about 5 amino acid residues to about 20 amino acid residues, about 5 amino acid residues to about 22 amino acid residues, about 5 amino acid residues to about 24 amino acid residues, about 5 amino acid residues to about 26 amino acid residues, about 5 amino acid residues to about 28 amino acid residues, about 5 amino acid residues to about 30 amino acid residues, about 5 amino acid residues to about 40 amino acid residues, about 5 amino acid residues to about 50 amino acid residues, about 10 amino acid residues to about 15 amino acid residues, about 10 amino acid residues to about 18 amino acid residues, about 10 amino acid residues to about 20 amino acid residues, about 10 amino acid residues to about 22 amino acid residues, about 10 amino acid residues to about 24 amino acid residues, about 10 amino acid residues to about 26 amino acid residues, about 10 amino acid residues to about 28 amino acid residues, about 10 amino acid residues to about 30 amino acid residues, about 10 amino acid residues to about 40 amino acid residues, about 10 amino acid residues to about 50 amino acid residues, about 15 amino acid residues to about 18 amino acid residues, about 15 amino acid residues to about 20 amino acid residues, about 15 amino acid residues to about 22 amino acid residues, about 15 amino acid residues to about 24 amino acid residues, about 15 amino acid residues to about 26 amino acid residues, about 15 amino acid residues to about 28 amino acid residues, about 15 amino acid residues to about 30 amino acid residues, about 15 amino acid residues to about 40 amino acid residues, about 15 amino acid residues to about 50 amino acid residues, about 18 amino acid residues to about 20 amino acid residues, about 18 amino acid residues to about 22 amino acid residues, about 18 amino acid residues to about 24 amino acid residues, about 18 amino acid residues to about 26 amino acid residues, about 18 amino acid residues to about 28 amino acid residues, about 18 amino acid residues to about 30 amino acid residues, about 18 amino acid residues to about 40 amino acid residues, about 18 amino acid residues to about 50 amino acid residues, about 20 amino acid residues to about 22 amino acid residues, about 20 amino acid residues to about 24 amino acid residues, about 20 amino acid residues to about 26 amino acid residues, about 20 amino acid residues to about 28 amino acid residues, about 20 amino acid residues to about 30 amino acid residues, about 20 amino acid residues to about 40 amino acid residues, about 20 amino acid residues to about 50 amino acid residues, about 22 amino acid residues to about 24 amino acid residues, about 22 amino acid residues to about 26 amino acid residues, about 22 amino acid residues to about 28 amino acid residues, about 22 amino acid residues to about 30 amino acid residues, about 22 amino acid residues to about 40 amino acid residues, about 22 amino acid residues to about 50 amino acid residues, about 24 amino acid residues to about 26 amino acid residues, about 24 amino acid residues to about 28 amino acid residues, about 24 amino acid residues to about 30 amino acid residues, about 24 amino acid residues to about 40 amino acid residues, about 24 amino acid residues to about 50 amino acid residues, about 26 amino acid residues to about 28 amino acid residues, about 26 amino acid residues to about 30 amino acid residues, about 26 amino acid residues to about 40 amino acid residues, about 26 amino acid residues to about 50 amino acid residues, about 28 amino acid residues to about 30 amino acid residues, about 28 amino acid residues to about 40 amino acid residues, about 28 amino acid residues to about 50 amino acid residues, about 30 amino acid residues to about 40 amino acid residues, about 30 amino acid residues to about 50 amino acid residues, or about 40 amino acid residues to about 50 amino acid residues.

In some embodiments, the polypeptide chain can have a size about 5 amino acid residues, about 10 amino acid residues, about 15 amino acid residues, about 18 amino acid residues, about 20 amino acid residues, about 22 amino acid residues, about 24 amino acid residues, about 26 amino acid residues, about 28 amino acid residues, about 30 amino acid residues, about 40 amino acid residues, or about 50 amino acid residues.

In some embodiments, the polypeptide chain can have a size at least about 5 amino acid residues, about 10 amino acid residues, about 15 amino acid residues, about 18 amino acid residues, about 20 amino acid residues, about 22 amino acid residues, about 24 amino acid residues, about 26 amino acid residues, about 28 amino acid residues, about 30 amino acid residues, or about 40 amino acid residues.

In some embodiments, the polypeptide chain can have a size at most about 10 amino acid residues, about 15 amino acid residues, about 18 amino acid residues, about 20 amino acid residues, about 22 amino acid residues, about 24 amino acid residues, about 26 amino acid residues, about 28 amino acid residues, about 30 amino acid residues, about 40 amino acid residues, or about 50 amino acid residues.

In some embodiments, the polypeptide chain disclosed herein can comprise a polypeptide sequence (e.g., an amino acid sequence) exhibiting at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 71%, at least or up to about 72%, at least or up to about 73%, at least or up to about 74%, at least or up to about 75%, at least or up to about 76%, at least or up to about 77%, at least or up to about 78%, at least or up to about 79%, at least or up to about 80%, at least or up to about 81%, at least or up to about 82%, at least or up to about 83%, at least or up to about 84%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% identical to the polypeptide sequence of SEQ ID NO: 1. For example, the polypeptide sequence of the gene modulator can be between about 80% and about 100% identical to the polypeptide sequence of SEQ ID NO: 1.

In some embodiments, the polypeptide chain can comprise about 1 GS residue to about 10 GS residues. In some embodiments, the polypeptide molecule can comprise about 1 GS residue to about 2 GS residues, about 1 GS residue to about 3 GS residues, about 1 GS residue to about 4 GS residues, about 1 GS residue to about 5 GS residues, about 1 GS residue to about 6 GS residues, about 1 GS residue to about 7 GS residues, about 1 GS residue to about 8 GS residues, about 1 GS residue to about 9 GS residues, about 1 GS residue to about 10 GS residues, about 2 GS residues to about 3 GS residues, about 2 GS residues to about 4 GS residues, about 2 GS residues to about 5 GS residues, about 2 GS residues to about 6 GS residues, about 2 GS residues to about 7 GS residues, about 2 GS residues to about 8 GS residues, about 2 GS residues to about 9 GS residues, about 2 GS residues to about 10 GS residues, about 3 GS residues to about 4 GS residues, about 3 GS residues to about 5 GS residues, about 3 GS residues to about 6 GS residues, about 3 GS residues to about 7 GS residues, about 3 GS residues to about 8 GS residues, about 3 GS residues to about 9 GS residues, about 3 GS residues to about 10 GS residues, about 4 GS residues to about 5 GS residues, about 4 GS residues to about 6 GS residues, about 4 GS residues to about 7 GS residues, about 4 GS residues to about 8 GS residues, about 4 GS residues to about 9 GS residues, about 4 GS residues to about 10 GS residues, about 5 GS residues to about 6 GS residues, about 5 GS residues to about 7 GS residues, about 5 GS residues to about 8 GS residues, about 5 GS residues to about 9 GS residues, about 5 GS residues to about 10 GS residues, about 6 GS residues to about 7 GS residues, about 6 GS residues to about 8 GS residues, about 6 GS residues to about 9 GS residues, about 6 GS residues to about 10 GS residues, about 7 GS residues to about 8 GS residues, about 7 GS residues to about 9 GS residues, about 7 GS residues to about 10 GS residues, about 8 GS residues to about 9 GS residues, about 8 GS residues to about 10 GS residues, or about 9 GS residues to about 10 GS residues. In some embodiments, the polypeptide molecule can comprise about 1 GS residue, about 2 GS residues, about 3 GS residues, about 4 GS residues, about 5 GS residues, about 6 GS residues, about 7 GS residues, about 8 GS residues, about 9 GS residues, or about 10 GS residues. In some embodiments, the polypeptide molecule can comprise at least about 1 GS residue, about 2 GS residues, about 3 GS residues, about 4 GS residues, about 5 GS residues, about 6 GS residues, about 7 GS residues, about 8 GS residues, or about 9 GS residues. In some embodiments, the polypeptide molecule can comprise at most about 2 GS residues, about 3 GS residues, about 4 GS residues, about 5 GS residues, about 6 GS residues, about 7 GS residues, about 8 GS residues, about 9 GS residues, or about 10 GS residues.

In some embodiments, the polypeptide chain disclosed herein can have a polypeptide sequence containing stretches of G, G and S (e.g., GGS) residues. In some embodiments, the polypeptide molecule can comprise about 1 GGS residue to about 10 GGS residues. In some embodiments, the polypeptide molecule can comprise about 1 GGS residue to about 2 GGS residues, about 1 GGS residue to about 3 GGS residues, about 1 GGS residue to about 4 GGS residues, about 1 GGS residue to about 5 GGS residues, about 1 GGS residue to about 6 GGS residues, about 1 GGS residue to about 7 GGS residues, about 1 GGS residue to about 8 GGS residues, about 1 GGS residue to about 9 GGS residues, about 1 GGS residue to about 10 GGS residues, about 2 GGS residues to about 3 GGS residues, about 2 GGS residues to about 4 GGS residues, about 2 GGS residues to about 5 GGS residues, about 2 GGS residues to about 6 GGS residues, about 2 GGS residues to about 7 GGS residues, about 2 GGS residues to about 8 GGS residues, about 2 GGS residues to about 9 GGS residues, about 2 GGS residues to about 10 GGS residues, about 3 GGS residues to about 4 GGS residues, about 3 GGS residues to about 5 GGS residues, about 3 GGS residues to about 6 GGS residues, about 3 GGS residues to about 7 GGS residues, about 3 GGS residues to about 8 GGS residues, about 3 GGS residues to about 9 GGS residues, about 3 GGS residues to about 10 GGS residues, about 4 GGS residues to about 5 GGS residues, about 4 GGS residues to about 6 GGS residues, about 4 GGS residues to about 7 GGS residues, about 4 GGS residues to about 8 GGS residues, about 4 GGS residues to about 9 GGS residues, about 4 GGS residues to about 10 GGS residues, about 5 GGS residues to about 6 GGS residues, about 5 GGS residues to about 7 GGS residues, about 5 GGS residues to about 8 GGS residues, about 5 GGS residues to about 9 GGS residues, about 5 GGS residues to about 10 GGS residues, about 6 GGS residues to about 7 GGS residues, about 6 GGS residues to about 8 GGS residues, about 6 GGS residues to about 9 GGS residues, about 6 GGS residues to about 10 GGS residues, about 7 GGS residues to about 8 GGS residues, about 7 GGS residues to about 9 GGS residues, about 7 GGS residues to about 10 GGS residues, about 8 GGS residues to about 9 GGS residues, about 8 GGS residues to about 10 GGS residues, or about 9 GGS residues to about 10 GGS residues. In some embodiments, the polypeptide molecule can comprise about 1 GGS residue, about 2 GGS residues, about 3 GGS residues, about 4 GGS residues, about 5 GGS residues, about 6 GGS residues, about 7 GGS residues, about 8 GGS residues, about 9 GGS residues, or about 10 GGS residues. In some embodiments, the polypeptide molecule can comprise at least about 1 GGS residue, about 2 GGS residues, about 3 GGS residues, about 4 GGS residues, about 5 GGS residues, about 6 GGS residues, about 7 GGS residues, about 8 GGS residues, or about 9 GGS residues. In some embodiments, the polypeptide molecule can comprise at most about 2 GGS residues, about 3 GGS residues, about 4 GGS residues, about 5 GGS residues, about 6 GGS residues, about 7 GGS residues, about 8 GGS residues, about 9 GGS residues, or about 10 GGS residues.

In some embodiments, a polypeptide molecule can comprise a plurality of the polypeptide chains. In some embodiments, a polypeptide molecule can comprise at least one, at least two, at least three, at least four, at least five, at least six, at least seven, at least eight, at least nine, or at least ten polypeptide chains disclosed herein.

Disclosed herein is a polypeptide chain derived from an unmodified polypeptide chain (e.g., comprising the polypeptide sequence of SEQ ID NO: 2), wherein the polypeptide chain is heterologous to the polypeptide sequence of SEQ ID NO:2. In some embodiments, the polypeptide chain described herein can have from about 1 amino acid to about 20 amino acids insertion, deletions, or substitution relative to the polypeptide sequence of SEQ ID NO:2. For example, the polypeptide chain disclosed herein can have stretches of glycine (G) and serine (S) residues insertion to the polypeptide sequence of SEQ ID NO:2. In some embodiments, the polypeptide chain disclosed herein can have serine (S) or threonine (T) inserted to the polypeptide sequence of SEQ ID NO:2. In some embodiments, a polypeptide chain can also contain additional amino acids such as threonine (T) and alanine (A), as well as polar amino acids such as lysine (K) and glutamine (Q).

In some embodiments, the polypeptide chain disclosed herein can comprise an insertion of about 1 G and/or S residue to about 10 G and/or S residues to the polypeptide sequence of SEQ ID NO:2. In some embodiments, the polypeptide molecule can comprise an insertion of about 1 G and/or S residue to about 2 G and/or S residues, about 1 G and/or S residue to about 3 G and/or S residues, about 1 G and/or S residue to about 4 G and/or S residues, about 1 G and/or S residue to about 5 G and/or S residues, about 1 G and/or S residue to about 6 G and/or S residues, about 1 G and/or S residue to about 7 G and/or S residues, about 1 G and/or S residue to about 8 G and/or S residues, about 1 G and/or S residue to about 9 G and/or S residues, about 1 G and/or S residue to about 10 G and/or S residues, about 2 G and/or S residues to about 3 G and/or S residues, about 2 G and/or S residues to about 4 G and/or S residues, about 2 G and/or S residues to about 5 G and/or S residues, about 2 G and/or S residues to about 6 G and/or S residues, about 2 G and/or S residues to about 7 G and/or S residues, about 2 G and/or S residues to about 8 G and/or S residues, about 2 G and/or S residues to about 9 G and/or S residues, about 2 G and/or S residues to about 10 G and/or S residues, about 3 G and/or S residues to about 4 G and/or S residues, about 3 G and/or S residues to about 5 G and/or S residues, about 3 G and/or S residues to about 6 G and/or S residues, about 3 G and/or S residues to about 7 G and/or S residues, about 3 G and/or S residues to about 8 G and/or S residues, about 3 G and/or S residues to about 9 G and/or S residues, about 3 G and/or S residues to about 10 G and/or S residues, about 4 G and/or S residues to about 5 G and/or S residues, about 4 G and/or S residues to about 6 G and/or S residues, about 4 G and/or S residues to about 7 G and/or S residues, about 4 G and/or S residues to about 8 G and/or S residues, about 4 G and/or S residues to about 9 G and/or S residues, about 4 G and/or S residues to about 10 G and/or S residues, about 5 G and/or S residues to about 6 G and/or S residues, about 5 G and/or S residues to about 7 G and/or S residues, about 5 G and/or S residues to about 8 G and/or S residues, about 5 G and/or S residues to about 9 G and/or S residues, about 5 G and/or S residues to about 10 G and/or S residues, about 6 G and/or S residues to about 7 G and/or S residues, about 6 G and/or S residues to about 8 G and/or S residues, about 6 G and/or S residues to about 9 G and/or S residues, about 6 G and/or S residues to about 10 G and/or S residues, about 7 G and/or S residues to about 8 G and/or S residues, about 7 G and/or S residues to about 9 G and/or S residues, about 7 G and/or S residues to about 10 G and/or S residues, about 8 G and/or S residues to about 9 G and/or S residues, about 8 G and/or S residues to about 10 G and/or S residues, or about 9 G and/or S residues to about 10 G and/or S residues to the polypeptide sequence of SEQ ID NO:2. In some embodiments, the polypeptide molecule can comprise an insertion of about 1 G and/or S residue, about 2 G and/or S residues, about 3 G and/or S residues, about 4 G and/or S residues, about 5 G and/or S residues, about 6 G and/or S residues, about 7 G and/or S residues, about 8 G and/or S residues, about 9 G and/or S residues, and/or about 10 G and/or S residues to the polypeptide sequence of SEQ ID NO:2. In some embodiments, the polypeptide molecule can comprise an insertion of at least about 1 G and/or S residue, about 2 G and/or S residues, about 3 G and/or S residues, about 4 G and/or S residues, about 5 G and/or S residues, about 6 G and/or S residues, about 7 G and/or S residues, about 8 G and/or S residues, and/or about 9 G and/or S residues to the polypeptide sequence of SEQ ID NO:2. In some embodiments, the polypeptide molecule can comprise an insertion of at most about 2 G and/or S residues, about 3 G and/or S residues, about 4 G and/or S residues, about 5 G and/or S residues, about 6 G and/or S residues, about 7 G and/or S residues, about 8 G and/or S residues, about 9 G and/or S residues, or about 10 G and/or S residues to the polypeptide sequence of SEQ ID NO:2.

In some embodiments, a polypeptide molecule can further comprise one or more linkers (e.g., in addition to the polypeptide chain disclosed herein). Any suitable linker can be used. A flexible linker can have a sequence containing stretches of glycine and serine residues. The small size of the glycine and serine residues provides flexibility and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid linker can have, for example, an alpha helix-structure. An alpha-helical rigid linker can act as a spacer between protein domains. Non-limiting examples of linkers include the sequences in Table 2, and repeats thereof, for example, at least or up to about 1, at least or up to about 2, at least or up to about 3, at least or up to about 4, at least or up to about 5, at least or up to about 6, at least or up to about 7, at least or up to about 8, at least or up to about 9, or at least or up to about 10 repeats (e.g., repeats with or without any intervening amino acid sequence(s) in between the repeats). SEQ ID NOs: 37-43 provide linkers (e.g., flexible linkers) or subunits thereof. SEQ ID NOs: 47-50 provide linkers (e.g., rigid linkers) or subunits thereof.

TABLE 2
Non-limiting linker

| SEQ ID NO: | Sequence |
|---|---|
| 37 | GSGGSGGSGGSG |
| 38 | GGGGS |
| 39 | GGGS |
| 40 | GG |
| 41 | EGKSSGSGSESKST |
| 42 | GSAGSAAGSGEF |
| 43 | AYPYDVPDYASLGSGSPKKKRKVEDPKKKRKVDGIGSGSS |
| | GSSGSGGGSGTR |
| 44 | GGPSSGAPPPSGGSPAGSPTSTEEGTSESATPESGPGTST |
| | EPSEGSAPGSPAGSPTSTEEGTSTEPSEGSAPGTSTEPSE |
| 45 | GGGGSKESGSVSSEQLAQFRSLDEGKSSGSGSESKSTGSA |
| | GSAAGSGEFGGGGS |
| 46 | YPYDVPDYASLGSGSPKKKRKVEDPKKKRKVDGIGSGSNG |
| | SSGSGGGSGTR |
| 47 | EAAAK |
| 48 | EAAAR |
| 49 | PAPAP |
| 50 | AEAAAKEAAAKA |

In some embodiments, a linker sequence as disclosed herein can be, for example, at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, at least or up to about 30, at least or up to about 31, at least or up to about 32, at least or up to about 33, at least or up to about 34, at least or up to about 35, at least or up to about 36, at least or up to about 37, at least or up to about 38, at least or up to about 39, at least or up to about 40, at least or up to about 41, at least or up to about 42, at least or up to about 43, at least or up to about 44, at least or up to about 45, at least or up to about 46, at least or up to about 47, at least or up to about 48, at least or up to about 49, at least or up to about or 50 amino acid residues in length.

In some embodiments, a linker sequence as disclosed herein can comprise at least 1, at least 2, at least 3, at least 5, at least 7, at least 9, at least 11, at least 13, at least 15, or at least 20 amino acids. In some embodiments, a linker sequence can comprise at most 5, at most 7, at most 9, at most 11, at most 13, at most 15, at most 20, at most 25, at most 30, at most 40, or at most 50 amino acids.

In some embodiments, non-peptide linkers are used. A non-peptide linker can be, for example, a chemical linker. Two parts of a complex of the disclosure can be connected by a chemical linker. Each chemical linker of the disclosure can be alkylene, alkenylene, alkynylene, heteroalkylene, cycloalkylene, heterocycloalkylene, arylene, or heteroarylene, any of which is optionally substituted. In some embodiments, a chemical linker of the disclosure can be an ester, ether, amide, thioether, or polyethyleneglycol (PEG). In some embodiments, a linker can reverse the order of the amino acids sequence in a compound, for example, so that the amino acid sequences linked by the linked are head-to-head, rather than head-to-tail. Non-limiting examples of such linkers include diesters of dicarboxylic acids, such as oxalyl diester, malonyl diester, succinyl diester, glutaryl diester, adipyl diester, pimetyl diester, fumaryl diester, maleyl diester, phthalyl diester, isophthalyl diester, and terephthalyl diester. Non-limiting examples of such linkers include diamides of dicarboxylic acids, such as oxalyl diamide, malonyl diamide, succinyl diamide, glutaryl diamide, adipyl diamide, pimetyl diamide, fumaryl diamide, maleyl diamide, phthalyl diamide, isophthalyl diamide, and terephthalyl diamide. Non-limiting examples of such linkers include diamides of diamino linkers, such as ethylene diamine, 1,2-di(methylamino)ethane, 1,3-diaminopropane, 1,3-di(methylamino)propane, 1,4-di(methylamino)butane, 1,5-di(methylamino)pentane, 1,6-di(methylamino)hexane, and pipyrizine. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

In some embodiments, the polypeptide molecule as disclosed herein can comprise at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or about 1 glycine-serine (GS) linker(s). In some embodiments, the polypeptide molecule as disclosed herein can comprise at least about 1, at least about 2, at least about 3, at least about 4, at least about 5, at least about 6, at least about 7, at least about, at least about 9, at least about 10, at least about 11, at least about 12, at least about 13, at least about 14, at least about 15, at least about 20, or more GS linker(s). In some cases, a GS linker may be a contiguous polynucleotide sequence that comprise no more than 1 glycine residue, no more than 2 contiguous glycine residues, no more than 3 contiguous glycine residues, no more than 4 contiguous glycine residues, or no more than 5 contiguous glycine residues. In some cases, a GS linker as provided herein may not be directly adjacent to another glycine residue at the 5′ end of the GS linker or at the 3′ end of the GS linker. A number of glycine residue(s) in the glycine linker may include all glycine residues disposed contiguously to another in the GS linker and directly adjacent to the serine residue of the GS linker.

In some embodiments, the polypeptide molecule as disclosed herein can comprise at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or about 1 glycine (G) linker(s). In some embodiments, the polypeptide molecule as disclosed herein can comprise at least about 1, at least about 2, at least about 3, at least about 4, at least about 5, at least about 6, at least about 7, at least about, at least about 9, at least about 10, at least about 11, at least about 12, at least about 13, at least about 14, at least about 15, at least about 20, or more G linker(s). In some cases, a G linker may be a contiguous polynucleotide sequence that comprise no more than 1 glycine residue, no more than 2 contiguous glycine residues, no more than 3 contiguous glycine residues, no more than 4 contiguous glycine residues, or no more than 5 contiguous glycine residues. In some cases, a G linker as provided herein may not be directly adjacent to another glycine residue at the 5′ end of the G linker or at the 3′ end of the G linker. A number of glycine residue(s) in the glycine linker may include all glycine residues disposed contiguously to another in the G linker.

### Polypeptide Domain

In some embodiments, a polypeptide molecule can comprise a polypeptide domain coupled to the polypeptide chain configured to effect (e.g., increasing or reducing) expression and/or activity level of the target gene (e.g., one or more target genes of Table 1), upon formation of a complex comprising the polypeptide molecule and a target polynucleotide sequence operatively coupled to the target gene of a cell. In some embodiments, the polypeptide molecule as disclosed herein can be introduced to a cell (e.g., a mammalian cell) to modulate (e.g., edit, activate, suppress) expression and/or activity level of the target gene.

In some embodiments, a polypeptide molecule can comprise a first polypeptide domain and a second polypeptide domain that are operatively coupled to one another via the polypeptide chain, wherein at least one of the first polypeptide domain and the second polypeptide domain of the polypeptide molecule can be at least a portion of an endonuclease, a gene modulator (e.g., a transcriptional activator or transcriptional suppressor), a gene editing moiety, a prime editor, an effector protein, or an effector protein (e.g., a protein, or functional domain or functional fragment thereof) configured to modulate (e.g., edit, activate, suppress) expression of a target gene in a cell.

### Polypeptide Domain—Gene Modulator

In some embodiments, a polypeptide molecule can comprise a gene modulator (e.g., a transcriptional activator or transcriptional repressor) coupled to the polypeptide chain disclosed herein. In some embodiments, a polypeptide molecule can comprise at least a portion of an endonuclease and a gene modulator that are coupled to one another via the polypeptide chain disclosed herein to modulate (e.g., edit, activate, suppress) gene expression of a target gene. In some embodiments, a polypeptide molecule can comprise a gene modulator and an additional gene modulator that are coupled to one another via the polypeptide chain disclosed herein to modulate (e.g., edit, activate, suppress) gene expression of a target gene. In some embodiments, the gene modulator and the additional gene modulator can be substantially the same. In some embodiments, the gene modulator and the additional gene modulator are substantially different. The gene modulator can be heterologous to the cell as provided herein.

In some embodiments, the gene modulator can be a histone epigenetic modifier (or a histone modifier). In some embodiments, the histone epigenetic modifier can modulate histones through methylation (e.g., a histone methylation modifier, such as an amino acid methyltransferase, e.g., KRAB). In some embodiments, the histone epigenetic modifier can modulate histones through acetylation. In some embodiments, the histone epigenetic modifier can modulate histones through phosphorylation. In some embodiments, the histone epigenetic modifier can modulate histones through ADP-ribosylation. In some embodiments, the histone epigenetic modifier can modulate histones through glycosylation. In some embodiments, the histone epigenetic modifier can modulate histones through SUMOylation. In some embodiments, the histone epigenetic modifier can modulate histones through ubiquitination. In some embodiments, the histone epigenetic modifier can modulate histones by remodeling histone structure, e.g., via an ATP hydrolysis-dependent process.

In some embodiments, the gene modulator can be a gene epigenetic modifier (or a gene modifier). In some embodiments, a gene modifier can modulate genes through methylation (e.g., a gene methylation modifier, such as a DNA methyltransferase or DNMT). In some embodiments, a gene modifier can modulate genes through acetylation.

In some embodiments, the gene modulator is from a family of related histone acetyltransferases. Non-limiting examples of histone acetyltransferases include GNAT subfamily, MYST subfamily, p300/CBP subfamily, HAT1 subfamily, GCN5, PCAF, Tip60, MOZ, MORF, MOF, HBO1, p300, CBP, HAT1, ATF-2, SRC1, and TAFII250.

In some embodiments, the gene modulator can comprise an epigenetic modifier. In some embodiments, the gene modulator comprises a histone epigenetic modifier (e.g., a histone lysine methyltransferase, a histone lysine demethylase, or a DNA methylase). Non-limiting examples of an epigenetic modifier can include EZH subfamily, Non-SET subfamily, Other SET subfamily, PRDM subfamily, SET1 subfamily, SET2 subfamily, SUV39 subfamily, SMYD subfamily, ASH1L, EHMT1, EHMT2, EZH1, EZH2, MLL, MLL2, MLL3, MLL4, MLL5, NSD1, NSD2, NSD3, PRDM1, PRDM10, PRDM11, PRDM12, PRDM13, PRDM14, PRDM15, PRDM16, PRDM2, PRDM4, PRDM5, PRDM6, PRDM7, PRDM8, PRDM9, SET1, SET1L, SET2L, SETD2, SETD3, SETD4, SETD5, SETD6, SETD7, SETD8, SETDB1, SETDB2, SETMAR, SUV39H1, SUV39H2, SUV420H1, SUV420H2, SMYD1, SMYD2, SMYD3, SMYD4, and SMYD5.

Examples of proteins (or fragments thereof) that can be used as a gene modulator to increase transcription include but are not limited to: transcriptional activators such as VP16, VP64, VP48, VP160, p65 subdomain (e.g., from NFkB), and activation domain of EDLL and/or TAL activation domain (e.g., for activity in plants), SET1A, SET1B, MLL1 to 5, ASH1, SMYD2, NSD1, JHDM2a/b, UTX, JMJD3, GCN5, PCAF, CBP, p300, TAF1, TIP60/PLIP, MOZMYST3, MORFMYST4, SRC1, ACTR, PI 60, CLOCK, Ten-Eleven Translocation (TET) dioxygenase 1 (TET1CD), TET1, DME, DML1, DML2, ROS1, etc. An additional example of such gene activating modulator is VP64-p65-Rta fusion polypeptide (VPR).

Examples of proteins (or fragments thereof) that can be used as a gene modulator to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g, for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARJD 1 A/RBP2, JARIDlB/PLU-1, JARID 1C/SMCX, JARIDID/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

### Polypeptide Domain—Endonuclease

In some embodiments, a polypeptide domain of the polypeptide molecule can comprise at least a portion of an endonuclease (e.g., enzymatically active Cas protein, enzymatically deactivated Cas protein, etc.) that is operatively coupled to the polypeptide chain disclosed herein.

Non-limiting examples of the at least the portion of the endonuclease as disclosed herein can include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcriptional activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof, any variant thereof and any fragment thereof.

In some embodiments, the at least the portion of the endonuclease as disclosed herein can have a length of at most about 1000 amino acids, at most about 950 amino acids, at most about 900 amino acids, at most about 850 amino acids, at most about 800 amino acids, at most about 750 amino acids, at most about 700 amino acids, at most about 650 amino acids, at most about 640 amino acids, at most about 630 amino acids, at most about 620 amino acids, at most about 610 amino acids, at most about 600 amino acids, at most about 590 amino acids, at most about 580 amino acids, at most about 570 amino acids, at most about 560 amino acids, at most about 550 amino acids, at most about 540 amino acids, at most about 530 amino acids, at most about 520 amino acids, at most about 510 amino acids, at most about 500 amino acids, at most about 490 amino acids, at most about 480 amino acids, at most about 470 amino acids, at most about 460 amino acids, at most about 450 amino acids, at most about 440 amino acids, at most about 430 amino acids, at most about 420 amino acids, at most about 410 amino acids, at most about 400 amino acids, at most about 350 amino acids, or at most about 300 amino acids.

In some embodiments, the at least the portion of the endonuclease as disclosed herein can be nuclease-deficient. In some embodiments, the at least the portion of the endonuclease can be a nuclease-null DNA binding protein that does not induce transcriptional activation or repression of a target DNA sequence unless it is present in a complex with one or more heterologous gene effectors of the disclosure. In some embodiments, the at least the portion of the endonuclease can be a nuclease-null DNA binding protein that can induce transcriptional activation or repression of a target DNA sequence (e.g., which can be altered or augmented by the presence of a heterologous gene effector as provided herein).

In some embodiments, the at least the portion of the endonuclease as disclosed herein can be an RNA nuclease such as an engineered (e.g., programmable or targetable) RNA nuclease. In some embodiments, the at least the portion of the endonuclease as disclosed herein can be a nuclease-null RNA binding protein that does not induce transcriptional activation or repression of a target RNA sequence unless it is present in a complex with one or more heterologous gene effectors of the disclosure. In some embodiments, the at least the portion of the endonuclease as disclosed herein can be a nuclease-null RNA binding protein that can induce transcriptional activation or repression of a target RNA sequence (e.g., which can be altered or augmented by the presence of a heterologous gene effector as provided herein).

Any suitable CRISPR/Cas system can be used. A CRISPR/Cas system can be referred to using a variety of naming systems. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpf1, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

The at least the portion of the endonuclease as disclosed herein can be an a Cas protein or derivative thereof, wherein the Cas protein or derivative thereof can be a Class 1 or a Class 2 Cas protein. A Cas protein can be a type I, type II, type III, type IV, type V Cas protein, or type VI Cas protein. A Cas protein can comprise one or more domains. Non-limiting examples of domains include, guide nucleic acid recognition and/or binding domain, nuclease domains (e.g., Dnase or Rnase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein or fragment thereof that is fused to other proteins or polypeptides. A Cas protein can be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csxl2), Cas10, CaslOd, Cas10, CaslOd, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cu1966, Cas13a, Cas13b, Cas13c, Cas13d, Cas13X, Cas13Y, Cas14 (e.g., Cas14 variants, such as Cas14a, Cas14b, Cas14c, etc.) and homologs or modified versions thereof.

A Cas protein or fragment or derivative thereof can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas nap hthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractorsalsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinellasuccinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein can be a wildtype or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein (e.g., a wild-type version of Cas14). A Cas protein can be a polypeptide with at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a wild type Cas protein. A Cas protein can be a polypeptide with at most about 5%, at most about 10%, at most about 20%, at most about 30%, at most about 40%, at most about 50%, at most about 60%, at most about 70%, at most about 80%, at most about 90%, or at most about 100% sequence identity and/or sequence similarity to a wild type exemplary Cas protein. Variants or fragments can comprise at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A Cas protein can comprise one or more nuclease domains, such as Dnase domains. For example, a Cas9 protein can comprise a RuvC-like nuclease domain and/or an HNH-like 20 nuclease domain. The in a nuclease active form of Cas9, RuvC and HNH domains can each cut a different strand of double-stranded DNA to make a double-stranded break in the DNA. A Cas protein can comprise only one nuclease domain (e.g., Cpf1 comprises RuvC domain but lacks HNH domain). In some embodiments, nuclease domains are absent. In some embodiments, nuclease domains are present but inactive or have reduced or minimal activity. In some embodiments, nuclease domains are present and active.

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. If all of the nuclease domains of a Cas protein (e.g., both RuvC and HNH nuclease domains in a Cas9 protein; RuvC nuclease domain in a Cpf1 protein) are deleted or mutated, the resulting Cas protein can have a reduced or no ability to cleave both strands of a double-stranded DNA. An example of a mutation that can convert a Cas9 protein into a nickase is a D10A (aspartate to alanine at position 10 of Cas9) mutation in the RuvC domain of Cas9 from S. pyogenes. H939A (histidine to alanine at amino acid position 839) or H840A (histidine to alanine at amino acid position 840) in the HNH domain of Cas9 from S. pyogenes can convert the Cas9 into a nickase. An example of a mutation that can convert a Cas9 protein into a dead Cas9 is a D10A (aspartate to alanine at position 10 of Cas9) mutation in the RuvC domain and H939A (histidine to alanine at amino acid position 839) or H840A (histidine to alanine at amino acid position 840) in the HNH domain of Cas9 from S. pyogenes.

A nuclease dead Cas protein can comprise one or more mutations relative to a wild-type version of the protein. The mutation can result in no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity in one or more of the plurality of nucleic acid-cleaving domains of the wild-type Cas protein. The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. The mutation can result in one or more of the plurality of nucleic acid-cleaving domains lacking the ability to cleave the complementary strand and the non-complementary strand of the target nucleic acid. The residues to be mutated in a nuclease domain can correspond to one or more catalytic residues of the nuclease. For example, residues in the wild type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated in a nuclease domain of a Cas protein can correspond to residues Asp10, His840, Asn854 and Asn856 in the wild type S. pyogenes Cas9 polypeptide, for example, as determined by sequence and/or structural alignment.

A Cas protein can comprise an amino acid sequence having at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a nuclease domain (e.g., RuvC domain, HNH domain) of a wild-type Cas protein.

A Cas protein, variant or derivative thereof can be modified to enhance regulation of gene expression by compositions and methods of the disclosure, e.g., as part of a complex disclosed herein. A Cas protein can be modified to increase or decrease nucleic acid binding affinity, nucleic acid binding specificity, enzymatic activity, and/or binding to other factors, such as heterodimerization or oligomerization domains and induce ligands. Cas proteins can also be modified to change any other activity or property of the protein, such as stability. For example, one or more nuclease domains of the Cas protein can be modified, deleted, or inactivated, or a Cas protein can be truncated to remove domains that are not essential for the desired function of the protein or complex. A Cas protein can be modified to modulate (e.g., enhance or reduce) the activity of the Cas protein for regulating gene expression by a complex of the disclosure that comprises a heterologous gene effector.

For example, a Cas protein can be coupled (e.g., fused, covalently coupled, or non-covalently coupled) to a heterologous gene effector (e.g., an epigenetic modification domain, a transcriptional activation domain, and/or a transcriptional repressor domain). A Cas protein can be coupled (e.g., fused, covalently coupled, or non-covalently coupled) to an oligomerization or dimerization domain as disclosed herein (e.g., a heterodimerization domain). A Cas protein can be coupled (e.g., fused, covalently coupled, or non-covalently coupled) to a heterologous polypeptide that provides increased or decreased stability. A Cas protein can be coupled (e.g., fused, covalently coupled, or non-covalently coupled) to a sequence that can facilitate degradation of the Cas protein or a complex containing the Cas protein, for example, a degron, such as an inducible degron (e.g., auxin inducible).

A Cas protein can be coupled (e.g., fused, covalently coupled, or non-covalently coupled) to any suitable number of partners, for example, at least one, at least two, at least three, at least four, or at least five, at least six, at least seven, or at least 8 partners. In some embodiments, a Cas protein of the disclosure is coupled (e.g., fused, covalently coupled, or non-covalently coupled) to at most two, at most three, at most four, at most five, at most six, at most seven, at most eight, or at most ten partners. In some embodiments, a Cas protein of the disclosure is coupled (e.g., fused, covalently coupled, or non-covalently coupled) to 1-5, 1-4, 1-3, 1-2, 2-5, 2-4, 2-3, 3-5, 3-4, or 4-5 partners. In some embodiments, a Cas protein of the disclosure is coupled (e.g., fused, covalently coupled, or non-covalently coupled) to one partner. In some embodiments, a Cas protein of the disclosure is coupled (e.g., fused, covalently coupled, or non-covalently coupled) to two partners. In some embodiments, a Cas protein of the disclosure is coupled (e.g., fused, covalently coupled, or non-covalently coupled) to three partners. In some embodiments, a Cas protein of the disclosure is coupled (e.g., fused, covalently coupled, or non-covalently coupled) to four partners. In some embodiments, a Cas protein of the disclosure is coupled (e.g., fused, covalently coupled, or non-covalently coupled) to five partners. In some embodiments, a Cas protein of the disclosure is coupled (e.g., fused, covalently coupled, or non-covalently coupled) to six partners.

A Cas protein can be provided in any form. For example, a Cas protein can be provided in the form of a protein, such as a Cas protein alone or complexed with a guide nucleic acid as a ribonucleoprotein. A Cas protein can be provided in a complex, for example, complexed with a guide nucleic acid and/or one or more heterologous gene effectors of the disclosure. A Cas protein can be provided in the form of a nucleic acid encoding the Cas protein, such as an RNA (e.g., messenger RNA (mRNA)), or DNA. The nucleic acid encoding the Cas protein can be codon optimized for efficient translation into protein in a particular cell or organism.

Nucleic acids encoding Cas proteins, fragments, or derivatives thereof can be stably integrated in the genome of a cell. Nucleic acids encoding Cas proteins can be operably linked to a promoter, for example, a promoter that is constitutively or inducibly active in the cell. Nucleic acids encoding Cas proteins can be operably linked to a promoter in an expression construct. Expression constructs can include any nucleic acid constructs capable of directing expression of a gene or other nucleic acid sequence of interest (e.g., a Cas gene) and which can transfer such a nucleic acid sequence of interest to a target cell.

In some embodiments, a Cas protein, variant or derivative thereof is a nuclease dead Cas (dCas) protein. A dead Cas protein can be a protein that lacks nucleic acid cleavage activity.

A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive, “deactivated” and/or “dead” (abbreviated by “d”). A dead Cas protein (e.g., dCas, dCas9, dCas14) can bind to a target polynucleotide but may not cleave or minimally cleaves the target polynucleotide. In some aspects, a dead Cas protein is a dead Cas14 protein. In some aspects, a dead Cas protein is a not a dead Cas14 protein.

A dCas polypeptide (e.g., dCas14 polypeptide) can associate with a single guide RNA (sgRNA) to activate or repress transcription of a target gene (e.g., target endogenous gene), for example, in combination with heterologous gene effector(s) disclosed herein. sgRNAs can be introduced into cells expressing the Cas or variant thereof, as provided herein. In some cases, such cells can contain one or more different sgRNAs that target the same target gene (e.g., target endogenous gene) or target gene regulatory sequence. In other cases, the sgRNAs target different nucleic acids in the cell (e.g., different target genes, different target gene regulatory sequences, or different sequences within the same target gene or target gene regulatory sequence).

Enzymatically inactive can refer to a nuclease that can bind to a nucleic acid sequence in a polynucleotide in a sequence-specific manner, but will not cleave a target polynucleotide or will cleave it at a substantially reduced frequency. An enzymatically inactive guide moiety can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a comparable wild-type activity (e.g., nucleic acid cleaving activity, wild-type Cas9 or wild-type Cas14 activity).

In some embodiments, the actuator moiety as disclosed herein does not contain a nucleic acid-guided targeting system. For example, the actuator moiety can include proteins that bind to a target gene (e.g., target endogenous gene) or target gene regulatory sequence based on protein structural features, such as certain nucleases disclosed herein.

In some embodiments, the wild-type Cas protein that the engineered Cas protein is a modification of has a native amino acid sequence with a length of less than 800 amino acids (e.g., Cas14 or a variant thereof). This relatively small size provides several advantages to the provided engineered Cas protein. For example, the small size can allow the Cas protein to be delivered to a host cell, e.g., a cell of a human patient, via a single adeno-associated virus delivery system that would be otherwise incapable of delivering a larger protein. The native amino acid sequence can have a length that is, for example, between 500 amino acids and 700 amino acids, e.g., between 500 amino acids and 620 amino acids, between 540 amino acids and 660 amino acids, between 560 amino acids and 680 amino acids, or between 580 amino acids and 700 amino acids. In terms of upper limits, the native amino acid sequence can have a length that is less than 700 amino acids, e.g., less than 680 amino acids, less than 660 amino acids, less than 640 amino acids, less than 620 amino acids, less than 600 amino acids, less than 580 amino acids, less than 560 amino acids, less than 540 amino acids, or less than 520 amino acids. In terms of lower limits, the native amino acid sequence can have an length that is greater than 500 amino acids, e.g., greater than 520 amino acids, greater than 540 amino acid, greater than 560 amino acids, greater than 580 amino acids, greater than 600 amino acids, greater than 620 amino acids, greater than 640 amino acids, greater than 660 amino acids, or greater than 700 amino acids. Larger lengths, e.g., greater than 700 amino acids, and smaller lengths, e.g., less than 500 amino acids, are also contemplated.

In some embodiments, the modified amino acid sequence of the engineered Cas protein includes one or more substitutions in the native amino acid sequence, where the positions of at least some of these substitutions follow one or more particular rules determined to have surprising advantages for the characteristics of the engineered Cas protein. For example, the particular substitution rules have been selected for their ability to produce engineered Cas proteins capable of functioning within eukaryotic cells. According to these particular rules, all or some of the one or more substitutions in the native amino acid sequence are either (1) within or no more than 30 amino acids downstream of a (D/E/K/N)X(R/F)(E/K)N motif of the native amino acid sequence, (2) at or no more than 30 amino acids upstream or downstream of position 241 of the native amino acid sequence, (3) at or no more than 30 amino acids upstream or downstream of position 516 of the native amino acid sequence, and/or (4) having an electrically charged amino acid in the native amino acid sequence.

In some embodiments, the native amino acid sequence includes a (D/E/K/N)X(R/F)(E/K)N motif, and the modified amino acid sequence includes one or more substitutions at positions within or no more than 30 amino acids upstream or downstream of the motif. The modified amino acid sequence can include, for example, one, two, three, four, five, six, seven, eight, nine, ten, or more than ten substitutions within or no more than 30 amino acids upstream or downstream of the motif. At least one of the one or more substitutions to the native amino acid sequence can be, for example, within or no more than 28 amino acids, 26 amino acids, 24 amino acids, 22 amino acids, 20 amino acids, 18 amino acids, 16 amino acids, 14 amino acids, 12 amino acids, or 10 amino acids of the motif. In some embodiments, at least one of the one or more substitutions within or no more than 30 amino acids upstream or downstream of the motif is to an R, A, S, or G. In some embodiments, each of the one or more substitutions within or no more than 30 amino acids upstream or downstream of the motif is independently to an R, A, S, or G. In some embodiments, all of the substitutions to the native amino acid sequence are at positions within or no more than 30 amino acids upstream or downstream of the motif.

Some embodiments of the present disclosure are directed to a Cas protein that is not a variant of CasX. Some embodiments of the present disclosure are directed to small Cas-based regulation of gene expression, such as at the transcriptional and/or translational level. Small Cas proteins can be targeted to DNA and/or RNA, and are much smaller than typical CRISPR effectors, e.g., ranging in size from about 400 amino acids to about 700 amino acids. The small size of can allow such Cas proteins and/or effector domain fusions thereof to be paired with a CRISPR array encoding multiple guide RNAs while remaining under the packaging size limit of various delivery vehicles, such as the versatile adeno-associated virus (AAV) delivery vehicle or non-viral delivery vehicles (e.g., lipid nanoparticles), for primary cell and in vivo delivery.

In some embodiments, the Cas protein or a variant thereof as provided herein (e.g., a variant of SEQ ID NO: 51 as disclosed herein) can have a size of at most about 800 amino acids, at most about 780 amino acids, at most about 760 amino acids, at most about 750 amino acids, at most about 740 amino acids, at most about 720 amino acids, at most about 700 amino acids, at most about 680 amino acids, at most about 660 amino acids, at most about 650 amino acids, at most about 640 amino acids, at most about 620 amino acids, at most about 600 amino acids, at most about 580 amino acids, at most about 560 amino acids, at most about 550 amino acids, at most about 540 amino acids, at most about 520 amino acids, at most about 500 amino acids, 480 amino acids, at most about 460 amino acids, at most about 450 amino acids, at most about 440 amino acids, at most about 420 amino acids, at most about 400 amino acids, or less.

Non-limiting examples of Cas protein are provided in Table 5. In some embodiments, the Cas protein or the deactivated Cas protein (dCas) as provided herein can comprise a polypeptide sequence (e.g., a consecutive polypeptide sequence) that exhibits at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or substantially about 100% sequence identity to the polypeptide sequence of one or more members selected from Table 5 (e.g., one or more members selected from the group consisting of SEQ ID Nos. 51-250).

In some embodiments, the Cas protein or a variant thereof, as provided herein, can comprise the amino acid sequence having at least about 60%, at least about 65%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to the amino acid sequence of SEQ ID NO: 51. Cas protein or a variant thereof, as provided herein, can comprise the amino acid sequence having at most about 100%, at most about 99%, at most about 98%, at most about 97%, at most about 96%, at most about 95%, at most about 94%, at most about 93%, at most about 92%, at most about 91%, at most about 90%, at most about 89%, at most about 88%, at most about 87%, at most about 86%, at most about 85%, at most about 84%, at most about 83%, at most about 82%, at most about 81%, at most about 80%, at most about 79%, at most about 78%, at most about 77%, at most about 76%, at most about 75%, at most about 74%, at most about 73%, at most about 72%, at most about 71%, at most about 70%, at most about 65%, at most about 60%, or less sequence identity to the amino acid sequence of SEQ ID NO: 51.

In some embodiments, a Cas protein or a variant thereof as disclosed herein can exhibit a greater cationic charge (e.g., at least 1, at least 2, at least 3, at least 4, at least 5, at least 6, at least 7, at least 8, at least 9, at least 10, or more cationic charges) as compared to the wild-type Cas14. The enhanced cationic charge can (i) enhance complexation of the Cas protein to the guide nucleic acid and/or (ii) enhance complexation of the Cas protein to the target polynucleotide sequence (e.g., endogenous target polynucleotide sequence). In some cases, the Cas protein can comprise one or more substitutions for the enhanced cationic charge. The one or more substitutions at positions within or no more than 30 amino acids upstream or downstream of the (D/E/K/N)X(R/F)(E/K)N motif of the native amino acid sequence can include, for example, one or more substitutions at positions selected from positions 143, 147, 151, and 154 of the native amino acid sequence. In some embodiments, e.g., when the native amino acid sequence is the sequence of SEQ ID NO: 51, the one or more substitutions include substitutions are at one or more positions selected from D143, T147, E151, and K154. In some embodiments, e.g., when the native amino acid sequence is the sequence of SEQ ID NO: 51, the one or more substitutions include one or more substitutions selected from D143R, T147R, E151R, and K154R.

In some embodiments, the modified amino acid sequence includes one or more substitutions at or no more than 30 amino acids upstream or downstream of position 241 of the native amino acid sequence. The modified amino acid sequence can include, for example, one, two, three, four, five, six, seven, eight, nine, ten, or more than ten substitutions within or no more than 30 amino acids upstream or downstream of position 241. At least one of the one or more substitutions to the native amino acid sequence can be, for example, within or no more than 28 amino acids, 26 amino acids, 24 amino acids, 22 amino acids, 20 amino acids, 18 amino acids, 16 amino acids, 14 amino acids, 12 amino acids, or 10 amino acids of position 241. In some embodiments, at least one of the one or more substitutions within or no more than 30 amino acids upstream or downstream of position 241 is to an R, A, S, or G. In some embodiments, each of the one or more substitutions within or no more than 30 amino acids upstream or downstream of position 241 is independently to an R, A, S, or G. In some embodiments, all of the substitutions to the native amino acid sequence are at positions within or no more than 30 amino acids upstream or downstream of position 241.

In some embodiments, e.g., when the native amino acid sequence is the sequence of SEQ ID NO: 51, the one or more substitutions at positions having an electrically charged amino include substitutions are at one or more positions selected from K11, K73, D143, E151, K154, E241, D318, K330, K457, E425, E462, E507, E527, and E528. In some embodiments, e.g., when the native amino acid sequence is the sequence of SEQ ID NO: 51, the one or more substitutions include one or more substitutions selected from K11R, K73R, D143R, E151R, K154R, E241R, D318R, K330R, E425N, K457R, E462R, E507R, E527R, and E528R. In some embodiments, the modified amino acid sequence includes a D143R substitution. In some embodiments, the only substitution in the modified amino acid sequence is D143R.

In some embodiments, the modified amino acid sequence of the engineered Cas protein includes two substitutions in the native amino acid sequence. In some embodiments, the modified amino acid sequence has exactly two substitutions in the native amino acid sequence. In some embodiments, the modified amino acid sequence includes two substitutions at positions selected from positions 143, 147, 151, 154, 241, 330, 425, 504, 507, 516, 519, 527, and 528. In some embodiments, the modified amino acid sequence has exactly two substitutions, where the exactly two substitutions are at positions selected from positions 143, 147, 151, 154, 241, 330, 425, 504, 507, 516, 519, 527, and 528. In some embodiments, e.g., when the native amino acid sequence is the sequence of SEQ ID NO: 51, the modified amino acid sequence includes two substitutions at positions selected from D143, T147, E151, K154, E241, K330, E425, N504, E507, N516, N519, E527, and E528. In some embodiments, e.g., when the native amino acid sequence is the sequence of SEQ ID NO: 51, the modified amino acid sequence has exactly two substitutions, where the exactly two substitutions are at positions selected from D143, T147, E151, K154, E241, K330, E425, N504, E507, N516, N519, E527, and E528.

In some embodiments, the modified amino acid sequence includes a substitution at position 143 and a substitution at a position selected from positions 147, 151, 154, 241, 330, 425, 504, 507, 516, 519, 527, and 528. In some embodiments, the modified amino acid includes a substitution at position 143 and exactly one other substitution, where the exactly one other substitution is at a position selected from positions 147, 151, 154, 241, 330, 425, 504, 507, 516, 519, 527, and 528. In some embodiments, e.g., when the native amino acid sequence is the sequence of SEQ ID NO: 51, the modified amino acid sequence includes a substitution at position D143 and a substitution at a position selected from positions T147, E151, K154, E241, K330R, E425N, N504, E507, N516, N519, E527, and E528. In some embodiments, e.g., when the native amino acid sequence is the sequence of SEQ ID NO: 51, the modified amino acid includes a substitution at position D143 and exactly one other substitution, where the exactly one other substitution is at a position selected from positions T147, E151, K154, E241, K330R, E425N, N504, E507, N516, N519, E527, and E528.

In some embodiments, e.g., when the native amino acid sequence is the sequence of SEQ ID NO: 51, the modified amino acid includes two substitutions selected from D143R, T147R, E151R, E151A, K154R, E241R, N504R, E507R, N516R, N519R, E527R, and E528R. In some embodiments, e.g., when the native amino acid sequence is the sequence of SEQ ID NO: 51, the modified amino acid includes exactly two substitutions, where the two substitutions are selected from D143R, T147R, E151R, E151A, K154R, E241R, N504R, E507R, N516R, N519R, E527R, and E528R. In some embodiments, e.g., when the native amino acid sequence is the sequence of SEQ ID NO: 51, the modified amino acid includes two substitutions selected from D143R/T147R, D143R/E15IR, D143R/E241R, D143R/E425N, D143R/E507R, D143R/N519R, D143R/E527R, D143R/E528R, D143R/R151S, D143/R151G, and D143R/E151A. In some embodiments, e.g., when the native amino acid sequence is the sequence of SEQ ID NO: 51, the modified amino acid includes exactly two substitutions, where the two substitutions are selected from D143R/T147R, D143R/E151R, D143R/E241R, D143R/E425N, D143R/E507R, D143R/N519R, D143R/E527R, D143R/E528R, D143R/R151S, D143/R151G, and D143R/E151A. In some embodiments, the modified amino acid sequence includes a D143R substitution and a T147R substitution. In some embodiments, the only substitutions in the modified amino acid sequence are a D143R substitution and a T147R substitution.

In some embodiments, provide herein is a dCas protein or a variant thereof where one or more amino acids of the parental Cas protein from which it is derived have been altered or otherwise removed to reduce or eliminate its nuclease activity. In some embodiments, the amino acids include D326 and D510 with respect to SEQ ID NO: 51. In some embodiments, one or both of D326 and D510 are substituted with an amino acid that reduces, substantially eliminates, or eliminates nuclease activity. In some embodiments, one or both of D326 and D510 are substituted with alanine (e.g., D326A and/or D510A based on SEQ ID NO: 51). In some embodiments, the dCas protein exhibits reduced or eliminated nuclease activity, or nuclease activity is absent or substantially absent within levels of detection.

In some embodiments, the dCas protein or a variant thereof comprises the amino acid sequence of SEQ ID NO: 51 or a variant thereof having at least about 70%, at least about 710%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to the amino acid sequence of SEQ ID NO: 51.

In some embodiments, according to any of the Cas protein systems described herein, the target nucleic acid is dsDNA. In such embodiments, dsDNA-targeting specificity is determined, at least in part, by two parameters: the gRNA spacer targeting a protospacer in the target dsDNA (the sequence in the target dsDNA corresponding to the gRNA spacer on the non-complementary DNA strand) and a short sequence, the protospacer-adjacent motif (PAM), located immediately 5′ (upstream) of the protospacer on the non-complementary DNA strand. In some embodiments, the PAM is 5′-TTTG-3′ or 5′-TTTA-3′. In some embodiments, the PAM is 5′-TTTG-3′. In some embodiments, the PAM is 5′-TTTA-3′. In some embodiments, the PAM can be a non-canonical PAM sequences (i.e., PAM sequence other than 5′-TTTG-3′ or 5′-TTTA-3′).

In some embodiments, according to any of the Cas protein systems described herein, the target nucleic acid is RNA. In such embodiments, RNA-targeting specificity is determined, at least in part, by the gRNA spacer targeting a protospacer-like sequence in the target RNA (the sequence in the target RNA complementary to the gRNA spacer), and is independent of the sequence located immediately 5′ (upstream) of the protospacer-like sequence. In some embodiments, the Cas protein system is also capable of targeting a dsDNA molecule, wherein the gRNA spacer is selected such that it targets a protospacer in the target dsDNA molecule having a PAM selected from 5′-TTTG-3′ and 5′-TTTA-3′. In other embodiments, the Cas protein system is incapable of targeting a dsDNA molecule, wherein the gRNA spacer is selected such that any protospacers in the dsDNA molecule targeted by the gRNA spacer do not have a PAM selected from 5′-TTTG-3′ and 5′-TTTA-3′.

In some embodiments, a actuator moiety can comprise a zinc finger nuclease (ZFN) or a variant, fragment, or derivative thereof. ZFN can refer to a fusion between a cleavage domain, such as a cleavage domain of Fokl, and at least one zinc finger motif (e.g., at least 2, at least 3, at least 4, or at least 5 zinc finger motifs) which can bind polynucleotides such as DNA and RNA. In some embodiments, a ZFN is used in a targeting moiety of the disclosure to bind a polynucleotide (e.g., target gene or target gene regulatory sequence), but the ZFN does not cleave or substantially does not cleave the polynucleotide, e.g., a nuclease dead ZFN. A ZFN or a variant, fragment, or derivative thereof can be fused to or associated with one of more heterologous gene effectors to form a complex of the disclosure.

The heterodimerization at certain positions in a polynucleotide of two individual ZFNs in certain orientation and spacing can lead to cleavage of the polynucleotide in nuclease-active ZFN. For example, a ZFN binding to DNA can induce a double-strand break in the DNA. In order to allow two cleavage domains to dimerize and cleave DNA, two individual ZFNs can bind opposite strands of DNA with their C-termini at a certain distance apart. In some cases, linker sequences between the zinc finger domain and the cleavage domain can require the 5′ edge of each binding site to be separated by about 5-7 base pairs. In some cases, a cleavage domain is fused to the C-terminus of each zinc finger domain.

In some embodiments, the cleavage domain of an actuator moiety comprising a ZFN comprises a modified form of a wild type cleavage domain. The modified form of the cleavage domain can comprise an amino acid change (e.g., deletion, insertion, or substitution) that reduces the nucleic acid-cleaving activity of the cleavage domain. For example, the modified form of the cleavage domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the corresponding wild-type cleavage domain. The modified form of the cleavage domain can have no substantial nucleic acid-cleaving activity. In some embodiments, the cleavage domain is enzymatically inactive.

In some embodiments, a actuator moiety can comprise a “TALEN” or “TAL-effector nuclease” or a variant, fragment, or derivative thereof. TALENs refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated Fok1 endonuclease or the catalytic domain of Fokl. In some embodiments, a TALEN is used in a targeting moiety of the disclosure to bind a polynucleotide (e.g., target gene or target gene regulatory sequence), but the TALEN does not cleave or substantially does not cleave the polynucleotide, e.g., a nuclease dead TALEN. A TALEN or a variant, fragment, or derivative thereof can be fused to or associated with one of more heterologous gene effectors to form a complex of the disclosure.

In some embodiments, a TALEN is engineered for reduced nuclease activity. In some embodiments, the nuclease domain of a TALEN comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive. A TALEN or a variant, fragment, or derivative thereof can be fused to or associated with one of more heterologous gene effectors to form a complex of the disclosure.

Several mutations to Fok1 have been made for its use in TALENs, which, for example, improve cleavage specificity or activity. Such TALENs can be engineered to bind any desired DNA sequence. TALENs can be used to generate gene modifications (e.g., nucleic acid sequence editing) by creating a double-strand break in a target DNA sequence, which in turn, undergoes NHEJ or HDR.

A TALE or a variant, fragment, or derivative thereof can be fused to or associated with one of more heterologous gene effectors to form a complex of the disclosure. In some embodiments, the transcription activator-like effector (TALE) protein is fused to a heterologous gene effector and does not comprise a nuclease. In some embodiments, a TALEN does not cleave or substantially does not cleave the polynucleotide, e.g., a nuclease dead TALE. A TALE or a variant, fragment, or derivative thereof can be fused to or associated with one of more heterologous gene effectors to form a complex of the disclosure.

In some embodiments, the complex of the transcription activator-like effector (TALE) protein and the heterologous gene effector is designed to function as a transcriptional activator. In some embodiments, the complex of the transcription activator-like effector (TALE) protein and the heterologous gene effector is designed to function as a transcriptional repressor. For example, the DNA-binding domain of the transcription activator-like effector (TALE) protein can be fused (e.g., linked) to one or more heterologous gene effectors that comprise transcriptional activation domains, or to one or more heterologous gene effectors that comprise transcriptional repression domains.

In some embodiments, a actuator moiety can comprise a meganuclease. Meganucleases generally refer to rare-cutting endonucleases or homing endonucleases that can be highly sequence specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. A nuclease-active meganuclease can generate a double-stranded break. In some embodiments, a meganuclease is used in a targeting moiety of the disclosure to bind a polynucleotide (e.g., target gene or target gene regulatory sequence), but the meganuclease does not cleave or substantially does not cleave the polynucleotide, e.g., a nuclease dead meganuclease. A meganuclease or a variant, fragment, or derivative thereof can be fused to or associated with one of more heterologous gene effectors to form a complex of the disclosure.

The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-Msol meganuclease, I-SceI meganuclease, variants thereof, derivatives thereof, and fragments thereof.

In some embodiments, the nuclease domain of a meganuclease comprises a modified form of a wild type nuclease domain. The modified form of the nuclease domain can comprise an amino acid change (e.g., deletion, insertion, or substitution) that reduces or eliminates the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive. In some embodiments, a meganuclease can bind DNA but cannot cleave the DNA. In some embodiments, a nuclease-inactive meganuclease is fused to or associated with one or more heterologous gene effectors to generate a complex of the disclosure.

In some embodiments, the heterologous polypeptide comprising the actuator moiety (e.g., and/or a complex comprising the heterologous polypeptide) can regulate expression and/or activity of a target gene (e.g., target endogenous gene). In some embodiments, the heterologous polypeptide and/or a complex thereof can edit the sequence of a nucleic acid (e.g., a gene and/or gene product). A nuclease-active Cas protein can edit a nucleic acid sequence by generating a double-stranded break or single-stranded break in a target polynucleotide.

In some embodiments, the heterologous polypeptide comprising the actuator moiety (e.g., and/or a complex comprising the heterologous polypeptide) can generate a double-strand break in a target polynucleotide, such as DNA. A double-strand break in DNA can result in DNA break repair which allows for the introduction of gene modification(s) (e.g., nucleic acid editing). In some embodiments, a nuclease induces site-specific single-strand DNA breaks or nicks, thus resulting in HDR.

A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided.

In some embodiments, the heterologous polypeptide comprising the actuator moiety (e.g., and/or a complex comprising the heterologous polypeptide) does not generate a double-strand break in a target polynucleotide, such as DNA. Binding of the heterologous polypeptide of the complex comprising the heterologous polypeptide (e.g., a complex comprising a dCas-effector and a guide RNA) without a nucleic acid break can be sufficient to regulate expression (e.g., enhance or suppress) of a target gene (e.g., endogenous target gene).

### Polypeptide Domain—Others

In some embodiments, a polypeptide molecule as described herein can comprise a gene editing moiety coupled to the polypeptide chain. In some embodiments, a polypeptide molecule can comprise at least a portion of an endonuclease and a gene editing moiety (e.g., a protein, or functional domain or functional fragment thereof) that are coupled to one another via a polypeptide chain that effects editing or mutating of a target polynucleotide (e.g., of a target gene). In some embodiments, the gene editing moiety (e.g., a protein, or functional domain or functional fragment thereof) that effects editing or mutating of a target polynucleotide is a gene editing moiety that changes one or more nucleotides to a different nucleotide. In some embodiments, the gene editing moiety (e.g., a protein, or functional domain or functional fragment thereof) that effects editing or mutating of a target polynucleotide is a gene editing moiety that changes a guanine (G) to a different nucleotide. In some embodiments, the gene editing moiety that effects editing or mutating of a target polynucleotide changes a guanine (G) to a cytosine I. In some embodiments, the gene editing moiety that effects editing or mutating of a target polynucleotide changes a guanine (G) to a thymine (T). In some embodiments, the gene editing moiety that effects editing or mutating of a target polynucleotide changes a guanine (G) to an adenine (A). In some embodiments, the gene editing moiety that effects editing or mutating of a target polynucleotide changes a guanine (G) to an uracil (U). In some embodiments, the gene editing moiety that effects editing or mutating of a target polynucleotide changes a guanine (G) to an inosine (I). In some embodiments, the gene editing moiety (e.g., a protein, or functional domain or functional fragment thereof) that effects editing or mutating of a target polynucleotide sequence is a gene editing moiety that changes a cytosine (C) to a different nucleotide. In some embodiments, the gene editing moiety that effects editing or mutating of a target polynucleotide changes a cytosine (C) to a guanine (G). In some embodiments, the gene editing moiety that effects editing or mutating of a target polynucleotide changes a cytosine (C) to a thymine (T). In some embodiments, the gene editing moiety that effects editing or mutating of a target polynucleotide changes a cytosine (C) to an adenine (A). In some embodiments, the gene editing moiety that effects editing or mutating of a target polynucleotide changes a cytosine (C) to an uracil (U). In some embodiments, the gene editing moiety that effects editing or mutating of a target polynucleotide changes a cytosine (C) to an inosine (I). In some embodiments, the gene editing moiety (e.g., a protein, or functional domain or functional fragment thereof) that effects editing or mutating of a target polynucleotide sequence is a gene editing moiety that changes a thymine (T) to a different nucleotide. In some embodiments, the gene editing moiety that effects editing or mutating of a target polynucleotide changes a thymine (T) to a cytosine (C). In some embodiments, the gene editing moiety that effects editing or mutating of a target polynucleotide changes a thymine (T) to a guanine (G). In some embodiments, the gene editing moiety that effects editing or mutating of a target polynucleotide changes a thymine (T) to an adenine (A). In some embodiments, the gene editing moiety that effects editing or mutating of a target polynucleotide changes a thymine (T) to an uracil (U). In some embodiments, the gene editing moiety that effects editing or mutating of a target polynucleotide changes a thymine (T) to an inosine (I). In some embodiments, the gene editing moiety (e.g., a protein, or functional domain or functional fragment thereof) that effects editing or mutating of a target polynucleotide sequence is a gene editing moiety that changes an adenine (A) to a different nucleotide. In some embodiments, the gene editing moiety that effects editing or mutating of a target polynucleotide changes an adenine (A) to a cytosine (C). In some embodiments, the gene editing moiety that effects editing or mutating of a target polynucleotide changes an adenine (A) to a thymine (T). In some embodiments, the gene editing moiety that effects editing or mutating of a target polynucleotide changes an adenine (A) to a guanine (G). In some embodiments, the gene editing moiety that effects editing or mutating of a target polynucleotide changes an adenine (A) to an uracil (U). In some embodiments, the gene editing moiety that effects editing or mutating of a target polynucleotide changes an adenine (A) to an inosine (I). In some embodiments, the gene editing moiety (e.g., a protein, or functional domain or functional fragment thereof) that effects editing or mutating of a target polynucleotide sequence is a gene editing moiety that changes an uracil (U) to a different nucleotide. In some embodiments, the gene editing moiety that effects editing or mutating of a target polynucleotide changes an uracil (U) to a cytosine (C). In some embodiments, the gene editing moiety that effects editing or mutating of a target polynucleotide changes an uracil (U) to a thymine (T). In some embodiments, the gene editing moiety that effects editing or mutating of a target polynucleotide changes an uracil (U) to an adenine (A). In some embodiments, the gene editing moiety that effects editing or mutating of a target polynucleotide changes an uracil (U) to a guanine (G). In some embodiments, the gene editing moiety that effects editing or mutating of a target polynucleotide changes an uracil (U) to an inosine (I). In some embodiments, the gene editing moiety (e.g., a protein, or functional domain or functional fragment thereof) that effects editing or mutating of a target polynucleotide sequence is a gene editing moiety that changes an inosine (I) to a different nucleotide. In some embodiments, the gene editing moiety that effects editing or mutating of a target polynucleotide changes an inosine (I) to a cytosine (C). In some embodiments, the gene editing moiety that effects editing or mutating of a target polynucleotide changes an inosine (I) to a thymine (T). In some embodiments, the gene editing moiety that effects editing or mutating of a target polynucleotide changes an inosine (I) to an adenine (A). In some embodiments, the gene editing moiety that effects editing or mutating of a target polynucleotide changes an inosine (I) to an uracil (U). In some embodiments, the gene editing moiety that effects editing or mutating of a target polynucleotide changes an inosine (I) to a guanine (G).

In some embodiments, a polypeptide molecule as described herein can comprise at least a portion of an endonuclease and one or more polypeptide domains (e.g., a gene editing moiety) that are operatively coupled to one another via a polypeptide chain(s) that introduces one or more point mutations into a target polynucleotide, upon formation of a complex comprising the polypeptide molecule and a target polynucleotide sequence operatively coupled to a target gene of a cell. In some embodiments, a polypeptide molecule as described herein can comprise at least a portion of an endonuclease and one or more polypeptide domains (e.g., a gene editing moiety) that are operatively coupled to one another via a polypeptide chain(s) that introduces one or more deletions (e.g., of one or more nucleotides) into a target polynucleotide, upon formation of a complex comprising the polypeptide molecule and a target polynucleotide sequence operatively coupled to a target gene of a cell. In some embodiments, a polypeptide molecule as described herein can comprise at least a portion of an endonuclease and one or more polypeptide domains (e.g., a gene editing moiety) that are operatively coupled to one another via a polypeptide chain(s) that introduces one or more insertions (e.g., of one or more nucleotides) into a target polynucleotide, upon formation of a complex comprising the polypeptide molecule and a target polynucleotide sequence operatively coupled to a target gene of a cell. In some embodiments, a polypeptide molecule as described herein can comprise at least a portion of an endonuclease and one or more polypeptide domains (e.g., a gene editing moiety) that are operatively coupled to one another via a polypeptide chain(s) that introduces one or more inversions (e.g., of two or more nucleotides) in a target polynucleotide, upon formation of a complex comprising the polypeptide molecule and a target polynucleotide sequence operatively coupled to a target gene of a cell. In some embodiments, a polypeptide molecule as described herein can comprise at least a portion of an endonuclease and one or more polypeptide domains (e.g., a gene editing moiety) that are operatively coupled to one another via a polypeptide chain(s) that introduces one or more translocations (e.g., of one or more nucleotides) in a target polynucleotide, upon formation of a complex comprising the polypeptide molecule and a target polynucleotide sequence operatively coupled to a target gene of a cell.

In some embodiments, the gene editing moiety may be a base-editing protein or a base-editing enzyme. In some embodiments, the base-editing protein or base-editing enzyme is a deaminase. In some embodiments, the deaminase is a cytidine deaminase. In some embodiments, the cytidine deaminase catalyzes the reaction of a cytosiI (C) to an uracil (U), which has the base-pairing properties of thymine. In some embodiments, for example where the polynucleotide is double-stranded (e.g., double-stranded DNA), the uridine base can then be substituted with a thymidine base (e.g., by cellular repair machinery) to give rise to a C·G to a T·A transition. In some embodiments, the deaminase is an adenine deaminase. In some embodiments, the adenine deaminase catalyzes the reaction of an adenosine (A) to an inosine (I). Non-limiting examples of deaminases suitable for use herein include, without limitation, APOBEC 1 deaminase, APOBEC2 deaminase, APOBEC3 deaminase, APOBEC3A deaminase, APOBEC3B deaminase, APOBEC3C deaminase, APOBEC3D deaminase, APOBEC3E deaminase, APOBEC3F deaminase, APOBEC3G deaminase, APOBEC3H deaminase, APOBEC4 deaminase, activation-induced cytidine deaminase (AID), adenosine deaminase 1 (ADAR1), adenosine deaminase 2 (ADAR2), adenosine deaminase 3 (ADAR3), or TadA.

In some embodiments, a polypeptide molecule as described herein can comprise a prime editor coupled to a polypeptide chain. In some embodiments, a polypeptide molecule can comprise at least a portion of an endonuclease, a reverse transcriptase enzyme (e.g., an engineered M-MLV reverse transcriptase), and a prime editing RNA (pegRNA) that are operatively coupled to one another via one or more polypeptide chains, one or more spacers, or one or more linkers. In such cases, the at least the portion of the endonuclease can comprise nickase activity. In some embodiments, prime editing may be used to mediate targeted insertions, deletions, or base-to-base conversions.

In some embodiments, the polypeptide molecule described herein can comprise an effector protein (e.g., a protein, or functional domain or functional fragment thereof) coupled to a polypeptide chain that methylates a target substrate. In some embodiments, the polypeptide molecule described herein can comprise at least a portion of an endonuclease coupled to an effector protein (e.g., a protein, or functional domain or functional fragment thereof) via a polypeptide chain. In some embodiments, the effector protein (e.g., a protein, or functional domain or functional fragment thereof) that methylates a target substrate is a methyltransferase. In some embodiments, the methyltransferase is a DNA methyltransferase, a histone methyltransferase, or an RNA methyltransferase. In some embodiments, the DNA methyltransferase is DNMT1 or DNMT3.

In some embodiments, the polypeptide molecule described herein can comprise an effector protein (e.g., a protein, or functional domain or functional fragment thereof) coupled to a polypeptide chain that has demethylase activity (e.g., can remove methyl groups from nucleic acids, proteins, or other molecules). In some embodiments, the polypeptide molecule described herein can comprise at least a portion of an endonuclease coupled to an effector protein (e.g., a protein, or functional domain or functional fragment thereof) via a polypeptide chain. In some embodiments, the effector protein (e.g., a protein, or functional domain or functional fragment thereof) that has demethylase activity is a histone lysine demethylase, such as, but not limited to KDM1, KDM2, KDM3, KDM4, KDM5, and KDM6.

In some embodiments, the polypeptide molecule described herein can comprise an effector protein (e.g., a protein, or functional domain or functional fragment thereof) coupled to a polypeptide chain that has dismutase activity. In some embodiments, the polypeptide molecule described herein can comprise at least a portion of an endonuclease coupled to an effector protein (e.g., a protein, or functional domain or functional fragment thereof) via a polypeptide chain. In some embodiments, the effector protein (e.g., a protein, or functional domain or functional fragment thereof) that has dismutase activity is superoxide dismutase, formaldehyde dismutase, or chlorite dismutase.

In some embodiments, the polypeptide molecule described herein can comprise an effector protein (e.g., a protein, or functional domain or functional fragment thereof) coupled to a polypeptide chain that has alkylation activity. In some embodiments, the polypeptide molecule described herein can comprise at least a portion of an endonuclease coupled to an effector protein (e.g., a protein, or functional domain or functional fragment thereof) via a polypeptide chain. In some embodiments, the effector protein (e.g., a protein, or functional domain or functional fragment thereof) that has alkylation activity is a prenyltransferase, a terpene cyclase, or a terpene synthase.

In some embodiments, the polypeptide molecule described herein can comprise an effector protein (e.g., a protein, or functional domain or functional fragment thereof) coupled to a polypeptide chain that has depurination activity. In some embodiments, the polypeptide molecule described herein can comprise at least a portion of an endonuclease coupled to an effector protein (e.g., a protein, or functional domain or functional fragment thereof) via a polypeptide chain. In some embodiments, the effector protein (e.g., a protein, or functional domain or functional fragment thereof) that has depurination activity is DNA glycosylase.

In some embodiments, the polypeptide molecule described herein can comprise an effector protein (e.g., a protein, or functional domain or functional fragment thereof) coupled to a polypeptide chain that has oxidation activity. In some embodiments, the polypeptide molecule described herein can comprise at least a portion of an endonuclease coupled to an effector protein (e.g., a protein, or functional domain or functional fragment thereof) via a polypeptide chain. In some embodiments, the effector protein (e.g., a protein, or functional domain or functional fragment thereof) that has oxidation activity is a peroxidase or an oxidase.

In some embodiments, the polypeptide molecule described herein can comprise an effector protein (e.g., a protein, or functional domain or functional fragment thereof) coupled to a polypeptide chain that has pyrimidine dimer forming activity. In some embodiments, the polypeptide molecule described herein can comprise at least a portion of an endonuclease coupled to an effector protein (e.g., a protein, or functional domain or functional fragment thereof) via a polypeptide chain.

In some embodiments, the polypeptide molecule described herein can comprise an effector protein (e.g., a protein, or functional domain or functional fragment thereof) coupled to a polypeptide chain that has integrase activity. In some embodiments, the polypeptide molecule described herein can comprise at least a portion of an endonuclease coupled to an effector protein (e.g., a protein, or functional domain or functional fragment thereof) via a polypeptide chain. In some embodiments, the effector protein (e.g., a protein, or functional domain or functional fragment thereof) that has integrase activity is retroviral integrase or HIV integrase.

In some embodiments, the polypeptide molecule described herein can comprise an effector protein (e.g., a protein, or functional domain or functional fragment thereof) coupled to a polypeptide chain that has transposase activity. In some embodiments, the polypeptide molecule described herein can comprise at least a portion of an endonuclease coupled to an effector protein (e.g., a protein, or functional domain or functional fragment thereof) via the polypeptide chain. In some embodiments, the effector protein (e.g., a protein, or functional domain or functional fragment thereof) that has transposase activity is ty1, Mariner transposase, Tn3, transposase (Tnp) Tn5, or Tn7 transposon.

In some embodiments, the polypeptide molecule described herein can comprise an effector protein (e.g., a protein, or functional domain or functional fragment thereof) coupled to a polypeptide chain that has recombinase activity. In some embodiments, the polypeptide molecule described herein can comprise at least a portion of an endonuclease coupled to an effector protein (e.g., a protein, or functional domain or functional fragment thereof) via the polypeptide chain. In some embodiments, the effector protein (e.g., a protein, or functional domain or functional fragment thereof) that has recombinase activity is tyrosine recombinase, Rad51 recombinase, RecA recombinase, or Dmc1 recombinase.

In some embodiments, the polypeptide molecule described herein can comprise an effector protein (e.g., a protein, or functional domain or functional fragment thereof) coupled to a polypeptide chain that has polymerase activity. In some embodiments, the polypeptide molecule described herein can comprise at least a portion of an endonuclease coupled to an effector protein (e.g., a protein, or functional domain or functional fragment thereof) that has polymerase activity via the polypeptide chain. In some embodiments, the effector protein (e.g., a protein, or functional domain or functional fragment thereof) that has polymerase activity is DNA polymerase, RNA polymerase, reverse transcriptase, or RdRp replicase.

In some embodiments, the polypeptide molecule described herein can comprise an effector protein (e.g., a protein, or functional domain or functional fragment thereof) coupled to a polypeptide chain that has ligase activity. In some embodiments, the polypeptide molecule described herein can comprise at least a portion of an endonuclease coupled to an effector protein (e.g., a protein, or functional domain or functional fragment thereof) that has ligase activity via the polypeptide chain. In some embodiments, the effector protein (e.g., a protein, or functional domain or functional fragment thereof) that has ligase activity is ubiquitin ligases, glutamate-cysteine ligase, aminoacyl tRNA synthetase, succinyl coenzyme A synthetase, acetyl-CoA synthetase, pyruvate carboxylase, acetyl-CoA carboxylase, propionyl-CoA carboxylase, methylcrotonyl-CoA carboxylase, DNA ligase, magnesium chelatase, cobalt chelatase, or DNA synthetase.

In some embodiments, the polypeptide molecule described herein can comprise an effector protein (e.g., a protein, or functional domain or functional fragment thereof) coupled to a polypeptide chain that has helicase activity. In some embodiments, the polypeptide molecule described herein can comprise at least a portion of an endonuclease coupled to an effector protein (e.g., a protein, or functional domain or functional fragment thereof) that has helicase activity via the polypeptide chain. In some embodiments, the effector protein (e.g., a protein, or functional domain or functional fragment thereof) that has helicase activity is a DNA helicase, an RNA helicase, chromodomain helicase, or DEAD box/DEAD/DEAH box helicase.

In some embodiments, the polypeptide molecule described herein can comprise an effector protein (e.g., a protein, or functional domain or functional fragment thereof) coupled to a polypeptide chain that has photolyase activity. In some embodiments, the polypeptide molecule described herein can comprise at least a portion of an endonuclease coupled to an effector protein (e.g., a protein, or functional domain or functional fragment thereof) that has photolyase activity via the polypeptide chain. In some embodiments, the effector protein (e.g., a protein, or functional domain or functional fragment thereof) that has is photoreactivating enzyme, DNA photolyase, DNA-photoreactivating enzyme, DNA cyclobutane dipyrimidine photolyase, DNA photolyase, deoxyribonucleic photolyase, deoxyribodipyrimidine photolyase, photolyase, PRE, PhrB photolyase, deoxyribonucleic cyclobutane dipyrimidine photolyase, phr A photolyase, dipyrimidine photolyase (photosensitive), or deoxyribonucleate pyrimidine dimer lyase (photosensitive).

In some embodiments, the polypeptide molecule described herein can comprise an effector protein (e.g., a protein, or functional domain or functional fragment thereof) coupled to a polypeptide chain that has glycosylase activity. In some embodiments, the polypeptide molecule described herein can comprise at least a portion of an endonuclease coupled to an effector protein (e.g., a protein, or functional domain or functional fragment thereof) that has glycosylase activity via the polypeptide chain. In some embodiments, the effector protein (e.g., a protein, or functional domain or functional fragment thereof) that has glycosylase activity is N-methylpurine DNA glycosylase, UNG, hOGG1, hNTH1, hNEIL1, hMYH, hSMUG1, TDG, MBD4, Mag1, Ung1, Ogg1, Ntg1, AlkE, Ntg2, hNEIL2, hNEIL3, AlkC, AlkD, MutY, Nei, Nth, Fpg, or UDG.

In some embodiments, the polypeptide molecule described herein can comprise an effector protein (e.g., a protein, or functional domain or functional fragment thereof) coupled to a polypeptide chain that has acetyltransferase activity. In some embodiments, the polypeptide molecule described herein can comprise at least a portion of an endonuclease coupled to an effector protein (e.g., a protein, or functional domain or functional fragment thereof) that has acetyltransferase activity via the polypeptide chain. In some embodiments, the effector protein (e.g., a protein, or functional domain or functional fragment thereof) that has acetyltransferase activity is CBP histone acetyltransferase, choline acetyltransferase, chloramphenicol acetyltransferase, serotonin N-acetyltransferase, NatA Acetyltransferase, or NatB acetyltransferase.

In some embodiments, the polypeptide molecule described herein can comprise an effector protein (e.g., a protein, or functional domain or functional fragment thereof) coupled to a polypeptide chain that has deacetylase activity. In some embodiments, the polypeptide molecule described herein can comprise at least a portion of an endonuclease coupled to an effector protein (e.g., a protein, or functional domain or functional fragment thereof) that has deacetylase activity via the polypeptide chain. In some embodiments, the effector protein (e.g., a protein, or functional domain or functional fragment thereof) that has deacetylase activity is HDAC-1, HDAC-2, HDAC-3, HDAC-4, HDAC-5, HDAC-6, HDAC-7, or HDAC-8.

In some embodiments, the polypeptide molecule described herein can comprise an effector protein (e.g., a protein, or functional domain or functional fragment thereof) coupled to a polypeptide chain that has kinase activity. In some embodiments, the polypeptide molecule described herein can comprise at least a portion of an endonuclease coupled to an effector protein (e.g., a protein, or functional domain or functional fragment thereof) that has kinase activity via the polypeptide chain. In some embodiments, the effector protein (e.g., a protein, or functional domain or functional fragment thereof) that has kinase activity is Ca2+/calmodulin-dependent protein kinase, cyclin-dependent kinase, nucleoside-diphosphate kinase, a phosphatidylinositol phosphate kinase, thymidine kinase, thymidylate kinase, or wall-associated kinase.

In some embodiments, the polypeptide molecule described herein can comprise an effector protein (e.g., a protein, or functional domain or functional fragment thereof) coupled to a polypeptide chain that has phosphatase activity. In some embodiments, the polypeptide molecule described herein can comprise at least a portion of an endonuclease coupled to an effector protein (e.g., a protein, or functional domain or functional fragment thereof) that has phosphatase activity via the polypeptide chain. In some embodiments, the effector protein (e.g., a protein, or functional domain or functional fragment thereof) that has phosphatase activity is acid phosphatase, alkaline phosphatase, endonuclease/exonuclease/phosphatase family, kinase, phosphatome, phosphotransferase, protein phosphatase, or protein phosphatase 2.

In some embodiments, the polypeptide molecule described herein can comprise an effector protein (e.g., a protein, or functional domain or functional fragment thereof) coupled to a polypeptide chain that has ubiquitin ligase activity. In some embodiments, the polypeptide molecule described herein can comprise at least a portion of an endonuclease coupled to an effector protein (e.g., a protein, or functional domain or functional fragment thereof) that has ubiquitin ligase activity via the polypeptide chain. In some embodiments, the effector protein (e.g., a protein, or functional domain or functional fragment thereof) that has ubiquitin ligase activity is E3A, mdm2, Anaphase-promoting complex (APC), UBR5 (EDD1), SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HERC5, HERC6, HUWEl, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL, WWP1, WWP2, Parkin, or MKRN1.

In some embodiments, the polypeptide molecule described herein can comprise an effector protein (e.g., a protein, or functional domain or functional fragment thereof) coupled to a polypeptide chain that has deubiquitinating activity. In some embodiments, the polypeptide molecule described herein can comprise at least a portion of an endonuclease coupled to an effector protein (e.g., a protein, or functional domain or functional fragment thereof) that has deubiquitinating activity via the polypeptide chain. In some embodiments, the effector protein (e.g., a protein, or functional domain or functional fragment thereof) that has deubiquitinating activity is a deubiquitinating peptidase, a deubiquitinating isopeptidase, a deubiquitinase, a ubiquitin protease, a ubiquitin hydrolase, or a ubiquitin isopeptidase.

In some embodiments, the polypeptide molecule described herein can comprise an effector protein (e.g., a protein, or functional domain or functional fragment thereof) coupled to a polypeptide chain that has adenylation activity. In some embodiments, the polypeptide molecule described herein can comprise at least a portion of an endonuclease coupled to an effector protein (e.g., a protein, or functional domain or functional fragment thereof) that has adenylation activity via the polypeptide chain. In some embodiments, the effector protein (e.g., a protein, or functional domain or functional fragment thereof) that has adenylation activity is carboxylic acid reductase.

In some embodiments, the polypeptide molecule described herein can comprise an effector protein (e.g., a protein, or functional domain or functional fragment thereof) coupled to a polypeptide chain that has deadenylation activity. In some embodiments, the polypeptide molecule described herein can comprise at least a portion of an endonuclease coupled to an effector protein (e.g., a protein, or functional domain or functional fragment thereof) that has deadenylation activity via the polypeptide chain. In some embodiments, the effector protein (e.g., a protein, or functional domain or functional fragment thereof) that has deadenylation activit' is 5′-deadenylase, CNOT6 deadenylase, CNOT6L deadenylase, or CCR4-NOT deadenylase,

In some embodiments, the polypeptide molecule described herein can comprise an effector protein (e.g., a protein, or functional domain or functional fragment thereof) coupled to a polypeptide chain that has SUMOylating activity. In some embodiments, the polypeptide molecule described herein can comprise at least a portion of an endonuclease coupled to an effector protein (e.g., a protein, or functional domain or functional fragment thereof) that has SUMOylating activity via the polypeptide chain. In some embodiments, the effector protein (e.g., a protein, or functional domain or functional fragment thereof) that has SUMOylating activity is small ubiquitin-related modifier (SUMO-1), SUMO-2, or SUMO-3.

In some embodiments, the polypeptide molecule described herein can comprise an effector protein (e.g., a protein, or functional domain or functional fragment thereof) coupled to a polypeptide chain that has deSUMOylating activity. In some embodiments, the polypeptide molecule described herein can comprise at least a portion of an endonuclease coupled to an effector protein (e.g., a protein, or functional domain or functional fragment thereof) that has deSUMOylating activity via the polypeptide chain. In some embodiments, the effector protein (e.g., a protein, or functional domain or functional fragment thereof) that has deSUMOylating activity is SENP1, SENP2, SENP3, or SENP5.

In some embodiments, the polypeptide molecule described herein can comprise an effector protein (e.g., a protein, or functional domain or functional fragment thereof) coupled to a polypeptide chain that has ribosylation activity. In some embodiments, the polypeptide molecule described herein can comprise at least a portion of an endonuclease coupled to an effector protein (e.g., a protein, or functional domain or functional fragment thereof) that has ribosylation activity via the polypeptide chain. In some embodiments, the effector protein (e.g., a protein, or functional domain or functional fragment thereof) that has ribosylation activity is a mono(ADP-ribosyl)transferase, a poly(ADP-ribose)polymerase, or histone ribosylase.

In some embodiments, the polypeptide molecule described herein can comprise an effector protein (e.g., a protein, or functional domain or functional fragment thereof) coupled to a polypeptide chain that has deribosylation activity. In some embodiments, the polypeptide molecule described herein can comprise at least a portion of an endonuclease coupled to an effector protein (e.g., a protein, or functional domain or functional fragment thereof) that has deribosylation activity via the polypeptide chain. In some embodiments, the effector protein (e.g., a protein, or functional domain or functional fragment thereof) that has deribosylation activity is histone lysine deribosylase or ADP-ribose deribosylase.

In some embodiments, the polypeptide molecule described herein can comprise an effector protein (e.g., a protein, or functional domain or functional fragment thereof) coupled to a polypeptide chain that has myristoylation activity. In some embodiments, the polypeptide molecule described herein can comprise at least a portion of an endonuclease coupled to an effector protein (e.g., a protein, or functional domain or functional fragment thereof) that has myristoylation activity via the polypeptide chain. In some embodiments, the effector protein (e.g., a protein, or functional domain or functional fragment thereof) that has myristoylation activity is N-myristoltransferase (NMT) 1, N-myristoltransferase (NMT) 2, or glycylpeptide N-tetradecanoyltransferase.

In some embodiments, the polypeptide molecule described herein can comprise an effector protein (e.g., a protein, or functional domain or functional fragment thereof) coupled to a polypeptide chain that has remodeling activity. In some embodiments, the polypeptide molecule described herein can comprise at least a portion of an endonuclease coupled to an effector protein (e.g., a protein, or functional domain or functional fragment thereof) that has remodeling activity via the polypeptide chain. In some embodiments, the effector protein (e.g., a protein, or functional domain or functional fragment thereof) that has remodeling activity is a histone acetyltransferase (HAT), a deacetylase, or a methyltransferase.

In some embodiments, the polypeptide molecule described herein can comprise an effector protein (e.g., a protein, or functional domain or functional fragment thereof) coupled to a polypeptide chain that has protease activity. In some embodiments, the polypeptide molecule described herein can comprise at least a portion of an endonuclease coupled to an effector protein (e.g., a protein, or functional domain or functional fragment thereof) that has protease activity via the polypeptide chain. In some embodiments, the effector protein (e.g., a protein, or functional domain or functional fragment thereof) that has protease activity is trypsin, chymotrypsin, elastase, papain, bromelain, a serine protease, a cysteine protease, a threonine protease, an aspartic protease, a glutamic protease, a metalloprotease, or an asparagine peptide lyase.

In some embodiments, the polypeptide molecule described herein can comprise an effector protein (e.g., a protein, or functional domain or functional fragment thereof) coupled to a polypeptide chain that has oxidoreductase activity. In some embodiments, the polypeptide molecule described herein can comprise at least a portion of an endonuclease coupled to an effector protein (e.g., a protein, or functional domain or functional fragment thereof) that has oxidoreductase activity via the polypeptide chain. In some embodiments, the effector protein (e.g., a protein, or functional domain or functional fragment thereof) that has oxidoreductase activity is donor dehydrogenase, peroxidase, reductase, dehydrogenase, oxidase, oxygenase, hydroxylase, luciferase, DMSO reductase, glucose oxidase, L-gulonolactone oxidase, thiamine oxidase, xanthine oxidase, acetaldehyde dehydrogenase, pyruvate dehydrogenase, oxoglutarate dehydrogenase, monoamine oxidase, biliverdin reductase, dihydrofolate reductase, methylenetetrahydrofolate reductase, sarcosine oxidase, or dihydrobenzophenanthridine oxidase.

In some embodiments, the polypeptide molecule described herein can comprise an effector protein (e.g., a protein, or functional domain or functional fragment thereof) coupled to a polypeptide chain that has transferase activity. In some embodiments, the polypeptide molecule described herein can comprise at least a portion of an endonuclease coupled to an effector protein (e.g., a protein, or functional domain or functional fragment thereof) that has transferase activity via the polypeptide chain. In some embodiments, the effector protein (e.g., a protein, or functional domain or functional fragment thereof) that has transferase activity is coenzyme A transferase, acyl transferase, peptidyl transferase, N-acetyltransferase, or pyruvate dehydrogenase.

In some embodiments, the polypeptide molecule described herein can comprise an effector protein (e.g., a protein, or functional domain or functional fragment thereof) coupled to a polypeptide chain that has hydrolase activity. In some embodiments, the polypeptide molecule described herein can comprise at least a portion of an endonuclease coupled to an effector protein (e.g., a protein, or functional domain or functional fragment thereof) that has hydrolase activity via the polypeptide chain. In some embodiments, the effector protein (e.g., a protein, or functional domain or functional fragment thereof) that has hydrolase activity is an esterase, a protease, a glycosidase, or a lipase.

In some embodiments, the polypeptide molecule described herein can comprise an effector protein (e.g., a protein, or functional domain or functional fragment thereof) coupled to a polypeptide chain that has lyase activity. In some embodiments, the polypeptide molecule described herein can comprise at least a portion of an endonuclease coupled to an effector protein (e.g., a protein, or functional domain or functional fragment thereof) that has lyase activity via the polypeptide chain. In some embodiments, the effector protein (e.g., a protein, or functional domain or functional fragment thereof) that has lyase activity is phenylalanine ammonia-lyase, citrate lyase, isocitrate lyase, hydroxynitrile, pectate lyase, argininosuccinate lyase, pyruvate formate lyase, alginate lyase, or pectin lyase.

In some embodiments, the polypeptide molecule described herein can comprise an effector protein (e.g., a protein, or functional domain or functional fragment thereof) coupled to a polypeptide chain that has isomerase activity. In some embodiments, the polypeptide molecule described herein can comprise at least a portion of an endonuclease coupled to an effector protein (e.g., a protein, or functional domain or functional fragment thereof) that has isomerase activity via the polypeptide chain. In some embodiments, the effector protein (e.g., a protein, or functional domain or functional fragment thereof) that has isomerase activity is ribose phosphate isomerase, bisphosphoglycerate mutase, or photoisomerase.

In some embodiments, the polypeptide molecule described herein can comprise an effector protein (e.g., a protein, or functional domain or functional fragment thereof) coupled to a polypeptide chain that has synthase activity. In some embodiments, the polypeptide molecule described herein can comprise at least a portion of an endonuclease coupled to an effector protein (e.g., a protein, or functional domain or functional fragment thereof) that has synthase activity via the polypeptide chain. In some embodiments, the effector protein (e.g., a protein, or functional domain or functional fragment thereof) that has synthase activity is ATP synthase, citrate synthase, tryptophan synthase, pseudouridine synthase, or fatty acid synthase.

In some embodiments, the polypeptide molecule described herein can comprise an effector protein (e.g., a protein, or functional domain or functional fragment thereof) coupled to a polypeptide chain that has demyristoylation activity. In some embodiments, the polypeptide molecule described herein can comprise at least a portion of an endonuclease coupled to an effector protein (e.g., a protein, or functional domain or functional fragment thereof) that has demyristoylation activity via the polypeptide chain. In some embodiments, the effector protein (e.g., a protein, or functional domain or functional fragment thereof) that has demyristoylation activity is T3SS effector protein.

In some embodiments, the polypeptide molecule described herein can comprise an effector protein (e.g., a protein, or functional domain or functional fragment thereof) coupled to a polypeptide chain that has transposition activity. In some embodiments, the polypeptide molecule described herein can comprise at least a portion of an endonuclease coupled to an effector protein (e.g., a protein, or functional domain or functional fragment thereof) that has transposition activity via the polypeptide chain. In some embodiments, the effector protein (e.g., a protein, or functional domain or functional fragment thereof) that has transposition activity is transposase Tn5 or Sleeping Beauty transposase.

In some embodiments, a polypeptide molecule can comprise a polypeptide domain (e.g., at least a portion of an endonuclease or one or more gene modulators) and an adaptor protein, or an aptamer sequence (e.g., capable of binding to the adaptor protein) that are operatively coupled to one another via the polypeptide chain disclosed herein. In some embodiments, the adaptor sequence is selected from the group consisting of MS2, PP7, Qβ, F2, GA, fr, JP501, M12, R17, BZ13, JP34, JP500, KU1, M11, MX1, TW18, VK, SP, FI, ID2, NL95, TW19, AP205, φCb5, φCb8r, φ12r, φCb23r, 7s and PRR1. For example, a polypeptide molecule can comprise at least a portion of an endonuclease coupled to an aptamer sequence via the polypeptide chain disclosed herein. In some embodiments, a gene modulator, a gene editing moiety, or an effector protein as disclosed herein can be coupled to an adaptor protein via the polypeptide chain disclosed herein to modulate (e.g., edit, activate, suppress) expression level of a target gene.

The polypeptide molecule (e.g., comprising at least a portion of an endonuclease and/or one or more gene modulator(s) that are operatively coupled to one another via the polypeptide chain) as disclosed herein can be provided in any form. For example, the polypeptide molecule can be provided in the form of a protein, such as the polypeptide molecule alone or complexed with a guide nucleic acid as a ribonucleoprotein. The polypeptide molecule can be provided in a complex, for example, complexed with a guide nucleic acid and other effector protein. The polypeptide molecule can be provided in the form of a nucleic acid encoding the polypeptide molecule, such as an RNA (e.g., messenger RNA (mRNA)), or DNA. The nucleic acid encoding the polypeptide molecule can be codon optimized for efficient translation into protein in a particular cell or organism (e.g., human codon optimized).

Nucleic acids encoding the polypeptide molecule (e.g., comprising at least a portion of an endonuclease and/or one or more gene modulator(s) that are operatively coupled to one another via the polypeptide chain) as disclosed herein, fragments, or derivatives thereof can be stably integrated in the genome of a cell. Nucleic acids encoding the polypeptide molecule can be operably linked to a promoter, for example, a promoter that is constitutively or inducibly active in the cell. Nucleic acids encoding the polypeptide molecule can be operably linked to a promoter in an expression construct. Expression constructs can include any nucleic acid constructs capable of directing expression of a gene or other nucleic acid sequence of interest (e.g., at least the at least the portion of the endonuclease) and which can transfer such a nucleic acid sequence of interest to a target cell.

In some embodiments, the polypeptide molecule as disclosed herein can associate with a single guide RNA (sgRNA) to activate or repress transcription of a target gene (e.g., target endogenous gene), for example, in combination with heterologous gene effector(s) disclosed herein. sgRNAs can be introduced into cells expressing the polypeptide molecule as provided herein. In some embodiments, such cells can contain one or more different sgRNAs that target the same target gene (e.g., target endogenous gene) or target gene regulatory sequence. In other cases, the sgRNAs target different nucleic acids in the cell (e.g., different target genes, different target gene regulatory sequences, or different sequences within the same target gene or target gene regulatory sequence).

Enzymatically inactive (e.g., nuclease deficient) can refer to a nuclease that can bind to a nucleic acid sequence in a polynucleotide in a sequence-specific manner, but may not cleave a target polynucleotide or will cleave it at a substantially reduced frequency. An enzymatically inactive guide moiety can comprise an enzymatically inactive domain (e.g., nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a comparable wild-type activity (e.g., nucleic acid cleaving activity, wild-type Cas activity).

In some embodiments, the target nucleic acid of the at least the portion of the endonuclease as disclosed herein can be dsDNA. In such embodiments, dsDNA-targeting specificity is determined, at least in part, by two parameters: the gRNA spacer targeting a protospacer in the target dsDNA (the sequence in the target dsDNA corresponding to the gRNA spacer on the non-complementary DNA strand) and a short sequence, the protospacer-adjacent motif (PAM), located immediately 5′ (upstream) of the protospacer on the non-complementary DNA strand. In some embodiments, the PA′ is 5′-′TTG-3′ or 5′-′TTA-3′. In some embodiments, the PA′ is 5′-′TTG-3′. In some embodiments, the PA′ is 5′-′TTA-3′.

In some embodiments, the target nucleic acid of the at least the portion of the endonuclease as disclosed herein can be RNA. In such embodiments, RNA-targeting specificity is determined, at least in part, by the gRNA spacer targeting a protospacer-like sequence in the target RNA (the sequence in the target RNA complementary to the gRNA spacer), and is independent of the sequence located immediately 5′ (upstream) of the protospacer-like sequence. In some embodiments, the at least the portion of the endonuclease can be further capable of targeting a dsDNA molecule, wherein the gRNA spacer is selected such that it targets a protospacer in the target dsDNA molecule having a PAM selected from 5′-′TTG-3″ and 5′-′TTA-3′. In other embodiments, the at least the portion of the endonuclease is incapable of targeting a dsDNA molecule, wherein the gRNA spacer is selected such that any protospacers in the dsDNA molecule targeted by the gRNA spacer do not have a PAM selected from 5′-′TTG-3″ and 5′-′TTA-3′.

In some embodiments, the polypeptide molecule comprising the gene modulator and/or the at least the portion of the endonuclease (e.g., and/or a complex comprising the heterologous polypeptide) can regulate expression and/or activity of a target gene (e.g., target endogenous gene). In some embodiments, the heterologous polypeptide and/or a complex thereof can edit the sequence of a nucleic acid (e.g., a gene and/or gene product). A nuclease-active variant of the at least the portion of the endonuclease can edit a nucleic acid sequence by generating a double-stranded break or single-stranded break in a target polynucleotide.

In some embodiments, the polypeptide molecule comprising the gene modulator and/or the at least the portion of the endonuclease (e.g., and/or a complex comprising the heterologous polypeptide) can generate a double-strand break in a target polynucleotide, such as DNA. A double-strand break in DNA can result in DNA break repair which allows for the introduction of gene modification(s) (e.g., nucleic acid editing). In some embodiments, a nuclease induces site-specific single-strand DNA breaks or nicks, thus resulting in HDR.

A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided.

In some embodiments, the polypeptide molecule comprising the gene modulator and/or the at least the portion of the endonuclease (e.g., and/or a complex comprising the polypeptide molecule) does not generate a double-strand break in a target polynucleotide, such as DNA. Binding of the heterologous polypeptide or the complex comprising the heterologous polypeptide (e.g., a complex comprising a nuclease deficient variant of the at least the portion of the endonuclease and a guide RNA) without a nucleic acid break can be sufficient to regulate expression (e.g., enhance or suppress) of a target gene (e.g., endogenous target gene).

### Guide Nucleic Acid Molecule

In some aspects, the present disclosure provides a guide nucleic acid molecule (e.g., an engineered guide nucleic acid molecule) configured to form a complex with a Cas protein. The Cas protein can be a naturally occurring protein. The Cas protein can be an engineered nuclease variant as provided herein. The guide nucleic acid molecule can comprise a spacer sequence exhibiting specific binding to a target polynucleotide sequence operatively coupled to a target gene (e.g., in a cell). The target polynucleotide sequence can be part of the target gene. Alternatively, the target polynucleotide sequence can be upstream (e.g., part of or adjacent to a promoter sequence of the target gene) or downstream of the target gene (e.g., part of or adjacent to a termination sequence of the target gene). The guide nucleic acid molecule can comprise a scaffold sequence for forming the complex with the Cas protein. The spacer sequence and the scaffold sequence can be part of a single polynucleotide sequence (e.g., a single guide nucleic acid molecule, such as sgRNA). Alternatively, the spacer sequence and the scaffold sequence can be separate molecules that are hybridize for forming the complex with the Cas protein.

Without wishing to be bound by theory, the guide nucleic acid molecule as disclosed herein can be operatively coupled to (e.g., can form a functional complex with) one or more Cas proteins. In some embodiments, the scaffold sequence as disclosed herein is not identical to the polynucleotide sequence of SEQ ID NO: 251. The scaffold sequence can comprise at least one deletion, as compared to (e.g., when aligned to) the polynucleotide sequence of SEQ ID NO: 251. Without wishing to be bound by theory, the at least one deletion of the scaffold sequence can be determined by performing a deletion landscape study (e.g., iterative and/or comprehensive deletion) of the control scaffold sequence of SEQ ID NO: 251. The scaffold sequence can comprise at least one mutation, as compared to (e.g., when aligned to) the polynucleotide sequence of SEQ ID NO: 251. Without wishing to be bound by theory, the at least one mutation of the scaffold sequence can be determined by performing a mutation landscape study (e.g., iterative and/or comprehensive mutation) of the control scaffold sequence of SEQ ID NO: 251. The at least one deletion as disclosed herein can be removal of a nucleotide. Alternatively, the at least one deletion can be replacement of a nucleotide with a different nucleotide (e.g., mutation).

In some embodiments, the scaffold sequence can comprise one or more nucleotide deletions when aligned to (or compared to) the control polynucleotide sequence of SEQ ID NO: 251. The one or more nucleotide deletions can comprise a single deletion. The one or more nucleotide deletions can comprise a plurality of nucleotide deletions, such as at least or up to about 2 deletions, at least or up to about 3 deletions, at least or up to about 4 deletions, at least or up to about 5 deletions, at least or up to about 6 deletions, at least or up to about 7 deletions, at least or up to about 8 deletions, at least or up to about 9 deletions, at least or up to about 10 deletions, at least or up to about 11 deletions, at least or up to about 12 deletions, at least or up to about 13 deletions, at least or up to about 14 deletions, at least or up to about 15 deletions, at least or up to about 16 deletions, at least or up to about 17 deletions, at least or up to about 18 deletions, at least or up to about 19 deletions, at least or up to about 20 deletions, at least or up to about 22 deletions, at least or up to about 24 deletions, at least or up to about 25 deletions, at least or up to about 26 deletions, at least or up to about 28 deletions, at least or up to about 30 deletions, at least or up to about 32 deletions, at least or up to about 34 deletions, at least or up to about 35 deletions, at least or up to about 36 deletions, at least or up to about 38 deletions, at least or up to about 40 deletions, at least or up to about 42 deletions, at least or up to about 44 deletions, at least or up to about 45 deletions, at least or up to about 46 deletions, at least or up to about 48 deletions, at least or up to about 50 deletions, at least or up to about 52 deletions, at least or up to about 54 deletions, at least or up to about 55 deletions, at least or up to about 56 deletions, at least or up to about 58 deletions, at least or up to about 60 deletions, at least or up to about 70 deletions, or at least or up to about 80 deletions. The plurality of nucleotide deletions can be adjacent to each other (e.g., consecutive), when aligned to the polynucleotide sequence of SEQ ID NO: 251. The scaffold sequence can comprise a single consecutive deletion. The scaffold sequence can comprise a plurality of consecutive deletion In which one consecutive deletion is not directly adjacent to another consecutive deletion when aligned to the polynucleotide sequence of SEQ ID NO: 251.

In some embodiments, when aligned to the control polynucleotide sequence of SEQ ID NO: 251, the scaffold sequence as disclosed herein can comprise one or more nucleotide deletions in one or more members from the nucleotides 1-10, the nucleotides 11-20, the nucleotides 21-30, the nucleotides 31-40, the nucleotides 41-50, the nucleotides 51-60, the nucleotides 61-70, the nucleotides 71-80, the nucleotides 81-90, the nucleotides 91-100, the nucleotides 101-110, the nucleotides 111-120, the nucleotides 121-130, the nucleotides 131-140, the nucleotides 141-150, and/or the nucleotides 151-159 of SEQ ID NO: 251.

In some embodiments, when aligned to the control polynucleotide sequence of SEQ ID NO: 251, the scaffold sequence as disclosed herein can comprise one or more nucleotide deletions in the nucleotides 1-25 of the polynucleotide sequence of SEQ ID NO: 251, such as the nucleotides 1-23, the nucleotides 3-23, the nucleotides 5-23, the nucleotides 7-23, the nucleotides 9-23, the nucleotides 11-23, the nucleotides 13-23, the nucleotides 15-23, the nucleotides 17-23, the nucleotides 19-23, and/or the nucleotides 21-23 of the polynucleotide sequence of SEQ ID NO: 251. When aligned to the control polynucleotide sequence of SEQ ID NO: 251, the scaffold sequence as disclosed herein can comprise one or more nucleotide deletions in the nucleotides 1-23, the nucleotides 1-21, the nucleotides 1-19, the nucleotides 1-17, the nucleotides 1-15, the nucleotides 1-13, the nucleotides 1-11, the nucleotides 1-9, the nucleotides 1-7, the nucleotides 1-5, and/or the nucleotides 1-3 of the polynucleotide sequence of SEQ ID NO: 251. When aligned to the control polynucleotide sequence of SEQ ID NO: 251, the scaffold sequence as disclosed herein can comprise one or more nucleotide deletions in the nucleotides 1-5, the nucleotides 6-10, the nucleotides 11-15, the nucleotides 16-20, and/or the nucleotides 21-23 of the polynucleotide sequence of SEQ ID NO: 251. When aligned to the control polynucleotide sequence of SEQ ID NO: 251, the scaffold sequence as disclosed herein can comprise one or more nucleotide deletions in the nucleotide 1, nucleotide 2, nucleotide 3, nucleotide 4, nucleotide 5, nucleotide 6, nucleotide 7, nucleotide 8, nucleotide 9, nucleotide 10, nucleotide 11, nucleotide 12, nucleotide 13, nucleotide 14, nucleotide 15, nucleotide 16, nucleotide 17, nucleotide 18, nucleotide 19, nucleotide 20, nucleotide 21, nucleotide 22, nucleotide 23, nucleotide 24, and/or nucleotide 25 of the polynucleotide sequence of SEQ ID NO: 251.

In some embodiments, when aligned to the control polynucleotide sequence of SEQ ID NO: 251, the scaffold sequence as disclosed herein can comprise one or more nucleotide deletions in the nucleotides 35-65 of the polynucleotide sequence of SEQ ID NO: 251, such as the nucleotides 35-61, the nucleotides 37-61, the nucleotides 39-61, the nucleotides 41-61, the nucleotides 43-61, the nucleotides 45-61, the nucleotides 47-61, the nucleotides 49-61, the nucleotides 51-61, the nucleotides 53-61, the nucleotides 55-61, the nucleotides 57-61, and/or the nucleotides 59-61 of the polynucleotide sequence of SEQ ID NO: 251. When aligned to the control polynucleotide sequence of SEQ ID NO: 251, the scaffold sequence as disclosed herein can comprise one or more nucleotide deletions in the nucleotides 35-61, the nucleotides 35-59, the nucleotides 35-57, the nucleotides 35-55, the nucleotides 35-53, the nucleotides 35-51, the nucleotides 35-49, the nucleotides 35-47, the nucleotides 35-45, the nucleotides 35-43, the nucleotides 35-41, the nucleotides 35-39, and/or the nucleotides 35-37 of the polynucleotide sequence of SEQ ID NO: 251. When aligned to the control polynucleotide sequence of SEQ ID NO scaffold sequence as disclosed herein can comprise one or more nucleotide deletions in the nucleotide 35, nucleotide 36, nucleotide 37, nucleotide 38, nucleotide 39, nucleotide 40, nucleotide 41, nucleotide 42, nucleotide 43, nucleotide 44, nucleotide 45, nucleotide 46, nucleotide 47, nucleotide 48, nucleotide 49, nucleotide 50, nucleotide 51, nucleotide 52, nucleotide 53, nucleotide 54, nucleotide 55, nucleotide 56, nucleotide 57, nucleotide 58, nucleotide 59, nucleotide 60, nucleotide 61, nucleotide 62, nucleotide 63, nucleotide 64, and/or nucleotide 65 of the polynucleotide sequence of SEQ ID NO: 251.

In some embodiments, when aligned to the control polynucleotide sequence of SEQ ID NO: 251, the scaffold sequence as disclosed herein can comprise one or more nucleotide deletions in the nucleotides 135-150 of the polynucleotide sequence of SEQ ID NO: 251, such as the nucleotides 136-149, the nucleotides 137-149, the nucleotides 139-149, the nucleotides 141-149, the nucleotides 143-149, the nucleotides 145-149, and/or the nucleotides 147-149 nucleotides of the polynucleotide sequence of SEQ ID NO: 251. When aligned to the control polynucleotide sequence of SEQ ID NO: 251, the scaffold sequence as disclosed herein can comprise one or more nucleotide deletions in the nucleotides 136-149, the nucleotides 136-147, the nucleotides 136-145, the nucleotides 136-143, the nucleotides 136-141, the nucleotides 136-139, and/or the nucleotides 136-137 of the polynucleotide sequence of SEQ ID NO: 251. When aligned to the control polynucleotide sequence of SEQ ID NO: 251, the scaffold sequence as disclosed herein can comprise one or more nucleotide deletions in the nucleotide 135, nucleotide 136, nucleotide 137, nucleotide 138, nucleotide 139, nucleotide 140, nucleotide 141, nucleotide 142, nucleotide 143, nucleotide 144, nucleotide 145, nucleotide 146, nucleotide 147, nucleotide 148, nucleotide 149, and/or nucleotide 150 of the polynucleotide sequence of SEQ ID NO: 251.

In some embodiments, when aligned to the control polynucleotide sequence of SEQ ID NO: 251, the scaffold sequence as disclosed herein can comprise one or more nucleotide deletions in the nucleotides 136-151 of the polynucleotide sequence of SEQ ID NO: 251. In some cases, the scaffold sequence as disclosed herein can comprise one or more nucleotide deletions in the nucleotide T136, T137, C138, A139, T140, T141, T142, G143, A144, A145, T146, G147, A148, A149, G150, and/or G151 of the polynucleotide sequence of SEQ ID NO: 251. In some cases, the scaffold sequence as disclosed herein can comprise one or more nucleotide deletions (e.g., at least 1, at least 2, at least 3, at least 4, at least 5, at least 6, at least 7, at least 8, at least 9, at least 10, at least 11, or all 12 of) in the nucleotide T136, T137, C138, A139, T140, T141, T142, A144, A145, T146, A148, and/or A149 of the polynucleotide sequence of SEQ ID NO: 251. In some cases, the scaffold sequence as disclosed herein can comprise at least or up to about 1 nucleotide, at least or up to about 2 nucleotides, at least or up to about 3 nucleotides, or all 4 nucleotides selected from the group consisting of G143, G147, G150, and G151, when aligned to the polynucleotide sequence of SEQ ID NO: 251.

In some embodiments, the scaffold sequence (e.g., a consecutive polynucleotide sequence of the scaffold sequence) can be characterized by exhibiting at least or up to about 60%, at least or up to about 65%, at least or up to about 70%, at least or up to about 71%, at least or up to about 72%, at least or up to about 73%, at least or up to about 74%, at least or up to about 75%, at least or up to about 76%, at least or up to about 77%, at least or up to about 78%, at least or up to about 79%, at least or up to about 80%, at least or up to about 810%, at least or up to about 82%, at least or up to about 83%, at least or up to about 84%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99, or substantially 100% sequence identity (or complementarity) to the polynucleotide sequence of a member selected from TABLE 6.

In some cases, the length of the scaffold sequence can be at least or up to about 80 nucleotides, at least or up to about 85 nucleotides, at least or up to about 90 nucleotides, at least or up to about 91 nucleotides, at least or up to about 92 nucleotides, at least or up to about 93 nucleotides, at least or up to about 94 nucleotides, at least or up to about 95 nucleotides, at least or up to about 96 nucleotides, at least or up to about 97 nucleotides, at least or up to about 98 nucleotides, at least or up to about 99 nucleotides, at least or up to about 100 nucleotides, at least or up to about 101 nucleotides, at least or up to about 102 nucleotides, at least or up to about 103 nucleotides, at least or up to about 104 nucleotides, at least or up to about 105 nucleotides, at least or up to about 106 nucleotides, at least or up to about 107 nucleotides, at least or up to about 108 nucleotides, at least or up to about 109 nucleotides, at least or up to about 110 nucleotides, at least or up to about 112 nucleotides, at least or up to about 114 nucleotides, at least or up to about 115 nucleotides, at least or up to about 116 nucleotides, at least or up to about 118 nucleotides, at least or up to about 120 nucleotides, at least or up to about 122 nucleotides, at least or up to about 124 nucleotides, at least or up to about 125 nucleotides, at least or up to about 126 nucleotides, at least or up to about 128 nucleotides, at least or up to about 130 nucleotides, at least or up to about 135 nucleotides, at least or up to about 140 nucleotides, at least or up to about 145 nucleotides, at least or up to about 150 nucleotides, at least or up to about 155 nucleotides, or at least or up to about 160 nucleotides.

In some embodiments, the spacer sequence of the guide nucleic acid molecule can have a length of at least or up to about 12 nucleotides, at least or up to about 13 nucleotides, at least or up to about 14 nucleotides, at least or up to about 15 nucleotides, at least or up to about 16 nucleotides, at least or up to about 17 nucleotides, at least or up to about 18 nucleotides, at least or up to about 19 nucleotides, at least or up to about 20 nucleotides, at least or up to about 21 nucleotides, or at least or up to about 22 nucleotides.

In some embodiments, the guide nucleic acid molecule (e.g., comprising scaffold and spacer sequences) can have a length of at least or up to about 80 nucleotides, at least or up to about 85 nucleotides, at least or up to about 90 nucleotides, at least or up to about 95 nucleotides, at least or up to about 96 nucleotides, at least or up to about 97 nucleotides, at least or up to about 98 nucleotides, at least or up to about 99 nucleotides, at least or up to about 100 nucleotides, at least or up to about 101 nucleotides, at least or up to about 102 nucleotides, at least or up to about 103 nucleotides, at least or up to about 104 nucleotides, at least or up to about 105 nucleotides, at least or up to about 106 nucleotides, at least or up to about 107 nucleotides, at least or up to about 108 nucleotides, at least or up to about 109 nucleotides, at least or up to about 110 nucleotides, at least or up to about 111 nucleotides, at least or up to about 112 nucleotides, at least or up to about 113 nucleotides, at least or up to about 114 nucleotides, at least or up to about 115 nucleotides, at least or up to about 116 nucleotides, at least or up to about 117 nucleotides, at least or up to about 118 nucleotides, at least or up to about 119 nucleotides, at least or up to about 120 nucleotides, at least or up to about 121 nucleotides, at least or up to about 122 nucleotides, at least or up to about 123 nucleotides, at least or up to about 124 nucleotides, at least or up to about 125 nucleotides, at least or up to about 130 nucleotides, at least or up to about 135 nucleotides, at least or up to about 140 nucleotides, at least or up to about 145 nucleotides, at least or up to about 150 nucleotides, at least or up to about 155 nucleotides, or at least or up to about 160 nucleotides.

### Heterologous Polynucleotide

In some embodiments, the systems and compositions of the present disclosure further comprises a heterologous polynucleotide (e.g., encoding a gene of interest, such as one or more genes selected from Table 1) that is introduced to the cell without being introduced into a genome of the cell via action of the at least the portion of the endonuclease of the present disclosure. In some embodiments, such heterologous polynucleotide encoding the gene of interest can be interested into the genome of the cell via other means, e.g., via adeno-associated virus vectors (e.g., AAV2 or AAV8). Alternatively, such heterologous polynucleotide encoding the gene of interest may be introduced to the intracellular portion of the cell and remain achromosomal (e.g., as an achromosomal plasmid).

Thus, the systems and compositions can comprise the non-disease causing wild type or variant of the target gene, as abovementioned. Alternatively, or in addition to, the systems and compositions can comprise a heterologous polynucleotide sequence encoding (or comprising) at least the non-disease causing wild type or variant of the target gene (e.g., that of the endogenous target gene) as disclosed herein.

### Systems and Composition

In some aspects, the present disclosure provides a system or a composition comprising (i) a polypeptide molecule comprising: at least a portion of an endonuclease and/or a gene modulator that is operatively coupled to the polypeptide disclosed herein or a heterologous polynucleotide encoding the polypeptide molecule, (ii) the guide nucleic acid as disclosed herein, and/or (iii) the heterologous polynucleotide (e.g., encoding one or more genes from Table 1), for use in any of the methods as disclosed herein. The system or the composition can comprise one or more polynucleotides that encode any of the members (i)-(iii) abovementioned. The subject composition can be usable for modifying a cell in vitro, ex vivo, or in vivo. The subject composition can be usable for treating or enhancing a condition of a subject, as disclosed herein.

The composition as disclosed herein can comprise an active ingredient (e.g., the polypeptide molecule, the guide nucleic acid, etc.) and optionally an additional ingredient (e.g., excipient). If necessary and/or desirable, the composition can be divided, shaped and/or packaged into a desired single- or multi-dose unit or single- or multi-implantation unit.

In some embodiments, the composition can comprise one or more heterologous polynucleotides encoding the active ingredients as disclosed herein. When there are different members within the active ingredients, each member can be encoded by a different heterologous polynucleotide. Alternatively, two or more (e.g., all of) the ingredients can be encoded by a single heterologous polynucleotide. In some embodiments, a heterologous polynucleotide can encode the polypeptide molecule as disclosed herein. In some embodiments, a single heterologous polynucleotide can encode (i) a polypeptide molecule (e.g., at least the portion of the endonuclease and/or a gene modulator operatively coupled to the polypeptide chain disclosed herein), and (ii) one or more guide nucleic acids (e.g., at least 1, at least 2, at least 3, at least 4, at least 5, or more guide nucleic acids) for targeting specific region(s) or sequence(s) of the target gene. The one or more heterologous polynucleotides can further comprise one or more promoters (or one or more transcriptional control elements, as used interchangeably herein). Different active ingredients encoded by the one or more heterologous polynucleotides can be under the control of the same promoter or different promoters. A promoter as disclosed herein can be active in a eukaryotic, mammalian, non-human mammalian, or human cell. The promoter can be an inducible or constitutively active promoter. Alternatively, or additionally, the promoter can be tissue or cell specific. Non-limiting examples of suitable eukaryotic promoters (i.e. promoters functional in a eukaryotic cell) can include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter can be a fungi promoter. The promoter can be a plant promoter. A database of plant promoters can be found (e.g., PlantProm). The expression vector may also contain a ribosome binding site for translation initiation and a transcription terminator. The expression vector may also include appropriate sequences for amplifying expression. In some embodiments, a promoter as disclosed herein can be a promoter specific for any of the tissues provided herein, or a promoter specific for any of the cell types provided herein.

A heterologous polynucleotide of the one or more heterologous polynucleotides (e.g., the single heterologous polynucleotide) can have a size of at least or up to about 2.5 kilobases, at least or up to about 2.6 kilobases, at least or up to about 2.7 kilobases, at least or up to about 2.8 kilobases, at least or up to about 2.9 kilobases, at least or up to about 3.0 kilobases, at least or up to about 3.1 kilobases, at least or up to about 3.2 kilobases, at least or up to about 3.3 kilobases, at least or up to about 3.4 kilobases, at least or up to about 3.5 kilobases, at least or up to about 3.6 kilobases, at least or up to about 3.7 kilobases, at least or up to about 3.8 kilobases, at least or up to about 3.9 kilobases, at least or up to about 4.0 kilobases, at least or up to about 4.1 kilobases, at least or up to about 4.2 kilobases, at least or up to about 4.3 kilobases, at least or up to about 4.4 kilobases, at least or up to about 4.5 kilobases, at least or up to about 4.6 kilobases, at least or up to about 4.7 kilobases, at least or up to about 4.8 kilobases, at least or up to about 4.9 kilobases, at least or up to about 5.0 kilobases, at least or up to about 5.5 kilobases, at least or up to about 6.0 kilobases, at least or up to about 6.5 kilobases, at least or up to about 7.0 kilobases, at least or up to about 7.5 kilobases, at least or up to about 8.0 kilobases, at least or up to about 9.0 kilobases, or at least or up to about 10 kilobases. In some embodiments, the heterologous polynucleotide of the one or more heterologous polynucleotides (e.g., the single heterologous polynucleotide) can have a size of between about 3 kilobases and about 5 kilobases, between about 3 kilobases and about 4.8 kilobases, between about 3 kilobases and about 4.6 kilobases, between about 3 kilobases and about 4.4 kilobases, between about 3 kilobases and about 4.2 kilobases, between about 3 kilobases and about 4.0 kilobases, between about 3 kilobases and about 3.5 kilobases, between about 3.5 kilobases and about 5 kilobases, between about 3.5 kilobases and about 4.8 kilobases, between about 3.5 kilobases and about 4.6 kilobases, between about 3.5 kilobases and about 4.4 kilobases, between about 3.5 kilobases and about 4.2 kilobases, between about 3.5 kilobases and about 4 kilobases, between about 4 kilobases and about 5 kilobases, between about 4 kilobases and about 4.9 kilobases, between about 4 kilobases and about 4.8 kilobases, between about 4 kilobases and about 4.7 kilobases, between about 4 kilobases and about 4.6 kilobases, between about 4 kilobases and about 4.5 kilobases, between about 4 kilobases and about 4.4 kilobases, between about 4 kilobases and about 4.3 kilobases, between about 4 kilobases and about 4.2 kilobases, or between about 4 kilobases and about 4.1 kilobases.

A method of delivery of the one or more heterologous polynucleotides provided herein to the cell can involve viral delivery methods or non-viral delivery methods. Thus, the one or more heterologous polynucleotides can be one or more viral vectors (e.g., one or more AAV vectors). Alternatively, the one or more heterologous polynucleotides can be non-viral vectors that are complexed with or encapsulated by non-viral delivery moieties, such as cationic lipids and/or lipid particles (e.g., lipid nanoparticles (LNP)).

Methods of non-viral delivery of nucleic acids can include lipofection, nucleofection, microinjection, biolistics, virosomes, liposomes, immunoliposomes, polycation or lipid:nucleic acid conjugates, naked DNA, artificial virions, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration).

In some embodiments, the compositions and systems provided herein are delivered to a subject using a viral vector. In some embodiments, the viral vector is an adeno-associated viral (AAV) vector. The term “AAV” is an abbreviation for adeno-associated virus, and may be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAV5, AAV6, AAV7, AAV8, AAV9, AAV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). The term rAAV vector encompasses both rAAV vector particles and rAAV vector plasmids. An rAAV vector may either be single-stranded (ssAAV) or self-complementary (scAAV). An “AAV virus” or “AAV viral particle” or “rAAV vector particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of rAAV particles necessarily includes production of rAAV vectors, as such a vector is contained within an rAAV particle. In some embodiments, the AAV vector is selected based on the tropism of viral vector. In some embodiments, an AAV vector with tropism for the target tissue may be used to deliver polynucleotides encoding the compositions and systems provided herein to the target tissue.

RNA or DNA viral based systems can be used to target specific cells in the body and trafficking the viral payload to the nucleus of the cell. Viral vectors can be administered directly (in vivo), or they can be used to treat cells in vitro, and the modified cells can optionally be administered (ex vivo). Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted transgene. High transduction efficiencies can be observed in many different cell types and target tissues.

The tropism of a retrovirus can be altered by incorporating foreign envelope proteins, expanding the potential target population of target cells. Lentiviral vectors are retroviral vectors that can transduce or infect non-dividing cells and produce high viral titers. Selection of a retroviral gene transfer system can depend on the target tissue. Retroviral vectors can comprise cis-acting long terminal repeats with packaging capacity for up to 6-10 kb of foreign sequence. The minimum cis-acting LTRs can be sufficient for replication and packaging of the vectors, which can be used to integrate the therapeutic gene into the target cell to provide permanent transgene expression. Retroviral vectors can include those based upon murine leukemia virus (MuLV), gibbon ape leukemia virus (GaLV), Simian Immuno deficiency virus (SIV), human immuno deficiency virus (HIV), and combinations thereof.

An adenoviral-based systems can be used. Adenoviral-based systems can lead to transient expression of the transgene. Adenoviral based vectors can have high transduction efficiency in cells and may not require cell division. High titer and levels of expression can be obtained with adenoviral based vectors. Adeno-associated virus (“AAV”) vectors can be used to transduce cells with target nucleic acids, e.g., in the in vitro production of nucleic acids and peptides, and for in vivo and ex vivo gene therapy procedures.

Packaging cells can be used to form virus particles capable of infecting a host cell. Such cells can include 293 cells, (e.g., for packaging adenovirus), and Psi2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. Viral DNA can be packaged in a cell line, which can contain a helper plasmid encoding the other AAV genes, namely rep and cap, while lacking ITR sequences. The cell line can also be infected with adenovirus as a helper. The helper virus can promote replication of the AAV vector and expression of AAV genes from the helper plasmid. Contamination with adenovirus can be reduced by, e.g., heat treatment to which adenovirus is more sensitive than AAV.

A host cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein is used to establish a new cell line comprising one or more vector-derived sequences. In some embodiments, a cell transiently transfected with the compositions of the disclosure (such as by transient transfection of one or more vectors, or transfection with RNA), and modified through the activity of the heterologous polypeptide comprising the engineered gene effector and the at least the portion of the endonuclease as disclosed herein, is used to establish a new cell line comprising cells containing the modification but lacking any other exogenous sequence.

Any suitable vector compatible with the host cell can be used with the methods of the disclosure. Non-limiting examples of vectors for eukaryotic host cells include pXT1, pSG5 (Stratagene™), pSVK3, pBPV, pMSG, and pSVLSV40 (Pharmacia™).

In some embodiments, the additional ingredient of the composition as disclosed herein can comprise an excipient. Non-limiting examples of the excipient can include solvents, dispersion media, diluents, or other liquid vehicles, dispersion or suspension aids, surface active agents, isotonic agents, thickening or emulsifying agents, preservatives, lipidoids, liposomes, lipid nanoparticles, polymers, lipoplexes, core-shell nanoparticles, peptides, proteins, hyaluronidase, nanoparticle mimics, inert diluents, buffering agents, lubricating agents, oils, and combinations thereof. In some examples, the composition as disclosed herein can include one or more excipients, each in an amount that together increases the stability of (i) the heterologous polypeptide or the heterologous gene encoding thereof and/or (ii) cells or modified cells.

In some aspects, the present disclosure provides a kit comprising such composition and instructions directing (i) contacting the cell with the composition (e.g., in vitro, ex vivo, or in vivo), or (ii) administration of cells comprising any one of the compositions disclosed herein to a subject. The subject may have or may be suspected of having a condition, such as a hereditary disease.

In some embodiments, any of the compositions as disclosed herein, can be administered to the subject via orally, intraperitoneally, intravenously, intraarterially, transdermally, intramuscularly, liposomally, via local delivery by catheter or stent, subcutaneously, intraadiposally, or intrathecally. In particular aspects, the compositions and systems provided herein (including polynucleotides encoding said compositions and systems, e.g., contained in an AAV vector) can be administered to a subject via intravenous administration.

Non-limiting examples of viral vectors that can be utilized to deliver the heterologous polypeptide and/or heterologous polynucleotide (or one or more genes encoding thereof) can include, but are not limited to, retroviral vectors, lentiviral vectors, adenovirus vectors, poxvirus vectors, herpesvirus vectors, adeno-associated virus (AAV) vectors. Non-limiting examples of AAV vectors can include AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu. 11, AAV16.3, AAV16.8/hu. 10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu. 17, AAV33.4/hu. 15, AAV33.8/hu. 16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-lb, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu. 19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LKO1, AAV-LK02, AAVLK03, AAV-LK03, AAV-LK04, AAV-LK05, AAV-LKO6, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and AAV SM 10-8. For example, AAVrh.74 can be used as a viral vector to deliver a polynucleotide sequence encoding the heterologous polypeptide and the heterologous polynucleotide (e.g., Cas protein-gene effector fusion and one or more guide nucleic acid molecules).

Methods of non-viral delivery of nucleic acids can include lipofection, nucleofection, microinjection, biolistics, virosomes, liposomes, immunoliposomes, polycation or lipid:nucleic acid conjugates, lipid nanoparticles (LNPs), naked DNA, artificial virions, and agent-enhanced uptake of DNA. Cationic and neutral lipids that are suitable for efficient receptor-recognition lipofection of polynucleotides can be used.

Any of the compositions disclosed herein (or one or more genes encoding any portion of the compositions), such as the heterologous gene effector(s) and/or the guide nucleic acid molecule(s), can be administered by any suitable administration route, including but not limited to, parenteral (e.g., intravenous, intratumoral, subcutaneous, intramuscular, intracerebral, intracerebroventricular, intra-articular, intraperitoneal, or intracranial), intranasal, buccal, sublingual, oral, or rectal administration routes. In some instances, the pharmaceutical composition is formulated for parenteral (e.g., intravenous, intratumoral, subcutaneous, intramuscular, intracerebral, intracerebroventricular, intra-articular, intraperitoneal, or intracranial) administration.

The compositions (e.g., pharmaceutical compositions) as disclosed herein can be suitable for administration to humans. In addition, such compositions can be suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

### Cells

In some embodiments, a cell as provided herein may be referred to as a target cell. In some embodiments, the systems, compositions, and methods as provided herein can be applied to modify a target cell (e.g., modify expression profile of a target gene of the target cell, such as one or genes in Table 1). A target cell can include a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem (iPS) cells) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can comprise a target nucleic acid. A target cell can be in a living organism. A target cell can be a genetically modified cell. A target cell can be a host cell.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and an apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy.

Non-limiting examples of cells which can be target cells include, but are not limited to, lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell' of Huxley's layer, Hair root sheath cel' of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), 'Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretio'), Bowman's gland cell in nose (washes olfactory epithelium', Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), 'Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

The cell (or target cell) can be engineered to comprise (or exhibit) any one of the systems or compositions as disclosed herein or can be treated by any one of the methods disclosed herein in vitro or ex vivo, then administered to the subject, e.g., to treat a condition of the subject. For example, any subject modified cell product can be administered to the subject to treat a condition of a bodily tissue of the subject. In some embodiments, the cell can be resident inside the subject's body, and any of the systems or compositions thereof can be administered to the subject, to contact the cell by the systems/compositions (e.g., to engineer the cell with the systems/compositions).

### Target Gene

The disclosure provides compositions, methods, and systems for modulating expression of one or more target genes. The target gene(s) can be one or more heterologous target genes. The target gene(s) can be one or more endogenous target genes, such as (i) a disease causing allele, e.g., a mutant allele, and/or (ii) a non-disease causing allele, e.g., a wild type allele. For example, disclosed herein are complexes that comprise a guide moiety (e.g., a guide nucleic acid) and the polypeptide molecule (e.g., comprising one or more gene modulator(s) and the at least the portion of the endonuclease) that can modulate (e.g., increase or decrease) an activity or expression level of a target gene (e.g., in a cell).

In some embodiments, a target gene or regulatory sequence thereof is endogenous to a cell, for example, present in the cell's genome, or endogenous to a subject, for example, present in the subject's genome. In some embodiments, a target gene or regulatory sequence thereof is not part of an engineered reporter system.

In some embodiments, a target gene is exogenous to a host subject, for example, a pathogen target gene or an exogenous gene expressed as a result of a therapeutic intervention, such as a gene therapy and/or cell therapy. In some embodiments, a target gene is an exogenous reporter gene. In some embodiments, a target gene is an exogenous synthetic gene.

In some embodiments, the systems and methods as disclosed herein can modulate (e.g., increase or decrease) expression of a target gene (e.g., upon introducing a complex comprising the polypeptide molecule disclosed herein and the heterologous polypeptide into a cell or population of cells). In some embodiments, an expression level is an RNA expression level that can be measured by, for example, RNAseq, qPCR, microarray, gene array, FISH, etc. In some embodiments, an expression level is a protein expression level that can be measured by, for example, Western Blot, ELISA, multiplex immunoassay, mass spectrometry, NMR, proteomics, flow cytometry, mass cytometry, etc.

In some embodiments, the systems and methods as disclosed herein can modulate (e.g., increase or decrease) expression of a target gene (e.g., upon introducing a complex comprising the polypeptide molecule disclosed herein and the heterologous polypeptide into a cell or population of cells) by at least about 10%, at least about 20%, at least about 30%, at least about 40%, at least about 50%, at least about 60%, at least about 70%, at least about 80%, at least about 90%, at least about 2-fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14, at least fold about 15 fold, at least about 20 fold, at least about 30 fold, at least about 40 fold, at least about 50 fold, at least about 60 fold, at least about 70 fold, at least about 80 fold, at least about 90 fold, at least about 100 fold, at least about 150 fold, at least about 200 fold, at least about 250 fold, at least about 300 fold, at least about 350 fold, at least about 400 fold, at least about 500 fold, at least about 600 fold, at least about 700 fold, at least about 800 fold, at least about 900 fold, at least about 1000 fold, at least about 1500 fold, at least about 2000 fold, or at least about 3000 fold.

In some embodiments, the systems and methods as disclosed herein can modulate (e.g., increase or decrease) expression of a target gene (e.g., upon introducing a complex comprising the polypeptide molecule disclosed herein and the heterologous polypeptide into a cell or population of cells) by at most about 50%, at most about 60%, at most about 70%, at most about 80%, at most about 90%, at most about 2-fold, at most about 3 fold, at most about 4 fold, at most about 5 fold, at most about 6 fold, at most about 7 fold, at most about 8 fold, at most about 9 fold, at most about 10 fold, at most about 11 fold, at most about 12 fold, at most about 13 fold, at most about 14, at most fold about 15 fold, at most about 20 fold, at most about 30 fold, at most about 40 fold, at most about 50 fold, at most about 60 fold, at most about 70 fold, at most about 80 fold, at most about 90 fold, at most about 100 fold, at most about 150 fold, at most about 200 fold, at most about 250 fold, at most about 300 fold, at most about 350 fold, at most about 400 fold, at most about 500 fold, at most about 600 fold, at most about 700 fold, at most about 800 fold, at most about 900 fold, at most about 1000 fold, at most about 1500 fold, at most about 2000 fold, at most about 3000 fold, at most about 5000 fold, or at most about 10000 fold.

In some embodiments, the systems and methods as disclosed herein can modulate (e.g., increase or decrease) expression of a target gene (e.g., upon introducing a complex comprising the polypeptide molecule disclosed herein and the heterologous polypeptide into a cell or population of cells) by about 10%, about 20%, about 30%, about 40%, about 50%, about 60%, about 70%, about 80%, about 90%, about 2-fold, about 3 fold, about 4 fold, about 5 fold, about 6 fold, about 7 fold, about 8 fold, about 9 fold, about 10 fold, about 11 fold, about 12 fold, about 13 fold, about 14, about 15 fold, about 20 fold, about 30 fold, about 40 fold, about 50 fold, about 60 fold, about 70 fold, about 80 fold, about 90 fold, about 100 fold, about 150 fold, about 200 fold, about 250 fold, about 300 fold, about 350 fold, about 400 fold, about 500 fold, about 600 fold, about 700 fold, about 800 fold, about 900 fold, about 1000 fold, about 1500 fold, about 2000 fold, about 3000 fold, about 5000 fold, or about 10000 fold.

In some embodiments, the systems and methods as disclosed herein can modulate (e.g., increase or decrease) expression of a target gene (e.g., upon introducing a complex comprising the polypeptide molecule disclosed herein and the heterologous polypeptide into a cell or population of cells) from below a limit of detection to a detectable level.

In some embodiments, the degree in change of expression is relative to before introducing the system of the present disclosure (e.g., a complex comprising the polypeptide molecule disclosed herein and the heterologous polypeptide) into the cell or population of cells. In some embodiments, the degree in change of expression is relative to a corresponding control cell or population of cells that are not treated with the system of the present disclosure. In some embodiments, the degree in change of expression is relative to a corresponding control cell or population of cells that are treated with an alternative to the system of the present disclosure.

In some embodiments, the systems and methods as disclosed herein can modulate (e.g., increase or decrease) an activity level of a target gene (e.g., upon introducing a complex comprising the polypeptide molecule disclosed herein and the heterologous polypeptide comprising the at least the portion of the endonuclease as disclosed herein into a cell or population of cells). An activity level can be determined by a suitable functional assay for the target gene in question depending on the functional characteristics of the target gene. For example, an activity level of a target gene that is a mitogen could be determined by measuring cell proliferation; an activity level of a target gene that induces apoptosis could be measured by an annexin V assay or other suitable cell death assay; an activity level of an anti-inflammatory cytokine could be measured by an LPS-induced cytokine release assay.

The systems and methods of the present disclosure can, in some embodiments, elicit changes in expression and/or activity level of a target gene (e.g., target endogenous gene) that persists for longer than can be achieved with alternative compositions and methods (e.g., suppression via RNAi, e.g., using siRNA). In some embodiments, persistent modulation of gene expression (e.g., durable gene activation or durable gene suppression) is advantageous as compared to transient modulation.

In some embodiments, the systems and methods as disclosed herein can modulate (e.g., increase or decrease) expression and/or activity level of a target gene for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 12 hours, at least about 14 hours, at least about 18 hours, at least about 20 hours, at least about 1 day, at least about 2 days, at least about 3 days, at least about 4 days, at least about 5 days, at least about 6 days, at least about 7 days, at least about 8 days, at least about 9 days, at least about 10 days, at least about 14 days, at least about 21 days, at least about 28 days, at least about 5 weeks, at least about 6 weeks, at least about 7 weeks, at least about 8 weeks, at least about 9 weeks, at least about 10 weeks, at least about 12 weeks, at least about 14 weeks, at least about 18 weeks, at least about 20 weeks, at least about 26 weeks, or at least about 5 months, at least about 6 months, at least about 9 months, at least about 12 months, or more.

In some embodiments the systems and methods as disclosed herein can modulate (e.g., increase or decrease) expression and/or activity level of a target gene (e.g., target endogenous gene) to above a certain threshold for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 12 hours, at most about 14 hours, at most about 18 hours, at most about 20 hours, at most about 1 day, at most about 2 days, at most about 3 days, at most about 4 days, at most about 5 days, at most about 6 days, at most about 7 days, at most about 8 days, at most about 9 days, at most about 10 days, at most about 14 days, at most about 21 days, at most about 28 days, at most about 5 weeks, at most about 6 weeks, at most about 7 weeks, at most about 8 weeks, at most about 9 weeks, at most about 10 weeks, at most about 12 weeks, at most about 14 weeks, at most about 18 weeks, at most about 20 weeks, at most about 26 weeks, or at most about 5 months, at most about 6 months, at most about 9 months, at most about 12 months, or more.

In some embodiments, the systems and methods as disclosed herein can modulate (e.g., increase or decrease) expression and/or activity level of a target gene (e.g., target endogenous gene) to above a certain threshold for about 1 hour, about 2 hours, about 3 hours, about 4 hours, about 5 hours, about 6 hours, about 7 hours, about 8 hours, about 9 hours, about 10 hours, about 12 hours, about 14 hours, about 18 hours, about 20 hours, about 1 day, about 2 days, about 3 days, about 4 days, about 5 days, about 6 days, about 7 days, about 8 days, about 9 days, about 10 days, about 14 days, about 21 days, about 28 days, about 5 weeks, about 6 weeks, about 7 weeks, about 8 weeks, about 9 weeks, about 10 weeks, about 12 weeks, about 14 weeks, about 18 weeks, about 20 weeks, about 26 weeks, about 5 months, about 6 months, about 9 months, or about 12 months.

In some embodiments, the target gene (e.g., endogenous target gene) can be a disease-causing allele, such as a mutant variant of a wild type allele. The disease can be a genetic disease, such as a hereditary disorder. Non-limiting examples of the genetic disorder can include Duchenne muscular dystrophy (DMD), hemophilia, cystic fibrosis, 'untington's chorea, familial hypercholesterolemia (LDL receptor defect), hepatoblasto'a, Wilson's disease, congenital hepatic porphyria, inherited disorders of hepatic metabolism, Lesch Nyhan syndrome, sickle cell anemia, thalassaemias, xeroderma pigmentosu', Fanconi's anemia, retinitis pigmentosa, ataxia telangiecta'ia, Bloom's syndrome, retinoblastoma, and Tay-Sachs disease. In some embodiments, the target gene can be a gene encoding a protein. In some embodiments, the target gene can be a gene regulatory sequence (e.g., promoters, enhancers, repressors, silencers, insulators, cis-regulatory elements, trans-regulatory elements, epigenetic modification (e.g., DNA methylation) sites, etc.) that can influence expression of a gene encoding a protein of interest as provided herein. For example, target gene regulatory sequences can be physically located outside of the transcriptional unit or open reading frame that encodes a product of the target gene.

In some embodiments, a target gene regulatory sequence does not contain a nucleotide sequence that is exogenous to the subject or host cell. In some embodiments, a target gene regulatory sequence does not contain an engineered or artificially generated or introduced nucleotide sequence.

In some embodiments, a target gene (e.g., target endogenous gene) is a gene that is over-expressed or under-expressed in a disease or condition. In some embodiments, a target gene is a gene that is over-expressed or under-expressed in a heritable genetic disease.

In some embodiments, a target gene (e.g., target endogenous gene) is a gene that is over-expressed or under-expressed in a cancer, for example, acute leukemia, astrocytomas, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewings sarcoma, gastric cancer, glioblastoma, glioblastoma multiforme, glioma, hematologic malignancies, hepatocellular (liver) carcinoma, hepatom', Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwanoma, skin cancer, spinal axis tumors, squamous cell carcinomas, stomach cancer, synovial sarcoma, testicular cancer, uterine cancer, or tumors and their metastases, including refractory versions of any of the above cancers, or a combination thereof.

Non-limiting examples of a target gene or a gene encoding a protein of interest, as disclosed herein, are included in Table 1.

TABLE 1
List of examples of target genes (e.g., encoding a protein of interest)
List of target genes/proteins of interest

A1BG, CCND3, FAM163A, KCNK10, NRG4, REM1, TECTB, A1CF, CCNDBP1, FAM163B,
KCNK12, NRGN, REM2, TEDDM1, A2M, CCNE1, FAM166A, KCNK13, NRIP1, REN, TEF,
A2ML1, CCNE2, FAM166B, KCNK15, NRIP2, RENBP, TEFM, A3GALT2, CCNF, FAM167A,
KCNK16, NRIP3, REP15, TEK, A4GALT, CCNG1, FAM167B, KCNK17, NRK, REPIN1, TEKT1,
A4GNT, CCNG2, FAM168A, KCNK18, NRL, REPS1, TEKT2, AAAS, CCNH, FAM168B,
KCNK2, NRM, REPS2, TEKT3, AACS, CCNI, FAM169A, KCNK3, NRN1, RER1, TEKT4,
AADAC, CCNI2, FAM169B, KCNK4, NRN1L, RERE, TEKT5, AADACL2, CCNJ, FAM170A,
KCNK5, NRP1, RERG, TELO2, AADACL3, CCNJL, FAM170B, KCNK6, NRP2, RERGL, TEN1,
AADACL4, CCNK, FAM171A1, KCNK7, NRROS, RESP18, TENC1, AADAT, CCNL1,
FAM171A2, KCNK9, NRSN1, REST, TENM1, AAED1, CCNL2, FAM171B, KCNMA1, NRSN2,
RET, TENM2, AAGAB, CCNO, FAM172A, KCNMB1, NRTN, RETN, TENM3, AAK1, CCNT1,
FAM173A, KCNMB2, NRXN1, RETNLB, TENM4, AAMDC, CCNT2, FAM173B, KCNMB3,
NRXN2, RETSAT, TEP1, AAMP, CCNY, FAM174A, KCNMB4, NRXN3, REV1, TEPP, AANAT,
CCNYL1, FAM174B, KCNN1, NSA2, REV3L, TERF1, AAR2, CCP110, FAM175A, KCNN2,
NSD1, REXO1, TERF2, AARD, CCPG1, FAM175B, KCNN3, NSDHL, REXO2, TERF2IP,
AARS, CCR1, FAM177A1, KCNN4, NSF, REXO4, TERT, AARS2, CCR10, FAM177B, KCNQ1,
NSFL1C, RFC1, TES, AARSD1, CCR2, FAM178A, KCNQ2, NSG1, RFC2, TESC, AASDH,
CCR3, FAM178B, KCNQ3, NSL1, RFC3, TESK1, AASDHPPT, CCR4, FAM179A, KCNQ4,
NSMAF, RFC4, TESK2, AASS, CCR5, FAM179B, KCNQ5, NSMCE1, RFC5, TESPA1, AATF,
CCR6, FAM180A, KCNRG, NSMCE2, RFESD, TET1, AATK, CCR7, FAM180B, KCNS1,
NSMCE4A, RFFL, TET2, ABAT, CCR8, FAM181A, KCNS2, NSMF, RFK, TET3, ABCA1,
CCR9, FAM181B, KCNS3, NSRP1, RFNG, TEX10, ABCA10, CCRL2, FAM183A, KCNT1,
NSUN2, RFPL1, TEX101, ABCA12, CCRN4L, FAM184A, KCNT2, NSUN3, RFPL2, TEX11,
ABCA13, CCS, FAM184B, KCNU1, NSUN4, RFPL3, TEX12, ABCA2, CCSAP, FAM185A,
KCNV1, NSUN5, RFPL4A, TEX13A, ABCA3, CCSER1, FAM186A, KCNV2, NSUN6,
RFPL4AL1, TEX13B, ABCA4, CCSER2, FAM186B, KCP, NSUN7, RFPL4B, TEX14, ABCA5,
CCT2, FAM187B, KCTD1, NT5C, RFT1, TEX15, ABCA6, CCT3, FAM188A, KCTD10,
NT5C1A, RFTN1, TEX19, ABCA7, CCT4, FAM188B, KCTD11, NT5C1B, RFTN2, TEX2,
ABCA8, CCT5, FAM189A1, KCTD12, NT5C1B-RDH14, RFWD2, TEX22, ABCA9, CCT6A,
FAM189A2, KCTD13, NT5C2, RFWD3, TEX26, ABCB1, CCT6B, FAM189B, KCTD14,
NT5C3A, RFX1, TEX261, ABCB10, CCT7, FAM192A, KCTD15, NT5C3B, RFX2, TEX264,
ABCB11, CCT8, FAM193A, KCTD16, NT5DC1, RFX3, TEX28, ABCB4, CCT8L2, FAM193B,
KCTD17, NT5DC2, RFX4, TEX29, ABCB5, CCZ1, FAM194A, KCTD18, NT5DC3, RFX5,
TEX30, ABCB6, CCZ1B, FAM194B, KCTD19, NT5E, RFX6, TEX33, ABCB7, CD101,
FAM195A, KCTD2, NT5M, RFX7, TEX35, ABCB8, CD109, FAM195B, KCTD20, NTAN1,
RFX8, TEX36, ABCB9, CD14, FAM196A, KCTD21, NTF3, RFXANK, TEX37, ABCC1, CD151,
FAM196B, KCTD3, NTF4, RFXAP, TEX38, ABCC10, CD160, FAM198A, KCTD4, NTHL1,
RGAG1, TEX40, ABCC11, CD163, FAM198B, KCTD5, NTM, RGAG4, TEX9, ABCC12,
CD163L1, FAM199X, KCTD6, NTMT1, RGCC, TF, ABCC2, CD164, FAM19A1, KCTD7, NTN1,
RGL1, TFAM, ABCC3, CD164L2, FAM19A2, KCTD8, NTN3, RGL2, TFAP2A, ABCC4, CD177,
FAM19A3, KCTD9, NTN4, RGL3, TFAP2B, ABCC5, CD180, FAM19A4, KDELC1, NTN5,
RGL4, TFAP2C, ABCC6, CD19, FAM19A5, KDELC2, NTNG1, RGMA, TFAP2D, ABCC8,
CD1A, FAM200A, KDELR1, NTNG2, RGMB, TFAP2E, ABCC9, CD1B, FAM203A, KDELR2,
NTPCR, RGN, TFAP4, ABCD1, CD1C, FAM203B, KDELR3, NTRK1, RGP1, TFB1M, ABCD2,
CD1D, FAM204A, KDM1A, NTRK2, RGPD1, TFB2M, ABCD3, CD1E, FAM205A, KDM1B,
NTRK3, RGPD2, TFCP2, ABCD4, CD2, FAM206A, KDM2A, NTS, RGPD3, TFCP2L1, ABCE1,
CD200, FAM207A, KDM2B, NTSR1, RGPD4, TFDP1, ABCF1, CD200R1, FAM208A, KDM3A,
NTSR2, RGPD5, TFDP2, ABCF2, CD200R1L, FAM208B, KDM3B, NUAK1, RGPD6, TFDP3,
ABCF3, CD207, FAM209A, KDM4A, NUAK2, RGPD8, TFE3, ABCG1, CD209, FAM209B,
KDM4B, NUB1, RGR, TFEB, ABCG2, CD22, FAM20A, KDM4C, NUBP1, RGS1, TFEC,
ABCG4, CD226, FAM20B, KDM4D, NUBP2, RGS10, TFF1, ABCG5, CD24, FAM20C, KDM4E,
NUBPL, RGS11, TFF2, ABCG8, CD244, FAM210A, KDM5A, NUCB1, RGS12, TFF3, ABHD1,
CD247, FAM210B, KDM5B, NUCB2, RGS13, TFG, ABHD10, CD248, FAM211A, KDM5C,
NUCKS1, RGS14, TFIP11, ABHD11, CD27, FAM211B, KDM5D, NUDC, RGS16, TFPI,
ABHD12, CD274, FAM212A, KDM6A, NUDCD1, RGS17, TFPI2, ABHD12B, CD276,
FAM212B, KDM6B, NUDCD2, RGS18, TFPT, ABHD13, CD28, FAM213A, KDM8, NUDCD3,
RGS19, TFR2, ABHD14A, CD2AP, FAM213B, KDR, NUDT1, RGS2, TFRC, ABHD14B,
CD2BP2, FAM214A, KDSR, NUDT10, RGS20, TG, ABHD15, CD300A, FAM214B, KEAP1,
NUDT11, RGS21, TGDS, ABHD16A, CD300C, FAM216A, KEL, NUDT12, RGS22, TGFA,
ABHD16B, CD300E, FAM216B, KERA, NUDT13, RGS3, TGFB1, ABHD17A, CD300LB,
FAM217A, KHDC1, NUDT14, RGS4, TGFB1I1, ABHD17B, CD300LD, FAM217B, KHDC1L,
NUDT15, RGS5, TGFB2, ABHD17C, CD300LF, FAM218A, KHDC3L, NUDT16, RGS6, TGFB3,
ABHD2, CD300LG, FAM219A, KHDRBS1, NUDT16L1, RGS7, TGFBI, ABHD3, CD302,
FAM219B, KHDRBS2, NUDT17, RGS7BP, TGFBR1, ABHD4, CD320, FAM21A, KHDRBS3,
NUDT18, RGS8, TGFBR2, ABHD5, CD33, FAM21B, KHK, NUDT19, RGS9, TGFBR3, ABHD6,
CD34, FAM21C, KHNYN, NUDT2, RGS9BP, TGFBR3L, ABHD8, CD36, FAM220A, KHSRP,
NUDT21, RGSL1, TGFBRAP1, ABI1, CD37, FAM221A, KIAA0020, NUDT22, RHAG, TGIF1,
ABI2, CD38, FAM221B, KIAA0040, NUDT3, RHBDD1, TGIF2, ABI3, CD3D, FAM222A,
KIAA0100, NUDT4, RHBDD2, TGIF2-C20orf24, ABI3BP, CD3E, FAM222B, KIAA0101,
NUDT5, RHBDD3, TGIF2LX, ABL1, CD3EAP, FAM227A, KIAA0141, NUDT6, RHBDF1,
TGIF2LY, ABL2, CD3G, FAM227B, KIAA0195, NUDT7, RHBDF2, TGM1, ABLIM1, CD4,
FAM228A, KIAA0196, NUDT8, RHBDL1, TGM2, ABLIM2, CD40, FAM228B, KIAA0226,
NUDT9, RHBDL2, TGM3, ABLIM3, CD40LG, FAM229A, KIAA0226L, NUF2, RHBDL3,
TGM4, ABO, CD44, FAM229B, KIAA0232, NUFIP1, RHBG, TGM5, ABR, CD46, FAM230A,
KIAA0247, NUFIP2, RHCE, TGM6, ABRA, CD47, FAM24A, KIAA0319, NUGGC, RHCG,
TGM7, ABRACL, CD48, FAM24B, KIAA0319L, NUMA1, RHD, TGOLN2, ABT1, CD5,
FAM25A, KIAA0355, NUMB, RHEB, TGS1, ABTB1, CD52, FAM25C, KIAA0368, NUMBL,
RHEBL1, TH, ABTB2, CD53, FAM25G, KIAA0391, NUP107, RHNO1, THADA, ACAA1, CD55,
FAM26D, KIAA0408, NUP133, RHO, THAP1, ACAA2, CD58, FAM26E, KIAA0430, NUP153,
RHOA, THAP10, ACACA, CD59, FAM26F, KIAA0513, NUP155, RHOB, THAP11, ACACB,
CD5L, FAM32A, KIAA0556, NUP160, RHOBTB1, THAP2, ACAD10, CD6, FAM35A,
KIAA0586, NUP188, RHOBTB2, THAP3, ACAD11, CD63, FAM3A, KIAA0753, NUP205,
RHOBTB3, THAP4, ACAD8, CD68, FAM3B, KIAA0754, NUP210, RHOC, THAP5, ACAD9,
CD69, FAM3C, KIAA0825, NUP210L, RHOD, THAP6, ACADL, CD7, FAM3D, KIAA0895,
NUP214, RHOF, THAP7, ACADM, CD70, FAM43A, KIAA0895L, NUP35, RHOG, THAP8,
ACADS, CD72, FAM43B, KIAA0907, NUP37, RHOH, THAP9, ACADSB, CD74, FAM45A,
KIAA0922, NUP43, RHOJ, THBD, ACADVL, CD79A, FAM46A, KIAA0930, NUP50, RHOQ,
THBS1, ACAN, CD79B, FAM46B, KIAA0947, NUP54, RHOT1, THBS2, ACAP1, CD80,
FAM46C, KIAA1009, NUP62, RHOT2, THBS3, ACAP2, CD81, FAM46D, KIAA1024,
NUP62CL, RHOU, THBS4, ACAP3, CD82, FAM47A, KIAA1024L, NUP85, RHOV, THEG,
ACAT1, CD83, FAM47B, KIAA1033, NUP88, RHOXF1, THEG5, ACAT2, CD84, FAM47C,
KIAA1045, NUP93, RHOXF2, THEGL, ACBD3, CD86, FAM47E, KIAA1107, NUP98,
RHOXF2B, THEM4, ACBD4, CD8A, FAM47E-STBD1, KIAA1109, NUPL1, RHPN1, THEM5,
ACBD5, CD8B, FAM49A, KIAA1143, NUPL2, RHPN2, THEM6, ACBD6, CD9, FAM49B,
KIAA1147, NUPR1, RIBC1, THEMIS, ACBD7, CD93, FAM50A, KIAA1161, NUPR1L, RIBC2,
THEMIS2, ACCS, CD96, FAM50B, KIAA1191, NUS1, RIC3, THG1L, ACCSL, CD97, FAM53A,
KIAA1199, NUSAP1, RIC8A, THNSL1, ACD, CD99, FAM53B, KIAA1210, NUTF2, RIC8B,
THNSL2, ACE, CD99L2, FAM53C, KIAA1211, NUTM1, RICTOR, THOC1, ACE2, CDA,
FAM57A, KIAA1211L, NUTM2A, RIF1, THOC2, ACER1, CDADC1, FAM57B, KIAA1217,
NUTM2B, RIIAD1, THOC3, ACER2, CDAN1, FAM58A, KIAA1239, NUTM2F, RILP, THOC5,
ACER3, CDC123, FAM60A, KIAA1244, NUTM2G, RILPL1, THOC6, ACHE, CDC14A,
FAM63A, KIAA1257, NVL, RILPL2, THOC7, ACIN1, CDC14B, FAM63B, KIAA1279, NWD1,
RIMBP2, THOP1, ACKR1, CDC16, FAM64A, KIAA1324, NXF1, RIMBP3, THPO, ACKR2,
CDC20, FAM65A, KIAA1324L, NXF2, RIMBP3B, THRA, ACKR3, CDC20B, FAM65B,
KIAA1328, NXF2B, RIMBP3C, THRAP3, ACKR4, CDC23, FAM65C, KIAA1377, NXF3,
RIMKLA, THRB, ACLY, CDC25A, FAM69A, KIAA1407, NXF5, RIMKLB, THRSP, ACMSD,
CDC25B, FAM69B, KIAA1429, NXN, RIMS1, THSD1, ACN9, CDC25C, FAM69C, KIAA1430,
NXNL1, RIMS2, THSD4, ACO1, CDC26, FAM71A, KIAA1432, NXNL2, RIMS3, THSD7A,
ACO2, CDC27, FAM71B, KIAA1456, NXPE1, RIMS4, THSD7B, ACOT1, CDC34, FAM71C,
KIAA1462, NXPE2, RIN1, THTPA, ACOT11, CDC37, FAM71D, KIAA1467, NXPE3, RIN2,
THUMPD1, ACOT12, CDC37L1, FAM71E1, KIAA1468, NXPE4, RIN3, THUMPD2, ACOT13,
CDC40, FAM71E2, KIAA1522, NXPH1, RING1, THUMPD3, ACOT2, CDC42, FAM71F1,
KIAA1524, NXPH2, RINL, THY1, ACOT4, CDC42BPA, FAM71F2, KIAA1549, NXPH3, RINT1,
THYN1, ACOT6, CDC42BPB, FAM72A, KIAA1549L, NXPH4, RIOK1, TIA1, ACOT7,
CDC42BPG, FAM72B, KIAA1551, NXT1, RIOK2, TIAF1, ACOT8, CDC42EP1, FAM72D,
KIAA1586, NXT2, RIOK3, TIAL1, ACOT9, CDC42EP2, FAM73A, KIAA1598, NYAP1, RIPK1,
TIAM1, ACOX1, CDC42EP3, FAM73B, KIAA1614, NYAP2, RIPK2, TIAM2, ACOX2,
CDC42EP4, FAM76A, KIAA1644, NYNRIN, RIPK3, TICAM1, ACOX3, CDC42EP5, FAM76B,
KIAA1671, NYX, RIPK4, TICAM2, ACOXL, CDC42SE1, FAM78A, KIAA1683, OAF, RIPPLY1,
TICRR, ACP1, CDC42SE2, FAM78B, KIAA1715, OARD1, RIPPLY2, TIE1, ACP2, CDC45,
FAM81A, KIAA1731, OAS1, RIPPLY3, TIFA, ACP5, CDC5L, FAM81B, KIAA1737, OAS2,
RIT1, TIFAB, ACP6, CDC6, FAM83A, KIAA1751, OAS3, RIT2, TIGD2, ACPL2, CDC7,
FAM83B, KIAA1755, OASL, RLBP1, TIGD3, ACPP, CDC73, FAM83C, KIAA1804, OAT, RLF,
TIGD4, ACPT, CDCA2, FAM83D, KIAA1841, OAZ1, RLIM, TIGD5, ACR, CDCA3, FAM83E,
KIAA1919, OAZ2, RLN1, TIGD6, ACRBP, CDCA4, FAM83F, KIAA1958, OAZ3, RLN2, TIGD7,
ACRC, CDCA5, FAM83G, KIAA1984, OBFC1, RLN3, TIGIT, ACRV1, CDCA7, FAM83H,
KIAA2013, OBP2A, RLTPR, TIMD4, ACSBG1, CDCA7L, FAM84A, KIAA2018, OBP2B,
RMDN1, TIMELESS, ACSBG2, CDCA8, FAM84B, KIAA2022, OBSCN, RMDN2, TIMM10,
ACSF2, CDCP1, FAM86A, KIAA2026, OBSL1, RMDN3, TIMM10B, ACSF3, CDCP2,
FAM86B1, KIDINS220, OC90, RMI1, TIMM13, ACSL1, CDH1, FAM86B2, KIF11, OCA2,
RMI2, TIMM17A, ACSL3, CDH10, FAM86C1, KIF12, OCEL1, RMND1, TIMM17B, ACSL4,
CDH11, FAM86KP, KIF13A, OCIAD1, RMND5A, TIMM21, ACSL5, CDH12, FAM89A,
KIF13B, OCIAD2, RMND5B, TIMM22, ACSL6, CDH13, FAM89B, KIF14, OCLM, RNASE1,
TIMM23, ACSM1, CDH15, FAM8A1, KIF15, OCLN, RNASE10, TIMM23B, ACSM2A, CDH16,
FAM90A1, KIF16B, OCM, RNASE11, TIMM44, ACSM2B, CDH17, FAM91A1, KIF17, OCM2,
RNASE12, TIMM50, ACSM3, CDH18, FAM92A1, KIF18A, OCRL, RNASE13, TIMM8A,
ACSM4, CDH19, FAM92B, KIF18B, OCSTAMP, RNASE2, TIMM8B, ACSM5, CDH2, FAM96A,
KIF19, ODAM, RNASE3, TIMM9, ACSS1, CDH20, FAM96B, KIF1A, ODC1, RNASE4,
TIMMDC1, ACSS2, CDH22, FAM98A, KIF1B, ODF1, RNASE6, TIMP1, ACSS3, CDH23,
FAM98B, KIF1C, ODF2, RNASE7, TIMP2, ACTA1, CDH24, FAM98C, KIF20A, ODF2L,
RNASE8, TIMP3, ACTA2, CDH26, FAM9A, KIF20B, ODF3, RNASE9, TIMP4, ACTB, CDH3,
FAM9B, KIF21A, ODF3B, RNASEH1, TINAG, ACTBL2, CDH4, FAM9C, KIF21B, ODF3L1,
RNASEH2A, TINAGL1, ACTC1, CDH5, FAN1, KIF22, ODF3L2, RNASEH2B, TINF2, ACTG1,
CDH6, FANCA, KIF23, ODF4, RNASEH2C, TIPARP, ACTG2, CDH7, FANCB, KIF24, OFCC1,
RNASEK, TIPIN, ACTL10, CDH8, FANCC, KIF25, OFD1, RNASEL, TIPRL, ACTL6A, CDH9,
FANCD2, KIF26A, OGDH, RNASET2, TIRAP, ACTL6B, CDHR1, FANCD2OS, KIF26B,
OGDHL, RND1, TJAP1, ACTL7A, CDHR2, FANCE, KIF27, OGFOD1, RND2, TJP1, ACTL7B,
CDHR3, FANCF, KIF28P, OGFOD2, RND3, TJP2, ACTL8, CDHR4, FANCG, KIF2A, OGFOD3,
RNF10, TJP3, ACTL9, CDHR5, FANCI, KIF2B, OGFR, RNF103, TK1, ACTN1, CDIP1, FANCL,
KIF2C, OGFRL1, RNF103-CHMP3, TK2, ACTN2, CDIPT, FANCM, KIF3A, OGG1, RNF11,
TKT, ACTN3, CDK1, FANK1, KIF3B, OGN, RNF111, TKTL1, ACTN4, CDK10, FAP, KIF3C,
OGT, RNF112, TKTL2, ACTR10, CDK11A, FAR1, KIF4A, OIP5, RNF113A, TLCD1, ACTR1A,
CDK11B, FAR2, KIF4B, OIT3, RNF113B, TLCD2, ACTR1B, CDK12, FARP1, KIF5A, OLA1,
RNF114, TLDC1, ACTR2, CDK13, FARP2, KIF5B, OLAH, RNF115, TLDC2, ACTR3, CDK14,
FARS2, KIF5C, OLFM1, RNF121, TLE1, ACTR3B, CDK15, FARSA, KIF6, OLFM2, RNF122,
TLE2, ACTR3C, CDK16, FARSB, KIF7, OLFM3, RNF123, TLE3, ACTR5, CDK17, FAS, KIF9,
OLFM4, RNF125, TLE4, ACTR6, CDK18, FASLG, KIFAP3, OLFML1, RNF126, TLE6, ACTR8,
CDK19, FASN, KIFC1, OLFML2A, RNF128, TLK1, ACTRT1, CDK2, FASTK, KIFC2,
OLFML2B, RNF13, TLK2, ACTRT2, CDK20, FASTKD1, KIFC3, OLFML3, RNF130, TLL1,
ACTRT3, CDK2AP1, FASTKD2, KIN, OLIG1, RNF133, TLL2, ACVR1, CDK2AP2, FASTKD3,
KIR2DL1, OLIG2, RNF135, TLN1, ACVR1B, CDK3, FASTKD5, KIR2DL3, OLIG3, RNF138,
TLN2, ACVR1C, CDK4, FAT1, KIR2DL4, OLR1, RNF139, TLR1, ACVR2A, CDK5, FAT2,
KIR2DS4, OMA1, RNF14, TLR10, ACVR2B, CDK5R1, FAT3, KIR3DL1, OMD, RNF141, TLR2,
ACVRL1, CDK5R2, FAT4, KIR3DL2, OMG, RNF144A, TLR3, ACY1, CDK5RAP1, FATE1,
KIR3DL3, OMP, RNF144B, TLR4, ACY3, CDK5RAP2, FAU, KIRREL, ONECUT1, RNF145,
TLR5, ACYP1, CDK5RAP3, FAXC, KIRREL2, ONECUT2, RNF146, TLR6, ACYP2, CDK6,
FAXDC2, KIRREL3, ONECUT3, RNF148, TLR7, ADA, CDK7, FBF1, KISS1, OOEP, RNF149,
TLR8, ADAD1, CDK8, FBL, KISS1R, OOSP2, RNF150, TLR9, ADAD2, CDK9, FBLIM1, KIT,
OPA1, RNF151, TLX1, ADAL, CDKAL1, FBLN1, KITLG, OPA3, RNF152, TLX1NB, ADAM10,
CDKL1, FBLN2, KL, OPALIN, RNF157, TLX2, ADAM11, CDKL2, FBLN5, KLB, OPCML,
RNF165, TLX3, ADAM12, CDKL3, FBLN7, KLC1, OPHN1, RNF166, TM2D1, ADAM15,
CDKL4, FBN1, KLC2, OPLAH, RNF167, TM2D2, ADAM17, CDKL5, FBN2, KLC3, OPN1LW,
RNF168, TM2D3, ADAM18, CDKN1A, FBN3, KLC4, OPN1MW, RNF169, TM4SF1, ADAM19,
CDKN1B, FBP1, KLF1, OPN1MW2, RNF17, TM4SF18, ADAM2, CDKN1C, FBP2, KLF10,
OPN1SW, RNF170, TM4SF19, ADAM20, CDKN2A, FBRS, KLF11, OPN3, RNF175, TM4SF20,
ADAM21, CDKN2AIP, FBRSL1, KLF12, OPN4, RNF180, TM4SF4, ADAM22, CDKN2AIPNL,
FBXL12, KLF13, OPN5, RNF181, TM4SF5, ADAM23, CDKN2B, FBXL13, KLF14, OPRD1,
RNF182, TM6SF1, ADAM28, CDKN2C, FBXL14, KLF15, OPRK1, RNF183, TM6SF2,
ADAM29, CDKN2D, FBXL15, KLF16, OPRL1, RNF185, TM7SF2, ADAM30, CDKN3, FBXL16,
KLF17, OPRM1, RNF186, TM7SF3, ADAM32, CDNF, FBXL17, KLF2, OPTC, RNF187,
TM9SF1, ADAM33, CDO1, FBXL18, KLF3, OPTN, RNF19A, TM9SF2, ADAM7, CDON,
FBXL19, KLF4, OR10A2, RNF19B, TM9SF3, ADAM8, CDPF1, FBXL2, KLF5, OR10A3, RNF2,
TM9SF4, ADAM9, CDR1, FBXL20, KLF6, OR10A4, RNF20, TMA16, ADAMDEC1, CDR2,
FBXL21, KLF7, OR10A5, RNF207, TMA7, ADAMTS1, CDR2L, FBXL22, KLF8, OR10A6,
RNF208, TMBIM1, ADAMTS10, CDRT1, FBXL3, KLF9, OR10A7, RNF212, TMBIM4,
ADAMTS12, CDRT15, FBXL4, KLHDC1, OR10AG1, RNF213, TMBIM6, ADAMTS13,
CDRT15L2, FBXL5, KLHDC10, OR10C1, RNF214, TMC1, ADAMTS14, CDRT4, FBXL6,
KLHDC2, OR10G2, RNF215, TMC2, ADAMTS15, CDS1, FBXL7, KLHDC3, OR10G3, RNF216,
TMC3, ADAMTS16, CDS2, FBXL8, KLHDC4, OR10G4, RNF217, TMC4, ADAMTS17, CDSN,
FBXO10, KLHDC7A, OR10G7, RNF219, TMC5, ADAMTS18, CDT1, FBXO11, KLHDC7B,
OR10G8, RNF220, TMC6, ADAMTS19, CDV3, FBXO15, KLHDC8A, OR10G9, RNF222, TMC7,
ADAMTS2, CDX1, FBXO16, KLHDC8B, OR10H1, RNF223, TMC8, ADAMTS20, CDX2,
FBXO17, KLHDC9, OR10H2, RNF224, TMCC1, ADAMTS3, CDX4, FBXO18, KLHL1, OR10H3,
RNF24, TMCC2, ADAMTS4, CDY1, FBXO2, KLHL10, OR10H4, RNF25, TMCC3, ADAMTS5,
CDY1B, FBXO21, KLHL11, OR10H5, RNF26, TMCO1, ADAMTS6, CDY2A, FBXO22,
KLHL12, OR10J1, RNF31, TMCO2, ADAMTS7, CDY2B, FBXO24, KLHL13, OR10J3, RNF32,
TMCO3, ADAMTS8, CDYL, FBXO25, KLHL14, OR10J5, RNF34, TMCO4, ADAMTS9, CDYL2,
FBXO27, KLHL15, OR10K1, RNF38, TMCO5A, ADAMTSL1, CEACAM1, FBXO28, KLHL17,
OR10K2, RNF39, TMCO6, ADAMTSL2, CEACAM16, FBXO3, KLHL18, OR10P1, RNF4,
TMED1, ADAMTSL3, CEACAM18, FBXO30, KLHL2, OR10Q1, RNF40, TMED10,
ADAMTSL4, CEACAM19, FBXO31, KLHL20, OR10R2, RNF41, TMED2, ADAMTSL5,
CEACAM20, FBXO32, KLHL21, OR10S1, RNF43, TMED3, ADAP1, CEACAM21, FBXO33,
KLHL22, OR10T2, RNF44, TMED4, ADAP2, CEACAM3, FBXO34, KLHL23, OR10V1, RNF5,
TMED5, ADAR, CEACAM4, FBXO36, KLHL24, OR10W1, RNF6, TMED6, ADARB1,
CEACAM5, FBXO38, KLHL25, OR10X1, RNF7, TMED7, ADARB2, CEACAM6, FBXO39,
KLHL26, OR10Z1, RNF8, TMED7-TICAM2, ADAT1, CEACAM7, FBXO4, KLHL28, OR11A1,
RNFT1, TMED8, ADAT2, CEACAM8, FBXO40, KLHL29, OR11G2, RNFT2, TMED9, ADAT3,
CEBPA, FBXO41, KLHL3, OR11H1, RNGTT, TMEFF1, ADC, CEBPB, FBXO42, KLHL30,
OR11H12, RNH1, TMEFF2, ADCK1, CEBPD, FBXO43, KLHL31, OR11H2, RNLS, TMEM100,
ADCK2, CEBPE, FBXO44, KLHL32, OR11H4, RNMT, TMEM101, ADCK3, CEBPG, FBXO45,
KLHL33, OR11H6, RNMTL1, TMEM102, ADCK4, CEBPZ, FBXO46, KLHL34, OR11L1,
RNPC3, TMEM104, ADCK5, CECR1, FBXO47, KLHL35, OR12D2, RNPEP, TMEM105,
ADCY1, CECR2, FBXO48, KLHL36, OR12D3, RNPEPL1, TMEM106A, ADCY10, CECR5,
FBXO5, KLHL38, OR13A1, RNPS1, TMEM106B, ADCY2, CECR6, FBXO6, KLHL4, OR13C2,
ROBO1, TMEM106C, ADCY3, CEL, FBXO7, KLHL40, OR13C3, ROBO2, TMEM107, ADCY4,
CELA1, FBXO8, KLHL41, OR13C4, ROBO3, TMEM108, ADCY5, CELA2A, FBXO9, KLHL42,
OR13C5, ROBO4, TMEM109, ADCY6, CELA2B, FBXW10, KLHL5, OR13C8, ROCK1,
TMEM11, ADCY7, CELA3A, FBXW11, KLHL6, OR13C9, ROCK2, TMEM110, ADCY8,
CELA3B, FBXW12, KLHL7, OR13D1, ROGDI, TMEM110-MUSTN1, ADCY9, CELF1, FBXW2,
KLHL8, OR13F1, ROM1, TMEM114, ADCYAP1, CELF2, FBXW4, KLHL9, OR13G1, ROMO1,
TMEM115, ADCYAP1R1, CELF3, FBXW5, KLK1, OR13H1, ROPN1, TMEM116, ADD1,
CELF4, FBXW7, KLK10, OR13J1, ROPN1B, TMEM117, ADD2, CELF5, FBXW8, KLK11,
OR14A16, ROPN1L, TMEM119, ADD3, CELF6, FBXW9, KLK12, OR14C36, ROR1,
TMEM120A, ADGB, CELSR1, FCAMR, KLK13, OR14J1, ROR2, TMEM120B, ADH1A,
CELSR2, FCAR, KLK14, OR1A1, RORA, TMEM121, ADH1B, CELSR3, FCER1A, KLK15,
OR1A2, RORB, TMEM123, ADH1C, CEMP1, FCER1G, KLK2, OR1B1, RORC, TMEM125,
ADH4, CEND1, FCER2, KLK3, OR1C1, ROS1, TMEM126A, ADH5, CENPA, FCF1, KLK4,
OR1D2, RP1, TMEM126B, ADH6, CENPB, FCGBP, KLK5, OR1D5, RP1L1, TMEM127, ADH7,
CENPBD1, FCGR1A, KLK6, OR1E1, RP2, TMEM128, ADHFE1, CENPC, FCGR1B, KLK7,
OR1E2, RP9, TMEM129, ADI1, CENPE, FCGR2A, KLK8, OR1F1, RPA1, TMEM130, ADIG,
CENPF, FCGR2B, KLK9, OR1G1, RPA2, TMEM131, ADIPOQ, CENPH, FCGR2C, KLKB1,
OR1I1, RPA3, TMEM132A, ADIPOR1, CENPI, FCGR3A, KLLN, OR1J1, RPA4, TMEM132B,
ADIPOR2, CENPJ, FCGR3B, KLRB1, OR1J4, RPAIN, TMEM132C, ADIRF, CENPK, FCGRT,
KLRC1, OR1K1, RPAP1, TMEM132D, ADK, CENPL, FCHO1, KLRC2, OR1L1, RPAP2,
TMEM132E, ADM, CENPM, FCHO2, KLRC3, OR1L3, RPAP3, TMEM133, ADM2, CENPN,
FCHSD1, KLRC4, OR1L4, RPE, TMEM134, ADM5, CENPO, FCHSD2, KLRC4-KLRK1, OR1L6,
RPE65, TMEM135, ADNP, CENPP, FCN1, KLRD1, OR1L8, RPF1, TMEM136, ADNP2, CENPQ,
FCN2, KLRF1, OR1M1, RPF2, TMEM138, ADO, CENPT, FCN3, KLRF2, OR1N1, RPGR,
TMEM139, ADORA1, CENPU, FCRL1, KLRG1, OR1N2, RPGRIP1, TMEM140, ADORA2A,
CENPV, FCRL2, KLRG2, OR1Q1, RPGRIP1L, TMEM141, ADORA2B, CENPW, FCRL3,
KLRK1, OR1S1, RPH3A, TMEM143, ADORA3, CEP104, FCRL4, KMO, OR1S2, RPH3AL,
TMEM144, ADPGK, CEP112, FCRL5, KMT2A, OR2A12, RPIA, TMEM145, ADPRH, CEP120,
FCRL6, KMT2B, OR2A14, RPL10, TMEM147, ADPRHL1, CEP128, FCRLA, KMT2C, OR2A2,
RPL10A, TMEM14A, ADPRHL2, CEP135, FCRLB, KMT2D, OR2A25, RPL10L, TMEM14B,
ADPRM, CEP152, FDCSP, KMT2E, OR2A4, RPL11, TMEM14C, ADRA1A, CEP164, FDFT1,
KNCN, OR2A5, RPL12, TMEM14E, ADRA1B, CEP170, FDPS, KNDC1, OR2A7, RPL13,
TMEM150A, ADRA1D, CEP170B, FDX1, KNG1, OR2AE1, RPL13A, TMEM150B, ADRA2A,
CEP19, FDX1L, KNOP1, OR2AG1, RPL14, TMEM150C, ADRA2B, CEP192, FDXACB1,
KNSTRN, OR2AG2, RPL15, TMEM151A, ADRA2C, CEP250, FDXR, KNTC1, OR2AK2, RPL17,
TMEM151B, ADRB1, CEP290, FECH, KPNA1, OR2AP1, RPL17-C18orf32, TMEM154, ADRB2,
CEP350, FEM1A, KPNA2, OR2AT4, RPL18, TMEM155, ADRB3, CEP41, FEM1B, KPNA3,
OR2B11, RPL18A, TMEM156, ADRBK1, CEP44, FEM1C, KPNA4, OR2B2, RPL19, TMEM158,
ADRBK2, CEP55, FEN1, KPNA5, OR2B3, RPL21, TMEM159, ADRM1, CEP57, FER, KPNA6,
OR2B6, RPL22, TMEM160, ADSL, CEP57L1, FER1L5, KPNA7, OR2C1, RPL22L1,
TMEM161A, ADSS, CEP63, FER1L6, KPNB1, OR2C3, RPL23, TMEM161B, ADSSL1, CEP68,
FERD3L, KPRP, OR2D2, RPL23A, TMEM163, ADTRP, CEP70, FERMT1, KPTN, OR2D3,
RPL24, TMEM164, AEBP1, CEP72, FERMT2, KRAS, OR2F1, RPL26, TMEM165, AEBP2,
CEP76, FERMT3, KRBA1, OR2F2, RPL26L1, TMEM167A, AEN, CEP78, FES, KRBA2, OR2G2,
RPL27, TMEM167B, AES, CEP85, FETUB, KRBOX1, OR2G3, RPL27A, TMEM168, AFAP1,
CEP85L, FEV, KRBOX4, OR2G6, RPL28, TMEM169, AFAP1L1, CEP89, FEZ1, KRCC1,
OR2H1, RPL29, TMEM17, AFAP1L2, CEP95, FEZ2, KREMEN1, OR2J2, RPL3, TMEM170A,
AFF1, CEP97, FEZF1, KREMEN2, OR2J3, RPL30, TMEM170B, AFF2, CEPT1, FEZF2, KRI1,
OR2K2, RPL31, TMEM171, AFF3, CER1, FFAR1, KRIT1, OR2L13, RPL32, TMEM173, AFF4,
CERCAM, FFAR2, KRR1, OR2L2, RPL34, TMEM174, AFG3L2, CERK, FFAR3, KRT1, OR2L3,
RPL35, TMEM175, AFM, CERKL, FFAR4, KRT10, OR2L5, RPL35A, TMEM176A, AFMID,
CERS1, FGA, KRT12, OR2L8, RPL36, TMEM176B, AFP, CERS2, FGB, KRT13, OR2M2,
RPL36A, TMEM177, AFTPH, CERS3, FGD1, KRT14, OR2M3, RPL36A-HNRNPH2,
TMEM178A, AGA, CERS4, FGD2, KRT15, OR2M4, RPL36AL, TMEM178B, AGAP1, CERS5,
FGD3, KRT16, OR2M5, RPL37, TMEM179, AGAP10, CERS6, FGD4, KRT17, OR2M7, RPL37A,
TMEM179B, AGAP11, CES1, FGD5, KRT18, OR2S2, RPL38, TMEM18, AGAP2, CES2, FGD6,
KRT19, OR2T1, RPL39, TMEM180, AGAP3, CES3, FGF1, KRT2, OR2T10, RPL39L, TMEM181,
AGAP4, CES4A, FGF10, KRT20, OR2T11, RPL3L, TMEM182, AGAP5, CES5A, FGF11,
KRT222, OR2T12, RPL4, TMEM183A, AGAP6, CETN1, FGF12, KRT23, OR2T2, RPL41,
TMEM183B, AGAP7, CETN2, FGF13, KRT24, OR2T27, RPL5, TMEM184A, AGAP8, CETN3,
FGF14, KRT25, OR2T29, RPL6, TMEM184B, AGAP9, CETP, FGF16, KRT26, OR2T3, RPL7,
TMEM184C, AGBL1, CFB, FGF17, KRT27, OR2T33, RPL7A, TMEM185A, AGBL2, CFC1,
FGF18, KRT28, OR2T34, RPL7L1, TMEM185B, AGBL3, CFC1B, FGF19, KRT3, OR2T35,
RPL8, TMEM186, AGBL4, CFD, FGF2, KRT31, OR2T4, RPL9, TMEM187, AGBL5, CFDP1,
FGF20, KRT32, OR2T5, RPLP0, TMEM189, AGER, CFH, FGF21, KRT33A, OR2T6, RPLP1,
TMEM189-UBE2V1, AGFG1, CFHR1, FGF22, KRT33B, OR2T8, RPLP2, TMEM19, AGFG2,
CFHR2, FGF23, KRT34, OR2V1, RPN1, TMEM190, AGGF1, CFHR3, FGF3, KRT35, OR2V2,
RPN2, TMEM191B, AGK, CFHR4, FGF4, KRT36, OR2W1, RPP14, TMEM191C, AGL, CFHR5,
FGF5, KRT37, OR2W3, RPP21, TMEM192, AGMAT, CFI, FGF6, KRT38, OR2W5, RPP25,
TMEM194A, AGMO, CFL1, FGF7, KRT39, OR2Y1, RPP25L, TMEM194B, AGO1, CFL2, FGF8,
KRT4, OR2Z1, RPP30, TMEM196, AGO2, CFLAR, FGF9, KRT40, OR3A1, RPP38, TMEM198,
AGO3, CFP, FGFBP1, KRT5, OR3A2, RPP40, TMEM199, AGO4, CFTR, FGFBP2, KRT6A,
OR3A3, RPRD1A, TMEM2, AGPAT1, CGA, FGFBP3, KRT6B, OR4A15, RPRD1B, TMEM200A,
AGPAT2, CGB, FGFR1, KRT6C, OR4A16, RPRD2, TMEM200B, AGPAT3, CGB1, FGFR1OP,
KRT7, OR4A47, RPRM, TMEM201, AGPAT4, CGB2, FGFR1OP2, KRT71, OR4A5, RPRML,
TMEM202, AGPAT5, CGB5, FGFR2, KRT72, OR4B1, RPS10, TMEM203, AGPAT6, CGB7,
FGFR3, KRT73, OR4C11, RPS10-NUDT3, TMEM204, AGPAT9, CGB8, FGFR4, KRT74,
OR4C12, RPS11, TMEM205, AGPS, CGGBP1, FGFRL1, KRT75, OR4C13, RPS12, TMEM206,
AGR2, CGN, FGG, KRT76, OR4C15, RPS13, TMEM207, AGR3, CGNL1, FGGY, KRT77,
OR4C16, RPS14, TMEM208, AGRN, CGREF1, FGL1, KRT78, OR4C3, RPS15, TMEM209,
AGRP, CGRRF1, FGL2, KRT79, OR4C45, RPS15A, TMEM210, AGT, CH25H, FGR, KRT8,
OR4C46, RPS16, TMEM211, AGTPBP1, CHAC1, FH, KRT80, OR4C6, RPS17, TMEM212,
AGTR1, CHAC2, FHAD1, KRT81, OR4D1, RPS17L, TMEM213, AGTR2, CHAD, FHDC1,
KRT82, OR4D10, RPS18, TMEM214, AGTRAP, CHADL, FHIT, KRT83, OR4D11, RPS19,
TMEM215, AGXT, CHAF1A, FHL1, KRT84, OR4D2, RPS19BP1, TMEM216, AGXT2, CHAF1B,
FHL2, KRT85, OR4D5, RPS2, TMEM217, AHCTF1, CHAMP1, FHL3, KRT86, OR4D6, RPS20,
TMEM218, AHCY, CHAT, FHL5, KRT9, OR4D9, RPS21, TMEM219, AHCYL1, CHCHD1,
FHOD1, KRTAP10-1, OR4E2, RPS23, TMEM220, AHCYL2, CHCHD10, FHOD3, KRTAP10-10,
OR4F15, RPS24, TMEM221, AHDC1, CHCHD2, FIBCD1, KRTAP10-11, OR4F16, RPS25,
TMEM222, AHI1, CHCHD3, FIBIN, KRTAP10-12, OR4F21, RPS26, TMEM223, AHNAK,
CHCHD4, FIBP, KRTAP10-2, OR4F29, RPS27, TMEM225, AHNAK2, CHCHD5, FICD,
KRTAP10-3, OR4F3, RPS27A, TMEM229A, AHR, CHCHD6, FIG4, KRTAP10-4, OR4F4,
RPS27L, TMEM229B, AHRR, CHCHD7, FIGF, KRTAP10-5, OR4F5, RPS28, TMEM230,
AHSA1, CHD1, FIGLA, KRTAP10-6, OR4F6, RPS29, TMEM231, AHSA2, CHD1L, FIGN,
KRTAP10-7, OR4K1, RPS3, TMEM232, AHSG, CHD2, FIGNL1, KRTAP10-8, OR4K13, RPS3A,
TMEM233, AHSP, CHD3, FIGNL2, KRTAP10-9, OR4K14, RPS4X, TMEM234, AICDA, CHD4,
FILIP1, KRTAP1-1, OR4K15, RPS4Y1, TMEM235, AIDA, CHD5, FILIP1L, KRTAP11-1,
OR4K17, RPS4Y2, TMEM236, AIF1, CHD6, FIP1L1, KRTAP12-1, OR4K2, RPS5, TMEM237,
AIF1L, CHD7, FIS1, KRTAP12-2, OR4K5, RPS6, TMEM238, AIFM1, CHD8, FITM1, KRTAP12-
3, OR4L1, RPS6KA1, TMEM239, AIFM2, CHD9, FITM2, KRTAP12-4, OR4M1, RPS6KA2,
TMEM240, AIFM3, CHDC2, FIZ1, KRTAP1-3, OR4M2, RPS6KA3, TMEM241, AIG1, CHDH,
FJX1, KRTAP13-1, OR4N2, RPS6KA4, TMEM242, AIM1, CHEK1, FKBP10, KRTAP13-2,
OR4N4, RPS6KA5, TMEM243, AIM1L, CHEK2, FKBP11, KRTAP13-3, OR4N5, RPS6KA6,
TMEM244, AIM2, CHERP, FKBP14, KRTAP13-4, OR4P4, RPS6KB1, TMEM245, AIMP1,
CHFR, FKBP15, KRTAP1-4, OR4Q3, RPS6KB2, TMEM246, AIMP2, CHGA, FKBP1A,
KRTAP1-5, OR4S1, RPS6KC1, TMEM247, AIP, CHGB, FKBP1B, KRTAP15-1, OR4S2,
RPS6KL1, TMEM248, AIPL1, CHI3L1, FKBP2, KRTAP16-1, OR4X1, RPS7, TMEM249, AIRE,
CHI3L2, FKBP3, KRTAP17-1, OR4X2, RPS8, TMEM25, AJAP1, CHIA, FKBP4, KRTAP19-1,
OR51A2, RPS9, TMEM251, AJUBA, CHIC1, FKBP5, KRTAP19-2, OR51A4, RPSA, TMEM252,
AK1, CHIC2, FKBP6, KRTAP19-3, OR51A7, RPTN, TMEM253, AK2, CHID1, FKBP7,
KRTAP19-4, OR51B2, RPTOR, TMEM254, AK3, CHIT1, FKBP8, KRTAP19-5, OR51B4,
RPUSD1, TMEM255A, AK4, CHKA, FKBP9, KRTAP19-6, OR51B5, RPUSD2, TMEM255B,
AK5, CHKB, FKBPL, KRTAP19-7, OR51B6, RPUSD3, TMEM256, AK6, CHL1, FKRP,
KRTAP19-8, OR51D1, RPUSD4, TMEM257, AK7, CHM, FKTN, KRTAP20-1, OR51E1, RQCD1,
TMEM258, AK8, CHML, FLAD1, KRTAP20-2, OR51E2, RRAD, TMEM259, AK9, CHMP1A,
FLCN, KRTAP20-3, OR51F1, RRAGA, TMEM26, AKAP1, CHMP1B, FLG, KRTAP2-1, OR51F2,
RRAGB, TMEM260, AKAP10, CHMP2A, FLG2, KRTAP21-1, OR51G1, RRAGC, TMEM261,
AKAP11, CHMP2B, FLI1, KRTAP21-2, OR51G2, RRAGD, TMEM27, AKAP12, CHMP3, FLII,
KRTAP21-3, OR51I1, RRAS, TMEM30A, AKAP13, CHMP4A, FLJ22184, KRTAP2-2, OR51I2,
RRAS2, TMEM30B, AKAP14, CHMP4B, FLJ25363, KRTAP22-1, OR51L1, RRBP1, TMEM31,
AKAP17A, CHMP4C, FLJ44313, KRTAP22-2, OR51M1, RREB1, TMEM33, AKAP2, CHMP5,
FLJ44635, KRTAP2-3, OR51Q1, RRH, TMEM35, AKAP3, CHMP6, FLJ45513, KRTAP23-1,
OR51S1, RRM1, TMEM37, AKAP4, CHMP7, FLNA, KRTAP2-4, OR51T1, RRM2, TMEM38A,
AKAP5, CHN1, FLNB, KRTAP24-1, OR51V1, RRM2B, TMEM38B, AKAP6, CHN2, FLNC,
KRTAP25-1, OR52A1, RRN3, TMEM39A, AKAP7, CHODL, FLOT1, KRTAP26-1, OR52A5,
RRNAD1, TMEM39B, AKAP8, CHORDC1, FLOT2, KRTAP27-1, OR52B2, RRP1, TMEM40,
AKAP8L, CHP1, FLRT1, KRTAP29-1, OR52B4, RRP12, TMEM41A, AKAP9, CHP2, FLRT2,
KRTAP3-1, OR52B6, RRP15, TMEM41B, AKIP1, CHPF, FLRT3, KRTAP3-2, OR52D1, RRP1B,
TMEM42, AKIRIN1, CHPF2, FLT1, KRTAP3-3, OR52E2, RRP36, TMEM43, AKIRIN2, CHPT1,
FLT3, KRTAP4-1, OR52E4, RRP7A, TMEM44, AKNA, CHRAC1, FLT3LG, KRTAP4-11,
OR52E6, RRP8, TMEM45A, AKNAD1, CHRD, FLT4, KRTAP4-12, OR52E8, RRP9, TMEM45B,
AKR1A1, CHRDL1, FLVCR1, KRTAP4-2, OR52H1, RRS1, TMEM47, AKR1B1, CHRDL2,
FLVCR2, KRTAP4-3, OR52I1, RS1, TMEM5, AKR1B10, CHRFAM7A, FLYWCH1, KRTAP4-4,
OR52I2, RSAD1, TMEM50A, AKR1B15, CHRM1, FLYWCH2, KRTAP4-5, OR52J3, RSAD2,
TMEM50B, AKR1C1, CHRM2, FMN1, KRTAP4-6, OR52K1, RSBN1, TMEM51, AKR1C2,
CHRM3, FMN2, KRTAP4-7, OR52K2, RSBN1L, TMEM52, AKR1C3, CHRM4, FMNL1,
KRTAP4-8, OR52L1, RSC1A1, TMEM52B, AKR1C4, CHRM5, FMNL2, KRTAP4-9, OR52M1,
RSF1, TMEM53, AKR1D1, CHRNA1, FMNL3, KRTAP5-1, OR52N1, RSG1, TMEM54, AKR1E2,
CHRNA10, FMO1, KRTAP5-10, OR52N4, RSL1D1, TMEM55A, AKR7A2, CHRNA2, FMO2,
KRTAP5-11, OR52N5, RSL24D1, TMEM55B, AKR7A3, CHRNA3, FMO3, KRTAP5-2, OR52R1,
RSPH1, TMEM56, AKT1, CHRNA4, FMO4, KRTAP5-3, OR52W1, RSPH10B, TMEM56-
RWDD3, AKT1S1, CHRNA5, FMO5, KRTAP5-4, OR56A1, RSPH10B2, TMEM57, AKT2,
CHRNA6, FMOD, KRTAP5-5, OR56A3, RSPH3, TMEM59, AKT3, CHRNA7, FMR1, KRTAP5-
6, OR56A4, RSPH4A, TMEM59L, AKTIP, CHRNA9, FMR1NB, KRTAP5-7, OR56A5, RSPH6A,
TMEM60, ALAD, CHRNB1, FN1, KRTAP5-8, OR56B1, RSPH9, TMEM61, ALAS1, CHRNB2,
FN3K, KRTAP5-9, OR56B4, RSPO1, TMEM62, ALAS2, CHRNB3, FN3KRP, KRTAP6-1,
OR5A1, RSPO2, TMEM63A, ALB, CHRNB4, FNBP1, KRTAP6-2, OR5A2, RSPO3, TMEM63B,
ALCAM, CHRND, FNBP1L, KRTAP6-3, OR5AC2, RSPO4, TMEM63C, ALDH16A1, CHRNE,
FNBP4, KRTAP7-1, OR5AK2, RSPRY1, TMEM64, ALDH18A1, CHRNG, FNDC1, KRTAP8-1,
OR5AN1, RSRC1, TMEM65, ALDH1A1, CHST1, FNDC3A, KRTAP9-1, OR5AP2, RSRC2,
TMEM66, ALDH1A2, CHST10, FNDC3B, KRTAP9-2, OR5AR1, RSU1, TMEM67, ALDH1A3,
CHST11, FNDC4, KRTAP9-3, OR5AS1, RTBDN, TMEM68, ALDH1B1, CHST12, FNDC5,
KRTAP9-4, OR5AU1, RTCA, TMEM69, ALDH1L1, CHST13, FNDC7, KRTAP9-6, OR5B12,
RTCB, TMEM70, ALDH1L2, CHST14, FNDC8, KRTAP9-7, OR5B17, RTDR1, TMEM71,
ALDH2, CHST15, FNDC9, KRTAP9- 8, OR5B2, RTEL1, TMEM72, ALDH3A1, CHST2, FNIP1,
KRTAP9-9, OR5B21, RTF1, TMEM74, ALDH3A2, CHST3, FNIP2, KRTCAP2, OR5B3,
RTFDC1, TMEM74B, ALDH3B1, CHST4, FNTA, KRTCAP3, OR5C1, RTKN, TMEM79,
ALDH3B2, CHST5, FNTB, KRTDAP, OR5D13, RTKN2, TMEM80, ALDH4A1, CHST6,
FOCAD, KSR1, OR5D14, RTL1, TMEM81, ALDH5A1, CHST7, FOLH1, KSR2, OR5D16, RTN1,
TMEM82, ALDH6A1, CHST8, FOLH1B, KTI12, OR5D18, RTN2, TMEM86A, ALDH7A1,
CHST9, FOLR1, KTN1, OR5F1, RTN3, TMEM86B, ALDH8A1, CHSY1, FOLR2, KXD1, OR5H1,
RTN4, TMEM87A, ALDH9A1, CHSY3, FOLR3, KY, OR5H14, RTN4IP1, TMEM87B, ALDOA,
CHTF18, FOLR4, KYNU, OR5H15, RTN4R, TMEM88, ALDOB, CHTF8, FOPNL, L1CAM,
OR5H2, RTN4RL1, TMEM88B, ALDOC, CHTOP, FOS, L1TD1, OR5H6, RTN4RL2, TMEM89,
ALG1, CHUK, FOSB, L2HGDH, OR5I1, RTP1, TMEM8A, ALG10, CHURC1, FOSL1,
L3HYPDH, OR5J2, RTP2, TMEM8B, ALG10B, CHURC1-FNTB, FOSL2, L3MBTL1, OR5K1,
RTP3, TMEM8C, ALG11, CIAO1, FOXA1, L3MBTL2, OR5K2, RTP4, TMEM9, ALG12,
CIAPIN1, FOXA2, L3MBTL3, OR5K3, RTTN, TMEM91, ALG13, CIB1, FOXA3, L3MBTL4,
OR5K4, RUFY1, TMEM92, ALG14, CIB2, FOXB1, LACC1, OR5L1, RUFY2, TMEM95, ALG1L,
CIB3, FOXB2, LACE1, OR5L2, RUFY3, TMEM97, ALG1L2, CIB4, FOXC1, LACRT, OR5M1,
RUFY4, TMEM98, ALG2, CIC, FOXC2, LACTB, OR5M10, RUNDC1, TMEM99, ALG3, CIDEA,
FOXD1, LACTB2, OR5M11, RUNDC3A, TMEM9B, ALG5, CIDEB, FOXD2, LACTBL1,
OR5M3, RUNDC3B, TMF1, ALG6, CIDEC, FOXD3, LAD1, OR5M8, RUNX1, TMIE, ALG8,
CIITA, FOXD4, LAG3, OR5M9, RUNX1T1, TMIGD1, ALG9, CILP, FOXD4L1, LAGE3, OR5P2,
RUNX2, TMIGD2, ALK, CILP2, FOXD4L2, LAIR1, OR5P3, RUNX3, TMLHE, ALKBH1, CINP,
FOXD4L3, LAIR2, OR5R1, RUSC1, TMOD1, ALKBH2, CIR1, FOXD4L4, LALBA, OR5T1,
RUSC1-AS1, TMOD2, ALKBH3, CIRBP, FOXD4L5, LAMA1, OR5T2, RUSC2, TMOD3,
ALKBH4, CIRH1A, FOXD4L6, LAMA2, OR5T3, RUVBL1, TMOD4, ALKBH5, CISD1, FOXE1,
LAMA3, OR5V1, RUVBL2, TMPO, ALKBH6, CISD2, FOXE3, LAMA4, OR5W2, RWDD1,
TMPPE, ALKBH7, CISD3, FOXF1, LAMA5, OR6A2, RWDD2A, TMPRSS11A, ALKBH8, CISH,
FOXF2, LAMB1, OR6B1, RWDD2B, TMPRSS11B, ALLC, CIT, FOXG1, LAMB2, OR6B3,
RWDD3, TMPRSS11D, ALMS1, CITED1, FOXH1, LAMB3, OR6C1, RWDD4, TMPRSS11E,
ALOX12, CITED2, FOXI1, LAMB4, OR6C2, RXFP1, TMPRSS11F, ALOX12B, CITED4, FOXI2,
LAMC1, OR6C3, RXFP2, TMPRSS12, ALOX15, CIZ1, FOXI3, LAMC2, OR6C4, RXFP3,
TMPRSS13, ALOX15B, CKAP2, FOXJ1, LAMC3, OR6C6, RXFP4, TMPRSS15, ALOX5,
CKAP2L, FOXJ2, LAMP1, OR6C65, RXRA, TMPRSS2, ALOX5AP, CKAP4, FOXJ3, LAMP2,
OR6C68, RXRB, TMPRSS3, ALOXE3, CKAP5, FOXK1, LAMP3, OR6C70, RXRG, TMPRSS4,
ALPI, CKB, FOXK2, LAMP5, OR6C74, RYBP, TMPRSS5, ALPK1, CKLF, FOXL1, LAMTOR1,
OR6C75, RYK, TMPRSS6, ALPK2, CKLF-CMTM1, FOXL2, LAMTOR2, OR6C76, RYR1,
TMPRSS7, ALPK3, CKM, FOXM1, LAMTOR3, OR6F1, RYR2, TMPRSS9, ALPL, CKMT1A,
FOXN1, LAMTOR4, OR6K2, RYR3, TMSB10, ALPP, CKMT1B, FOXN2, LAMTOR5, OR6K3,
S100A1, TMSB15A, ALPPL2, CKMT2, FOXN3, LANCL1, OR6K6, S100A10, TMSB15B, ALS2,
CKS1B, FOXN4, LANCL2, OR6M1, S100A11, TMSB4X, ALS2CL, CKS2, FOXO1, LANCL3,
OR6N1, S100A12, TMSB4Y, ALS2CR11, CLASP1, FOXO3, LAP3, OR6N2, S100A13, TMTC1,
ALS2CR12, CLASP2, FOXO4, LAPTM4A, OR6P1, S100A14, TMTC2, ALX1, CLASRP, FOXO6,
LAPTM4B, OR6Q1, S100A16, TMTC3, ALX3, CLC, FOXP1, LAPTM5, OR6S1, S100A2,
TMTC4, ALX4, CLCA1, FOXP2, LARGE, OR6T1, S100A3, TMUB1, ALYREF, CLCA2, FOXP3,
LARP1, OR6V1, S100A4, TMUB2, AMACR, CLCA4, FOXP4, LARP1B, OR6X1, S100A5,
TMX1, AMBN, CLCC1, FOXQ1, LARP4, OR6Y1, S100A6, TMX2, AMBP, CLCF1, FOXR1,
LARP4B, OR7A10, S100A7, TMX3, AMBRA1, CLCN1, FOXR2, LARP6, OR7A17, S100A7A,
TMX4, AMD1, CLCN2, FOXRED1, LARP7, OR7A5, S100A7L2, TNC, AMDHD1, CLCN3,
FOXRED2, LARS, OR7C1, S100A8, TNF, AMDHD2, CLCN4, FOXS1, LARS2, OR7C2, S100A9,
TNFAIP1, AMELX, CLCN5, FPGS, LAS1L, OR7D2, S100B, TNFAIP2, AMELY, CLCN6, FPGT,
LASP1, OR7D4, S100G, TNFAIP3, AMER1, CLCN7, FPGT-TNNI3K, LAT, OR7E24, S100P,
TNFAIP6, AMER2, CLCNKA, FPR1, LAT2, OR7G1, S100PBP, TNFAIP8, AMER3, CLCNKB,
FPR2, LATS1, OR7G2, S100Z, TNFAIP8L1, AMFR, CLDN1, FPR3, LATS2, OR7G3, S1PR1,
TNFAIP8L2, AMH, CLDN10, FRA10AC1, LAX1, OR8A1, S1PR2, TNFAIP8L2-SCNM1,
AMHR2, CLDN11, FRAS1, LAYN, OR8B12, S1PR3, TNFAIP8L3, AMICA1, CLDN12, FRAT1,
LBH, OR8B2, S1PR4, TNFRSF10A, AMIGO1, CLDN14, FRAT2, LBP, OR8B3, S1PR5,
TNFRSF10B, AMIGO2, CLDN15, FREM1, LBR, OR8B4, SAA1, TNFRSF10C, AMIGO3,
CLDN16, FREM2, LBX1, OR8B8, SAA2, TNFRSF10D, AMMECR1, CLDN17, FREM3, LBX2,
OR8D1, SAA2-SAA4, TNFRSF11A, AMMECR1L, CLDN18, FRG1, LCA5, OR8D2, SAA4,
TNFRSF11B, AMN, CLDN19, FRG2, LCA5L, OR8D4, SAAL1, TNFRSF12A, AMN1, CLDN2,
FRG2B, LCAT, OR8G1, SAC3D1, TNFRSF13B, AMOT, CLDN20, FRG2C, LCE1A, OR8G2,
SACM1L, TNFRSF13C, AMOTL1, CLDN22, FRK, LCE1B, OR8G5, SACS, TNFRSF14,
AMOTL2, CLDN23, FRMD1, LCE1C, OR8H1, SAE1, TNFRSF17, AMPD1, CLDN24, FRMD3,
LCE1D, OR8H2, SAFB, TNFRSF18, AMPD2, CLDN25, FRMD4A, LCE1E, OR8H3, SAFB2,
TNFRSF19, AMPD3, CLDN3, FRMD4B, LCE1F, OR8I2, SAG, TNFRSF1A, AMPH, CLDN4,
FRMD5, LCE2A, OR8J1, SAGE1, TNFRSF1B, AMT, CLDN5, FRMD6, LCE2B, OR8J3, SALL1,
TNFRSF21, AMTN, CLDN6, FRMD7, LCE2C, OR8K1, SALL2, TNFRSF25, AMY1A, CLDN7,
FRMD8, LCE2D, OR8K3, SALL3, TNFRSF4, AMY1B, CLDN8, FRMPD1, LCE3A, OR8K5,
SALL4, TNFRSF6B, AMY1C, CLDN9, FRMPD2, LCE3B, OR8S1, SAMD1, TNFRSF8, AMY2A,
CLDND1, FRMPD3, LCE3C, OR8U1, SAMD10, TNFRSF9, AMY2B, CLDND2, FRMPD4,
LCE3D, OR8U8, SAMD11, TNFSF10, AMZ1, CLEC10A, FRRS1, LCE3E, OR9A2, SAMD12,
TNFSF11, AMZ2, CLEC11A, FRRS1L, LCE4A, OR9A4, SAMD13, TNFSF12, ANAPC1,
CLEC12A, FRS2, LCE5A, OR9G1, SAMD14, TNFSF12-TNFSF13, ANAPC10, CLEC12B, FRS3,
LCE6A, OR9G4, SAMD15, TNFSF13, ANAPC11, CLEC14A, FRY, LCK, OR9G9, SAMD3,
TNFSF13B, ANAPC13, CLEC16A, FRYL, LCLAT1, OR9I1, SAMD4A, TNFSF14, ANAPC15,
CLEC17A, FRZB, LCMT1, OR9K2, SAMD4B, TNFSF15, ANAPC16, CLEC18A, FSBP, LCMT2,
OR9Q1, SAMD5, TNFSF18, ANAPC2, CLEC18B, FSCB, LCN1, OR9Q2, SAMD7, TNFSF4,
ANAPC4, CLEC18C, FSCN1, LCN10, ORAI1, SAMD8, TNFSF8, ANAPC5, CLEC19A, FSCN2,
LCN12, ORAI2, SAMD9, TNFSF9, ANAPC7, CLEC1A, FSCN3, LCN15, ORAI3, SAMD9L,
TNIK, ANG, CLEC1B, FSD1, LCN2, ORAOV1, SAMHD1, TNIP1, ANGEL1, CLEC2A, FSD1L,
LCN6, ORC1, SAMM50, TNIP2, ANGEL2, CLEC2B, FSD2, LCN8, ORC2, SAMSN1, TNIP3,
ANGPT1, CLEC2D, FSHB, LCN9, ORC3, SAP130, TNK1, ANGPT2, CLEC2L, FSHR, LCNL1,
ORC4, SAP18, TNK2, ANGPT4, CLEC3A, FSIP1, LCOR, ORC5, SAP25, TNKS, ANGPTL1,
CLEC3B, FSIP2, LCORL, ORC6, SAP30, TNKS1BP1, ANGPTL2, CLEC4A, FST, LCP1, ORM1,
SAP30BP, TNKS2, ANGPTL3, CLEC4C, FSTL1, LCP2, ORM2, SAP30L, TNMD, ANGPTL4,
CLEC4D, FSTL3, LCT, ORMDL1, SAPCD1, TNN, ANGPTL5, CLEC4E, FSTL4, LCTL,
ORMDL2, SAPCD2, TNNC1, ANGPTL6, CLEC4F, FSTL5, LDB1, ORMDL3, SAR1A, TNNC2,
ANGPTL7, CLEC4G, FTCD, LDB2, OS9, SAR1B, TNNI1, ANHX, CLEC4M, FTH1, LDB3,
OSBP, SARDH, TNNI2, ANK1, CLEC5A, FTH1P18, LDHA, OSBP2, SARM1, TNNI3, ANK2,
CLEC6A, FTHL17, LDHAL6A, OSBPL10, SARNP, TNNI3K, ANK3, CLEC7A, FTL, LDHAL6B,
OSBPL11, SARS, TNNT1, ANKAR, CLEC9A, FTMT, LDHB, OSBPL1A, SARS2, TNNT2,
ANKDD1A, CLECL1, FTO, LDHC, OSBPL2, SART1, TNNT3, ANKDD1B, CLGN, FTSJ1,
LDHD, OSBPL3, SART3, TNP1, ANKEF1, CLHC1, FTSJ2, LDLR, OSBPL5, SASH1, TNP2,
ANKFN1, CLIC1, FTSJ3, LDLRAD1, OSBPL6, SASH3, TNPO1, ANKFY1, CLIC2, FUBP1,
LDLRAD2, OSBPL7, SASS6, TNPO2, ANKH, CLIC3, FUBP3, LDLRAD3, OSBPL8, SAT1,
TNPO3, ANKHD1, CLIC4, FUCA1, LDLRAD4, OSBPL9, SAT2, TNR, ANKHD1-EIF4EBP3,
CLIC5, FUCA2, LDLRAP1, OSCAR, SATB1, TNRC18, ANKIB1, CLIC6, FUK, LDOC1, OSCP1,
SATB2, TNRC6A, ANKK1, CLINT1, FUNDC1, LDOC1L, OSER1, SATL1, TNRC6B, ANKLE1,
CLIP1, FUNDC2, LEAP2, OSGEP, SAV1, TNRC6C, ANKLE2, CLIP2, FUOM, LECT1,
OSGEPL1, SAYSD1, TNS1, ANKMY1, CLIP3, FURIN, LECT2, OSGIN1, SBDS, TNS3,
ANKMY2, CLIP4, FUS, LEF1, OSGIN2, SBF1, TNS4, ANKRA2, CLK1, FUT1, LEFTY1, OSM,
SBF2, TNXB, ANKRD1, CLK2, FUT10, LEFTY2, OSMR, SBK1, TOB1, ANKRD10, CLK3,
FUT11, LEKR1, OSR1, SBK2, TOB2, ANKRD11, CLK4, FUT2, LELP1, OSR2, SBK3, TOE1,
ANKRD12, CLLU1, FUT3, LEMD1, OST4, SBNO1, TOLLIP, ANKRD13A, CLLU1OS, FUT4,
LEMD2, OSTC, SBNO2, TOM1, ANKRD13B, CLMN, FUT5, LEMD3, OSTF1, SBSN, TOM1L1,
ANKRD13C, CLMP, FUT6, LENEP, OSTM1, SBSPON, TOM1L2, ANKRD13D, CLN3, FUT7,
LENG1, OSTN, SC5D, TOMM20, ANKRD16, CLN5, FUT8, LENG8, OTC, SCAF1, TOMM20L,
ANKRD17, CLN6, FUT9, LENG9, OTOA, SCAF11, TOMM22, ANKRD18A, CLN8, FUZ, LEO1,
OTOF, SCAF4, TOMM34, ANKRD18B, CLNK, FXN, LEP, OTOG, SCAF8, TOMM40,
ANKRD2, CLNS1A, FXR1, LEPR, OTOGL, SCAI, TOMM40L, ANKRD20A1, CLOCK, FXR2,
LEPRE1, OTOL1, SCAMP1, TOMM5, ANKRD20A2, CLP1, FXYD1, LEPREL1, OTOP1,
SCAMP2, TOMM6, ANKRD20A3, CLPB, FXYD2, LEPREL2, OTOP2, SCAMP3, TOMM7,
ANKRD20A4, CLPP, FXYD3, LEPREL4, OTOP3, SCAMP4, TOMM70A, ANKRD22, CLPS,
FXYD4, LEPROT, OTOR, SCAMP5, TONSL, ANKRD23, CLPSL1, FXYD5, LEPROTL1, OTOS,
SCAND1, TOP1, ANKRD24, CLPSL2, FXYD6, LETM1, OTP, SCAND3, TOP1MT, ANKRD26,
CLPTM1, FXYD6-FXYD2, LETM2, OTUB1, SCAP, TOP2A, ANKRD27, CLPTM1L, FXYD7,
LETMD1, OTUB2, SCAPER, TOP2B, ANKRD28, CLPX, FYB, LEUTX, OTUD1, SCARA3,
TOP3A, ANKRD29, CLRN1, FYCO1, LFNG, OTUD3, SCARA5, TOP3B, ANKRD30A, CLRN2,
FYN, LGALS1, OTUD4, SCARB1, TOPAZ1, ANKRD30B, CLRN3, FYTTD1, LGALS12,
OTUD5, SCARB2, TOPBP1, ANKRD31, CLSPN, FZD1, LGALS13, OTUD6A, SCARF1,
TOPORS, ANKRD32, CLSTN1, FZD10, LGALS14, OTUD6B, SCARF2, TOR1A, ANKRD33,
CLSTN2, FZD2, LGALS16, OTUD7A, SCCPDH, TOR1AIP1, ANKRD33B, CLSTN3, FZD3,
LGALS2, OTUD7B, SCD, TOR1AIP2, ANKRD34A, CLTA, FZD4, LGALS3, OTX1, SCD5,
TOR1B, ANKRD34B, CLTB, FZD5, LGALS3BP, OTX2, SCEL, TOR2A, ANKRD34C, CLTC,
FZD6, LGALS4, OVCA2, SCFD1, TOR3A, ANKRD35, CLTCL1, FZD7, LGALS7, OVCH1,
SCFD2, TOR4A, ANKRD36, CLU, FZD8, LGALS7B, OVCH2, SCG2, TOX, ANKRD36B,
CLUAP1, FZD9, LGALS8, OVGP1, SCG3, TOX2, ANKRD36C, CLUH, FZR1, LGALS9,
OVOL1, SCG5, TOX3, ANKRD37, CLUL1, G0S2, LGALS9B, OVOL2, SCGB1A1, TOX4,
ANKRD39, CLVS1, G2E3, LGALS9C, OVOL3, SCGB1C1, TP53, ANKRD40, CLVS2, G3BP1,
LGALSL, OVOS, SCGB1D1, TP53AIP1, ANKRD42, CLYBL, G3BP2, LGI1, OVOS2, SCGB1D2,
TP53BP1, ANKRD44, CMA1, G6PC, LGI2, OXA1L, SCGB1D4, TP53BP2, ANKRD45, CMAS,
G6PC2, LGI3, OXCT1, SCGB2A1, TP53I11, ANKRD46, CMBL, G6PC3, LGI4, OXCT2,
SCGB2A2, TP53I13, ANKRD49, CMC1, G6PD, LGMN, OXER1, SCGB2B2, TP53I3, ANKRD50,
CMC2, GAA, LGR4, OXGR1, SCGB3A1, TP53INP1, ANKRD52, CMC4, GAB1, LGR5, OXLD1,
SCGB3A2, TP53INP2, ANKRD53, CMIP, GAB2, LGR6, OXNAD1, SCGN, TP53RK, ANKRD54,
CMKLR1, GAB3, LGSN, OXR1, SCHIP1, TP53TG3, ANKRD55, CMPK1, GAB4, LHB, OXSM,
SCIMP, TP53TG3B, ANKRD6, CMPK2, GABARAP, LHCGR, OXSR1, SCIN, TP53TG3C,
ANKRD60, CMSS1, GABARAPL1, LHFP, OXT, SCLT1, TP53TG3D, ANKRD61, CMTM1,
GABARAPL2, LHFPL1, OXTR, SCLY, TP53TG5, ANKRD62, CMTM2, GABBR1, LHFPL2,
P2RX1, SCMH1, TP63, ANKRD63, CMTM3, GABBR2, LHFPL3, P2RX2, SCML1, TP73,
ANKRD65, CMTM4, GABPA, LHFPL4, P2RX3, SCML2, TPBG, ANKRD66, CMTM5, GABPB1,
LHFPL5, P2RX4, SCML4, TPBGL, ANKRD7, CMTM6, GABPB2, LHPP, P2RX5, SCN10A,
TPCN1, ANKRD9, CMTM7, GABRA1, LHX1, P2RX6, SCN11A, TPCN2, ANKS1A, CMTM8,
GABRA2, LHX2, P2RX7, SCN1A, TPD52, ANKS1B, CMTR1, GABRA3, LHX3, P2RY1,
SCN1B, TPD52L1, ANKS3, CMTR2, GABRA4, LHX4, P2RY10, SCN2A, TPD52L2, ANKS4B,
CMYA5, GABRA5, LHX5, P2RY11, SCN2B, TPD52L3, ANKS6, CNBD1, GABRA6, LHX6,
P2RY12, SCN3A, TPGS1, ANKUB1, CNBD2, GABRB1, LHX8, P2RY13, SCN3B, TPGS2,
ANKZF1, CNBP, GABRB2, LHX9, P2RY14, SCN4A, TPH1, ANLN, CNDP1, GABRB3, LIAS,
P2RY2, SCN4B, TPH2, ANO1, CNDP2, GABRD, LIF, P2RY4, SCN5A, TPI1, ANO10, CNEP1R1,
GABRE, LIFR, P2RY6, SCN7A, TPK1, ANO2, CNFN, GABRG1, LIG1, P2RY8, SCN8A, TPM1,
ANO3, CNGA1, GABRG2, LIG3, P4HA1, SCN9A, TPM2, ANO4, CNGA2, GABRG3, LIG4,
P4HA2, SCNM1, TPM3, ANO5, CNGA3, GABRP, LILRA1, P4HA3, SCNN1A, TPM4, ANO6,
CNGA4, GABRQ, LILRA2, P4HB, SCNN1B, TPMT, ANO7, CNGB1, GABRR1, LILRA3,
P4HTM, SCNN1D, TPO, ANO8, CNGB3, GABRR2, LILRA4, PA2G4, SCNN1G, TPP1, ANO9,
CNIH1, GABRR3, LILRA5, PAAF1, SCO1, TPP2, ANP32A, CNIH2, GAD1, LILRA6, PABPC1,
SCO2, TPPP, ANP32B, CNIH3, GAD2, LILRB1, PABPC1L, SCOC, TPPP2, ANP32C, CNIH4,
GADD45A, LILRB2, PABPC1L2A, SCP2, TPPP3, ANP32D, CNKSR1, GADD45B, LILRB3,
PABPC1L2B, SCP2D1, TPR, ANP32E, CNKSR2, GADD45G, LILRB4, PABPC3, SCPEP1,
TPRA1, ANPEP, CNKSR3, GADD45GIP1, LILRB5, PABPC4, SCRG1, TPRG1, ANTXR1,
CNN1, GADL1, LIM2, PABPC4L, SCRIB, TPRG1L, ANTXR2, CNN2, GAGE1, LIMA1,
PABPC5, SCRN1, TPRKB, ANTXRL, CNN3, GAGE10, LIMCH1, PABPN1, SCRN2, TPRN,
ANXA1, CNNM1, GAGE12B, LIMD1, PABPN1L, SCRN3, TPRX1, ANXA10, CNNM2,
GAGE12C, LIMD2, PACRG, SCRT1, TPSAB1, ANXA11, CNNM3, GAGE12D, LIME1,
PACRGL, SCRT2, TPSB2, ANXA13, CNNM4, GAGE12E, LIMK1, PACS1, SCT, TPSD1,
ANXA2, CNOT1, GAGE12F, LIMK2, PACS2, SCTR, TPSG1, ANXA2R, CNOT10, GAGE12G,
LIMS1, PACSIN1, SCUBE1, TPST1, ANXA3, CNOT11, GAGE12H, LIMS2, PACSIN2, SCUBE2,
TPST2, ANXA4, CNOT2, GAGE12I, LIMS3, PACSIN3, SCUBE3, TPT1, ANXA5, CNOT3,
GAGE12J, LIN28A, PADI1, SCXA, TPTE, ANXA6, CNOT4, GAGE13, LIN28B, PADI2, SCXB,
TPTE2, ANXA7, CNOT6, GAGE2A, LIN37, PADI3, SCYL1, TPX2, ANXA8, CNOT6L,
GAGE2B, LIN52, PADI4, SCYL2, TRA2A, ANXA8L1, CNOT7, GAGE2C, LIN54, PADI6,
SCYL3, TRA2B, ANXA8L2, CNOT8, GAGE2D, LIN7A, PAEP, SDAD1, TRABD, ANXA9, CNP,
GAGE2E, LIN7B, PAF1, SDC1, TRABD2A, AOAH, CNPPD1, GAGE4, LIN7C, PAFAH1B1,
SDC2, TRABD2B, AOC1, CNPY1, GAGE5, LIN9, PAFAH1B2, SDC3, TRADD, AOC2, CNPY2,
GAGE6, LINC00452, PAFAH1B3, SDC4, TRAF1, AOC3, CNPY3, GAGE7, LINC00984,
PAFAH2, SDCBP, TRAF2, AOX1, CNPY4, GAGE8, LINGO1, PAG1, SDCBP2, TRAF3, AP1AR,
CNR1, GAK, LINGO2, PAGE1, SDCCAG3, TRAF3IP1, AP1B1, CNR2, GAL, LINGO3, PAGE2,
SDCCAG8, TRAF3IP2, AP1G1, CNRIP1, GAL3ST1, LINGO4, PAGE2B, SDE2, TRAF3IP3,
AP1G2, CNST, GAL3ST2, LINS, PAGE4, SDF2, TRAF4, AP1M1, CNTD1, GAL3ST3, LIPA,
PAGE5, SDF2L1, TRAF5, AP1M2, CNTD2, GAL3ST4, LIPC, PAGR1, SDF4, TRAF6, AP1S1,
CNTF, GALC, LIPE, PAH, SDHA, TRAF7, AP1S2, CNTFR, GALE, LIPF, PAICS, SDHAF1,
TRAFD1, AP1S3, CNTLN, GALK1, LIPG, PAIP1, SDHAF2, TRAIP, AP2A1, CNTN1, GALK2,
LIPH, PAIP2, SDHB, TRAK1, AP2A2, CNTN2, GALM, LIPI, PAIP2B, SDHC, TRAK2, AP2B1,
CNTN3, GALNS, LIPJ, PAK1, SDHD, TRAM1, AP2M1, CNTN4, GALNT1, LIPK, PAK1IP1,
SDK1, TRAM1LI, AP2S1, CNTN5, GALNT10, LIPM, PAK2, SDK2, TRAM2, AP3B1, CNTN6,
GALNT11, LIPN, PAK3, SDPR, TRANK1, AP3B2, CNTNAP1, GALNT12, LIPT1, PAK4,
SDR16C5, TRAP1, AP3D1, CNTNAP2, GALNT13, LIPT2, PAK6, SDR39U1, TRAPPC1, AP3M1,
CNTNAP3, GALNT14, LITAF, PAK7, SDR42E1, TRAPPC10, AP3M2, CNTNAP3B, GALNT15,
LIX1, PALB2, SDR9C7, TRAPPC11, AP3S1, CNTNAP4, GALNT16, LIX1L, PALD1, SDS,
TRAPPC12, AP3S2, CNTNAP5, GALNT18, LLGL1, PALLD, SDSL, TRAPPC13, AP4B1,
CNTRL, GALNT2, LLGL2, PALM, SEBOX, TRAPPC2, AP4E1, CNTROB, GALNT3, LLPH,
PALM2, SEC11A, TRAPPC2L, AP4M1, COA1, GALNT4, LMAN1, PALM2-AKAP2, SEC11C,
TRAPPC3, AP4S1, COA3, GALNT5, LMAN1L, PALM3, SEC13, TRAPPC3L, AP5B1, COA4,
GALNT6, LMAN2, PALMD, SEC14L1, TRAPPC4, AP5M1, COA5, GALNT7, LMAN2L, PAM,
SEC14L2, TRAPPC5, AP5S1, COA6, GALNT8, LMBR1, PAM16, SEC14L3, TRAPPC6A,
AP5Z1, COASY, GALNT9, LMBR1L, PAMR1, SEC14L4, TRAPPC6B, APAF1, COBL,
GALNTL5, LMBRD1, PAN2, SEC14L5, TRAPPC8, APBA1, COBLL1, GALNTL6, LMBRD2,
PAN3, SEC14L6, TRAPPC9, APBA2, COCH, GALP, LMCD1, PANK1, SEC16A, TRAT1,
APBA3, COG1, GALR1, LMF1, PANK2, SEC16B, TRDMT1, APBB1, COG2, GALR2, LMF2,
PANK3, SEC22A, TRDN, APBB1IP, COG3, GALR3, LMLN, PANK4, SEC22B, TREH, APBB2,
COG4, GALT, LMNA, PANX1, SEC22C, TREM1, APBB3, COG5, GAMT, LMNB1, PANX2,
SEC23A, TREM2, APC, COG6, GAN, LMNB2, PANX3, SEC23B, TREML1, APC2, COG7,
GANAB, LMO1, PAOX, SEC23IP, TREML2, APCDD1, COG8, GANC, LMO2, PAPD4, SEC24A,
TREML4, APCDD1L, COIL, GAP43, LMO3, PAPD5, SEC24B, TRERF1, APCS, COL10A1,
GAPDH, LMO4, PAPD7, SEC24C, TREX1, APEH, COL11A1, GAPDHS, LMO7, PAPL,
SEC24D, TREX2, APEX1, COL11A2, GAPT, LMOD1, PAPLN, SEC31A, TRH, APEX2,
COL12A1, GAPVD1, LMOD2, PAPOLA, SEC31B, TRHDE, APH1A, COL13A1, GAR1,
LMOD3, PAPOLB, SEC61A1, TRHR, APH1B, COL14A1, GAREM, LMTK2, PAPOLG,
SEC61A2, TRIAP1, API5, COL15A1, GAREML, LMTK3, PAPPA, SEC61B, TRIB1, APIP,
COL16A1, GARNL3, LMX1A, PAPPA2, SEC61G, TRIB2, APITD1, COL17A1, GARS, LMX1B,
PAPSS1, SEC62, TRIB3, APITD1-CORT, COL18A1, GART, LNP1, PAPSS2, SEC63, TRIL,
APLF, COL19A1, GAS1, LNPEP, PAQR3, SECISBP2, TRIM10, APLN, COL1A1, GAS2, LNX1,
PAQR4, SECISBP2L, TRIM11, APLNR, COL1A2, GAS2L1, LNX2, PAQR5, SECTM1, TRIM13,
APLP1, COL20A1, GAS2L2, LOH12CR1, PAQR6, SEH1L, TRIM14, APLP2, COL21A1,
GAS2L3, LONP1, PAQR7, SEL1L, TRIM15, APMAP, COL22A1, GAS6, LONP2, PAQR8,
SEL1L2, TRIM16, APOA1, COL23A1, GAS7, LONRF1, PAQR9, SEL1L3, TRIM16L, APOA1BP,
COL24A1, GAS8, LONRF2, PARD3, SELE, TRIM17, APOA2, COL25A1, GAST, LONRF3,
PARD3B, SELENBP1, TRIM2, APOA4, COL26A1, GATA1, LOR, PARD6A, SELK, TRIM21,
APOA5, COL27A1, GATA2, LOX, PARD6B, SELL, TRIM22, APOB, COL28A1, GATA3,
LOXHD1, PARD6G, SELM, TRIM23, APOBEC1, COL2A1, GATA4, LOXL1, PARG, SELO,
TRIM24, APOBEC2, COL3A1, GATA5, LOXL2, PARK2, SELP, TRIM25, APOBEC3A,
COL4A1, GATA6, LOXL3, PARK7, SELPLG, TRIM26, APOBEC3B, COL4A2, GATAD1,
LOXL4, PARL, SELRC1, TRIM27, APOBEC3C, COL4A3, GATAD2A, LPA, PARM1, SELT,
TRIM28, APOBEC3D, COL4A3BP, GATAD2B, LPAR1, PARN, SELV, TRIM29, APOBEC3F,
COL4A4, GATC, LPAR2, PARP1, SEMA3A, TRIM3, APOBEC3G, COL4A5, GATM, LPAR3,
PARP10, SEMA3B, TRIM31, APOBEC3H, COL4A6, GATS, LPAR4, PARP11, SEMA3C,
TRIM32, APOBEC4, COL5A1, GATSL1, LPAR5, PARP12, SEMA3D, TRIM33, APOBR,
COL5A2, GATSL2, LPAR6, PARP14, SEMA3E, TRIM34, APOC1, COL5A3, GATSL3, LPCAT1,
PARP15, SEMA3F, TRIM35, APOC2, COL6A1, GBA, LPCAT2, PARP16, SEMA3G, TRIM36,
APOC3, COL6A2, GBA2, LPCAT3, PARP2, SEMA4A, TRIM37, APOC4, COL6A3, GBA3,
LPCAT4, PARP3, SEMA4B, TRIM38, APOD, COL6A5, GBAS, LPGAT1, PARP4, SEMA4C,
TRIM39, APOE, COL6A6, GBE1, LPHN1, PARP6, SEMA4D, TRIM39-RPP21, APOF, COL7A1,
GBF1, LPHN2, PARP8, SEMA4F, TRIM4, APOH, COL8A1, GBGT1, LPHN3, PARP9, SEMA4G,
TRIM40, APOL1, COL8A2, GBP1, LPIN1, PARPBP, SEMA5A, TRIM41, APOL2, COL9A1,
GBP2, LPIN2, PARS2, SEMA5B, TRIM42, APOL3, COL9A2, GBP3, LPIN3, PARVA, SEMA6A,
TRIM43, APOL4, COL9A3, GBP4, LPL, PARVB, SEMA6B, TRIM43B, APOL5, COLCA2,
GBP5, LPO, PARVG, SEMA6C, TRIM44, APOL6, COLEC10, GBP6, LPP, PASD1, SEMA6D,
TRIM45, APOLD1, COLEC11, GBP7, LPPR1, PASK, SEMA7A, TRIM46, APOM, COLEC12,
GBX1, LPPR2, PATE1, SEMG1, TRIM47, APOO, COLGALT1, GBX2, LPPR3, PATE2, SEMG2,
TRIM48, APOOL, COLGALT2, GC, LPPR4, PATE3, SENP1, TRIM49, APOPT1, COLQ, GCA,
LPPR5, PATE4, SENP2, TRIM49B, APP, COMMD1, GCAT, LPXN, PATL1, SENP3, TRIM49C,
APPBP2, COMMD10, GCC1, LRAT, PATL2, SENP5, TRIM49D1, APPL1, COMMD2, GCC2,
LRBA, PATZ1, SENP6, TRIM49D2P, APPL2, COMMD3, GCDH, LRCH1, PAWR, SENP7,
TRIM5, APRT, COMMD3-BMI1, GCFC2, LRCH2, PAX1, SENP8, TRIM50, APTX, COMMD4,
GCG, LRCH3, PAX2, SEPHS1, TRIM51, AQP1, COMMD5, GCGR, LRCH4, PAX3, SEPHS2,
TRIM52, AQP10, COMMD6, GCH1, LRCOL1, PAX4, SEPN1, TRIM54, AQP11, COMMD7,
GCHFR, LRFN1, PAX5, SEPP1, TRIM55, AQP12A, COMMD8, GCK, LRFN2, PAX6, SEPSECS,
TRIM56, AQP12B, COMMD9, GCKR, LRFN3, PAX7, SEPT1, TRIM58, AQP2, COMP, GCLC,
LRFN4, PAX8, SEPT10, TRIM59, AQP3, COMT, GCLM, LRFN5, PAX9, SEPT11, TRIM6,
AQP4, COMTD1, GCM1, LRG1, PAXBP1, SEPT12, TRIM60, AQP5, COPA, GCM2, LRGUK,
PAXIP1, SEPT14, TRIM61, AQP6, COPB1, GCN1L1, LRIF1, PBDC1, SEPT15, TRIM62, AQP7,
COPB2, GCNT1, LRIG1, PBK, SEPT2, TRIM63, AQP8, COPE, GCNT2, LRIG2, PBLD, SEPT3,
TRIM64, AQP9, COPG1, GCNT3, LRIG3, PBOV1, SEPT4, TRIM64B, AQPEP, COPG2, GCNT4,
LRIT1, PBRM1, SEPT5, TRIM64C, AQR, COPRS, GCNT7, LRIT2, PBX1, SEPT6, TRIM65, AR,
COPS2, GCOM1, LRIT3, PBX2, SEPT7, TRIM66, ARAF, COPS3, GCSAM, LRMP, PBX3,
SEPT8, TRIM67, ARAP1, COPS4, GCSAML, LRP1, PBX4, SEPT9, TRIM68, ARAP2, COPS5,
GCSH, LRP10, PBXIP1, SEPW1, TRIM69, ARAP3, COPS6, GDA, LRP11, PC, SERAC1, TRIM6-
TRIM34, ARC, COPS7A, GDAP1, LRP12, PCBD1, SERBP1, TRIM7, ARCN1, COPS7B,
GDAP1L1, LRP1B, PCBD2, SERF1A, TRIM71, AREG, COPS8, GDAP2, LRP2, PCBP1,
SERF1B, TRIM72, AREGB, COPZ1, GDE1, LRP2BP, PCBP2, SERF2, TRIM73, AREL1, COPZ2,
GDF1, LRP3, PCBP3, SERGEF, TRIM74, ARF1, COQ10A, GDF10, LRP4, PCBP4, SERHL2,
TRIM77, ARF3, COQ10B, GDF11, LRP5, PCCA, SERINC1, TRIM8, ARF4, COQ2, GDF15,
LRP5L, PCCB, SERINC2, TRIM9, ARF5, COQ3, GDF2, LRP6, PCDH1, SERINC3, TRIML1,
ARF6, COQ4, GDF3, LRP8, PCDH10, SERINC4, TRIML2, ARFGAP1, COQ5, GDF5, LRPAP1,
PCDH11X, SERINC5, TRIO, ARFGAP2, COQ6, GDF6, LRPPRC, PCDH11Y, SERP1, TRIOBP,
ARFGAP3, COQ7, GDF7, LRR1, PCDH12, SERP2, TRIP10, ARFGEF1, COQ9, GDF9, LRRC1,
PCDH15, SERPINA1, TRIP11, ARFGEF2, CORIN, GDI1, LRRC10, PCDH17, SERPINA10,
TRIP12, ARFIP1, CORO1A, GDI2, LRRC10B, PCDH18, SERPINA11, TRIP13, ARFIP2,
CORO1B, GDNF, LRRC14, PCDH19, SERPINA12, TRIP4, ARFRP1, CORO1C, GDNF-AS1,
LRRC14B, PCDH20, SERPINA3, TRIP6, ARG1, CORO2A, GDPD1, LRRC15, PCDH7,
SERPINA4, TRIQK, ARG2, CORO2B, GDPD2, LRRC16A, PCDH8, SERPINA5, TRIT1, ARGFX,
CORO6, GDPD3, LRRC16B, PCDH9, SERPINA6, TRMT1, ARGLU1, CORO7, GDPD4,
LRRC17, PCDHA1, SERPINA7, TRMT10A, ARHGAP1, CORO7-PAM16, GDPD5, LRRC18,
PCDHA10, SERPINA9, TRMT10B, ARHGAP10, CORT, GDPGP1, LRRC19, PCDHA11,
SERPINB1, TRMT10C, ARHGAP11A, COTL1, GEM, LRRC2, PCDHA12, SERPINB10,
TRMT11, ARHGAP11B, COX10, GEMIN2, LRRC20, PCDHA13, SERPINB11, TRMT112,
ARHGAP12, COX11, GEMIN4, LRRC23, PCDHA2, SERPINB12, TRMT12, ARHGAP15,
COX14, GEMIN5, LRRC24, PCDHA3, SERPINB13, TRMT13, ARHGAP17, COX15, GEMIN6,
LRRC25, PCDHA4, SERPINB2, TRMT1L, ARHGAP18, COX16, GEMIN7, LRRC26, PCDHA5,
SERPINB3, TRMT2A, ARHGAP19, COX17, GEMIN8, LRRC27, PCDHA6, SERPINB4,
TRMT2B, ARHGAP20, COX18, GEN1, LRRC28, PCDHA7, SERPINB5, TRMT44, ARHGAP21,
COX19, GET4, LRRC29, PCDHA8, SERPINB6, TRMT5, ARHGAP22, COX20, GFAP, LRRC3,
PCDHA9, SERPINB7, TRMT6, ARHGAP23, COX4I1, GFER, LRRC30, PCDHAC1, SERPINB8,
TRMT61A, ARHGAP24, COX4I2, GFI1, LRRC31, PCDHAC2, SERPINB9, TRMT61B,
ARHGAP25, COX5A, GFI1B, LRRC32, PCDHB1, SERPINC1, TRMU, ARHGAP26, COX5B,
GFM1, LRRC34, PCDHB10, SERPIND1, TRNAU1AP, ARHGAP27, COX6A1, GFM2, LRRC36,
PCDHB11, SERPINE1, TRNP1, ARHGAP28, COX6A2, GFOD1, LRRC37A2, PCDHB12,
SERPINE2, TRNT1, ARHGAP29, COX6B1, GFOD2, LRRC37A3, PCDHB13, SERPINE3, TRO,
ARHGAP30, COX6B2, GFPT1, LRRC37B, PCDHB14, SERPINF1, TROAP, ARHGAP31,
COX6C, GFPT2, LRRC38, PCDHB15, SERPINF2, TROVE2, ARHGAP32, COX7A1, GFRA1,
LRRC39, PCDHB16, SERPING1, TRPA1, ARHGAP33, COX7A2, GFRA2, LRRC3B, PCDHB2,
SERPINH1, TRPC1, ARHGAP35, COX7A2L, GFRA3, LRRC3C, PCDHB3, SERPINI1, TRPC3,
ARHGAP36, COX7B, GFRA4, LRRC4, PCDHB4, SERPINI2, TRPC4, ARHGAP39, COX7B2,
GFRAL, LRRC40, PCDHB5, SERTAD1, TRPC4AP, ARHGAP4, COX7C, GGA1, LRRC41,
PCDHB6, SERTAD2, TRPC5, ARHGAP40, COX8A, GGA2, LRRC42, PCDHB7, SERTAD3,
TRPC5OS, ARHGAP42, COX8C, GGA3, LRRC43, PCDHB8, SERTAD4, TRPC6, ARHGAP44,
CP, GGACT, LRRC45, PCDHB9, SERTM1, TRPC7, ARHGAP5, CPA1, GGCT, LRRC46,
PCDHGA1, SESN1, TRPM1, ARHGAP6, CPA2, GGCX, LRRC47, PCDHGA10, SESN2, TRPM2,
ARHGAP8, CPA3, GGH, LRRC48, PCDHGA11, SESN3, TRPM3, ARHGAP9, CPA4, GGN,
LRRC49, PCDHGA12, SESTD1, TRPM4, ARHGDIA, CPA5, GGNBP2, LRRC4B, PCDHGA2,
SET, TRPM5, ARHGDIB, CPA6, GGPS1, LRRC4C, PCDHGA3, SETBP1, TRPM6, ARHGDIG,
CPAMD8, GGT1, LRRC52, PCDHGA4, SETD1A, TRPM7, ARHGEF1, CPB1, GGT2, LRRC55,
PCDHGA5, SETD1B, TRPM8, ARHGEF10, CPB2, GGT5, LRRC56, PCDHGA6, SETD2, TRPS1,
ARHGEF10L, CPD, GGT6, LRRC57, PCDHGA7, SETD3, TRPT1, ARHGEF11, CPE, GGT7,
LRRC58, PCDHGA8, SETD4, TRPV1, ARHGEF12, CPEB1, GGTLC1, LRRC59, PCDHGA9,
SETD5, TRPV2, ARHGEF15, CPEB2, GGTLC2, LRRC6, PCDHGB1, SETD6, TRPV3,
ARHGEF16, CPEB3, GH1, LRRC61, PCDHGB2, SETD7, TRPV4, ARHGEF17, CPEB4, GH2,
LRRC63, PCDHGB3, SETD8, TRPV5, ARHGEF18, CPED1, GHDC, LRRC66, PCDHGB4,
SETD9, TRPV6, ARHGEF19, CPLX1, GHITM, LRRC69, PCDHGB5, SETDB1, TRRAP,
ARHGEF2, CPLX2, GHR, LRRC7, PCDHGB6, SETDB2, TRUB1, ARHGEF25, CPLX3, GHRH,
LRRC70, PCDHGB7, SETMAR, TRUB2, ARHGEF26, CPLX4, GHRHR, LRRC71, PCDHGC3,
SETSIP, TSACC, ARHGEF28, CPM, GHRL, LRRC72, PCDHGC4, SETX, TSC1, ARHGEF3,
CPN1, GHSR, LRRC73, PCDHGC5, SEZ6, TSC2, ARHGEF33, CPN2, GID4, LRRC8A, PCDP1,
SEZ6L, TSC22D1, ARHGEF35, CPNE1, GID8, LRRC8B, PCED1A, SEZ6L2, TSC22D2,
ARHGEF37, CPNE2, GIF, LRRC8C, PCED1B, SF1, TSC22D3, ARHGEF38, CPNE3, GIGYF1,
LRRC8D, PCF11, SF3A1, TSC22D4, ARHGEF39, CPNE4, GIGYF2, LRRC8E, PCGF1, SF3A2,
TSEN15, ARHGEF4, CPNE5, GIMAP1, LRRCC1, PCGF2, SF3A3, TSEN2, ARHGEF40, CPNE6,
GIMAP1-GIMAP5, LRRD1, PCGF3, SF3B1, TSEN34, ARHGEF5, CPNE7, GIMAP2, LRRFIP1,
PCGF5, SF3B14, TSEN54, ARHGEF6, CPNE8, GIMAP4, LRRFIP2, PCGF6, SF3B2, TSFM,
ARHGEF7, CPNE9, GIMAP5, LRRIQ1, PCID2, SF3B3, TSG101, ARHGEF9, CPO, GIMAP6,
LRRIQ3, PCIF1, SF3B4, TSGA10, ARID1A, CPOX, GIMAP7, LRRIQ4, PCK1, SF3B5,
TSGA10IP, ARID1B, CPPED1, GIMAP8, LRRK1, PCK2, SFI1, TSGA13, ARID2, CPQ, GIMD1,
LRRK2, PCLO, SFMBT1, TSHB, ARID3A, CPS1, GIN1, LRRN1, PCM1, SFMBT2, TSHR,
ARID3B, CPSF1, GINM1, LRRN2, PCMT1, SFN, TSHZ1, ARID3C, CPSF2, GINS1, LRRN3,
PCMTD1, SFPQ, TSHZ2, ARID4A, CPSF3, GINS2, LRRN4, PCMTD2, SFR1, TSHZ3, ARID4B,
CPSF3L, GINS3, LRRN4CL, PCNA, SFRP1, TSKS, ARID5A, CPSF4, GINS4, LRRTM1, PCNP,
SFRP2, TSKU, ARID5B, CPSF4L, GIP, LRRTM2, PCNT, SFRP4, TSLP, ARIH1, CPSF6, GIPC1,
LRRTM3, PCNX, SFRP5, TSN, ARIH2, CPSF7, GIPC2, LRRTM4, PCNXL2, SFSWAP,
TSNARE1, ARIH2OS, CPT1A, GIPC3, LRSAM1, PCNXL3, SFT2D1, TSNAX, ARL1, CPT1B,
GIPR, LRTM1, PCNXL4, SFT2D2, TSNAXIP1, ARL10, CPT1C, GIT1, LRTM2, PCOLCE,
SFT2D3, TSPAN1, ARL11, CPT2, GIT2, LRTOMT, PCOLCE2, SFTA2, TSPAN10, ARL13A,
CPVL, GJA1, LRWD1, PCP2, SFTA3, TSPAN11, ARL13B, CPXCR1, GJA10, LSAMP, PCP4,
SFTPA1, TSPAN12, ARL14, CPXM1, GJA3, LSG1, PCP4L1, SFTPA2, TSPAN13, ARL14EP,
CPXM2, GJA4, LSM1, PCSK1, SFTPB, TSPAN14, ARL14EPL, CPZ, GJA5, LSM10, PCSK1N,
SFTPC, TSPAN15, ARL15, CR1, GJA8, LSM11, PCSK2, SFTPD, TSPAN16, ARL16, CR1L,
GJA9, LSM12, PCSK4, SFXN1, TSPAN17, ARL17A, CR2, GJB1, LSM14A, PCSK5, SFXN2,
TSPAN18, ARL17B, CRABP1, GJB2, LSM14B, PCSK6, SFXN3, TSPAN19, ARL2, CRABP2,
GJB3, LSM2, PCSK7, SFXN4, TSPAN2, ARL2BP, CRADD, GJB4, LSM3, PCSK9, SFXN5,
TSPAN3, ARL3, CRAMP1L, GJB5, LSM4, PCTP, SGCA, TSPAN31, ARL4A, CRAT, GJB6,
LSM5, PCYOX1, SGCB, TSPAN32, ARL4C, CRB1, GJB7, LSM6, PCYOX1L, SGCD, TSPAN33,
ARL4D, CRB2, GJC1, LSM7, PCYT1A, SGCE, TSPAN4, ARL5A, CRB3, GJC2, LSMD1,
PCYT1B, SGCG, TSPAN5, ARL5B, CRBN, GJC3, LSMEM1, PCYT2, SGCZ, TSPAN6, ARL5C,
CRCP, GJD2, LSMEM2, PDAP1, SGIP1, TSPAN7, ARL6, CRCT1, GJD3, LSP1, PDC, SGK1,
TSPAN8, ARL6IP1, CREB1, GJD4, LSR, PDCD1, SGK2, TSPAN9, ARL6IP4, CREB3, GK, LSS,
PDCD10, SGK223, TSPEAR, ARL6IP5, CREB3L1, GK2, LST1, PDCD11, SGK3, TSPO,
ARL6IP6, CREB3L2, GK5, LTA, PDCD1LG2, SGK494, TSPO2, ARL8A, CREB3L3, GKAP1,
LTA4H, PDCD2, SGMS1, TSPY1, ARL8B, CREB3L4, GKN1, LTB, PDCD2L, SGMS2, TSPY10,
ARL9, CREB5, GKN2, LTB4R, PDCD4, SGOL1, TSPY2, ARMC1, CREBBP, GLA, LTB4R2,
PDCD5, SGOL2, TSPY3, ARMC10, CREBL2, GLB1, LTBP1, PDCD6, SGPL1, TSPY4,
ARMC12, CREBRF, GLB1L, LTBP2, PDCD6IP, SGPP1, TSPY8, ARMC2, CREBZF, GLB1L2,
LTBP3, PDCD7, SGPP2, TSPYL1, ARMC3, CREG1, GLB1L3, LTBP4, PDCL, SGSH, TSPYL2,
ARMC4, CREG2, GLCCI1, LTBR, PDCL2, SGSM1, TSPYL4, ARMC5, CRELD1, GLCE, LTC4S,
PDCL3, SGSM2, TSPYL5, ARMC6, CRELD2, GLDC, LTF, PDDC1, SGSM3, TSPYL6, ARMC7,
CREM, GLDN, LTK, PDE10A, SGTA, TSR1, ARMC8, CRH, GLE1, LTN1, PDE11A, SGTB,
TSR2, ARMC9, CRHBP, GLG1, LTV1, PDE12, SH2B1, TSR3, ARMCX1, CRHR1, GLI1,
LUC7L, PDE1A, SH2B2, TSSC1, ARMCX2, CRHR2, GLI2, LUC7L2, PDE1B, SH2B3, TSSC4,
ARMCX3, CRIM1, GLI3, LUC7L3, PDE1C, SH2D1A, TSSK1B, ARMCX4, CRIP1, GLI4, LUM,
PDE2A, SH2D1B, TSSK2, ARMCX5, CRIP2, GLIPR1, LURAP1, PDE3A, SH2D2A, TSSK3,
ARMCX5-GPRASP2, CRIP3, GLIPR1L1, LURAP1L, PDE3B, SH2D3A, TSSK4, ARMCX6,
CRIPAK, GLIPR1L2, LUZP1, PDE4A, SH2D3C, TSSK6, ARMS2, CRIPT, GLIPR2, LUZP2,
PDE4B, SH2D4A, TST, ARNT, CRISP1, GLIS1, LUZP4, PDE4C, SH2D4B, TSTA3, ARNT2,
CRISP2, GLIS2, LUZP6, PDE4D, SH2D5, TSTD1, ARNTL, CRISP3, GLIS3, LXN, PDE4DIP,
SH2D6, TSTD2, ARNTL2, CRISPLD1, GLMN, LY6D, PDE5A, SH2D7, TSTD3, ARPC1A,
CRISPLD2, GLO1, LY6E, PDE6A, SH3BGR, TTBK1, ARPC1B, CRK, GLOD4, LY6G5B,
PDE6B, SH3BGRL, TTBK2, ARPC2, CRKL, GLOD5, LY6G5C, PDE6C, SH3BGRL2, TTC1,
ARPC3, CRLF1, GLP1R, LY6G6C, PDE6D, SH3BGRL3, TTC12, ARPC4, CRLF2, GLP2R,
LY6G6D, PDE6G, SH3BP1, TTC13, ARPC4-TTLL3, CRLF3, GLRA1, LY6G6F, PDE6H,
SH3BP2, TTC14, ARPC5, CRLS1, GLRA2, LY6H, PDE7A, SH3BP4, TTC16, ARPC5L, CRMP1,
GLRA3, LY6K, PDE7B, SH3BP5, TTC17, ARPP19, CRNKL1, GLRA4, LY75, PDE8A,
SH3BP5L, TTC18, ARPP21, CRNN, GLRB, LY75-CD302, PDE8B, SH3D19, TTC19, ARR3,
CROCC, GLRX, LY86, PDE9A, SH3D21, TTC21A, ARRB1, CROT, GLRX2, LY9, PDF,
SH3GL1, TTC21B, ARRB2, CRP, GLRX3, LY96, PDGFA, SH3GL2, TTC22, ARRDC1,
CRTAC1, GLRX5, LYAR, PDGFB, SH3GL3, TTC23, ARRDC2, CRTAM, GLS, LYG1, PDGFC,
SH3GLB1, TTC23L, ARRDC3, CRTAP, GLS2, LYG2, PDGFD, SH3GLB2, TTC24, ARRDC4,
CRTC1, GLT1D1, LYL1, PDGFRA, SH3KBP1, TTC25, ARRDC5, CRTC2, GLT6D1, LYN,
PDGFRB, SH3PXD2A, TTC26, ARSA, CRTC3, GLT8D1, LYNX1, PDGFRL, SH3PXD2B,
TTC27, ARSB, CRX, GLT8D2, LYPD1, PDHA1, SH3RF1, TTC28, ARSD, CRY1, GLTP, LYPD2,
PDHA2, SH3RF2, TTC29, ARSE, CRY2, GLTPD1, LYPD3, PDHB, SH3RF3, TTC3, ARSF,
CRYAA, GLTPD2, LYPD4, PDHX, SH3TC1, TTC30A, ARSG, CRYAB, GLTSCR1, LYPD5,
PDIA2, SH3TC2, TTC30B, ARSH, CRYBA1, GLTSCR1L, LYPD6, PDIA3, SH3YL1, TTC31,
ARSI, CRYBA2, GLTSCR2, LYPD6B, PDIA4, SHANK1, TTC32, ARSJ, CRYBA4, GLUD1,
LYPD8, PDIA5, SHANK2, TTC33, ARSK, CRYBB1, GLUD2, LYPLA1, PDIA6, SHANK3,
TTC34, ART1, CRYBB2, GLUL, LYPLA2, PDIK1L, SHARPIN, TTC36, ART3, CRYBB3,
GLYAT, LYPLAL1, PDILT, SHB, TTC37, ART4, CRYBG3, GLYATL1, LYRM1, PDK1, SHBG,
TTC38, ART5, CRYGA, GLYATL2, LYRM2, PDK2, SHC1, TTC39A, ARTN, CRYGB,
GLYATL3, LYRM4, PDK3, SHC2, TTC39B, ARV1, CRYGC, GLYCTK, LYRM5, PDK4, SHC3,
TTC39C, ARVCF, CRYGD, GLYR1, LYRM7, PDLIM1, SHC4, TTC4, ARX, CRYGN, GM2A,
LYRM9, PDLIM2, SHCBP1, TTC40, AS3MT, CRYGS, GMCL1, LYSMD1, PDLIM3, SHCBP1L,
TTC5, ASAH1, CRYL1, GMDS, LYSMD2, PDLIM4, SHD, TTC6, ASAH2, CRYM, GMEB1,
LYSMD3, PDLIM5, SHE, TTC7A, ASAH2B, CRYZ, GMEB2, LYSMD4, PDLIM7, SHF, TTC7B,
ASAP1, CRYZL1, GMFB, LYST, PDP1, SHFM1, TTC8, ASAP2, CS, GMFG, LYVE1, PDP2,
SHH, TTC9, ASAP3, CSAD, GMIP, LYZ, PDPK1, SHISA2, TTC9B, ASB1, CSAG1, GML,
LYZL1, PDPN, SHISA3, TTC9C, ASB10, CSAG2, GMNC, LYZL2, PDPR, SHISA4, TTF1,
ASB11, CSAG3, GMNN, LYZL4, PDRG1, SHISA5, TTF2, ASB12, CSDC2, GMPPA, LYZL6,
PDS5A, SHISA6, TTI1, ASB13, CSDE1, GMPPB, LZIC, PDS5B, SHISA7, TTI2, ASB14, CSE1L,
GMPR, LZTFL1, PDSS1, SHISA8, TTK, ASB15, CSF1, GMPR2, LZTR1, PDSS2, SHISA9, TTL,
ASB16, CSF1R, GMPS, LZTS1, PDX1, SHKBP1, TTLL1, ASB17, CSF2, GNA11, LZTS2,
PDXDC1, SHMT1, TTLL10, ASB18, CSF2RA, GNA12, LZTS3, PDXK, SHMT2, TTLL11, ASB2,
CSF2RB, GNA13, M1AP, PDXP, SHOC2, TTLL12, ASB3, CSF3, GNA14, M6PR, PDYN, SHOX,
TTLL13, ASB4, CSF3R, GNA15, MAATS1, PDZD11, SHOX2, TTLL2, ASB5, CSGALNACT1,
GNAI1, MAB21L1, PDZD2, SHPK, TTLL3, ASB6, CSGALNACT2, GNAI2, MAB21L2, PDZD3,
SHPRH, TTLL4, ASB7, CSH1, GNAI3, MAB21L3, PDZD4, SHQ1, TTLL5, ASB8, CSH2, GNAL,
MACC1, PDZD7, SHROOM1, TTLL6, ASB9, CSHL1, GNAO1, MACF1, PDZD8, SHROOM2,
TTLL7, ASCC1, CSK, GNAQ, MACROD1, PDZD9, SHROOM3, TTLL8, ASCC2, CSMD1,
GNAS, MACROD2, PDZK1, SHROOM4, TTLL9, ASCC3, CSMD2, GNAT1, MAD1L1,
PDZK1IP1, SI, TTN, ASCL1, CSMD3, GNAT2, MAD2L1, PDZRN3, SIAE, TTPA, ASCL2,
CSN1S1, GNAT3, MAD2L1BP, PDZRN4, SIAH1, TTPAL, ASCL3, CSN2, GNAZ, MAD2L2,
PEA15, SIAH2, TTR, ASCL4, CSN3, GNB1, MADCAM1, PEAK1, SIAH3, TTYH1, ASCL5,
CSNK1A1, GNB1L, MADD, PEAR1, SIDT1, TTYH2, ASF1A, CSNK1A1L, GNB2, MAEA,
PEBP1, SIDT2, TTYH3, ASF1B, CSNK1D, GNB2L1, MAEL, PEBP4, SIGIRR, TUB, ASGR1,
CSNK1E, GNB3, MAF, PECAM1, SIGLEC1, TUBA1A, ASGR2, CSNK1G1, GNB4, MAF1,
PECR, SIGLEC10, TUBA1B, ASH1L, CSNK1G2, GNB5, MAFA, PEF1, SIGLEC11, TUBA1C,
ASH2L, CSNK1G3, GNE, MAFB, PEG10, SIGLEC12, TUBA3C, ASIC1, CSNK2A1, GNG10,
MAFF, PEG3, SIGLEC14, TUBA3D, ASIC2, CSNK2A2, GNG11, MAFG, PELI1, SIGLEC15,
TUBA3E, ASIC3, CSNK2A3, GNG12, MAFK, PELI2, SIGLEC5, TUBA4A, ASIC4, CSNK2B,
GNG13, MAG, PELI3, SIGLEC6, TUBA8, ASIC5, CSPG4, GNG2, MAGEA1, PELO, SIGLEC7,
TUBAL3, ASIP, CSPG5, GNG3, MAGEA10, PELP1, SIGLEC8, TUBB, ASL, CSPP1, GNG4,
MAGEA10-MAGEA5, PEMT, SIGLEC9, TUBB1, ASMT, CSRNP1, GNG5, MAGEA11, PENK,
SIGLECL1, TUBB2A, ASMTL, CSRNP2, GNG7, MAGEA12, PEPD, SIGMAR1, TUBB2B,
ASNA1, CSRNP3, GNG8, MAGEA3, PER1, SIK1, TUBB3, ASNS, CSRP1, GNGT1, MAGEA4,
PER2, SIK2, TUBB4A, ASNSD1, CSRP2, GNGT2, MAGEA5, PER3, SIK3, TUBB4B, ASPA,
CSRP2BP, GNL1, MAGEA6, PERM1, SIKE1, TUBB6, ASPDH, CSRP3, GNL2, MAGEA8,
PERP, SIL1, TUBB8, ASPG, CST1, GNL3, MAGEB1, PES1, SIM1, TUBD1, ASPH, CST11,
GNL3L, MAGEB10, PET100, SIM2, TUBE1, ASPHD1, CST2, GNLY, MAGEB16, PET112,
SIMC1, TUBG1, ASPHD2, CST3, GNMT, MAGEB17, PET117, SIN3A, TUBG2, ASPM, CST4,
GNPAT, MAGEB18, PEX1, SIN3B, TUBGCP2, ASPN, CST5, GNPDA1, MAGEB2, PEX10,
SIPA1, TUBGCP3, ASPRV1, CST6, GNPDA2, MAGEB3, PEX11A, SIPA1L1, TUBGCP4,
ASPSCR1, CST7, GNPNAT1, MAGEB4, PEX11B, SIPA1L2, TUBGCP5, ASRGL1, CST8,
GNPTAB, MAGEB5, PEX11G, SIPA1L3, TUBGCP6, ASS1, CST9, GNPTG, MAGEB6, PEX12,
SIRPA, TUFM, ASTE1, CST9L, GNRH1, MAGEC1, PEX13, SIRPB1, TUFT1, ASTL, CSTA,
GNRH2, MAGEC2, PEX14, SIRPB2, TULP1, ASTN1, CSTB, GNRHR, MAGEC3, PEX16,
SIRPD, TULP2, ASTN2, CSTF1, GNS, MAGED1, PEX19, SIRPG, TULP3, ASUN, CSTF2,
GOLGA1, MAGED2, PEX2, SIRT1, TULP4, ASXL1, CSTF2T, GOLGA2, MAGEE1, PEX26,
SIRT2, TUSC1, ASXL2, CSTF3, GOLGA3, MAGEE2, PEX3, SIRT3, TUSC2, ASXL3, CSTL1,
GOLGA4, MAGEF1, PEX5, SIRT4, TUSC3, ASZ1, CT45A1, GOLGA5, MAGEH1, PEX5L,
SIRT5, TUSC5, ATAD1, CT45A2, GOLGA6A, MAGEL2, PEX6, SIRT6, TUT1, ATAD2,
CT45A3, GOLGA6B, MAGI1, PEX7, SIRT7, TVP23A, ATAD2B, CT45A4, GOLGA6C, MAGI2,
PF4, SIT1, TVP23B, ATAD3A, CT45A5, GOLGA6D, MAGI3, PF4V1, SIVA1, TVP23C,
ATAD3B, CT45A6, GOLGA6L1, MAGIX, PFAS, SIX1, TVP23C-CDRT4, ATAD3C, CT47A1,
GOLGA6L10, MAGOH, PFDN1, SIX2, TWF1, ATAD5, CT47A10, GOLGA6L2, MAGOHB,
PFDN2, SIX3, TWF2, ATAT1, CT47A11, GOLGA6L3, MAGT1, PFDN4, SIX4, TWIST1,
ATCAY, CT47A12, GOLGA6L4, MAK, PFDN5, SIX5, TWIST2, ATE1, CT47A2, GOLGA6L6,
MAK16, PFDN6, SIX6, TWISTNB, ATF1, CT47A3, GOLGA6L9, MAL, PFKFB1, SKA1,
TWSG1, ATF2, CT47A4, GOLGA7, MAL2, PFKFB2, SKA2, TXK, ATF3, CT47A5, GOLGA7B,
MALL, PFKFB3, SKA3, TXLNA, ATF4, CT47A6, GOLGA8A, MALSU1, PFKFB4, SKAP1,
TXLNB, ATF5, CT47A7, GOLGA8B, MALT1, PFKL, SKAP2, TXLNG, ATF6, CT47A8,
GOLGA8H, MAMDC2, PFKM, SKI, TXN, ATF6B, CT47A9, GOLGA8J, MAMDC4, PFKP,
SKIDA1, TXN2, ATF7, CT47B1, GOLGA8K, MAML1, PFN1, SKIL, TXNDC11, ATF7IP, CT62,
GOLGA8M, MAML2, PFN2, SKIV2L, TXNDC12, ATF7IP2, CTAG1A, GOLGA8O, MAML3,
PFN3, SKIV2L2, TXNDC15, ATG10, CTAG1B, GOLGA8R, MAMLD1, PFN4, SKOR1,
TXNDC16, ATG12, CTAG2, GOLGB1, MAMSTR, PGA3, SKOR2, TXNDC17, ATG13,
CTAGE1, GOLIM4, MAN1A1, PGA4, SKP1, TXNDC2, ATG14, CTAGE15, GOLM1, MAN1A2,
PGA5, SKP2, TXNDC5, ATG16L1, CTAGE4, GOLPH3, MAN1B1, PGAM1, SLA, TXNDC8,
ATG16L2, CTAGE5, GOLPH3L, MAN1C1, PGAM2, SLA2, TXNDC9, ATG2A, CTAGE6,
GOLT1A, MAN2A1, PGAM4, SLAIN1, TXNIP, ATG2B, CTAGE8, GOLT1B, MAN2A2,
PGAM5, SLAIN2, TXNL1, ATG3, CTAGE9, GON4L, MAN2B1, PGAP1, SLAMF1, TXNL4A,
ATG4A, CTBP1, GOPC, MAN2B2, PGAP2, SLAMF6, TXNL4B, ATG4B, CTBP2, GORAB,
MAN2C1, PGAP3, SLAMF7, TXNRD1, ATG4C, CTBS, GORASP1, MANBA, PGBD1, SLAMF8,
TXNRD2, ATG4D, CTC1, GORASP2, MANBAL, PGBD2, SLAMF9, TXNRD3, ATG5, CTCF,
GOSR1, MANEA, PGBD3, SLBP, TXNRD3NB, ATG7, CTCFL, GOSR2, MANEAL, PGBD4,
SLC10A1, TYK2, ATG9A, CTDNEP1, GOT1, MANF, PGBD5, SLC10A2, TYMP, ATG9B,
CTDP1, GOT1L1, MANSC1, PGC, SLC10A3, TYMS, ATHL1, CTDSP1, GOT2, MANSC4, PGD,
SLC10A4, TYR, ATIC, CTDSP2, GP1BA, MAOA, PGF, SLC10A5, TYRO3, ATL1, CTDSPL,
GP1BB, MAOB, PGGT1B, SLC10A6, TYROBP, ATL2, CTDSPL2, GP2, MAP10, PGK1,
SLC10A7, TYRP1, ATL3, CTF1, GP5, MAP1A, PGK2, SLC11A1, TYSND1, ATM, CTGF, GP6,
MAP1B, PGLS, SLC11A2, TYW1, ATMIN, CTH, GP9, MAP1LC3A, PGLYRP1, SLC12A1,
TYW1B, ATN1, CTHRC1, GPA33, MAP1LC3B, PGLYRP2, SLC12A2, TYW3, ATOH1, CTIF,
GPAA1, MAP1LC3B2, PGLYRP3, SLC12A3, TYW5, ATOH7, CTLA4, GPALPP1, MAP1LC3C,
PGLYRP4, SLC12A4, U2AF1, ATOH8, CTNNA1, GPAM, MAP1S, PGM1, SLC12A5, U2AF1L4,
ATOX1, CTNNA2, GPANK1, MAP2, PGM2, SLC12A6, U2AF2, ATP10A, CTNNA3, GPAT2,
MAP2K1, PGM2L1, SLC12A7, U2SURP, ATP10B, CTNNAL1, GPATCH1, MAP2K2, PGM3,
SLC12A8, UACA, ATP10D, CTNNB1, GPATCH11, MAP2K3, PGM5, SLC12A9, UAP1,
ATP11A, CTNNBIP1, GPATCH2, MAP2K4, PGP, SLC13A1, UAP1L1, ATP11B, CTNNBL1,
GPATCH2L, MAP2K5, PGPEP1, SLC13A2, UBA1, ATP11C, CTNND1, GPATCH3, MAP2K6,
PGPEP1L, SLC13A3, UBA2, ATP12A, CTNND2, GPATCH4, MAP2K7, PGR, SLC13A4, UBA3,
ATP13A1, CTNS, GPATCH8, MAP3K1, PGRMC1, SLC13A5, UBA5, ATP13A2, CTPS1,
GPBAR1, MAP3K10, PGRMC2, SLC14A1, UBA52, ATP13A3, CTPS2, GPBP1, MAP3K11,
PGS1, SLC14A2, UBA6, ATP13A4, CTR9, GPBP1L1, MAP3K12, PHACTR1, SLC15A1, UBA7,
ATP13A5, CTRB1, GPC1, MAP3K13, PHACTR2, SLC15A2, UBAC1, ATP1A1, CTRB2, GPC2,
MAP3K14, PHACTR3, SLC15A3, UBAC2, ATP1A2, CTRC, GPC3, MAP3K15, PHACTR4,
SLC15A4, UBALD1, ATP1A3, CTRL, GPC4, MAP3K19, PHAX, SLC15A5, UBALD2, ATP1A4,
CTSA, GPC5, MAP3K2, PHB, SLC16A1, UBAP1, ATP1B1, CTSB, GPC6, MAP3K3, PHB2,
SLC16A10, UBAP1L, ATP1B2, CTSC, GPCPD1, MAP3K4, PHC1, SLC16A11, UBAP2, ATP1B3,
CTSD, GPD1, MAP3K5, PHC2, SLC16A12, UBAP2L, ATP1B4, CTSE, GPD1L, MAP3K6, PHC3,
SLC16A13, UBASH3A, ATP2A1, CTSF, GPD2, MAP3K7, PHEX, SLC16A14, UBASH3B,
ATP2A2, CTSG, GPER1, MAP3K7CL, PHF1, SLC16A2, UBB, ATP2A3, CTSH, GPHA2,
MAP3K8, PHF10, SLC16A3, UBC, ATP2B1, CTSK, GPHB5, MAP3K9, PHF11, SLC16A4, UBD,
ATP2B2, CTSL, GPHN, MAP4, PHF12, SLC16A5, UBE2A, ATP2B3, CTSO, GPI, MAP4K1,
PHF13, SLC16A6, UBE2B, ATP2B4, CTSS, GPIHBP1, MAP4K2, PHF14, SLC16A7, UBE2C,
ATP2C1, CTSV, GPKOW, MAP4K3, PHF19, SLC16A8, UBE2D1, ATP2C2, CTSW, GPLD1,
MAP4K4, PHF2, SLC16A9, UBE2D2, ATP4A, CTSZ, GPM6A, MAP4K5, PHF20, SLC17A1,
UBE2D3, ATP4B, CTTN, GPM6B, MAP6, PHF20L1, SLC17A2, UBE2D4, ATP5A1, CTTNBP2,
GPN1, MAP6D1, PHF21A, SLC17A3, UBE2E1, ATP5B, CTTNBP2NL, GPN2, MAP7, PHF21B,
SLC17A4, UBE2E2, ATP5C1, CTU1, GPN3, MAP7D1, PHF23, SLC17A5, UBE2E2-AS1,
ATP5D, CTU2, GPNMB, MAP7D2, PHF3, SLC17A6, UBE2E3, ATP5E, CTXN1, GPR1,
MAP7D3, PHF5A, SLC17A7, UBE2F, ATP5F1, CTXN2, GPR101, MAP9, PHF6, SLC17A8,
UBE2G1, ATP5G1, CTXN3, GPR107, MAPK1, PHF7, SLC17A9, UBE2G2, ATP5G2, CUBN,
GPR108, MAPK10, PHF8, SLC18A1, UBE2H, ATP5G3, CUEDC1, GPR110, MAPK11, PHGDH,
SLC18A2, UBE2I, ATP5H, CUEDC2, GPR111, MAPK12, PHGR1, SLC18A3, UBE2J1, ATP5I,
CUL1, GPR112, MAPK13, PHIP, SLC18B1, UBE2J2, ATP5J, CUL2, GPR113, MAPK14, PHKA1,
SLC19A1, UBE2K, ATP5J2, CUL3, GPR114, MAPK15, PHKA2, SLC19A2, UBE2L3, ATP5J2-
PTCD1, CUL4A, GPR115, MAPK1IP1L, PHKB, SLC19A3, UBE2L6, ATP5L, CUL4B, GPR116,
MAPK3, PHKG1, SLC1A1, UBE2M, ATP5L2, CUL5, GPR119, MAPK4, PHKG2, SLC1A2,
UBE2N, ATP5O, CUL7, GPR12, MAPK6, PHLDA1, SLC1A3, UBE2NL, ATP5S, CUL9, GPR123,
MAPK7, PHLDA2, SLC1A4, UBE2O, ATP5SL, CUTA, GPR124, MAPK8, PHLDA3, SLC1A5,
UBE2Q1, ATP6AP1, CUTC, GPR125, MAPK8IP1, PHLDB1, SLC1A6, UBE2Q2, ATP6AP1L,
CUX1, GPR126, MAPK8IP2, PHLDB2, SLC1A7, UBE2QL1, ATP6AP2, CUX2, GPR128,
MAPK8IP3, PHLDB3, SLC20A1, UBE2R2, ATP6V0A1, CUZD1, GPR132, MAPK9, PHLPP1,
SLC20A2, UBE2S, ATP6V0A2, CWC15, GPR133, MAPKAP1, PHLPP2, SLC22A1, UBE2T,
ATP6V0A4, CWC22, GPR135, MAPKAPK2, PHOSPHO1, SLC22A10, UBE2U, ATP6V0B,
CWC25, GPR137, MAPKAPK3, PHOSPHO2, SLC22A11, UBE2V1, ATP6V0C, CWC27,
GPR137B, MAPKAPK5, PHOSPHO2-KLHL23, SLC22A12, UBE2V2, ATP6V0D1, CWF19L1,
GPR137C, MAPKBP1, PHOX2A, SLC22A13, UBE2W, ATP6V0D2, CWF19L2, GPR139,
MAPRE1, PHOX2B, SLC22A14, UBE2Z, ATP6V0E1, CWH43, GPR142, MAPRE2, PHPT1,
SLC22A15, UBE3A, ATP6V0E2, CX3CL1, GPR143, MAPRE3, PHRF1, SLC22A16, UBE3B,
ATP6V1A, CX3CR1, GPR148, MAPT, PHTF1, SLC22A17, UBE3C, ATP6V1B1, CXADR,
GPR149, 1-Mar, PHTF2, SLC22A18, UBE3D, ATP6V1B2, CXCL1, GPR15, 10-Mar, PHYH,
SLC22A18AS, UBE4A, ATP6V1C1, CXCL10, GPR150, 11- Mar, PHYHD1, SLC22A2, UBE4B,
ATP6V1C2, CXCL11, GPR151, 2-Mar, PHYHIP, SLC22A20, UBFD1, ATP6V1D, CXCL12,
GPR152, 3-Mar, PHYHIPL, SLC22A23, UBIAD1, ATP6V1E1, CXCL13, GPR153, 4- Mar,
PHYKPL, SLC22A24, UBL3, ATP6V1E2, CXCL14, GPR155, 5-Mar, PI15, SLC22A25, UBL4A,
ATP6V1F, CXCL16, GPR156, 6-Mar, PI16, SLC22A3, UBL4B, ATP6V1G1, CXCL17, GPR157,
7-Mar, PI3, SLC22A31, UBL5, ATP6V1G2, CXCL2, GPR158, 8-Mar, PI4K2A, SLC22A4, UBL7,
ATP6V1G3, CXCL3, GPR160, 9-Mar, PI4K2B, SLC22A5, UBLCP1, ATP6V1H, CXCL5,
GPR161, MARCKS, PI4KA, SLC22A6, UBN1, ATP7A, CXCL6, GPR162, MARCKSL1, PI4KB,
SLC22A7, UBN2, ATP7B, CXCL9, GPR17, MARCO, PIANP, SLC22A8, UBOX5, ATP8A1,
CXCR1, GPR171, MARK1, PIAS1, SLC22A9, UBP1, ATP8A2, CXCR2, GPR173, MARK2,
PIAS2, SLC23A1, UBQLN1, ATP8B1, CXCR3, GPR174, MARK3, PIAS3, SLC23A2, UBQLN2,
ATP8B2, CXCR4, GPR176, MARK4, PIAS4, SLC23A3, UBQLN3, ATP8B3, CXCR5, GPR179,
MARS, PIBF1, SLC24A1, UBQLN4, ATP8B4, CXCR6, GPR18, MARS2, PICALM, SLC24A2,
UBQLNL, ATP9A, CXorf21, GPR180, MARVELD1, PICK1, SLC24A3, UBR1, ATP9B, CXorf22,
GPR182, MARVELD2, PID1, SLC24A4, UBR2, ATPAF1, CXorf23, GPR183, MARVELD3,
PIDD, SLC24A5, UBR3, ATPAF2, CXorf27, GPR19, MAS1, PIEZO1, SLC25A1, UBR4, ATPIF1,
CXorf30, GPR20, MAS1L, PIEZO2, SLC25A10, UBr5, ATR, CXorf36, GPR21, MASP1, PIF1,
SLC25A11, UBR7, ATRAID, CXorf38, GPR22, MASP2, PIFO, SLC25A12, UBTD1, ATRIP,
CXorf40A, GPR25, MAST1, PIGA, SLC25A13, UBTD2, ATRN, CXorf40B, GPR26, MAST2,
PIGB, SLC25A14, UBTF, ATRNL1, CXorf48, GPR27, MAST3, PIGC, SLC25A15, UBTFL1,
ATRX, CXorf49, GPR3, MAST4, PIGF, SLC25A16, UBXN1, ATXN1, CXorf49B, GPR31,
MASTL, PIGG, SLC25A17, UBXN10, ATXN10, CXorf51A, GPR32, MAT1A, PIGH, SLC25A18,
UBXN11, ATXN1L, CXorf51B, GPR33, MAT2A, PIGK, SLC25A19, UBXN2A, ATXN2,
CXorf56, GPR34, MAT2B, PIGL, SLC25A2, UBXN2B, ATXN2L, CXorf57, GPR35, MATK,
PIGM, SLC25A20, UBXN4, ATXN3, CXorf58, GPR37, MATN1, PIGN, SLC25A21, UBXN6,
ATXN3L, CXorf61, GPR37L1, MATN2, PIGO, SLC25A22, UBXN7, ATXN7, CXorf64, GPR39,
MATN3, PIGP, SLC25A23, UBXN8, aTXN7L1, CXorf65, GPR4, MATN4, PIGQ, SLC25A24,
UCHL1, aTXN7L2, CXorf66, GPR45, MATR3, PIGR, SLC25A25, UCHL3, ATXN7L3, CXXC1,
GPR50, MAU2, PIGS, SLC25A26, UCHL5, ATXN7L3B, CXXC11, GPR52, MAVS, PIGT,
SLC25A27, UCK1, AUH, CXXC4, GPR55, MAX, PIGU, SLC25A28, UCK2, AUNIP, CXXC5,
GPR56, MAZ, PIGV, SLC25A29, UCKL1, AUP1, CYB561, GPR6, MB, PIGW, SLC25A3,
UCMA, AURKA, CYB561A3, GPR61, MB21D1, PIGX, SLC25A30, UCN, AURKAIP1,
CYB561D1, GPR62, MB21D2, PIGY, SLC25A31, UCN2, AURKB, CYB561D2, GPR63, MBD1,
PIGZ, SLC25A32, UCN3, AURKC, CYB5A, GPR64, MBD2, PIH1D1, SLC25A33, UCP1,
AUTS2, CYB5B, GPR65, MBD3, PIH1D2, SLC25A34, UCP2, AVEN, CYB5D1, GPR68,
MBD3L1, PIH1D3, SLC25A35, UCP3, AVIL, CYB5D2, GPR75, MBD4, PIK3AP1, SLC25A36,
UEVLD, AVL9, CYB5R1, GPR75-ASB3, MBD5, PIK3C2A, SLC25A37, UFC1, AVP, CYB5R2,
GPR78, MBD6, PIK3C2B, SLC25A38, UFD1L, AVPI1, CYB5R3, GPR82, MBIP, PIK3C2G,
SLC25A39, UFL1, AVPR1A, CYB5R4, GPR83, MBL2, PIK3C3, SLC25A4, UFM1, AVPR1B,
CYB5RL, GPR84, MBLAC1, PIK3CA, SLC25A40, UFSP1, AVPR2, CYBA, GPR85, MBLAC2,
PIK3CB, SLC25A41, UFSP2, AWAT1, CYBB, GPR87, MBNL1, PIK3CD, SLC25A42, UGCG,
AWAT2, CYBRD1, GPR88, MBNL2, PIK3CG, SLC25A43, UGDH, AXDND1, CYC1, GPR89A,
MBNL3, PIK3IP1, SLC25A44, UGGT1, AXIN1, CYCS, GPR89B, MBOAT1, PIK3R1,
SLC25A45, UGGT2, AXIN2, CYFIP1, GPR89C, MBOAT2, PIK3R2, SLC25A46, UGP2, AXL,
CYFIP2, GPR97, MBOAT4, PIK3R3, SLC25A47, UGT1A1, AZGP1, CYGB, GPR98, MBOAT7,
PIK3R4, SLC25A48, UGT1A10, AZI1, CYHR1, GPRASP1, MBP, PIK3R5, SLC25A5, UGT1A3,
AZI2, CYLC1, GPRASP2, MBTD1, PIK3R6, SLC25A51, UGT1A4, AZIN1, CYLC2, GPRC5A,
MBTPS1, PIKFYVE, SLC25A52, UGT1A5, AZU1, CYLD, GPRC5B, MBTPS2, PILRA,
SLC25A53, UGT1a6, B2M, CYorf17, GPRC5C, MC1R, PILRB, SLC25A6, UGT1A7,
B3GALNT1, CYP11A1, GPRC5D, MC2R, PIM1, SLC26A1, UGT1A8, B3GALNT2, CYP11B1,
GPRC6A, MC3R, PIM2, SLC26A10, UGT1A9, B3GALT1, CYP11B2, GPRIN1, MC4R, PIM3,
SLC26A11, UGT2A1, B3GALT2, CYP17A1, GPRIN2, MC5R, PIN1, SLC26A2, UGT2A2,
B3GALT4, CYP19A1, GPRIN3, MCAM, PIN4, SLC26A3, UGT2A3, B3GALT5, CYP1A1, GPS1,
MCAT, PINK1, SLC26A4, UGT2B10, B3GALT6, CYP1A2, GPS2, MCC, PINLYP, SLC26A5,
UGT2B11, B3GALTL, CYP1B1, GPSM1, MCCC1, PINX1, SLC26A6, UGT2B15, B3GAT1,
CYP20A1, GPSM2, MCCC2, PIP, SLC26A7, UGT2B17, B3GAT2, CYP21A2, GPSM3, MCCD1,
PIP4K2A, SLC26A8, UGT2B28, B3GAT3, CYP24A1, GPT, MCEE, PIP4K2B, SLC26A9,
UGT2B4, B3GNT1, CYP26A1, GPT2, MCF2, PIP4K2C, SLC27A1, UGT2B7, B3GNT2,
CYP26B1, GPX1, MCF2L, PIP5K1A, SLC27A2, UGT3A1, B3GNT3, CYP26C1, GPX2, MCF2L2,
PIP5K1B, SLC27A3, UGT3A2, B3GNT4, CYP27A1, GPX3, MCFD2, PIP5K1C, SLC27A4,
UGT8, B3GNT5, CYP27B1, GPX4, MCHR1, PIP5KL1, SLC27A5, UHMK1, B3GNT6, CYP27C1,
GPX5, MCHR2, PIPOX, SLC27A6, UHRF1, B3GNT7, CYP2A13, GPX6, MCIDAS, PIR,
SLC28A1, UHRF1BP1, B3GNT8, CYP2A6, GPX7, MCL1, PIRT, SLC28A2, UHRF1BP1L,
B3GNT9, CYP2A7, GPX8, MCM10, PISD, SLC28A3, UHRF2, B3GNTL1, CYP2B6, GRAMD1A,
MCM2, PITHD1, SLC29A1, UIMC1, B4GALNT1, CYP2C18, GRAMD1B, MCM3, PITPNA,
SLC29A2, ULBP1, B4GALNT2, CYP2C19, GRAMD1C, MCM3AP, PITPNB, SLC29A3, ULBP2,
B4GALNT3, CYP2C8, GRAMD2, MCM4, PITPNC1, SLC29A4, ULBP3, B4GALNT4, CYP2C9,
GRAMD3, MCM5, PITPNM1, SLC2A1, ULK1, B4GALT1, CYP2D6, GRAMD4, MCM6,
PITPNM2, SLC2A10, ULK2, B4GALT2, CYP2E1, GRAP, MCM7, PITPNM3, SLC2A11, ULK3,
B4GALT3, CYP2F1, GRAP2, MCM8, PITRM1, SLC2A12, ULK4, B4GALT4, CYP2J2, GRAPL,
MCM9, PITX1, SLC2A13, UMOD, B4GALT5, CYP2R1, GRASP, MCMBP, PITX2, SLC2A14,
UMODL1, B4GALT6, CYP2S1, GRB10, MCMDC2, PITX3, SLC2A2, UMPS, B4GALT7,
CYP2U1, GRB14, MCOLN1, PIWIL1, SLC2A3, UNC119, B9D1, CYP2W1, GRB2, MCOLN2,
PIWIL2, SLC2A4, UNC119B, B9D2, CYP39A1, GRB7, MCOLN3, PIWIL3, SLC2A4RG,
UNC13A, BAALC, CYP3A4, GREB1, MCPH1, PIWIL4, SLC2A5, UNC13B, BAAT, CYP3A43,
GREM1, MCRS1, PJA1, SLC2A6, UNC13C, BABAM1, CYP3A5, GREM2, MCTP1, PJA2,
SLC2A7, UNC13D, BACE1, CYP3A7, GRHL1, MCTP2, PKD1, SLC2A8, UNC45A, BACE2,
CYP3A7- CYP3AP1, GRHL2, MCTS1, PKD1L1, SLC2A9, UNC45B, BACH1, CYP46A1,
GRHL3, MCU, PKD1L2, SLC30A1, UNC50, BACH2, CYP4A11, GRHPR, MCUR1, PKD1L3,
SLC30A10, UNC5A, BAD, CYP4A22, GRIA1, MDC1, PKD2, SLC30A2, UNC5B, BAG1,
CYP4B1, GRIA2, MDFI, PKD2L1, SLC30A3, UNC5C, BAG2, CYP4F11, GRIA3, MDFIC,
PKD2L2, SLC30A4, UNC5CL, BAG3, CYP4F12, GRIA4, MDGA1, PKDCC, SLC30A5, UNC5D,
BAG4, CYP4F2, GRID1, MDGA2, PKDREJ, SLC30A6, UNC79, BAG5, CYP4F22, GRID2,
MDH1, PKHD1, SLC30A7, UNC80, BAG6, CYP4F3, GRID2IP, MDH1B, PKHD1L1, SLC30A8,
UNC93A, BAGE, CYP4F8, GRIFIN, MDH2, PKIA, SLC30A9, UNC93B1, BAGE2, CYP4V2,
GRIK1, MDK, PKIB, SLC31A1, UNCX, BAGE3, CYP4X1, GRIK2, MDM1, PKIG, SLC31A2,
UNG, BAHCC1, CYP4Z1, GRIK3, MDM2, PKLR, SLC32A1, UNK, BAHD1, CYP51A1, GRIK4,
MDM4, PKM, SLC33A1, UNKL, BAI1, CYP7A1, GRIK5, MDN1, PKMYT1, SLC34A1, UPB1,
BAI2, CYP7B1, GRIN1, MDP1, PKN1, SLC34A2, UPF1, BAI3, CYP8B1, GRIN2A, ME1, PKN2,
SLC34A3, UPF2, BAIAP2, CYR61, GRIN2B, ME2, PKN3, SLC35A1, UPF3A, BAIAP2L1, CYS1,
GRIN2C, ME3, PKNOX1, SLC35A2, UPF3B, BAIAP2L2, CYSLTR1, GRIN2D, MEA1,
PKNOX2, SLC35A3, UPK1A, BAIAP3, CYSLTR2, GRIN3A, MEAF6, PKP1, SLC35A4, UPK1B,
BAK1, CYSTM1, GRIN3B, MECOM, PKP2, SLC35A5, UPK2, BAMBI, CYTH1, GRINA,
MECP2, PKP3, SLC35B1, UPK3A, BANF1, CYTH2, GRIP1, MECR, PKP4, SLC35B2, UPK3B,
BANF2, CYTH3, GRIP2, MED1, PLA1A, SLC35B3, UPK3BL, BANK1, CYTH4, GRIPAP1,
MED10, PLA2G10, SLC35B4, UPP1, BANP, CYTIP, GRK1, MED11, PLA2G12A, SLC35C1,
UPP2, BAP1, CYTL1, GRK4, MED12, PLA2G12B, SLC35C2, UPRT, BARD1, CYYR1, GRK5,
MED12L, PLA2G15, SLC35D1, UQCC1, BARHL1, D2HGDH, GRK6, MED13, PLA2G16,
SLC35D2, UQCC2, BARHL2, DAAM1, GRK7, MED13L, PLA2G1B, SLC35D3, UQCR10,
BARX1, DAAM2, GRM1, MED14, PLA2G2A, SLC35E1, UQCR11, BARX2, DAB1, GRM2,
MED15, PLA2G2C, SLC35E2, UQCRB, BASP1, DAB2, GRM3, MED16, PLA2G2D, SLC35E2B,
UQCRC1, BATF, DAB2IP, GRM4, MED17, PLA2G2E, SLC35E3, UQCRC2, BATF2, DACH1,
GRM5, MED18, PLA2G2F, SLC35E4, UQCRFS1, BATF3, DACH2, GRM6, MED19, PLA2G3,
SLC35F1, UQCRH, BAX, DACT1, GRM7, MED20, PLA2G4A, SLC35F2, UQCRHL, BAZ1A,
DACT2, GRM8, MED21, PLA2G4B, SLC35F3, UQCRQ, BAZ1B, DACT3, GRN, MED22,
PLA2G4C, SLC35F4, URAD, BAZ2A, DAD1, GRP, MED23, PLA2G4D, SLC35F5, URB1,
BAZ2B, DAG1, GRPEL1, MED24, PLA2G4E, SLC35F6, URB2, BBC3, DAGLA, GRPEL2,
MED25, PLA2G4F, SLC35G1, URGCP, BBIP1, DAGLB, GRPR, MED26, PLA2G5, SLC35G2,
URGCP-MRPS24, BBOX1, DAK, GRSF1, MED27, PLA2G6, SLC35G3, URI1, BBS1, DALRD3,
GRTP1, MED28, PLA2G7, SLC35G4, URM1, BBS10, DAND5, GRWD1, MED29, PLA2R1,
SLC35G5, UROC1, BBS12, DAO, GRXCR1, MED30, PLAA, SLC35G6, UROD, BBS2, DAOA,
GRXCR2, MED31, PLAC1, SLC36A1, UROS, BBS4, DAP, GSAP, MED4, PLAC4, SLC36A2,
USB1, BBS5, DAP3, GSC, MED6, PLAC8, SLC36A3, USE1, BBS7, DAPK1, GSC2, MED7,
PLAC8L1, SLC36A4, USF1, BBS9, DAPK2, GSDMA, MED8, PLAC9, SLC37A1, USF2, BBX,
DAPK3, GSDMB, MED9, PLAG1, SLC37A2, USH1C, BCAM, DAPL1, GSDMC, MEDAG,
PLAGL1, SLC37A3, USH1G, BCAN, DAPP1, GSDMD, MEF2A, PLAGL2, SLC37A4, USH2A,
BCAP29, DARS, GSE1, MEF2B, PLAT, SLC38A1, USHBP1, BCAP31, DARS2, GSG1,
MEF2BNB, PLAU, SLC38A10, USMG5, BCAR1, DAW1, GSG1L, MEF2BNB-MEF2B, PLAUR,
SLC38A11, USO1, BCAR3, DAXX, GSG2, MEF2C, PLB1, SLC38A2, USP1, BCAS1, DAZ1,
GSK3A, MEF2D, PLBD1, SLC38A3, USP10, BCAS2, DAZ2, GSK3B, MEFV, PLBD2, SLC38A4,
USP11, BCAS3, DAZ3, GSKIP, MEGF10, PLCB1, SLC38A5, USP12, BCAS4, DAZ4, GSN,
MEGF11, PLCB2, SLC38A6, USP13, BCAT1, DAZAP1, GSPT1, MEGF6, PLCB3, SLC38A7,
USP14, BCAT2, DAZAP2, GSPT2, MEGF8, PLCB4, SLC38A8, USP15, BCCIP, DAZL, GSR,
MEGF9, PLCD1, SLC38A9, USP16, BCDIN3D, DBF4, GSS, MEI1, PLCD3, SLC39A1,
USP17L24, BCHE, DBF4B, GSTA1, MEI4, PLCD4, SLC39A10, USP17L25, BCKDHA, DBH,
GSTA2, MEIG1, PLCE1, SLC39A11, USP17L26, BCKDHB, DBI, GSTA3, MEIOB, PLCG1,
SLC39A12, USP17L28, BCKDK, DBN1, GSTA4, MEIS1, PLCG2, SLC39A13, USP17L29,
BCL10, DBNDD1, GSTA5, MEIS2, PLCH1, SLC39A14, USP18, BCL11A, DBNDD2, GSTCD,
MEIS3, PLCH2, SLC39A2, USP19, BCL11B, DBNL, GSTK1, MELK, PLCL1, SLC39A3, USP2,
BCL2, DBP, GSTM1, MEMO1, PLCL2, SLC39A4, USP20, BCL2A1, DBR1, GSTM2, MEN1,
PLCXD1, SLC39A5, USP21, BCL2L1, DBT, GSTM3, MEOX1, PLCXD2, SLC39A6, USP22,
BCL2L10, DBX1, GSTM4, MEOX2, PLCXD3, SLC39A7, USP24, BCL2L11, DBX2, GSTM5,
MEP1A, PLCZ1, SLC39A8, USP25, BCL2L12, DCAF10, GSTO1, MEP1B, PLD1, SLC39A9,
USP26, BCL2L13, DCAF11, GSTO2, MEPCE, PLD2, SLC3A1, USP27X, BCL2L14, DCAF12,
GSTP1, MEPE, PLD3, SLC3A2, USP28, BCL2L15, DCAF12L1, GSTT1, MERTK, PLD4,
SLC40A1, USP29, BCL2L2, DCAF12L2, GSTT2, MESDC1, PLD5, SLC41A1, USP3, BCL2L2-
PABPN1, DCAF13, GSTT2B, MESDC2, PLD6, SLC41A2, USP30, BCL3, DCAF15, GSTZ1,
MESP1, PLEC, SLC41A3, USP31, BCL6, DCAF16, GSX1, MESP2, PLEK, SLC43A1, USP32,
BCL6B, DCAF17, GSX2, MEST, PLEK2, SLC43A2, USP33, BCL7A, DCAF4, GTDC1, MET,
PLEKHA1, SLC43A3, USP34, BCL7B, DCAF4L1, GTF2A1, METAP1, PLEKHA2, SLC44A1,
USP35, BCL7C, DCAF4L2, GTF2A1L, METAP1D, PLEKHA3, SLC44A2, USP36, BCL9,
DCAF5, GTF2A2, METAP2, PLEKHA4, SLC44A3, USP37, BCL9L, DCAF6, GTF2B, METRN,
PLEKHA5, SLC44A4, USP38, BCLAF1, DCAF7, GTF2E1, METRNL, PLEKHA6, SLC44A5,
USP39, BCMO1, DCAF8, GTF2E2, METTL1, PLEKHA7, SLC45A1, USP4, BCO2, DCAF8L1,
GTF2F1, METTL10, PLEKHA8, SLC45A2, USP40, BCOR, DCAKD, GTF2F2, METTL11B,
PLEKHB1, SLC45A3, USP41, BCORL1, DCBLD1, GTF2H1, METTL12, PLEKHB2, SLC45A4,
USP42, BCR, DCBLD2, GTF2H2, METTL13, PLEKHD1, SLC46A1, USP43, BCS1L, DCC,
GTF2H2C, METTL14, PLEKHF1, SLC46A2, USP44, BDH1, DCD, GTF2H3, METTL15,
PLEKHF2, SLC46A3, USP45, BDH2, DCDC1, GTF2H4, METTL16, PLEKHG1, SLC47A1,
USP46, BDKRB1, DCDC2, GTF2H5, METTL17, PLEKHG2, SLC47A2, USP47, BDKRB2,
DCDC2B, GTF2I, METTL18, PLEKHG3, SLC48A1, USP48, BDNF, DCDC2C, GTF2IRD1,
METTL20, PLEKHG4, SLC4A1, USP49, BDP1, DCDC5, GTF2IRD2, METTL21A, PLEKHG4B,
SLC4A10, USP5, BEAN1, DCHS1, GTF2IRD2B, METTL21B, PLEKHG5, SLC4A11, USP50,
BECN1, DCHS2, GTF3A, METTL21C, PLEKHG6, SLC4A1AP, USP51, BECN1P1, DCK,
GTF3C1, METTL22, PLEKHG7, SLC4A2, USP53, BEGAIN, DCLK1, GTF3C2, METTL23,
PLEKHH1, SLC4A3, USP54, BEND2, DCLK2, GTF3C3, METTL24, PLEKHH2, SLC4A4, USP6,
BEND3, DCLK3, GTF3C4, METTL25, PLEKHH3, SLC4A5, USP6NL, BEND4, DCLRE1A,
GTF3C5, METTL2A, PLEKHJ1, SLC4A7, USP7, BEND5, DCLRE1B, GTF3C6, METTL2B,
PLEKHM1, SLC4A8, USP8, BEND6, DCLRE1C, GTPBP1, METTL3, PLEKHM2, SLC4A9,
USP9X, BEND7, DCN, GTPBP10, METTL4, PLEKHM3, SLC50A1, USP9Y, BEST1, DCP1A,
GTPBP2, METTL5, PLEKHN1, SLC51A, USPL1, BEST2, DCP1B, GTPBP3, METTL6,
PLEKHO1, SLC51B, UST, BEST3, DCP2, GTPBP4, METTL7A, PLEKHO2, SLC52A1, UTF1,
BEST4, DCPS, GTPBP6, METTL7B, PLEKHS1, SLC52A2, UTP11L, BET1, DCST1, GTPBP8,
METTL8, PLET1, SLC52A3, UTP14A, BET1L, DCST2, GTSCR1, METTL9, PLG, SLC5A1,
UTP14C, BEX1, DCSTAMP, GTSE1, MEX3A, PLGLB1, SLC5A10, UTP15, BEX2, DCT, GTSF1,
MEX3B, PLGLB2, SLC5A11, UTP18, BEX4, DCTD, GTSF1L, MEX3C, PLGRKT, SLC5A12,
UTP20, BEX5, DCTN1, GUCA1A, MEX3D, PLIN1, SLC5A2, UTP23, BFAR, DCTN2, GUCA1B,
MFAP1, PLIN2, SLC5A3, UTP3, BFSP1, DCTN3, GUCA1C, MFAP2, PLIN3, SLC5A4, UTP6,
BFSP2, DCTN4, GUCA2A, MFAP3, PLIN4, SLC5A5, UTRN, BGLAP, DCTN5, GUCA2B,
MFAP3L, PLIN5, SLC5A6, UTS2, BGN, DCTN6, GUCD1, MFAP4, PLK1, SLC5A7, UTS2B,
BHLHA15, DCTPP1, GUCY1A2, MFAP5, PLK1S1, SLC5A8, UTY, BHLHA9, DCUN1D1,
GUCY1A3, MFF, PLK2, SLC5A9, UVRAG, BHLHB9, DCUN1D2, GUCY1B3, MFGE8, PLK3,
SLC6A1, UVSSA, BHLHE22, DCUN1D3, GUCY2C, MFHAS1, PLK4, SLC6A11, UXS1,
BHLHE23, DCUN1D4, GUCY2D, MFI2, PLK5, SLC6A12, UXT, BHLHE40, DCUN1D5,
GUCY2F, MFN1, PLLP, SLC6A13, VAC14, BHLHE41, DCX, GUF1, MFN2, PLN, SLC6A14,
VAMP1, BHMT, DCXR, GUK1, MFNG, PLOD1, SLC6A15, VAMP2, BHMT2, DDA1, GULP1,
MFRP, PLOD2, SLC6A16, VAMP3, BICC1, DDAH1, GUSB, MFSD1, PLOD3, SLC6A17,
VAMP4, BICD1, DDAH2, GXYLT1, MFSD10, PLP1, SLC6A18, VAMP5, BICD2, DDB1,
GXYLT2, MFSD11, PLP2, SLC6A19, VAMP7, BID, DDB2, GYG1, MFSD12, PLRG1, SLC6A2,
VAMP8, BIK, DDC, GYG2, MFSD2A, PLS1, SLC6A20, VANGL1, BIN1, DDHD1, GYLTL1B,
MFSD2B, PLS3, SLC6A3, VANGL2, BIN2, DDHD2, GYPA, MFSD3, PLSCR1, SLC6A4, VAPA,
BIN3, DDI1, GYPB, MFSD4, PLSCR2, SLC6A5, VAPB, BIRC2, DDI2, GYPC, MFSD5, PLSCR3,
SLC6A6, VARS, BIRC3, DDIT3, GYPE, MFSD6, PLSCR4, SLC6A7, VARS2, BIRC5, DDIT4,
GYS1, MFSD6L, PLSCR5, SLC6A8, VASH1, BIRC6, DDIT4L, GYS2, MFSD7, PLTP, SLC6A9,
VASH2, BIRC7, DDN, GZF1, MFSD8, PLVAP, SLC7A1, VASN, BIRC8, DDO, GZMA, MFSD9,
PLXDC1, SLC7A10, VASP, BIVM, DDOST, GZMB, MGA, PLXDC2, SLC7A11, VAT1, BIVM-
ERCC5, DDR1, GZMH, MGAM, PLXNA1, SLC7A13, VAT1L, BLCAP, DDR2, GZMK, MGARP,
PLXNA2, SLC7A14, VAV1, BLID, DDRGK1, GZMM, MGAT1, PLXNA3, SLC7A2, VAV2,
BLK, DDT, H1F0, MGAT2, PLXNA4, SLC7A3, VAV3, BLM, DDTL, H1FNT, MGAT3,
PLXNB1, SLC7A4, VAX1, BLMH, DDX1, H1FOO, MGAT4A, PLXNB2, SLC7A5, VAX2,
BLNK, DDX10, H1FX, MGAT4B, PLXNB3, SLC7A6, VBP1, BLOC1S1, DDX11, H2AFB1,
MGAT4C, PLXNC1, SLC7A6OS, VCAM1, BLOC1S2, DDX17, H2AFB2, MGAT5, PLXND1,
SLC7A7, VCAN, BLOC1S3, DDX18, H2AFB3, MGAT5B, PM20D1, SLC7A8, VCL, BLOC1S4,
DDX19A, H2AFJ, MGEA5, PM20D2, SLC7A9, VCP, BLOC1S5, DDX19B, H2AFV, MGLL,
PMAIP1, SLC8A1, VCPIP1, BLOC1S6, DDX20, H2AFX, MGME1, PMCH, SLC8A2, VCPKMT,
BLVRA, DDX21, H2AFY, MGMT, PMEL, SLC8A3, VCX, BLVRB, DDX23, H2AFY2, MGP,
PMEPA1, SLC8B1, VCX2, BLZF1, DDX24, H2AFZ, MGRN1, PMF1, SLC9A1, VCX3A, BMF,
DDX25, H2BFM, MGST1, PMF1-BGLAP, SLC9A2, VCX3B, BMI1, DDX26B, H2BFWT,
MGST2, PMFBP1, SLC9A3, VCY, BMP1, DDX27, H3F3A, MGST3, PML, SLC9A3R1, VCY1B,
BMP10, DDX28, H3F3B, MIA, PMM1, SLC9A3R2, VDAC1, BMP15, DDX31, H3F3C, MIA2,
PMM2, SLC9A4, VDAC2, BMP2, DDX39A, H6PD, MIA3, PMP2, SLC9A5, VDAC3, BMP2K,
DDX39B, HAAO, MIB1, PMP22, SLC9A6, VDR, BMP3, DDX3X, HABP2, MIB2, PMPCA,
SLC9A7, VEGFA, BMP4, DDX3Y, HABP4, MICA, PMPCB, SLC9A8, VEGFB, BMP5, DDX4,
HACE1, MICAL1, PMS1, SLC9A9, VEGFC, BMP6, DDX41, HACL1, MICAL2, PMS2, SLC9B1,
VENTX, BMP7, DDX42, HADH, MICAL3, PMVK, SLC9B2, VEPH1, BMP8A, DDX43,
HADHA, MICALCL, PNCK, SLC9C1, VEZF1, BMP8B, DDX46, HADHB, MICALL1, PNISR,
SLC9C2, VEZT, BMPER, DDX47, HAGH, MICALL2, PNKD, SLCO1A2, VGF, BMPR1A,
DDX49, HAGHL, MICB, PNKP, SLCO1B1, VGLL1, BMPR1B, DDX5, HAL, MICU1, PNLDC1,
SLCO1B3, VGLL2, BMPR2, DDX50, HAMP, MICU2, PNLIP, SLCO1B7, VGLL3, BMS1,
DDX51, HAND1, MICU3, PNLIPRP1, SLCO1C1, VGLL4, BMX, DDX52, HAND2, MID1,
PNLIPRP2, SLCO2A1, VHL, BNC1, DDX53, HAO1, MID1IP1, PNLIPRP3, SLCO2B1, VHLL,
BNC2, DDX54, HAO2, MID2, PNMA2, SLCO3A1, VIL1, BNIP1, DDX55, HAP1, MIDN,
PNMA3, SLCO4A1, VILL, BNIP2, DDX56, HAPLN1, MIEF1, PNMA5, SLCO4C1, VIM, BNIP3,
DDX58, HAPLN2, MIEF2, PNMA6A, SLCO5A1, VIMP, BNIP3L, DDX59, HAPLN3, MIEN1,
PNMA6C, SLCO6A1, VIP, BNIPL, DDX6, HAPLN4, MIER1, PNMAL1, SLFN11, VIPAS39,
BOC, DDX60, HARBI1, MIER2, PNMAL2, SLFN12, VIPR1, BOD1, DDX60L, HARS, MIER3,
PNMT, SLFN12L, VIPR2, BOD1L1, DEAF1, HARS2, MIF, PNN, SLFN13, VIT, BOD1L2, 1-Dec,
HAS1, MIF4GD, PNO1, SLFN14, VKORC1, BOK, DECR1, HAS2, MIIP, PNOC, SLFN5,
VKORC1L1, BOLA1, DECR2, HAS3, MILR1, PNP, SLFNL1, VLDLR, BOLA2, DEDD, HAT1,
MINA, PNPLA1, SLIRP, VMA21, BOLA2B, DEDD2, HAUS1, MINK1, PNPLA2, SLIT1, VMAC,
BOLA3, DEF6, HAUS2, MINOS1, PNPLA3, SLIT2, VMO1, BOLL, DEF8, HAUS3, MINOS1-
NBL1, PNPLA4, SLIT3, VMP1, BOP1, DEFA1, HAUS4, MINPP1, PNPLA5, SLITRK1, VN1R1,
BORA, DEFA1B, HAUS5, MIOS, PNPLA6, SLITRK2, VN1R2, BPGM, DEFA3, HAUS6, MIOX,
PNPLA7, SLITRK3, VN1R4, BPHL, DEFA4, HAUS7, MIP, PNPLA8, SLITRK4, VN1R5, BPI,
DEFA5, HAUS8, MIPEP, PNPO, SLITRK5, VNN1, BPIFA1, DEFA6, HAVCR1, MIPOL1,
PNPT1, SLITRK6, VNN2, BPIFA2, DEFB1, HAVCR2, MIR205HG, PNRC1, SLK, VOPP1,
BPIFA3, DEFB103A, HAX1, MIS12, PNRC2, SLMAP, VPRBP, BPIFB1, DEFB103B, HBA1,
MIS18A, POC1A, SLMO1, VPREB1, BPIFB2, DEFB104A, HBA2, MIS18BP1, POC1B, SLMO2,
VPREB3, BPIFB3, DEFB104B, HBB, MISP, POC1B-GALNT4, SLN, VPS11, BPIFB4,
DEFB105A, HBD, MITD1, POC5, SLPI, VPS13A, BPIFB6, DEFB105B, HBE1, MITF, PODN,
SLTM, VPS13B, BPIFC, DEFB106A, HBEGF, MIXL1, PODNL1, SLU7, VPS13C, BPNT1,
DEFB106B, HBG1, MKI67, PODXL, SLURP1, VPS13D, BPTF, DEFB107A, HBG2, MKKS,
PODXL2, SLX1A, VPS16, BPY2, DEFB107B, HBM, MKL1, POF1B, SLX1B, VPS18, BPY2B,
DEFB108B, HBP1, MKL2, POFUT1, SLX4, VPS25, BPY2C, DEFB110, HBQ1, MKLN1,
POFUT2, SLX4IP, VPS26A, BRAF, DEFB112, HBS1L, MKNK1, POGK, SMAD1, VPS26B,
BRAP, DEFB113, HBZ, MKNK2, POGLUT1, SMAD2, VPS28, BRAT1, DEFB114, HCAR1,
MKRN1, POGZ, SMAD3, VPS29, BRCA1, DEFB115, HCAR2, MKRN2, POLA1, SMAD4,
VPS33A, BRCA2, DEFB116, HCAR3, MKRN3, POLA2, SMAD5, VPS33B, BRCC3, DEFB118,
HCCS, MKS1, POLB, SMAD6, VPS35, BRD1, DEFB119, HCFC1, MKX, POLD1, SMAD7,
VPS36, BRD2, DEFB121, HCFC1R1, MLANA, POLD2, SMAD9, VPS37A, BRD3, DEFB123,
HCFC2, MLC1, POLD3, SMAGP, VPS37B, BRD4, DEFB124, HCK, MLEC, POLD4, SMAP1,
VPS37C, BRD7, DEFB125, HCLS1, MLF1, POLDIP2, SMAP2, VPS37D, BRD8, DEFB126,
HCN1, MLF2, POLDIP3, SMARCA1, VPS39, BRD9, DEFB127, HCN2, MLH1, POLE,
SMARCA2, VPS41, BRDT, DEFB128, HCN3, MLH3, POLE2, SMARCA4, VPS45, BRE,
DEFB129, HCN4, MLIP, POLE3, SMARCA5, VPS4A, BRF1, DEFB130, HCRT, MLKL, POLE4,
SMARCAD1, VPS4B, BRF2, DEFB131, HCRTR1, MLLT1, POLG, SMARCAL1, VPS51, BRI3,
DEFB132, HCRTR2, MLLT10, POLG2, SMARCB1, VPS52, BRI3BP, DEFB133, HCST,
MLLT11, POLH, SMARCC1, VPS53, BRICD5, DEFB134, HDAC1, MLLT3, POLI, SMARCC2,
VPS54, BRINP1, DEFB135, HDAC10, MLLT4, POLK, SMARCD1, VPS72, BRINP2, DEFB136,
HDAC11, MLLT6, POLL, SMARCD2, VPS8, BRINP3, DEFB4A, HDAC2, MLN, POLM,
SMARCD3, VPS9D1, BRIP1, DEFB4B, HDAC3, MLNR, POLN, SMARCE1, VRK1, BRIX1,
DEGS1, HDAC4, MLPH, POLQ, SMC1A, VRK2, BRK1, DEGS2, HDAC5, MLST8, POLR1A,
SMC1B, VRK3, BRMS1, DEK, HDAC6, MLX, POLR1B, SMC2, VRTN, BRMS1L, DENND1A,
HDAC7, MLXIP, POLR1C, SMC3, VSIG1, BROX, DENND1B, HDAC8, MLXIPL, POLR1D,
SMC4, VSIG10, BRPF1, DENND1C, HDAC9, MLYCD, POLR1E, SMC5, VSIG10L, BRPF3,
DENND2A, HDC, MMAA, POLR2A, SMC6, VSIG2, BRS3, DENND2C, HDDC2, MMAB,
POLR2B, SMCHD1, VSIG4, BRSK1, DENND2D, HDDC3, MMACHC, POLR2C, SMCO2,
VSIG8, BRSK2, DENND3, HDGF, MMADHC, POLR2D, SMCO3, VSNL1, BRWD1, DENND4A,
HDGFL1, MMD, POLR2E, SMCO4, VSTM1, BRWD3, DENND4B, HDGFRP2, MMD2,
POLR2F, SMCP, VSTM2A, BSCL2, DENND4C, HDGFRP3, MME, POLR2G, SMCR8,
VSTM2B, BSDC1, DENND5A, HDHD1, MMEL1, POLR2H, SMCR9, VSTM2L, BSG,
DENND5B, HDHD2, MMGT1, POLR2I, SMDT1, VSTM4, BSN, DENND6A, HDHD3, MMP1,
POLR2J, SMEK1, VSTM5, BSND, DENND6B, HDLBP, MMP10, POLR2J2, SMEK2, VSX1,
BSPH1, DENR, HDX, MMP11, POLR2J3, SMG1, VSX2, BSPRY, DEPDC1, HEATR1, MMP12,
POLR2K, SMG5, VTA1, BST1, DEPDC1B, HEATR2, MMP13, POLR2L, SMG6, VTCN1, BST2,
DEPDC4, HEATR3, MMP14, POLR2M, SMG7, VTI1A, BSX, DEPDC5, HEATR4, MMP15,
POLR3A, SMG8, VTI1B, BTAF1, DEPDC7, HEATR5A, MMP16, POLR3B, SMG9, VTN,
BTBD1, DEPTOR, HEATR5B, MMP17, POLR3C, SMIM1, VWA1, BTBD10, DERA, HEATR6,
MMP19, POLR3D, SMIM10, VWA2, BTBD11, DERL1, HEBP1, MMP2, POLR3E, SMIM11,
VWA3A, BTBD16, DERL2, HEBP2, MMP20, POLR3F, SMIM12, VWA3B, BTBD17, DERL3,
HECA, MMP21, POLR3G, SMIM13, VWA5A, BTBD18, DES, HECTD1, MMP23B, POLR3GL,
SMIM14, VWA5B1, BTBD19, DESI1, HECTD2, MMP24, POLR3H, SMIM15, VWA5B2,
BTBD2, DESI2, HECTD3, MMP25, POLR3K, SMIM17, VWA7, BTBD3, DET1, HECTD4,
MMP26, POLRMT, SMIM18, VWA8, BTBD6, DEXI, HECW1, MMP27, POM121, SMIM19,
VWA9, BTBD7, DFFA, HECW2, MMP28, POM121C, SMIM2, VWC2, BTBD8, DFFB, HEG1,
MMP3, POM121L12, SMIM20, VWC2L, BTBD9, DFNA5, HELB, MMP7, POM121L2, SMIM21,
VWCE, BTC, DFNB31, HELLS, MMP8, POMC, SMIM22, VWDE, BTD, DFNB59, HELQ,
MMP9, POMGNT1, SMIM3, VWF, BTF3, DGAT1, HELT, MMRN1, POMGNT2, SMIM4, WAC,
BTF3L4, DGAT2, HELZ, MMRN2, POMK, SMIM5, WAPAL, BTG1, DGAT2L6, HELZ2,
MMS19, POMP, SMIM6, WARS, BTG2, DGCR14, HEMGN, MMS22L, POMT1, SMIM7,
WARS2, BTG3, DGCR2, HEMK1, MN1, POMT2, SMIM8, WAS, BTG4, DGCR6, HENMT1,
MNAT1, POMZP3, SMIM9, WASF1, BTK, DGCR6L, HEPACAM, MND1, PON1, SMKR1,
WASF2, BTLA, DGCR8, HEPACAM2, MNDA, PON2, SMLR1, WASF3, BTN1A1, DGKA,
HEPH, MNS1, PON3, SMN1, WASH1, BTN2A1, DGKB, HEPHL1, MNT, POP1, SMN2, WASL,
BTN2A2, DGKD, HEPN1, MNX1, POP4, SMNDC1, WBP1, BTN3A1, DGKE, HERC1, MOAP1,
POP5, SMO, WBP11, BTN3A2, DGKG, HERC2, MOB1A, POP7, SMOC1, WBP1L, BTN3A3,
DGKH, HERC3, MOB1B, POPDC2, SMOC2, WBP2, BTNL10, DGKI, HERC4, MOB2, POPDC3,
SMOX, WBP2NL, BTNL2, DGKK, HERC5, MOB3A, POR, SMPD1, WBP4, BTNL3, DGKQ,
HERC6, MOB3B, PORCN, SMPD2, WBP5, BTNL8, DGKZ, HERPUD1, MOB3C, POSTN,
SMPD3, WBSCR16, BTNL9, DGUOK, HERPUD2, MOB4, POT1, SMPD4, WBSCR17, BTRC,
DHCR24, HES1, MOBP, POTEA, SMPD5, WBSCR22, BUB1, DHCR7, HES2, MOCOS, POTEB,
SMPDL3A, WBSCR27, BUB1B, DHDDS, HES3, MOCS1, POTEB2, SMPDL3B, WBSCR28,
BUB3, DHDH, HES4, MOCS2, POTEC, SMPX, WDFY1, BUD13, DHFR, HES5, MOCS3,
POTED, SMR3A, WDFY2, BUD31, DHFRL1, HES6, MOG, POTEE, SMR3B, WDFY3, BVES,
DHH, HES7, MOGAT1, POTEF, SMS, WDFY4, BYSL, DHODH, HESX1, MOGAT2, POTEG,
SMTN, WDHD1, BZRAP1, DHPS, HEXA, MOGAT3, POTEH, SMTNL1, WDPCP, BZW1,
DHRS1, HEXB, MOGS, POTEI, SMTNL2, WDR1, BZW2, DHRS11, HEXDC, MOK, POTEJ,
SMU1, WDR11, C10orf10, DHRS12, HEXIM1, MON1A, POTEM, SMUG1, WDR12, C10orf105,
DHRS13, HEXIM2, MON1B, POU1F1, SMURF1, WDR13, C10orf107, DHRS2, HEY1, MON2,
POU2AF1, SMURF2, WDR16, C10orf11, DHRS3, HEY2, MORC1, POU2F1, SMYD1, WDR17,
C10orf111, DHRS4, HEYL, MORC2, POU2F2, SMYD2, WDR18, C10orf112, DHRS4L1, HFE,
MORC3, POU2F3, SMYD3, WDR19, C10orf113, DHRS4L2, HFE2, MORC4, POU3F1, SMYD4,
WDR20, C10orf118, DHRS7, HFM1, MORF4L1, POU3F2, SMYD5, WDR24, C10orf12,
DHRS7B, HGC6.3, MORF4L2, POU3F3, SNAI1, WDR25, C10orf120, DHRS7C, HGD, MORN1,
POU3F4, SNAI2, WDR26, C10orf126, DHRS9, HGF, MORN2, POU4F1, SNAI3, WDR27,
C10orf128, DHRSX, HGFAC, MORN3, POU4F2, SNAP23, WDR3, C10orf129, DHTKD1, HGS,
MORN4, POU4F3, SNAP25, WDR31, C10orf131, DHX15, HGSNAT, MORN5, POU5F1,
SNAP29, WDR33, C10orf137, DHX16, HHAT, MOS, POU5F1B, SNAP47, WDR34, C10orf2,
DHX29, HHATL, MOSPD1, POU5F2, SNAP91, WDR35, C10orf25, DHX30, HHEX, MOSPD2,
POU6F1, SNAPC1, WDR36, C10orf32, DHX32, HHIP, MOSPD3, POU6F2, SNAPC2, WDR37,
C10orf35, DHX33, HHIPL1, MOV10, PP2D1, SNAPC3, WDR38, C10orf53, DHX34, HHIPL2,
MOV10L1, PPA1, SNAPC4, WDR4, C10orf54, DHX35, HHLA1, MOXD1, PPA2, SNAPC5,
WDR41, C10orf55, DHX36, HHLA2, MPC1, PPAN, SNAPIN, WDR43, C10orf62, DHX37,
HHLA3, MPC1L, PPAN-P2RY11, SNCA, WDR44, C10orf67, DHX38, HIAT1, MPC2, PPAP2A,
SNCAIP, WDR45, C10orf68, DHX40, HIATL1, MPDU1, PPAP2B, SNCB, WDR45B, C10orf71,
DHX57, HIBADH, MPDZ, PPAP2C, SNCG, WDR46, C10orf76, DHX58, HIBCH, MPEG1,
PPAPDC1A, SND1, WDR47, C10orf82, DHX8, HIC1, MPG, PPAPDC1B, SNED1, WDR48,
C10orf88, DHX9, HIC2, MPHOSPH10, PPAPDC2, SNF8, WDR49, C10orf90, DIABLO, HID1,
MPHOSPH6, PPAPDC3, SNIP1, WDR5, C10orf91, DIAPH1, HIF1A, MPHOSPH8, PPARA, SNN,
WDR52, C10orf95, DIAPH2, HIF1AN, MPHOSPH9, PPARD, SNPH, WDR53, C10orf99,
DIAPH3, HIF3A, MPI, PPARG, SNRK, WDR54, C11orf1, DICER1, HIGD1A, MPL, PPARGC1A,
SNRNP200, WDR55, C11orf16, DIDO1, HIGD1B, MPLKIP, PPARGC1B, SNRNP25, WDR59,
C11orf21, DIEXF, HIGD1C, MPND, PPAT, SNRNP27, WDR5B, C11orf24, DIMT1, HIGD2A,
MPO, PPBP, SNRNP35, WDR6, C11orf30, DIO1, HILPDA, MPP1, PPCDC, SNRNP40, WDR60,
C11orf31, DIO2, HINFP, MPP2, PPCS, SNRNP48, WDR61, C11orf35, DIO3, HINT1, MPP3,
PPDPF, SNRNP70, WDR62, C11orf40, DIP2A, HINT2, MPP4, PPEF1, SNRPA, WDR63,
C11orf42, DIP2B, HINT3, MPP5, PPEF2, SNRPA1, WDR64, C11orf44, DIP2C, HIP1, MPP6,
PPFIA1, SNRPB, WDR65, C11orf45, DIRAS1, HIP1R, MPP7, PPFIA2, SNRPB2, WDR66,
C11orf48, DIRAS2, HIPK1, MPPE1, PPFIA3, SNRPC, WDR7, C11orf49, DIRAS3, HIPK2,
MPPED1, PPFIA4, SNRPD1, WDR70, C11orf52, DIRC1, HIPK3, MPPED2, PPFIBP1, SNRPD2,
WDR72, C11orf53, DIRC2, HIPK4, MPRIP, PPFIBP2, SNRPD3, WDR73, C11orf54, DIS3, HIRA,
MPST, PPHLN1, SNRPE, WDR74, C11orf57, DIS3L, HIRIP3, MPV17, PPIA, SNRPF, WDR75,
C11orf58, DIS3L2, HIST1H1A, MPV17L, PPIAL4A, SNRPG, WDR76, C11orf63, DISC1,
HIST1H1B, MPV17L2, PPIAL4B, SNRPN, WDR77, C11orf65, DISP1, HIST1H1C, MPZ,
PPIAL4C, SNTA1, WDR78, C11orf68, DISP2, HIST1H1D, MPZL1, PPIAL4D, SNTB1, WDR81,
C11orf70, DIXDC1, HIST1H1E, MPZL2, PPIAL4E, SNTB2, WDR82, C11orf71, DKC1,
HIST1H1T, MPZL3, PPIAL4F, SNTG1, WDR83, C11orf73, DKK1, HIST1H2AA, MR1,
PPIAL4G, SNTG2, WDR83OS, C11orf74, DKK2, HIST1H2AB, MRAP, PPIB, SNTN, WDR86,
C11orf80, DKK3, HIST1H2AC, MRAP2, PPIC, SNUPN, WDR87, C11orf82, DKK4, HIST1H2AD,
MRAS, PPID, SNURF, WDR88, C11orf83, DKKL1, HIST1H2AE, MRC2, PPIE, SNW1, WDR89,
C11orf84, DLAT, HIST1H2AG, MRE11A, PPIF, SNX1, WDR90, C11orf85, DLC1, HIST1H2AH,
MREG, PPIG, SNX10, WDR91, C11orf86, DLD, HIST1H2AI, MRFAP1, PPIH, SNX11, WDR92,
C11orf87, DLEC1, HIST1H2AJ, MRFAP1L1, PPIL1, SNX12, WDR93, C11orf88, DLEU7,
HIST1H2AK, MRGBP, PPIL2, SNX13, WDR96, C11orf91, DLG1, HIST1H2AL, MRGPRD,
PPIL3, SNX14, WDSUB1, C11orf94, DLG2, HIST1H2AM, MRGPRE, PPIL4, SNX15, WDTC1,
C11orf95, DLG3, HIST1H2BA, MRGPRF, PPIL6, SNX16, WDYHV1, C11orf96, DLG4,
HIST1H2BB, MRGPRG, PPIP5K1, SNX17, WEE1, C12orf10, DLG5, HIST1H2BC, MRGPRX1,
PPIP5K2, SNX18, WEE2, C12orf23, DLGAP1, HIST1H2BD, MRGPRX2, PPL, SNX19, WFDC1,
C12orf29, DLGAP2, HIST1H2BE, MRGPRX3, PPM1A, SNX2, WFDC10A, C12orf39, DLGAP4,
HIST1H2BF, MRGPRX4, PPM1B, SNX20, WFDC10B, C12orf4, DLGAP5, HIST1H2BG, MRI1,
PPM1D, SNX21, WFDC11, C12orf40, DLK1, HIST1H2BH, MRM1, PPM1E, SNX22, WFDC12,
C12orf42, DLK2, HIST1H2BI, MRO, PPM1F, SNX24, WFDC13, C12orf43, DLL1, HIST1H2BJ,
MROH1, PPM1G, SNX25, WFDC2, C12orf44, DLL3, HIST1H2BK, MROH2A, PPM1H, SNX27,
WFDC3, C12orf45, DLL4, HIST1H2BL, MROH2B, PPM1J, SNX29, WFDC5, C12orf49, DLST,
HIST1H2BM, MROH5, PPM1K, SNX3, WFDC6, C12orf5, DLX1, HIST1H2BN, MROH6,
PPM1L, SNX30, WFDC8, C12orf50, DLX2, HIST1H2BO, MROH7, PPM1M, SNX31, WFDC9,
C12orf52, DLX3, HIST1H3A, MROH8, PPM1N, SNX32, WFIKKN1, C12orf54, DLX4,
HIST1H3B, MROH9, PPME1, SNX33, WFIKKN2, C12orf55, DLX5, HIST1H3C, MRP63, PPOX,
SNX4, WFS1, C12orf56, DLX6, HIST1H3D, MRPL1, PPP1CA, SNX5, WHAMM, C12orf57,
DMAP1, HIST1H3E, MRPL10, PPP1CB, SNX6, WHSC1, C12orf60, DMBT1, HIST1H3F,
MRPL11, PPP1CC, SNX7, WHSC1L1, C12orf61, DMBX1, HIST1H3G, MRPL12, PPP1R10,
SNX8, WIBG, C12orf65, DMC1, HIST1H3H, MRPL13, PPP1R11, SNX9, WIF1, C12orf66, DMD,
HIST1H3I, MRPL14, PPP1R12A, SOAT1, WIPF1, C12orf68, DMGDH, HIST1H3J, MRPL15,
PPP1R12B, SOAT2, WIPF2, C12orf71, DMKN, HIST1H4A, MRPL16, PPP1R12C, SOBP, WIPF3,
C12orf73, DMP1, HIST1H4B, MRPL17, PPP1R13B, SOCS1, WIPI1, C12orf74, DMPK,
HIST1H4C, MRPL18, PPP1R13L, SOCS2, WIPI2, C12orf75, DMRT1, HIST1H4D, MRPL19,
PPP1R14A, SOCS3, WISP1, C12orf76, DMRT2, HIST1H4E, MRPL2, PPP1R14B, SOCS4,
WISP2, C12orf77, DMRT3, HIST1H4F, MRPL20, PPP1R14C, SOCS5, WISP3, C12orf79,
DMRTA1, HIST1H4G, MRPL21, PPP1R14D, SOCS6, WIZ, C12orf80, DMRTA2, HIST1H4H,
MRPL22, PPP1R15A, SOCS7, WLS, C13orf35, DMRTB1, HIST1H4I, MRPL23, PPP1R15B,
SOD1, WNK1, C13orf45, DMRTC1, HIST1H4J, MRPL24, PPP1R16A, SOD2, WNK2, C14orf1,
DMRTC1B, HIST1H4K, MRPL27, PPP1R16B, SOD3, WNK3, C14orf105, DMRTC2, HIST1H4L,
MRPL28, PPP1R17, SOGA1, WNK4, C14orf119, DMTF1, HIST2H2AA3, MRPL3, PPP1R18,
SOGA2, WNT1, C14orf132, DMTN, HIST2H2AA4, MRPL30, PPP1R1A, SOGA3, WNT10A,
C14orf142, DMWD, HIST2H2AB, MRPL32, PPP1R1B, SOHLH1, WNT10B, C14orf159, DMXL1,
HIST2H2AC, MRPL33, PPP1R1C, SOHLH2, WNT11, C14orf164, DMXL2, HIST2H2BE,
MRPL34, PPP1R2, SON, WNT16, C14orf166, DNA2, HIST2H2BF, MRPL35, PPP1R21, SORBS1,
WNT2, C14orf166B, DNAAF1, HIST2H3A, MRPL36, PPP1R26, SORBS2, WNT2B, C14orf169,
DNAAF2, HIST2H3C, MRPL37, PPP1R27, SORBS3, WNT3, C14orf177, DNAAF3, HIST2H3D,
MRPL38, PPP1R32, SORCS1, WNT3A, C14orf178, DNAH1, HIST2H4A, MRPL39, PPP1R35,
SORCS2, WNT4, C14orf180, DNAH10, HIST2H4B, MRPL4, PPP1R36, SORCS3, WNT5A,
C14orf182, DNAH11, HIST3H2A, MRPL40, PPP1R37, SORD, WNT5B, C14orf183, DNAH12,
HIST3H2BB, MRPL41, PPP1R3A, SORL1, WNT6, C14orf2, DNAH14, HIST3H3, MRPL42,
PPP1R3B, SORT1, WNT7A, C14orf28, DNAH17, HIST4H4, MRPL43, PPP1R3C, SOS1, WNT7B,
C14orf37, DNAH2, HIVEP1, MRPL44, PPP1R3D, SOS2, WNT8A, C14orf39, DNAH3, HIVEP2,
MRPL45, PPP1R3E, SOST, WNT8B, C14orf79, DNAH5, HIVEP3, MRPL46, PPP1R3F,
SOSTDC1, WNT9A, C14orf80, DNAH6, HJURP, MRPL47, PPP1R3G, SOWAHA, WNT9B,
C14orf93, DNAH7, HK1, MRPL48, PPP1R42, SOWAHB, WRAP53, C15orf26, DNAH8, HK2,
MRPL49, PPP1R7, SOWAHC, WRAP73, C15orf27, DNAH9, HK3, MRPL50, PPP1R8,
SOWAHD, WRB, C15orf32, DNAI1, HKDC1, MRPL51, PPP1R9A, SOX1, WRN, C15orf38,
DNAI2, HKR1, MRPL52, PPP1R9B, SOX10, WRNIP1, C15orf38-AP3S2, DNAJA1, HLA-A,
MRPL53, PPP2CA, SOX11, WSB1, C15orf39, DNAJA2, HLA-B, MRPL54, PPP2CB, SOX12,
WSB2, C15orf40, DNAJA3, HLA-C, MRPL55, PPP2R1A, SOX13, WSCD1, C15orf41, DNAJA4,
HLA-DMA, MRPL9, PPP2R1B, SOX14, WSCD2, C15orf43, DNAJB1, HLA-DMB, MRPS10,
PPP2R2A, SOX15, WT1, C15orf48, DNAJB11, HLA-DOA, MRPS11, PPP2R2B, SOX17, WTAP,
C15orf52, DNAJB12, HLA-DOB, MRPS12, PPP2R2C, SOX18, WTH3DI, C15orf53, DNAJB13,
HLA-DPA1, MRPS14, PPP2R2D, SOX2, WTIP, C15orf54, DNAJB14, HLA-DPB1, MRPS15,
PPP2R3A, SOX21, WWC1, C15orf56, DNAJB2, HLA- DQA1, MRPS16, PPP2R3B, SOX3,
WWC2, C15orf57, DNAJB3, HLA-DQA2, MRPS17, PPP2R3C, SOX30, WWC3, C15orf59,
DNAJB4, HLA-DQB1, MRPS18A, PPP2R4, SOX4, WWOX, C15orf60, DNAJB5, HLA- DQB2,
MRPS18B, PPP2R5A, SOX5, WWP1, C15orf61, DNAJB6, HLA-DRA, MRPS18C, PPP2R5B,
SOX6, WWP2, C15orf62, DNAJB7, HLA-DRB1, MRPS2, PPP2R5C, SOX7, WWTR1, C15orf65,
DNAJB8, HLA- DRB5, MRPS21, PPP2R5D, SOX8, XAB2, C16orf11, DNAJB9, HLA-E,
MRPS22, PPP2R5E, SOX9, XAF1, C16orf13, DNAJC1, HLA-F, MRPS23, PPP3CA, SP1,
XAGE1D, C16orf3, DNAJC10, HLA-G, MRPS24, PPP3CB, SP100, XAGE2, C16orf45, DNAJC11,
HLCS, MRPS25, PPP3CC, SP110, XAGE3, C16orf46, DNAJC12, HLF, MRPS26, PPP3R1, SP140,
XAGE5, C16orf47, DNAJC13, HLTF, MRPS27, PPP3R2, SP140L, XBP1, C16orf52, DNAJC14,
HLX, MRPS28, PPP4C, SP2, XCL1, C16orf54, DNAJC15, HM13, MRPS30, PPP4R1, SP3, XCL2,
C16orf58, DNAJC16, HMBOX1, MRPS31, PPP4R2, SP4, XCR1, C16orf59, DNAJC17, HMBS,
MRPS33, PPP4R4, SP5, XDH, C16orf62, DNAJC18, HMCES, MRPS34, PPP5C, SP6, XG,
C16orf70, DNAJC19, HMCN1, MRPS35, PPP5D1, SP7, XIAP, C16orf71, DNAJC2, HMCN2,
MRPS36, PPP6C, SP8, XIRP1, C16orf72, DNAJC21, HMG20A, MRPS5, PPP6R1, SP9, XIRP2,
C16orf74, DNAJC22, HMG20B, MRPS6, PPP6R2, SPA17, XK, C16orf78, DNAJC24, HMGA1,
MRPS7, PPP6R3, SPACA1, XKR3, C16orf80, DNAJC25, HMGA2, MRPS9, PPRC1, SPACA3,
XKR4, C16orf82, DNAJC25-GNG10, HMGB1, MRRF, PPT1, SPACA4, XKR5, C16orf86,
DNAJC27, HMGB2, MRS2, PPT2, SPACA5, XKR6, C16orf87, DNAJC28, HMGB3, MRTO4,
PPTC7, SPACA5B, XKR7, C16orf89, DNAJC3, HMGB4, MRVI1, PPWD1, SPACA7, XKR8,
C16orf90, DNAJC30, HMGCL, MS4A1, PPY, SPAG1, XKR9, C16orf91, DNAJC4, HMGCLL1,
MS4A10, PQBP1, SPAG11A, XKRX, C16orf92, DNAJC5, HMGCR, MS4A12, PQLC1,
SPAG11B, XKRY, C16orf93, DNAJC5B, HMGCS1, MS4A13, PQLC2, SPAG16, XKRY2,
C16orf95, DNAJC5G, HMGCS2, MS4A14, PQLC3, SPAG17, XPA, C16orf96, DNAJC6, HMGN1,
MS4A15, PRAC1, SPAG4, XPC, C16orf97, DNAJC7, HMGN2, MS4A2, PRAC2, SPAG5,
XPNPEP1, C17orf100, DNAJC8, HMGN3, MS4A3, PRADC1, SPAG6, XPNPEP2, C17orf102,
DNAJC9, HMGN4, MS4A4A, PRAF2, SPAG7, XPNPEP3, C17orf103, DNAL1, HMGN5,
MS4A4E, PRAM1, SPAG8, XPO1, C17orf104, DNAL4, HMGXB3, MS4A5, PRAME, SPAG9,
XPO4, C17orf105, DNALI1, HMGXB4, MS4A6A, PRAMEF1, SPAM1, XPO5, C17orf107,
DNASE1, HMHA1, MS4A6E, PRAMEF10, SPANXA1, XPO6, C17orf112, DNASE1L1, HMHB1,
MS4A7, PRAMEF11, SPANXA2, XPO7, C17orf47, DNASE1L2, HMMR, MS4A8, PRAMEF12,
SPANXB1, XPOT, C17orf49, DNASE1L3, HMOX1, MSANTD1, PRAMEF13, SPANXB2, XPR1,
C17orf50, DNASE2, HMOX2, MSANTD2, PRAMEF14, SPANXC, XRCC1, C17orf51,
DNASE2B, HMP19, MSANTD3, PRAMEF15, SPANXD, XRCC2, C17orf53, DND1, HMSD,
MSANTD3-TMEFF1, PRAMEF16, SPANXN1, XRCC3, C17orf58, DNER, HMX1, MSANTD4,
PRAMEF17, SPANXN2, XRCC4, C17orf59, DNHD1, HMX2, MSC, PRAMEF18, SPANXN3,
XRCC5, C17orf62, DNLZ, HMX3, MSGN1, PRAMEF19, SPANXN4, XRCC6, C17orf64, DNM1,
HN1, MSH2, PRAMEF2, SPANXN5, XRCC6BP1, C17orf66, DNM1L, HN1L, MSH3,
PRAMEF20, SPARC, XRN1, C17orf67, DNM2, HNF1A, MSH4, PRAMEF21, SPARCL1, XRN2,
C17orf70, DNM3, HNF1B, MSH5, PRAMEF22, SPAST, XRRA1, C17orf72, DNMBP, HNF4A,
MSH6, PRAMEF23, SPATA1, XXYLT1, C17orf74, DNMT1, HNF4G, MSI1, PRAMEF25,
SPATA12, XYLB, C17orf75, DNMT3A, HNMT, MSI2, PRAMEF3, SPATA13, XYLT1, C17orf77,
DNMT3B, HNRNPA0, MSL1, PRAMEF4, SPATA16, XYLT2, C17orf78, DNMT3L, HNRNPA1,
MSL2, PRAMEF5, SPATA17, YAE1D1, C17orf80, DNPEP, HNRNPA1L2, MSL3, PRAMEF6,
SPATA18, YAF2, C17orf82, DNPH1, HNRNPA2B1, MSLN, PRAMEF7, SPATA19, YAP1,
C17orf85, DNTT, HNRNPA3, MSMB, PRAMEF8, SPATA2, YARS, C17orf89, DNTTIP1,
HNRNPAB, MSMO1, PRAMEF9, SPATA20, YARS2, C17orf96, DNTTIP2, HNRNPC, MSMP,
PRAP1, SPATA21, YBEY, C17orf97, DOC2A, HNRNPCL1, MSN, PRB1, SPATA22, YBX1,
C17orf98, DOC2B, HNRNPCP5, MSR1, PRB2, SPATA24, YBX2, C17orf99, DOCK1, HNRNPD,
MSRA, PRB3, SPATA25, YBX3, C18orf21, DOCK10, HNRNPDL, MSRB1, PRB4, SPATA2L,
YDJC, C18orf25, DOCK11, HNRNPF, MSRB2, PRC1, SPATA3, YEATS2, C18orf32, DOCK2,
HNRNPH1, MSRB3, PRCC, SPATA31A1, YEATS4, C18orf42, DOCK3, HNRNPH2, MSS51,
PRCD, SPATA31A2, YES1, C18orf54, DOCK4, HNRNPH3, MST1, PRCP, SPATA31A3, YIF1A,
C18orf56, DOCK5, HNRNPK, MST1L, PRDM1, SPATA31A4, YIF1B, C18orf63, DOCK6,
HNRNPL, MST1R, PRDM10, SPATA31A5, YIPF1, C18orf8, DOCK7, HNRNPLL, MST4,
PRDM11, SPATA31A6, YIPF2, C19orf10, DOCK8, HNRNPM, MSTN, PRDM12, SPATA31A7,
YIPF3, C19orf12, DOCK9, HNRNPR, MSTO1, PRDM13, SPATA31C1, YIPF4, C19orf18, DOHH,
HNRNPU, MSX1, PRDM14, SPATA31C2, YIPF5, C19orf24, DOK1, HNRNPUL1, MSX2,
PRDM15, SPATA31D1, YIPF6, C19orf25, DOK2, HNRNPUL2, MT1A, PRDM16, SPATA31D3,
YIPF7, C19orf26, DOK3, HOGA1, MT1B, PRDM2, SPATA31D4, YJEFN3, C19orf33, DOK4,
HOMER1, MT1E, PRDM4, SPATA31E1, YKT6, C19orf35, DOK5, HOMER2, MT1F, PRDM5,
SPATA32, YLPM1, C19orf38, DOK6, HOMER3, MT1G, PRDM6, SPATA33, YME1L1,
C19orf40, DOK7, HOMEZ, MT1H, PRDM7, SPATA4, YOD1, C19orf43, DOLK, HOOK1,
MT1HL1, PRDM8, SPATA5, YPEL1, C19orf44, DOLPP1, HOOK2, MT1M, PRDM9, SPATA5L1,
YPEL2, C19orf45, DONSON, HOOK3, MT1X, PRDX1, SPATA6, YPEL3, C19orf47, DOPEY1,
HOPX, MT2A, PRDX2, SPATA6L, YPEL4, C19orf48, DOPEY2, HORMAD1, MT3, PRDX3,
SPATA7, YPEL5, C19orf52, DOT1L, HORMAD2, MT4, PRDX4, SPATA8, YRDC, C19orf53,
DPAGT1, HOXA1, MTA1, PRDX5, SPATA9, YTHDC1, C19orf54, DPCD, HOXA10, MTA2,
PRDX6, SPATC1, YTHDC2, C19orf55, DPCR1, HOXA11, MTA3, PREB, SPATC1L, YTHDF1,
C19orf57, DPEP1, HOXA13, MTAP, PRELID1, SPATS1, YTHDF2, C19orf59, DPEP2, HOXA2,
MTBP, PRELID2, SPATS2, YTHDF3, C19orf60, DPEP3, HOXA3, MTCH1, PRELP, SPATS2L,
YWHAB, C19orf66, DPF1, HOXA4, MTCH2, PREP, SPC24, YWHAE, C19orf67, DPF2, HOXA5,
MTCP1, PREPL, SPC25, YWHAG, C19orf68, DPF3, HOXA6, MTDH, PREX1, SPCS1, YWHAH,
C19orf69, DPH1, HOXA7, MTERF, PREX2, SPCS2, YWHAQ, C19orf70, DPH2, HOXA9,
MTERFD1, PRF1, SPCS3, YWHAZ, C19orf71, DPH3, HOXB1, MTERFD2, PRG2, SPDEF, YY1,
C19orf73, DPH3P1, HOXB13, MTERFD3, PRG3, SPDL1, YY1AP1, C19orf77, DPH5, HOXB2,
MTF1, PRG4, SPDYA, YY2, C19orf80, DPH6, HOXB3, MTF2, PRH1, SPDYC, ZACN, C19orf81,
DPH7, HOXB4, MTFMT, PRH2, SPDYE1, ZADH2, C19orf82, DPM1, HOXB5, MTFP1,
PRICKLE1, SPDYE2, ZAK, C1D, DPM2, HOXB6, MTFR1, PRICKLE2, SPDYE2B, ZAN,
C1GALT1, DPM3, HOXB7, MTFR1L, PRICKLE3, SPDYE3, ZAP70, C1GALT1C1, DPP10,
HOXB8, MTFR2, PRICKLE4, SPDYE4, ZAR1, C1orf100, DPP3, HOXB9, MTG1, PRIM1,
SPDYE5, ZAR1L, C1orf101, DPP4, HOXC10, MTG2, PRIM2, SPDYE6, ZBBX, C1orf105, DPP6,
HOXC11, MTHFD1, PRIMA1, SPECC1, ZBED1, C1orf106, DPP7, HOXC12, MTHFD1L,
PRIMPOL, SPECC1L, ZBED2, C1orf109, DPP8, HOXC13, MTHFD2, PRKAA1, SPEF1, ZBED3,
C1orf110, DPP9, HOXC4, MTHFD2L, PRKAA2, SPEF2, ZBED4, C1orf111, DPPA2, HOXC5,
MTHFR, PRKAB1, SPEG, ZBED5, C1orf112, DPPA3, HOXC6, MTHFS, PRKAB2, SPEM1,
ZBED6, C1orf115, DPPA4, HOXC8, MTHFSD, PRKACA, SPEN, ZBED6CL, C1orf116, DPPA5,
HOXC9, MTIF2, PRKACB, SPERT, ZBP1, C1orf122, DPRX, HOXD1, MTIF3, PRKACG,
SPESP1, ZBTB1, C1orf123, DPT, HOXD10, MTL5, PRKAG1, SPG11, ZBTB10, C1orf127,
DPY19L1, HOXD11, MTM1, PRKAG2, SPG20, ZBTB11, C1orf131, DPY19L2, HOXD12,
MTMR1, PRKAG3, SPG21, ZBTB12, C1orf137, DPY19L3, HOXD13, MTMR10, PRKAR1A,
SPG7, ZBTB14, C1orf141, DPY19L4, HOXD3, MTMR11, PRKAR1B, SPHAR, ZBTB16,
C1orf146, DPY30, HOXD4, MTMR12, PRKAR2A, SPHK1, ZBTB17, C1orf158, DPYD, HOXD8,
MTMR14, PRKAR2B, SPHK2, ZBTB18, C1orf159, DPYS, HOXD9, MTMR2, PRKCA, SPHKAP,
ZBTB2, C1orf162, DPYSL2, HP, MTMR3, PRKCB, SPI1, ZBTB20, C1orf167, DPYSL3, HP1BP3,
MTMR4, PRKCD, SPIB, ZBTB21, C1orf168, DPYSL4, HPCA, MTMR6, PRKCDBP, SPIC,
ZBTB22, C1orf172, DPYSL5, HPCAL1, MTMR7, PRKCE, SPICE1, ZBTB24, C1orf173, DQX1,
HPCAL4, MTMR8, PRKCG, SPIDR, ZBTB25, C1orf174, DR1, HPD, MTMR9, PRKCH, SPIN1,
ZBTB26, C1orf177, DRAM1, HPDL, MTNR1A, PRKCI, SPIN2A, ZBTB3, C1orf185, DRAM2,
HPGD, MTNR1B, PRKCQ, SPIN2B, ZBTB32, C1orf186, DRAP1, HPGDS, MTO1, PRKCSH,
SPIN3, ZBTB33, C1orf189, DRAXIN, HPN, MTOR, PRKCZ, SPIN4, ZBTB34, C1orf192, DRC1,
HPR, MTPAP, PRKD1, SPINK1, ZBTB37, C1orf194, DRD1, HPRT1, MTPN, PRKD2, SPINK13,
ZBTB38, C1orf195, DRD2, HPS1, MTR, PRKD3, SPINK14, ZBTB39, C1orf198, DRD3, HPS3,
MTRF1, PRKDC, SPINK2, ZBTB4, C1orf204, DRD4, HPS4, MTRF1L, PRKG1, SPINK4,
ZBTB40, C1orf21, DRD5, HPS5, MTRNR2L10, PRKG2, SPINK5, ZBTB41, C1orf210, DRG1,
HPS6, MTRNR2L2, PRKRA, SPINK6, ZBTB42, C1orf216, DRG2, HPSE, MTRNR2L3,
PRKRIP1, SPINK7, ZBTB43, C1orf226, DRGX, HPSE2, MTRNR2L4, PRKRIR, SPINK8,
ZBTB44, C1orf227, DROSHA, HPX, MTRNR2L5, PRKX, SPINK9, ZBTB45, C1orf228, DRP2,
HR, MTRNR2L6, PRL, SPINT1, ZBTB46, C1orf229, DSC1, HRAS, MTRNR2L7, PRLH, SPINT2,
ZBTB47, C1orf233, DSC2, HRASLS, MTRNR2L9, PRLHR, SPINT3, ZBTB48, C1orf234, DSC3,
HRASLS2, MTRR, PRLR, SPINT4, ZBTB49, C1orf27, DSCAM, HRASLS5, MTSS1, PRM1,
SPIRE1, ZBTB5, C1orf35, DSCAML1, HRC, MTSS1L, PRM2, SPIRE2, ZBTB6, C1orf43,
DSCC1, HRCT1, MTTP, PRM3, SPN, ZBTB7A, C1orf50, DSCR3, HRG, MTURN, PRMT1,
SPNS1, ZBTB7B, C1orf51, DSCR4, HRH1, MTUS1, PRMT10, SPNS2, ZBTB7C, C1orf52, DSE,
HRH2, MTUS2, PRMT2, SPNS3, ZBTB8A, C1orf53, DSEL, HRH3, MTX1, PRMT3, SPO11,
ZBTB8B, C1orf54, DSG1, HRH4, MTX2, PRMT5, SPOCD1, ZBTB8OS, C1orf56, DSG2, HRK,
MTX3, PRMT6, SPOCK1, ZBTB9, C1orf61, DSG3, HRNR, MUC1, PRMT7, SPOCK2,
ZC2HC1A, C1orf63, DSG4, HRSP12, MUC12, PRMT8, SPOCK3, ZC2HC1B, C1orf64, DSN1,
HS1BP3, MUC13, PRND, SPON1, ZC2HC1C, C1orf65, DSP, HS2ST1, MUC15, PRNP, SPON2,
ZC3H10, C1orf68, DSPP, HS3ST1, MUC16, PROB1, SPOP, ZC3H11A, C1orf74, DST, HS3ST2,
MUC17, PROC, SPOPL, ZC3H12A, C1orf85, DSTN, HS3ST3A1, MUC19, PROCA1, SPP1,
ZC3H12B, C1orf86, DSTNP4, HS3ST3B1, MUC2, PROCR, SPP2, ZC3H12C, C1orf87, DSTYK,
HS3ST4, MUC20, PRODH, SPPL2A, ZC3H12D, C1orf94, DTD1, HS3ST5, MUC21, PRODH2,
SPPL2B, ZC3H13, C1orf95, DTD2, HS3ST6, MUC22, PROK1, SPPL2C, ZC3H14, C1QA,
DTHD1, HS6ST1, MUC3A, PROK2, SPPL3, ZC3H15, C1QB, DTL, HS6ST2, MUC4, PROKR1,
SPR, ZC3H18, C1QBP, DTNA, HS6ST3, MUC5AC, PROKR2, SPRED1, ZC3H3, C1QC, DTNB,
HSBP1, MUC5B, PROL1, SPRED2, ZC3H4, C1QL1, DTNBP1, HSBP1L1, MUC6, PROM1,
SPRED3, ZC3H6, C1QL2, DTWD1, HSCB, MUC7, PROM2, SPRN, ZC3H7A, C1QL3, DTWD2,
HSD11B1, MUC8, PROP1, SPRR1A, ZC3H7B, C1QL4, DTX1, HSD11B1L, MUCL1, PROS1,
SPRR1B, ZC3H8, C1QTNF1, DTX2, HSD11B2, MUL1, PROSC, SPRR2A, ZC3HAV1,
C1QTNF2, DTX3, HSD17B1, MUM1, PROSER1, SPRR2B, ZC3HAV1L, C1QTNF3, DTX3L,
HSD17B10, MUM1L1, PROSER2, SPRR2D, ZC3HC1, C1QTNF4, DTX4, HSD17B11, MURC,
PROX1, SPRR2E, ZC4H2, C1QTNF5, DTYMK, HSD17B12, MUS81, PROX2, SPRR2F,
ZCCHC10, C1QTNF6, DUOX1, HSD17B13, MUSK, PROZ, SPRR2G, ZCCHC11, C1QTNF7,
DUOX2, HSD17B14, MUSTN1, PRPF18, SPRR3, ZCCHC12, C1QTNF8, DUOXA1, HSD17B2,
MUT, PRPF19, SPRR4, ZCCHC13, C1QTNF9, DUOXA2, HSD17B3, MUTYH, PRPF3, SPRTN,
ZCCHC14, C1QTNF9B, DUPD1, HSD17B4, MVB12A, PRPF31, SPRY1, ZCCHC16,
C1QTNF9B-AS1, DUS1L, HSD17B6, MVB12B, PRPF38A, SPRY2, ZCCHC17, C1R, DUS2,
HSD17B7, MVD, PRPF38B, SPRY3, ZCCHC18, C1RL, DUS3L, HSD17B8, MVK, PRPF39,
SPRY4, ZCCHC2, C1S, DUS4L, HSD3B1, MVP, PRPF4, SPRYD3, ZCCHC24, C2, DUSP1,
HSD3B2, MX1, PRPF40A, SPRYD4, ZCCHC3, C20orf112, DUSP10, HSD3B7, MX2, PRPF40B,
SPRYD7, ZCCHC4, C20orf141, DUSP11, HSDL1, MXD1, PRPF4B, SPSB1, ZCCHC5,
C20orf144, DUSP12, HSDL2, MXD3, PRPF6, SPSB2, ZCCHC6, C20orf166, DUSP13, HSF1,
MXD4, PRPF8, SPSB3, ZCCHC7, C20orf173, DUSP14, HSF2, MXI1, PRPH, SPSB4, ZCCHC8,
C20orf194, DUSP15, HSF2BP, MXRA5, PRPH2, SPTA1, ZCCHC9, C20orf195, DUSP16, HSF4,
MXRA7, PRPS1, SPTAN1, ZCRB1, C20orf196, DUSP18, HSF5, MXRA8, PRPS1L1, SPTB,
ZCWPW1, C20orf197, DUSP19, HSFX1, MYADM, PRPS2, SPTBN1, ZCWPW2, C20orf201,
DUSP2, HSFX2, MYADML2, PRPSAP1, SPTBN2, ZDBF2, C20orf202, DUSP21, HSFY1, MYB,
PRPSAP2, SPTBN4, ZDHHC1, C20orf203, DUSP22, HSFY2, MYBBP1A, PRR11, SPTBN5,
ZDHHC11, C20orf24, DUSP23, HSH2D, MYBL1, PRR12, SPTLC1, ZDHHC11B, C20orf26,
DUSP26, HSP90AA1, MYBL2, PRR13, SPTLC2, ZDHHC12, C20orf27, DUSP27, HSP90AB1,
MYBPC1, PRR14, SPTLC3, ZDHHC13, C20orf62, DUSP28, HSP90B1, MYBPC2, PRR14L,
SPTSSA, ZDHHC14, C20orf78, DUSP3, HSPA12A, MYBPC3, PRR15, SPTSSB, ZDHHC15,
C20orf85, DUSP4, HSPA12B, MYBPH, PRR15L, SPTY2D1, ZDHHC16, C20orf96, DUSP5,
HSPA13, MYBPHL, PRR16, SPZ1, ZDHHC17, C21orf140, DUSP6, HSPA14, MYC, PRR18,
SQLE, ZDHHC18, C21orf2, DUSP7, HSPA1A, MYCBP, PRR19, SQRDL, ZDHHC19, C21orf33,
DUSP8, HSPA1B, MYCBP2, PRR20A, SQSTM1, ZDHHC2, C21orf58, DUSP9, HSPA1L,
MYCBPAP, PRR20B, SRA1, ZDHHC20, C21orf59, DUT, HSPA2, MYCL, PRR20C, SRBD1,
ZDHHC21, C21orf62, DUXA, HSPA4, MYCN, PRR20D, SRC, ZDHHC22, C21orf91, DVL1,
HSPA4L, MYCT1, PRR20E, SRCAP, ZDHHC23, C22orf15, DVL2, HSPA5, MYD88, PRR21,
SRCIN1, ZDHHC24, C22orf23, DVL3, HSPA6, MYEF2, PRR22, SRCRB4D, ZDHHC3, C22orf24,
DXO, HSPA8, MYEOV, PRR23A, SRD5A1, ZDHHC4, C22orf26, DYDC1, HSPA9, MYEOV2,
PRR23B, SRD5A2, ZDHHC5, C22orf29, DYDC2, HSPB1, MYF5, PRR23C, SRD5A3, ZDHHC6,
C22orf31, DYM, HSPB11, MYF6, PRR24, SREBF1, ZDHHC7, C22orf39, DYNAP, HSPB2,
MYH1, PRR25, SREBF2, ZDHHC8, C22orf42, DYNC1H1, HSPB3, MYH10, PRR3, SREK1,
ZDHHC9, C22orf43, DYNC1I1, HSPB6, MYH11, PRR30, SREK1IP1, ZEB1, C22orf46,
DYNC1I2, HSPB7, MYH13, PRR4, SRF, ZEB2, C2CD2, DYNC1LI1, HSPB8, MYH14, PRR5,
SRFBP1, ZER1, C2CD2L, DYNC1LI2, HSPB9, MYH15, PRR5-ARHGAP8, SRGAP1, ZFAND1,
C2CD3, DYNC2H1, HSPBAP1, MYH2, PRR5L, SRGAP2, ZFAND2A, C2CD4A, DYNC2LI1,
HSPBP1, MYH3, PRR7, SRGAP2B, ZFAND2B, C2CD4B, DYNLL1, HSPD1, MYH4, PRR9,
SRGAP2C, ZFAND3, C2CD4C, DYNLL2, HSPE1, MYH6, PRRC1, SRGAP3, ZFAND4,
C2CD4D, DYNLRB1, HSPE1-MOB4, MYH7, PRRC2A, SRGN, ZFAND5, C2CD5, DYNLRB2,
HSPG2, MYH7B, PRRC2B, SRI, ZFAND6, C2orf15, DYNLT1, HSPH1, MYH8, PRRC2C, SRL,
ZFAT, C2orf16, DYNLT3, HTATIP2, MYH9, PRRG1, SRM, ZFC3H1, C2orf27A, DYRK1A,
HTATSF1, MYL1, PRRG2, SRMS, ZFHX2, C2orf27B, DYRK1B, HTN1, MYL10, PRRG3,
SRP14, ZFHX3, C2orf40, DYRK2, HTN3, MYL12A, PRRG4, SRP19, ZFHX4, C2orf42, DYRK3,
HTR1A, MYL12B, PRRT1, SRP54, ZFP1, C2orf43, DYRK4, HTR1B, MYL2, PRRT2, SRP68,
ZFP14, C2orf44, DYSF, HTR1D, MYL3, PRRT3, SRP72, ZFP2, C2orf47, DYTN, HTR1E, MYL4,
PRRT4, SRP9, ZFP28, C2orf48, DYX1C1, HTR1F, MYL5, PRRX1, SRPK1, ZFP3, C2orf49,
DZANK1, HTR2A, MYL6, PRRX2, SRPK2, ZFP30, C2orf50, DZIP1, HTR2B, MYL6B, PRSS1,
SRPK3, ZFP36, C2orf53, DZIP1L, HTR2C, MYL7, PRSS12, SRPR, ZFP36L1, C2orf54, DZIP3,
HTR3A, MYL9, PRSS16, SRPRB, ZFP36L2, C2orf57, E2F1, HTR3B, MYLIP, PRSS2, SRPX,
ZFP37, C2orf61, E2F2, HTR3C, MYLK, PRSS21, SRPX2, ZFP41, C2orf62, E2F3, HTR3D,
MYLK2, PRSS22, SRR, ZFP42, C2orf66, E2F4, HTR3E, MYLK3, PRSS23, SRRD, ZFP57,
C2orf68, E2F5, HTR4, MYLK4, PRSS27, SRRM1, ZFP62, C2orf69, E2F6, HTR5A, MYLPF,
PRSS3, SRRM2, ZFP64, C2orf70, E2F7, HTR6, MYNN, PRSS33, SRRM3, ZFP69, C2orf71, E2F8,
HTR7, MYO10, PRSS35, SRRM4, ZFP69B, C2orf72, E4F1, HTRA1, MYO15A, PRSS36, SRRM5,
ZFP82, C2orf73, EAF1, HTRA2, MYO16, PRSS37, SRRT, ZFP90, C2orf74, EAF2, HTRA3,
MYO18A, PRSS38, SRSF1, ZFP91, C2orf76, EAPP, HTRA4, MYO18B, PRSS41, SRSF10,
ZFP92, C2orf78, EARS2, HTT, MYO19, PRSS42, SRSF11, ZFPL1, C2orf80, EBAG9, HUNK,
MYO1A, PRSS44, SRSF12, ZFPM1, C2orf81, EBF1, HUS1, MYO1B, PRSS45, SRSF2, ZFPM2,
C2orf82, EBF2, HUS1B, MYO1C, PRSS46, SRSF3, ZFR, C2orf83, EBF3, HUWE1, MYO1D,
PRSS48, SRSF4, ZFR2, C2orf88, EBF4, HVCN1, MYO1E, PRSS50, SRSF5, ZFX, C2orf91, EBI3,
HYAL1, MYO1F, PRSS53, SRSF6, ZFY, C3, EBLN1, HYAL2, MYO1G, PRSS54, SRSF7,
ZFYVE1, C3AR1, EBLN2, HYAL3, MYO1H, PRSS55, SRSF8, ZFYVE16, C3orf14, EBNA1BP2,
HYAL4, MYO3A, PRSS56, SRSF9, ZFYVE19, C3orf17, EBP, HYDIN, MYO3B, PRSS57,
SRXN1, ZFYVE20, C3orf18, EBPL, HYI, MYO5A, PRSS58, SRY, ZFYVE21, C3orf20, ECD,
HYKK, MYO5B, PRSS8, SS18, ZFYVE26, C3orf22, ECE1, HYLS1, MYO5C, PRTFDC1,
SS18L1, ZFYVE27, C3orf27, ECE2, HYOU1, MYO6, PRTG, SS18L2, ZFYVE28, C3orf30,
ECEL1, HYPK, MYO7A, PRTN3, SSB, ZFYVE9, C3orf33, ECH1, IAH1, MYO7B, PRUNE,
SSBP1, ZG16, C3orf35, ECHDC1, IAPP, MYO9A, PRUNE2, SSBP2, ZG16B, C3orf36, ECHDC2,
IARS, MYO9B, PRX, SSBP3, ZGLP1, C3orf38, ECHDC3, IARS2, MYOC, PRY, SSBP4, ZGPAT,
C3orf43, ECHS1, IBA57, MYOCD, PRY2, SSC5D, ZHX1, C3orf52, ECI1, IBSP, MYOD1, PSAP,
SSFA2, ZHX1-C8ORF76, C3orf55, ECI2, IBTK, MYOF, PSAPL1, SSH1, ZHX2, C3orf56, ECM1,
ICA1, MYOG, PSAT1, SSH2, ZHX3, C3orf58, ECM2, ICA1L, MYOM1, PSCA, SSH3, ZIC1,
C3orf62, ECSCR, ICAM1, MYOM2, PSD, SSMEM1, ZIC2, C3orf67, ECSIT, ICAM2, MYOM3,
PSD2, SSNA1, ZIC3, C3orf70, ECT2, ICAM3, MYOT, PSD3, SSPN, ZIC4, C3orf72, ECT2L,
ICAM4, MYOZ1, PSD4, SSPO, ZIC5, C3orf79, EDA, ICAM5, MYOZ2, PSEN1, SSR1, ZIK1,
C3orf80, EDA2R, ICK, MYOZ3, PSEN2, SSR2, ZIM2, C3orf83, EDAR, ICMT, MYPN, PSENEN,
SSR3, ZIM3, C3orf84, EDARADD, ICOS, MYPOP, PSG1, SSR4, ZKSCAN1, C4A, EDC3,
ICOSLG, MYRF, PSG11, SSRP1, ZKSCAN2, C4B, EDC4, ICT1, MYRFL, PSG2, SSSCA1,
ZKSCAN3, C4B_2, EDDM3A, ID1, MYRIP, PSG3, SST, ZKSCAN4, C4BPA, EDDM3B, ID2,
MYSM1, PSG4, SSTR1, ZKSCAN5, C4BPB, EDEM1, ID3, MYT1, PSG5, SSTR2, ZKSCAN7,
C4orf17, EDEM2, ID4, MYT1L, PSG6, SSTR3, ZKSCAN8, C4orf19, EDEM3, IDE, MYZAP,
PSG7, SSTR4, ZMAT1, C4orf21, EDF1, IDH1, MZB1, PSG8, SSTR5, ZMAT2, C4orf22, EDIL3,
IDH2, MZF1, PSG9, SSU72, ZMAT3, C4orf26, EDN1, IDH3A, MZT1, PSIP1, SSUH2, ZMAT4,
C4orf27, EDN2, IDH3B, MZT2A, PSKH1, SSX1, ZMAT5, C4orf29, EDN3, IDH3G, MZT2B,
PSKH2, SSX2, ZMIZ1, C4orf3, EDNRA, IDI1, N4BP1, PSMA1, SSX2B, ZMIZ2, C4orf32,
EDNRB, IDI2, N4BP2, PSMA2, SSX2IP, ZMPSTE24, C4orf33, EEA1, IDNK, N4BP2L1, PSMA3,
SSX3, ZMYM1, C4orf36, EED, IDO1, N4BP2L2, PSMA4, SSX4, ZMYM2, C4orf40, EEF1A1,
IDO2, N4BP3, PSMA5, SSX4B, ZMYM3, C4orf45, EEF1A2, IDS, N6AMT1, PSMA6, SSX5,
ZMYM4, C4orf46, EEF1B2, IDUA, N6AMT2, PSMA7, SSX7, ZMYM5, C4orf47, EEF1D, IER2,
NAA10, PSMA8, ST13, ZMYM6, C4orf48, EEF1E1, IER3, NAA11, PSMB1, ST14, ZMYM6NB,
C4orf50, EEF1G, IER3IP1, NAA15, PSMB10, ST18, ZMYND10, C4orf51, EEF2, IER5, NAA16,
PSMB11, ST20, ZMYND11, C4orf6, EEF2K, IER5L, NAA20, PSMB2, ST20-MTHFS,
ZMYND12, C5, EEFSEC, IFFO1, NAA25, PSMB3, ST3GAL1, ZMYND15, C5AR1, EEPD1,
IFFO2, NAA30, PSMB4, ST3GAL2, ZMYND19, C5AR2, EFCAB1, IFI16, NAA35, PSMB5,
ST3GAL3, ZMYND8, C5orf15, EFCAB11, IFI27, NAA38, PSMB6, ST3GAL4, ZNF10, C5orf20,
EFCAB12, IFI27L1, NAA40, PSMB7, ST3GAL5, ZNF100, C5orf22, EFCAB13, IFI27L2, NAA50,
PSMB8, ST3GAL6, ZNF101, C5orf24, EFCAB14, IFI30, NAA60, PSMB9, ST5, ZNF106, C5orf28,
EFCAB2, IFI35, NAAA, PSMC1, ST6GAL1, ZNF107, C5orf30, EFCAB3, IFI44, NAALAD2,
PSMC2, ST6GAL2, ZNF112, C5orf34, EFCAB4A, IFI44L, NAALADL1, PSMC3, ST6GALNAC1,
ZNF114, C5orf38, EFCAB4B, IFI6, NAALADL2, PSMC3IP, ST6GALNAC2, ZNF117, C5orf42,
EFCAB5, IFIH1, NAB1, PSMC4, ST6GALNAC3, ZNF12, C5orf45, EFCAB6, IFIT1, NAB2,
PSMC5, ST6GALNAC4, ZNF121, C5orf46, EFCAB7, IFIT1B, NABP1, PSMC6, ST6GALNAC5,
ZNF124, C5orf47, EFCAB8, IFIT2, NABP2, PSMD1, ST6GALNAC6, ZNF131, C5orf48,
EFCAB9, IFIT3, NACA, PSMD10, ST7, ZNF132, C5orf49, EFCC1, IFIT5, NACA2, PSMD11,
ST7L, ZNF133, C5orf50, EFEMP1, IFITM1, NACAD, PSMD12, ST8SIA1, ZNF134, C5orf51,
EFEMP2, IFITM10, NACC1, PSMD13, ST8SIA2, ZNF135, C5orf52, EFHB, IFITM2, NACC2,
PSMD14, ST8SIA3, ZNF136, C5orf55, EFHC1, IFITM3, NADK, PSMD2, ST8SIA4, ZNF138,
C5orf58, EFHC2, IFITM5, NADK2, PSMD3, ST8SIA5, ZNF14, C5orf60, EFHD1, IFLTD1,
NADSYN1, PSMD4, ST8SIA6, ZNF140, C5orf63, EFHD2, IFNA1, NAE1, PSMD5, STAB1,
ZNF141, C5orf64, EFNA1, IFNA10, NAF1, PSMD6, STAB2, ZNF142, C6, EFNA2, IFNA13,
NAGA, PSMD7, STAC, ZNF143, C6orf1, EFNA3, IFNA14, NAGK, PSMD8, STAC2, ZNF146,
C6orf10, EFNA4, IFNA16, NAGLU, PSMD9, STAC3, ZNF148, C6orf106, EFNA5, IFNA17,
NAGPA, PSME1, STAG1, ZNF154, C6orf118, EFNB1, IFNA2, NAGS, PSME2, STAG2, ZNF155,
C6orf132, EFNB2, IFNA21, NAIF1, PSME3, STAG3, ZNF157, C6orf136, EFNB3, IFNA4, NAIP,
PSME4, STAM, ZNF16, C6orf141, EFR3A, IFNA5, NALCN, PSMF1, STAM2, ZNF160, C6orf15,
EFR3B, IFNA6, NAMPT, PSMG1, STAMBP, ZNF165, C6orf163, EFS, IFNA7, NANOG, PSMG2,
STAMBPL1, ZNF169, C6orf165, EFTUD1, IFNA8, NANOGNB, PSMG3, STAP1, ZNF17,
C6orf183, EFTUD2, IFNAR1, NANOS1, PSMG4, STAP2, ZNF174, C6orf195, EGF, IFNAR2,
NANOS2, PSORS1C1, STAR, ZNF175, C6orf201, EGFL6, IFNB1, NANOS3, PSORS1C2,
STARD10, ZNF177, C6orf203, EGFL7, IFNE, NANP, PSPC1, STARD13, ZNF18, C6orf211,
EGFL8, IFNG, NANS, PSPH, STARD3, ZNF180, C6orf222, EGFLAM, IFNGR1, NAP1L1, PSPN,
STARD3NL, ZNF181, C6orf223, EGFR, IFNGR2, NAP1L2, PSRC1, STARD4, ZNF182,
C6orf226, EGLN1, IFNK, NAP1L3, PSTK, STARD5, ZNF184, C6orf25, EGLN2, IFNL1,
NAP1L4, PSTPIP1, STARD6, ZNF185, C6orf47, EGLN3, IFNL2, NAP1L5, PSTPIP2, STARD7,
ZNF189, C6orf48, EGR1, IFNL3, NAPA, PTAFR, STARD8, ZNF19, C6orf52, EGR2, IFNL4,
NAPB, PTAR1, STARD9, ZNF195, C6orf57, EGR3, IFNLR1, NAPEPLD, PTBP1, STAT1,
ZNF197, C6orf58, EGR4, IFNW1, NAPG, PTBP2, STAT2, ZNF2, C6orf62, EHBP1, IFRD1,
NAPRT1, PTBP3, STAT3, ZNF20, C6orf7, EHBP1L1, IFRD2, NAPSA, PTCD1, STAT4, ZNF200,
C6orf89, EHD1, IFT122, NARF, PTCD2, STAT5A, ZNF202, C6orf99, EHD2, IFT140, NARFL,
PTCD3, STAT5B, ZNF205, C7, EHD3, IFT172, NARG2, PTCH1, STAT6, ZNF207, C7orf10,
EHD4, IFT20, NARR, PTCH2, STATH, ZNF208, C7orf25, EHF, IFT27, NARS, PTCHD1, STAU1,
ZNF211, C7orf26, EHHADH, IFT43, NARS2, PTCHD2, STAU2, ZNF212, C7orf31, EHMT1,
IFT46, NASP, PTCHD3, STBD1, ZNF213, C7orf33, EHMT2, IFT52, NAT1, PTCHD4, STC1,
ZNF214, C7orf34, EI24, IFT57, NAT10, PTCRA, STC2, ZNF215, C7orf43, EID1, IFT74, NAT14,
PTDSS1, STEAP1, ZNF217, C7orf49, EID2, IFT80, NAT16, PTDSS2, STEAP1B, ZNF219,
C7orf50, EID2B, IFT81, NAT2, PTEN, STEAP2, ZNF22, C7orf55, EID3, IFT88, NAT6, PTER,
STEAP3, ZNF221, C7orf55- LUC7L2, EIF1, IGBP1, NAT8, PTF1A, STEAP4, ZNF222, C7orf57,
EIF1AD, IGDCC3, NAT8B, PTGDR, STH, ZNF223, C7orf60, EIF1AX, IGDCC4, NAT8L,
PTGDR2, STIL, ZNF224, C7orf61, EIF1AY, IGF1, NAT9, PTGDS, STIM1, ZNF225, C7orf62,
EIF1B, IGF1R, NAV1, PTGER1, STIM2, ZNF226, C7orf63, EIF2A, IGF2, NAV2, PTGER2,
STIP1, ZNF227, C7orf65, EIF2AK1, IGF2BP1, NAV3, PTGER3, STK10, ZNF229, C7orf66,
EIF2AK2, IGF2BP2, NBAS, PTGER4, STK11, ZNF23, C7orf69, EIF2AK3, IGF2BP3, NBEA,
PTGES, STK11IP, ZNF230, C7orf71, EIF2AK4, IGF2R, NBEAL1, PTGES2, STK16, ZNF232,
C7orf72, EIF2B1, IGFALS, NBEAL2, PTGES3, STK17A, ZNF233, C7orf73, EIF2B2, IGFBP1,
NBL1, PTGES3L, STK17B, ZNF234, C7orf76, EIF2B3, IGFBP2, NBN, PTGES3L-AARSD1,
STK19, ZNF235, C8A, EIF2B4, IGFBP3, NBPF14, PTGFR, STK24, ZNF236, C8B, EIF2B5,
IGFBP4, NBPF3, PTGFRN, STK25, ZNF239, C8G, EIF2D, IGFBP5, NBPF4, PTGIR, STK3,
ZNF24, C8orf22, EIF2S1, IGFBP6, NBPF6, PTGIS, STK31, ZNF248, C8orf31, EIF2S2, IGFBP7,
NBPF7, PTGR1, STK32A, ZNF25, C8orf33, EIF2S3, IGFBPL1, NBR1, PTGR2, STK32B,
ZNF250, C8orf34, EIF3A, IGFL1, NCALD, PTGS1, STK32C, ZNF251, C8orf37, EIF3B, IGFL2,
NCAM1, PTGS2, STK33, ZNF253, C8orf4, EIF3C, IGFL3, NCAM2, PTH, STK35, ZNF254,
C8orf44, EIF3CL, IGFL4, NCAN, PTH1R, STK36, ZNF256, C8orf44-SGK3, EIF3D, IGFLR1,
NCAPD2, PTH2, STK38, ZNF257, C8orf46, EIF3E, IGFN1, NCAPD3, PTH2R, STK38L, ZNF259,
C8orf47, EIF3F, IGHMBP2, NCAPG, PTHLH, STK39, ZNF26, C8orf48, EIF3G, IGIP, NCAPG2,
PTK2, STK4, ZNF260, C8orf58, EIF3H, IGJ, NCAPH, PTK2B, STK40, ZNF263, C8orf59, EIF3I,
IGLL1, NCAPH2, PTK6, STMN1, ZNF264, C8orf74, EIF3J, IGLL5, NCBP1, PTK7, STMN2,
ZNF266, C8orf76, EIF3K, IGLON5, NCBP2, PTMA, STMN3, ZNF267, C8orf82, EIF3L, IGSF1,
NCCRP1, PTMS, STMN4, ZNF268, C8orf86, EIF3M, IGSF10, NCDN, PTN, STMND1, ZNF273,
C8orf87, EIF4A1, IGSF11, NCEH1, PTOV1, STOM, ZNF274, C9, EIF4A2, IGSF21, NCF1,
PTP4A1, STOML1, ZNF275, C9orf106, EIF4A3, IGSF22, NCF2, PTP4A2, STOML2, ZNF276,
C9orf114, EIF4B, IGSF23, NCF4, PTP4A3, STOML3, ZNF277, C9orf116, EIF4E, IGSF3, NCK1,
PTPDC1, STON1, ZNF28, C9orf117, EIF4E1B, IGSF5, NCK2, PTPLA, STON1-GTF2A1L,
ZNF280A, C9orf129, EIF4E2, IGSF6, NCKAP1, PTPLAD1, STON2, ZNF280B, C9orf131,
EIF4E3, IGSF8, NCKAP1L, PTPLAD2, STOX1, ZNF280C, C9orf135, EIF4EBP1, IGSF9,
NCKAP5, PTPLB, STOX2, ZNF280D, C9orf139, EIF4EBP2, IGSF9B, NCKAP5L, PTPMT1,
STPG1, ZNF281, C9orf142, EIF4EBP3, IHH, NCKIPSD, PTPN1, STPG2, ZNF282, C9orf152,
EIF4ENIF1, IK, NCL, PTPN11, STRA13, ZNF283, C9orf153, EIF4G1, IKBIP, NCLN, PTPN12,
STRA6, ZNF284, C9orf156, EIF4G2, IKBKAP, NCMAP, PTPN13, STRA8, ZNF285, C9orf16,
EIF4G3, IKBKB, NCOA1, PTPN14, STRADA, ZNF286A, C9orf163, EIF4H, IKBKE, NCOA2,
PTPN18, STRADB, ZNF286B, C9orf169, EIF5, IKBKG, NCOA3, PTPN2, STRAP, ZNF287,
C9orf170, EIF5A, IKZF1, NCOA4, PTPN20A, STRBP, ZNF292, C9orf171, EIF5A2, IKZF2,
NCOA5, PTPN20B, STRC, ZNF296, C9orf173, EIF5AL1, IKZF3, NCOA6, PTPN21, STRIP1,
ZNF3, C9orf24, EIF5B, IKZF4, NCOA7, PTPN22, STRIP2, ZNF30, C9orf3, EIF6, IKZF5,
NCOR1, PTPN23, STRN, ZNF300, C9orf37, ELAC1, INTERLEUKIN, IL10, NCOR2, PTPN3,
STRN3, ZNF302, C9orf40, ELAC2, IL10RA, NCR1, PTPN4, STRN4, ZNF304, C9orf41, ELANE,
IL10RB, NCR2, PTPN5, STS, ZNF311, C9orf43, ELAVL1, IL11, NCR3, PTPN6, STT3A,
ZNF316, C9orf47, ELAVL2, IL11RA, NCR3LG1, PTPN7, STT3B, ZNF317, C9orf50, ELAVL3,
IL12A, NCS1, PTPN9, STUB1, ZNF318, C9orf57, ELAVL4, IL12B, NCSTN, PTPRA, STX10,
ZNF319, C9orf62, ELF1, IL12RB1, NDC1, PTPRB, STX11, ZNF32, C9orf64, ELF2, IL12RB2,
NDC80, PTPRC, STX12, ZNF320, C9orf66, ELF3, IL13, NDE1, PTPRCAP, STX16, ZNF322,
C9orf69, ELF4, IL13RA1, NDEL1, PTPRD, STX17, ZNF324, C9orf72, ELF5, IL13RA2, NDFIP1,
PTPRE, STX18, ZNF324B, C9orf78, ELFN1, IL15, NDFIP2, PTPRF, STX19, ZNF326, C9orf84,
ELFN2, IL15RA, NDN, PTPRG, STX1A, ZNF329, C9orf85, ELK1, IL16, NDNF, PTPRH, STX1B,
ZNF330, C9orf89, ELK3, IL17A, NDNL2, PTPRJ, STX2, ZNF331, C9orf9, ELK4, IL17B,
NDOR1, PTPRK, STX3, ZNF333, C9orf91, ELL, IL17C, NDP, PTPRM, STX4, ZNF334, C9orf92,
ELL2, IL17D, NDRG1, PTPRN, STX5, ZNF335, C9orf96, ELL3, IL17F, NDRG2, PTPRN2, STX6,
ZNF337, CA1, ELMO1, IL17RA, NDRG3, PTPRO, STX7, ZNF33A, CA10, ELMO2, IL17RB,
NDRG4, PTPRQ, STX8, ZNF33B, CA11, ELMO3, IL17RC, NDST1, PTPRR, STXBP1, ZNF34,
CA12, ELMOD1, IL17RD, NDST2, PTPRS, STXBP2, ZNF341, CA13, ELMOD2, IL17RE,
NDST3, PTPRT, STXBP3, ZNF343, CA14, ELMOD3, IL17REL, NDST4, PTPRU, STXBP4,
ZNF345, CA2, ELMSAN1, IL18, NDUFA1, PTPRZ1, STXBP5, ZNF346, CA3, ELN, IL18BP,
NDUFA10, PTRF, STXBP5L, ZNF347, CA4, ELOF1, IL18R1, NDUFA11, PTRH1, STXBP6,
ZNF35, CA5A, ELOVL1, IL18RAP, NDUFA12, PTRH2, STYK1, ZNF350, CA5B, ELOVL2,
IL19, NDUFA13, PTRHD1, STYX, ZNF354A, CA6, ELOVL3, IL1A, NDUFA2, PTS, STYXL1,
ZNF354B, CA7, ELOVL4, IL1B, NDUFA3, PTTG1, SUB1, ZNF354C, CA8, ELOVL5, IL1F10,
NDUFA4, PTTG1IP, SUCLA2, ZNF358, CA9, ELOVL6, IL1R1, NDUFA4L2, PTTG2, SUCLG1,
ZNF362, CAAP1, ELOVL7, IL1R2, NDUFA5, PTX3, SUCLG2, ZNF365, CAB39, ELP2, IL1RAP,
NDUFA6, PTX4, SUCNR1, ZNF366, CAB39L, ELP3, IL1RAPL1, NDUFA7, PUF60, SUCO,
ZNF367, CABIN1, ELP4, IL1RAPL2, NDUFA8, PUM1, SUDS3, ZNF37A, CABLES1, ELP5,
IL1RL1, NDUFA9, PUM2, SUFU, ZNF382, CABLES2, ELP6, IL1RL2, NDUFAB1, PURA,
SUGP1, ZNF383, CABP1, ELSPBP1, IL1RN, NDUFAF1, PURB, SUGP2, ZNF384, CABP2,
ELTD1, IL2, NDUFAF2, PURG, SUGT1, ZNF385A, CABP4, EMB, IL20, NDUFAF3, PUS1,
SULF1, ZNF385B, CABP5, EMC1, IL20RA, NDUFAF4, PUS10, SULF2, ZNF385C, CABP7,
EMC10, IL20RB, NDUFAF5, PUS3, SULT1A1, ZNF385D, CABS1, EMC2, IL21, NDUFAF6,
PUS7, SULT1A2, ZNF391, CABYR, EMC3, IL21R, NDUFAF7, PUS7L, SULT1A3, ZNF394,
CACFD1, EMC4, IL22, NDUFB1, PUSL1, SULT1A4, ZNF395, CACHD1, EMC6, IL22RA1,
NDUFB10, PVALB, SULT1B1, ZNF396, CACNA1A, EMC7, IL22RA2, NDUFB11, PVR,
SULT1C2, ZNF397, CACNA1B, EMC8, IL23A, NDUFB2, PVRIG, SULT1C3, ZNF398,
CACNA1C, EMC9, IL23R, NDUFB3, PVRL1, SULT1C4, ZNF404, CACNA1D, EMCN, IL24,
NDUFB4, PVRL2, SULT1E1, ZNF407, CACNA1E, EMD, IL25, NDUFB5, PVRL3, SULT2A1,
ZNF408, CACNA1F, EME1, IL26, NDUFB6, PVRL4, SULT2B1, ZNF41, CACNA1G, EME2,
IL27, NDUFB7, PWP1, SULT4A1, ZNF410, CACNA1H, EMG1, IL27RA, NDUFB8, PWP2,
SULT6B1, ZNF414, CACNA1I, EMID1, IL2RA, NDUFB9, PWWP2A, SUMF1, ZNF415,
CACNA1S, EMILIN1, IL2RB, NDUFC1, PWWP2B, SUMF2, ZNF416, CACNA2D1, EMILIN2,
IL2RG, NDUFC2, PXDC1, SUMO1, ZNF417, CACNA2D2, EMILIN3, IL3, NDUFC2-KCTD14,
PXDN, SUMO2, ZNF418, CACNA2D3, EML1, IL31, NDUFS1, PXDNL, SUMO3, ZNF419,
CACNA2D4, EML2, IL31RA, NDUFS2, PXK, SUMO4, ZNF420, CACNB1, EML3, IL32,
NDUFS3, PXMP2, SUN1, ZNF423, CACNB2, EML4, IL33, NDUFS4, PXMP4, SUN2, ZNF425,
CACNB3, EML5, IL34, NDUFS5, PXN, SUN3, ZNF426, CACNB4, EML6, IL36A, NDUFS6,
PXT1, SUN5, ZNF428, CACNG1, EMP1, IL36B, NDUFS7, PYCARD, SUOX, ZNF429,
CACNG2, EMP2, IL36G, NDUFS8, PYCR1, SUPT16H, ZNF43, CACNG3, EMP3, IL36RN,
NDUFV1, PYCR2, SUPT20H, ZNF430, CACNG4, EMR1, IL37, NDUFV2, PYCRL, SUPT3H,
ZNF431, CACNG5, EMR2, IL3RA, NDUFV3, PYDC1, SUPT4H1, ZNF432, CACNG6, EMR3,
IL4, NEB, PYDC2, SUPT5H, ZNF433, CACNG7, EMX1, IL4I1, NEBL, PYGB, SUPT6H,
ZNF436, CACNG8, EMX2, IL4R, NECAB1, PYGL, SUPT7L, ZNF438, CACTIN, EN1, IL5,
NECAB2, PYGM, SUPV3L1, ZNF439, CACUL1, EN2, IL5RA, NECAB3, PYGO1, SURF1,
ZNF44, CACYBP, ENAH, IL6, NECAP1, PYGO2, SURF2, ZNF440, CAD, ENAM, IL6R,
NECAP2, PYHIN1, SURF4, ZNF441, CADM1, ENC1, IL6ST, NEDD1, PYROXD1, SURF6,
ZNF442, CADM2, ENDOD1, IL7, NEDD4, PYROXD2, SUSD1, ZNF443, CADM3, ENDOG,
IL7R, NEDD4L, PYURF, SUSD2, ZNF444, CADM4, ENDOU, IL8, NEDD8, PYY, SUSD3,
ZNF445, CADPS, ENDOV, IL9, NEDD8-MDP1, PZP, SUSD4, ZNF446, CADPS2, ENG, IL9R,
NEDD9, QARS, SUSD5, ZNF449, CAGE1, ENGASE, ILDR1, NEFH, QDPR, SUV39H1, ZNF45,
CALB1, ENHO, ILDR2, NEFL, QKI, SUV39H2, ZNF451, CALB2, ENKD1, ILF2, NEFM, QPCT,
SUV420H1, ZNF454, CALCA, ENKUR, ILF3, NEGR1, QPCTL, SUV420H2, ZNF460, CALCB,
ENO1, ILK, NEIL1, QPRT, SUZ12, ZNF461, CALCOCO1, ENO2, ILKAP, NEIL2, QRFP, SV2A,
ZNF462, CALCOCO2, ENO3, ILVBL, NEIL3, QRFPR, SV2B, ZNF467, CALCR, ENO4,
IMMP1L, NEK1, QRICH1, SV2C, ZNF468, CALCRL, ENOPH1, IMMP2L, NEK10, QRICH2,
SVEP1, ZNF469, CALD1, ENOSF1, IMMT, NEK11, QRSL1, SVIL, ZNF470, CALHM1, ENOX1,
IMP3, NEK2, QSER1, SVIP, ZNF471, CALHM2, ENOX2, IMP4, NEK3, QSOX1, SVOP,
ZNF473, CALHM3, ENPEP, IMPA1, NEK4, QSOX2, SVOPL, ZNF474, CALM1, ENPP1, IMPA2,
NEK5, QTRT1, SWAP70, ZNF479, CALM2, ENPP2, IMPACT, NEK6, QTRTD1, SWI5, ZNF48,
CALM3, ENPP3, IMPAD1, NEK7, R3HCC1, SWSAP1, ZNF480, CALML3, ENPP4, IMPDH1,
NEK8, R3HCC1L, SWT1, ZNF483, CALML4, ENPP5, IMPDH2, NEK9, R3HDM1, SYAP1,
ZNF484, CALML5, ENPP6, IMPG1, NELFA, R3HDM2, SYBU, ZNF485, CALML6, ENPP7,
IMPG2, NELFB, R3HDM4, SYCE1, ZNF486, CALN1, ENSA, INA, NELFCD, R3HDML,
SYCE1L, ZNF488, CALR, ENTHD1, INADL, NELFE, RAB10, SYCE2, ZNF490, CALR3,
ENTHD2, INCA1, NELL1, RAB11A, SYCE3, ZNF491, CALU, ENTPD1, INCENP, NELL2,
RAB11B, SYCN, ZNF492, CALY, ENTPD2, INF2, NEMF, RAB11FIP1, SYCP1, ZNF493,
CAMK1, ENTPD3, ING1, NENF, RAB11FIP2, SYCP2, ZNF496, CAMK1D, ENTPD4, ING2,
NEO1, RAB11FIP3, SYCP2L, ZNF497, CAMK1G, ENTPD5, ING3, NES, RAB11FIP4, SYCP3,
ZNF500, CAMK2A, ENTPD6, ING4, NET1, RAB11FIP5, SYDE1, ZNF501, CAMK2B, ENTPD7,
ING5, NETO1, RAB12, SYDE2, ZNF502, CAMK2D, ENTPD8, INHA, NETO2, RAB13, SYF2,
ZNF503, CAMK2G, ENY2, INHBA, NEU1, RAB14, SYK, ZNF506, CAMK2N1, EOGT, INHBB,
NEU2, RAB15, SYMPK, ZNF507, CAMK2N2, EOMES, INHBC, NEU3, RAB17, SYN1, ZNF510,
CAMK4, EP300, INHBE, NEU4, RAB18, SYN2, ZNF511, CAMKK1, EP400, INIP, NEURL1,
RAB19, SYN3, ZNF512, CAMKK2, EPAS1, INMT, NEURL1B, RAB1A, SYNC, ZNF512B,
CAMKMT, EPB41, INO80, NEURL2, RAB1B, SYNCRIP, ZNF513, CAMKV, EPB41L1,
INO80B, NEURL3, RAB20, SYNDIG1, ZNF514, CAMLG, EPB41L2, INO80C, NEURL4,
RAB21, SYNDIG1L, ZNF516, CAMP, EPB41L3, INO80D, NEUROD1, RAB22A, SYNE1,
ZNF517, CAMSAP1, EPB41L4A, INO80E, NEUROD2, RAB23, SYNE2, ZNF518A, CAMSAP2,
EPB41L4B, INPP1, NEUROD4, RAB24, SYNE3, ZNF518B, CAMSAP3, EPB41L5, INPP4A,
NEUROD6, RAB25, SYNE4, ZNF519, CAMTA1, EPB42, INPP4B, NEUROG1, RAB26,
SYNGAP1, ZNF521, CAMTA2, EPC1, INPP5A, NEUROG2, RAB27A, SYNGR1, ZNF524,
CAND1, EPC2, INPP5B, NEUROG3, RAB27B, SYNGR2, ZNF526, CAND2, EPCAM, INPP5D,
NEXN, RAB28, SYNGR3, ZNF527, CANT1, EPDR1, INPP5E, NF1, RAB2A, SYNGR4, ZNF528,
CANX, EPG5, INPP5F, NF2, RAB2B, SYNJ1, ZNF529, CAP1, EPGN, INPP5J, NFAM1, RAB30,
SYNJ2, ZNF530, CAP2, EPHA1, INPP5K, NFASC, RAB31, SYNJ2BP, ZNF532, CAPG, EPHA10,
INPPL1, NFAT5, RAB32, SYNJ2BP-COX16, ZNF534, CAPN1, EPHA2, INS, NFATC1,
RAB33A, SYNM, ZNF536, CAPN10, EPHA3, INSC, NFATC2, RAB33B, SYNPO, ZNF540,
CAPN11, EPHA4, INSIG1, NFATC2IP, RAB34, SYNPO2, ZNF541, CAPN12, EPHA5, INSIG2,
NFATC3, RAB35, SYNPO2L, ZNF543, CAPN13, EPHA6, INS-IGF2, NFATC4, RAB36, SYNPR,
ZNF544, CAPN14, EPHA7, INSL3, NFE2, RAB37, SYNRG, ZNF546, CAPN15, EPHA8, INSL4,
NFE2L1, RAB38, SYP, ZNF547, CAPN2, EPHB1, INSL5, NFE2L2, RAB39A, SYPL1, ZNF548,
CAPN3, EPHB2, INSL6, NFE2L3, RAB39B, SYPL2, ZNF549, CAPN5, EPHB3, INSM1, NFIA,
RAB3A, SYS1, ZNF550, CAPN6, EPHB4, INSM2, NFIB, RAB3B, SYT1, ZNF551, CAPN7,
EPHB6, INSR, NFIC, RAB3C, SYT10, ZNF552, CAPN8, EPHX1, INSRR, NFIL3, RAB3D,
SYT11, ZNF554, CAPN9, EPHX2, INTS1, NFIX, RAB3GAP1, SYT12, ZNF555, CAPNS1,
EPHX3, INTS10, NFKB1, RAB3GAP2, SYT13, ZNF556, CAPNS2, EPHX4, INTS12, NFKB2,
RAB3IL1, SYT14, ZNF557, CAPRIN1, EPM2A, INTS2, NFKBIA, RAB3IP, SYT15, ZNF558,
CAPRIN2, EPM2AIP1, INTS3, NFKBIB, RAB40A, SYT16, ZNF559, CAPS, EPN1, INTS4,
NFKBID, RAB40AL, SYT17, ZNF559-ZNF177, CAPS2, EPN2, INTS5, NFKBIE, RAB40B,
SYT2, ZNF560, CAPSL, EPN3, INTS6, NFKBIL1, RAB40C, SYT3, ZNF561, CAPZA1, EPO,
INTS7, NFKBIZ, RAB41, SYT4, ZNF562, CAPZA2, EPOR, INTS8, NFRKB, RAB42, SYT5,
ZNF563, CAPZA3, EPPIN, INTS9, NFS1, RAB43, SYT6, ZNF564, CAPZB, EPPIN-WFDC6,
INTU, NFU1, RAB44, SYT7, ZNF565, CARD10, EPPK1, INVS, NFX1, RAB4A, SYT8, ZNF566,
CARD11, EPRS, IP6K1, NFXL1, RAB4B, SYT9, ZNF567, CARD14, EPS15, IP6K2, NFYA,
RAB5A, SYTL1, ZNF568, CARD16, EPS15L1, IP6K3, NFYB, RAB5B, SYTL2, ZNF569,
CARD17, EPS8, IPCEF1, NFYC, RAB5C, SYTL3, ZNF57, CARD18, EPS8L1, IPMK, NGB,
RAB6A, SYTL4, ZNF570, CARD6, EPS8L2, IPO11, NGDN, RAB6B, SYTL5, ZNF571, CARD8,
EPS8L3, IPO13, NGEF, RAB6C, SYVN1, ZNF572, CARD9, EPSTI1, IPO4, NGF, RAB7A, SZT2,
ZNF573, CARF, EPT1, IPO5, NGFR, RAB7L1, T, ZNF574, CARHSP1, EPX, IPO7, NGFRAP1,
RAB8A, TAAR1, ZNF575, CARKD, EPYC, IPO8, NGLY1, RAB8B, TAAR2, ZNF576, CARM1,
EQTN, IPO9, NGRN, RAB9A, TAAR5, ZNF577, CARNS1, ERAL1, IPP, NHEJ1, RAB9B,
TAAR6, ZNF578, CARS, ERAP1, IPPK, NHLH1, RABAC1, TAAR8, ZNF579, CARS2, ERAP2,
IQCA1, NHLH2, RABEP1, TAAR9, ZNF580, CARTPT, ERAS, IQCB1, NHLRC1, RABEP2,
TAB1, ZNF581, CASC1, ERBB2, IQCC, NHLRC2, RABEPK, TAB2, ZNF582, CASC10,
ERBB2IP, IQCD, NHLRC3, RABGAP1, TAB3, ZNF583, CASC3, ERBB3, IQCE, NHLRC4,
RABGAP1L, TAC1, ZNF584, CASC4, ERBB4, IQCF1, NHP2, RABGEF1, TAC3, ZNF585A,
CASC5, ERC1, IQCF2, NHP2L1, RABGGTA, TAC4, ZNF585B, CASD1, ERC2, IQCF3, NHS,
RABGGTB, TACC1, ZNF586, CASK, ERCC1, IQCF5, NHSL1, RABIF, TACC2, ZNF587,
CASKIN1, ERCC2, IQCF6, NHSL2, RABL2A, TACC3, ZNF587B, CASKIN2, ERCC3, IQCG,
NICN1, RABL2B, TACO1, ZNF589, CASP1, ERCC4, IQCH, NID1, RABL3, TACR1, ZNF592,
CASP10, ERCC5, IQCJ, NID2, RABL5, TACR2, ZNF593, CASP12, ERCC6, IQCJ-SCHIP1,
NIF3L1, RABL6, TACR3, ZNF594, CASP14, ERCC6L, IQCK, NIFK, RAC1, TACSTD2, ZNF595,
CASP16, ERCC6L2, IQGAP1, NIM1, RAC2, TADA1, ZNF596, CASP2, ERCC6-PGBD3,
IQGAP2, NIN, RAC3, TADA2A, ZNF597, CASP3, ERCC8, IQGAP3, NINJ1, RACGAP1,
TADA2B, ZNF598, CASP4, EREG, IQSEC1, NINJ2, RAD1, TADA3, ZNF599, CASP5, ERF,
IQSEC2, NINL, RAD17, TAF1, ZNF600, CASP6, ERG, IQSEC3, NIP7, RAD18, TAF10, ZNF605,
CASP7, ERGIC1, IQUB, NIPA1, RAD21, TAF11, ZNF606, CASP8, ERGIC2, IRAK1, NIPA2,
RAD21L1, TAF12, ZNF607, CASP8AP2, ERGIC3, IRAK1BP1, NIPAL1, RAD23A, TAF13,
ZNF608, CASP9, ERH, IRAK2, NIPAL2, RAD23B, TAF15, ZNF609, CASQ1, ERI1, IRAK3,
NIPAL3, RAD50, TAF1A, ZNF610, CASQ2, ERI2, IRAK4, NIPAL4, RAD51, TAF1B, ZNF611,
CASR, ERI3, IREB2, NIPBL, RAD51AP1, TAF1C, ZNF613, CASS4, ERICH1, IRF1, NIPSNAP1,
RAD51AP2, TAF1D, ZNF614, CAST, ERICH2, IRF2, NIPSNAP3A, RAD51B, TAF1L, ZNF615,
CASZ1, ERLEC1, IRF2BP1, NIPSNAP3B, RAD51C, TAF2, ZNF616, CAT, ERLIN1, IRF2BP2,
NISCH, RAD51D, TAF3, ZNF618, CATSPER1, ERLIN2, IRF2BPL, NIT1, RAD52, TAF4,
ZNF619, CATSPER2, ERMAP, IRF3, NIT2, RAD54B, TAF4B, ZNF620, CATSPER3, ERMARD,
IRF4, NKAIN1, RAD54L, TAF5, ZNF621, CATSPER4, ERMN, IRF5, NKAIN2, RAD54L2,
TAF5L, ZNF622, CATSPERB, ERMP1, IRF6, NKAIN3, RAD9A, TAF6, ZNF623, CATSPERD,
ERN1, IRF7, NKAIN4, RAD9B, TAF6L, ZNF624, CATSPERG, ERN2, IRF8, NKAP, RADIL,
TAF7, ZNF625, CAV1, ERO1L, IRF9, NKAPL, RAE1, TAF7L, ZNF626, CAV2, ERO1LB, IRG1,
NKD1, RAET1E, TAF8, ZNF627, CAV3, ERP27, IRGC, NKD2, RAET1G, TAF9, ZNF628,
CBFA2T2, ERP29, IRGM, NKG7, RAET1L, TAF9B, ZNF629, CBFA2T3, ERP44, IRGQ,
NKIRAS1, RAF1, TAGAP, ZNF630, CBFB, ERRFI1, IRS1, NKIRAS2, RAG1, TAGLN, ZNF638,
CBL, ERVFRD-1, IRS2, NKPD1, RAG2, TAGLN2, ZNF639, CBLB, ERVMER34-1, IRS4, NKRF,
RAI1, TAGLN3, ZNF641, CBLC, ERVV-1, IRX1, NKTR, RAI14, TAL1, ZNF644, CBLL1,
ERVV-2, IRX2, NKX1-2, RAI2, TAL2, ZNF645, CBLN1, ESAM, IRX3, NKX2-1, RALA,
TALDO1, ZNF646, CBLN2, ESCO1, IRX4, NKX2-2, RALB, TAMM41, ZNF648, CBLN3,
ESCO2, IRX5, NKX2-3, RALBP1, TANC1, ZNF649, CBLN4, ESD, IRX6, NKX2-4, RALGAPA1,
TANC2, ZNF652, CBR1, ESF1, ISCA1, NKX2-5, RALGAPA2, TANGO2, ZNF653, CBR3, ESM1,
ISCA2, NKX2-6, RALGAPB, TANGO6, ZNF654, CBR4, ESPL1, ISCU, NKX2-8, RALGDS,
TANK, ZNF655, CBS, ESPN, ISG15, NKX3- 1, RALGPS1, TAOK1, ZNF658, CBWD1, ESPNL,
ISG20, NKX3-2, RALGPS2, TAOK2, ZNF660, CBWD2, ESR1, ISG20L2, NKX6-1, RALY,
TAOK3, ZNF662, CBWD3, ESR2, ISL1, NKX6-2, RALYL, TAP1, ZNF664, CBWD5, ESRP1,
ISL2, NKX6-3, RAMP1, TAP2, ZNF664-FAM101A, CBWD6, ESRP2, ISLR, NLE1, RAMP2,
TAPBP, ZNF665, CBWD7, ESRRA, ISLR2, NLGN1, RAMP3, TAPBPL, ZNF667, CBX1,
ESRRB, ISM1, NLGN2, RAN, TAPT1, ZNF668, CBX2, ESRRG, ISM2, NLGN3, RANBP1,
TARBP1, ZNF669, CBX3, ESX1, ISOC1, NLGN4X, RANBP10, TARBP2, ZNF670, CBX4,
ESYT1, ISOC2, NLGN4Y, RANBP17, TARDBP, ZNF671, CBX5, ESYT2, ISPD, NLK, RANBP2,
TARM1, ZNF672, CBX6, ESYT3, IST1, NLN, RANBP3, TARP, ZNF674, CBX7, ETAA1, ISX,
NLRC3, RANBP3L, TARS, ZNF675, CBX8, ETF1, ISY1, NLRC4, RANBP6, TARS2, ZNF676,
CBY1, ETFA, ISY1-RAB43, NLRC5, RANBP9, TARSL2, ZNF677, CBY3, ETFB, ISYNA1,
NLRP1, RANGAP1, TAS1R1, ZNF678, CC2D1A, ETFDH, ITCH, NLRP10, RANGRF, TAS1R2,
ZNF679, CC2D1B, ETHE1, ITFG1, NLRP11, RAP1A, TAS1R3, ZNF680, CC2D2A, ETNK1,
ITFG2, NLRP12, RAP1B, TAS2R1, ZNF681, CC2D2B, ETNK2, ITFG3, NLRP13, RAP1GAP,
TAS2R10, ZNF682, CCAR1, ETNPPL, ITGA1, NLRP14, RAP1GAP2, TAS2R13, ZNF683,
CCAR2, ETS1, ITGA10, NLRP2, RAP1GDS1, TAS2R14, ZNF684, CCBE1, ETS2, ITGA11,
NLRP3, RAP2A, TAS2R16, ZNF687, CCBL1, ETV1, ITGA2, NLRP4, RAP2B, TAS2R19,
ZNF688, CCBL2, ETV2, ITGA2B, NLRP5, RAP2C, TAS2R20, ZNF689, CCDC101, ETV3,
ITGA3, NLRP6, RAPGEF1, TAS2R3, ZNF69, CCDC102A, ETV3L, ITGA4, NLRP7, RAPGEF2,
TAS2R30, ZNF691, CCDC102B, ETV4, ITGA5, NLRP8, RAPGEF3, TAS2R31, ZNF692,
CCDC103, ETV5, ITGA6, NLRP9, RAPGEF4, TAS2R38, ZNF695, CCDC104, ETV6, ITGA7,
NLRX1, RAPGEF5, TAS2R39, ZNF696, CCDC105, ETV7, ITGA8, NMB, RAPGEF6, TAS2R4,
ZNF697, CCDC106, EVA1A, ITGA9, NMBR, RAPGEFL1, TAS2R40, ZNF699, CCDC107,
EVA1B, ITGAD, NMD3, RAPH1, TAS2R41, ZNF7, CCDC108, EVA1C, ITGAE, NME1, RAPSN,
TAS2R42, ZNF70, CCDC109B, EVC, ITGAL, NME1-NME2, RARA, TAS2R43, ZNF700,
CCDC11, EVC2, ITGAM, NME2, RARB, TAS2R46, ZNF701, CCDC110, EVI2A, ITGAV,
NME3, RARG, TAS2R5, ZNF703, CCDC112, EVI2B, ITGAX, NME4, RARRES1, TAS2R50,
ZNF704, CCDC113, EVI5, ITGB1, NME5, RARRES2, TAS2R60, ZNF705A, CCDC114, EVI5L,
ITGB1BP1, NME6, RARRES3, TAS2R7, ZNF705B, CCDC115, EVL, ITGB1BP2, NME7, RARS,
TAS2R8, ZNF705D, CCDC116, EVPL, ITGB2, NME8, RARS2, TAS2R9, ZNF705E, CCDC117,
EVPLL, ITGB3, NME9, RASA1, TASP1, ZNF705G, CCDC12, EVX1, ITGB3BP, NMI, RASA2,
TAT, ZNF706, CCDC120, EVX2, ITGB4, NMNAT1, RASA3, TATDN1, ZNF707, CCDC121,
EWSR1, ITGB5, NMNAT2, RASA4, TATDN2, ZNF708, CCDC122, EXD1, ITGB6, NMNAT3,
RASA4B, TATDN3, ZNF709, CCDC124, EXD2, ITGB7, NMRAL1, RASAL1, TAX1BP1, ZNF71,
CCDC125, EXD3, ITGB8, NMRK1, RASAL2, TAX1BP3, ZNF710, CCDC126, EXO1, ITGBL1,
NMRK2, RASAL3, TAZ, ZNF711, CCDC127, EXO5, ITIH1, NMS, RASD1, TBATA, ZNF713,
CCDC129, EXOC1, ITIH2, NMT1, RASD2, TBC1D1, ZNF714, CCDC13, EXOC2, ITIH3, NMT2,
RASEF, TBC1D10A, ZNF716, CCDC130, EXOC3, ITIH4, NMU, RASGEF1A, TBC1D10B,
ZNF717, CCDC132, EXOC3L1, ITIH5, NMUR1, RASGEF1B, TBC1D10C, ZNF718, CCDC134,
EXOC3L2, ITIH6, NMUR2, RASGEF1C, TBC1D12, ZNF720, CCDC135, EXOC3L4, ITK,
NNAT, RASGRF1, TBC1D13, ZNF721, CCDC136, EXOC4, ITLN1, NNMT, RASGRF2,
TBC1D14, ZNF726, CCDC137, EXOC5, ITLN2, NNT, RASGRP1, TBC1D15, ZNF727,
CCDC138, EXOC6, ITM2A, NOA1, RASGRP2, TBC1D16, ZNF728, CCDC14, EXOC6B, ITM2B,
NOB1, RASGRP3, TBC1D17, ZNF729, CCDC140, EXOC7, ITM2C, NOBOX, RASGRP4,
TBC1D19, ZNF730, CCDC141, EXOC8, ITPA, NOC2L, RASIP1, TBC1D2, ZNF732, CCDC142,
EXOG, ITPK1, NOC3L, RASL10A, TBC1D20, ZNF735, CCDC144A, EXOSC1, ITPKA, NOC4L,
RASL10B, TBC1D21, ZNF736, CCDC144NL, EXOSC10, ITPKB, NOD1, RASL11A,
TBC1D22A, ZNF737, CCDC146, EXOSC2, ITPKC, NOD2, RASL11B, TBC1D22B, ZNF74,
CCDC147, EXOSC3, ITPR1, NODAL, RASL12, TBC1D23, ZNF740, CCDC148, EXOSC4,
ITPR2, NOG, RASSF1, TBC1D24, ZNF746, CCDC149, EXOSC5, ITPR3, NOL10, RASSF10,
TBC1D25, ZNF747, CCDC15, EXOSC6, ITPRIP, NOL11, RASSF2, TBC1D26, ZNF749,
CCDC150, EXOSC7, ITPRIPL1, NOL12, RASSF3, TBC1D27, ZNF750, CCDC151, EXOSC8,
ITPRIPL2, NOL3, RASSF4, TBC1D28, ZNF75A, CCDC152, EXOSC9, ITSN1, NOL4, RASSF5,
TBC1D29, ZNF75D, CCDC153, EXPH5, ITSN2, NOL6, RASSF6, TBC1D2B, ZNF76, CCDC154,
EXT1, IVD, NOL7, RASSF7, TBC1D3, ZNF761, CCDC155, EXT2, IVL, NOL8, RASSF8,
TBC1D30, ZNF763, CCDC157, EXTL1, IVNS1ABP, NOL9, RASSF9, TBC1D31, ZNF764,
CCDC158, EXTL2, IWS1, NOLC1, RAVER1, TBC1D32, ZNF765, CCDC159, EXTL3, IYD,
NOM1, RAVER2, TBC1D3B, ZNF766, CCDC160, EYA1, IZUMO1, NOMO1, RAX, TBC1D3C,
ZNF768, CCDC166, EYA2, IZUMO2, NOMO2, RAX2, TBC1D3F, ZNF77, CCDC167, EYA3,
IZUMO3, NOMO3, RB1, TBC1D3G, ZNF770, CCDC168, EYA4, IZUMO4, NONO, RB1CC1,
TBC1D3H, ZNF771, CCDC169, EYS, JADE1, NOP10, RBAK, TBC1D4, ZNF772, CCDC169-
SOHLH2, EZH1, JADE2, NOP14, RBAK-RBAKDN, TBC1D5, ZNF773, CCDC17, EZH2, JADE3,
NOP16, RBBP4, TBC1D7, ZNF774, CCDC170, EZR, JAG1, NOP2, RBBP5, TBC1D8, ZNF775,
CCDC171, F10, JAG2, NOP56, RBBP6, TBC1D8B, ZNF776, CCDC172, F11, JAGN1, NOP58,
RBBP7, TBC1D9, ZNF777, CCDC173, F11R, JAK1, NOP9, RBBP8, TBC1D9B, ZNF778,
CCDC174, F12, JAK2, NOS1, RBBP8NL, TBCA, ZNF780A, CCDC175, F13A1, JAK3, NOS1AP,
RBBP9, TBCB, ZNF780B, CCDC176, F13B, JAKMIP1, NOS2, RBCK1, TBCC, ZNF781,
CCDC177, F2, JAKMIP2, NOS3, RBFA, TBCCD1, ZNF782, CCDC178, F2R, JAKMIP3, NOSIP,
RBFOX1, TBCD, ZNF783, CCDC179, F2RL1, JAM2, NOSTRIN, RBFOX2, TBCE, ZNF784,
CCDC18, F2RL2, JAM3, NOTCH1, RBFOX3, TBCEL, ZNF785, CCDC180, F2RL3, JARID2,
NOTCH2, RBKS, TBCK, ZNF786, CCDC181, F3, JAZF1, NOTCH2NL, RBL1, TBK1, ZNF787,
CCDC19, F5, JDP2, NOTCH3, RBL2, TBKBP1, ZNF789, CCDC22, F7, JHDM1D, NOTCH4,
RBM10, TBL1X, ZNF79, CCDC23, F8, JKAMP, NOTO, RBM11, TBL1XR1, ZNF790, CCDC24,
F8A1, JMJD1C, NOTUM, RBM12, TBL1Y, ZNF791, CCDC25, F8A2, JMJD4, NOV, RBM12B,
TBL2, ZNF792, CCDC27, F8A3, JMJD6, NOVA1, RBM14, TBL3, ZNF793, CCDC28A, F9,
JMJD7, NOVA2, RBM14-RBM4, TBP, ZNF799, CCDC28B, FA2H, JMJD7-PLA2G4B, NOX1,
RBM15, TBPL1, ZNF8, CCDC3, FAAH, JMJD8, NOX3, RBM15B, TBPL2, ZNF80, CCDC30,
FAAH2, JMY, NOX4, RBM17, TBR1, ZNF800, CCDC33, FABP1, JOSD1, NOX5, RBM18,
TBRG1, ZNF804A, CCDC34, FABP12, JOSD2, NOXA1, RBM19, TBRG4, ZNF804B, CCDC36,
FABP2, JPH1, NOXO1, RBM20, TBX1, ZNF805, CCDC37, FABP3, JPH2, NOXRED1, RBM22,
TBX10, ZNF806, CCDC38, FABP4, JPH3, NPAP1, RBM23, TBX15, ZNF808, CCDC39, FABP5,
JPH4, NPAS1, RBM24, TBX18, ZNF81, CCDC40, FABP6, JRK, NPAS2, RBM25, TBX19,
ZNF812, CCDC41, FABP7, JRKL, NPAS3, RBM26, TBX2, ZNF813, CCDC42, FABP9, JSRP1,
NPAS4, RBM27, TBX20, ZNF814, CCDC42B, FADD, JTB, NPAT, RBM28, TBX21, ZNF816,
CCDC43, FADS1, JUN, NPB, RBM3, TBX22, ZNF816-ZNF321P, CCDC47, FADS2, JUNB,
NPBWR1, RBM33, TBX3, ZNF821, CCDC50, FADS3, JUND, NPBWR2, RBM34, TBX4,
ZNF823, CCDC51, FADS6, JUP, NPC1, RBM38, TBX5, ZNF827, CCDC53, FAF1, KAAG1,
NPC1L1, RBM39, TBX6, ZNF829, CCDC54, FAF2, KAL1, NPC2, RBM4, TBXA2R, ZNF83,
CCDC57, FAH, KALRN, NPDC1, RBM41, TBXAS1, ZNF830, CCDC58, FAHD1, KANK1,
NPEPL1, RBM42, TC2N, ZNF831, CCDC59, FAHD2A, KANK2, NPEPPS, RBM43, TCAIM,
ZNF835, CCDC6, FAHD2B, KANK3, NPFF, RBM46, TCAP, ZNF836, CCDC60, FAIM, KANK4,
NPFFR1, RBM47, TCEA1, ZNF837, CCDC61, FAIM2, KANSL1, NPFFR2, RBM48, TCEA2,
ZNF839, CCDC62, FAIM3, KANSL1L, NPHP1, RBM4B, TCEA3, ZNF84, CCDC63, FAM101A,
KANSL2, NPHP3, RBM5, TCEAL1, ZNF841, CCDC64, FAM101B, KANSL3, NPHP4, RBM6,
TCEAL2, ZNF843, CCDC64B, FAM102A, KARS, NPHS1, RBM7, TCEAL3, ZNF844, CCDC65,
FAM102B, KAT2A, NPHS2, RBM8A, TCEAL4, ZNF845, CCDC66, FAM103A1, KAT2B,
NPIPA1, RBMS1, TCEAL5, ZNF846, CCDC67, FAM104A, KAT5, NPIPA2, RBMS2, TCEAL6,
ZNF85, CCDC68, FAM104B, KAT6A, NPIPA3, RBMS3, TCEAL7, ZNF850, CCDC69,
FAM105A, KAT6B, NPIPA5, RBMX, TCEAL8, ZNF852, CCDC7, FAM105B, KAT7, NPIPA7,
RBMX2, TCEANC, ZNF853, CCDC70, FAM107A, KAT8, NPIPA8, RBMXL1, TCEANC2,
ZNF860, CCDC71, FAM107B, KATNA1, NPIPB11, RBMXL2, TCEB1, ZNF862, CCDC71L,
FAM109A, KATNAL1, NPIPB15, RBMXL3, TCEB2, ZNF865, CCDC73, FAM109B, KATNAL2,
NPIPB3, RBMY1A1, TCEB3, ZNF878, CCDC74A, FAM110A, KATNB1, NPIPB4, RBMY1B,
TCEB3B, ZNF879, CCDC74B, FAM110B, KATNBL1, NPIPB5, RBMY1D, TCEB3C, ZNF880,
CCDC77, FAM110C, KAZALD1, NPIPB6, RBMY1E, TCEB3CL, ZNF883, CCDC78, FAM110D,
KAZN, NPIPB8, RBMY1F, TCEB3CL2, ZNF888, CCDC79, FAM111A, KBTBD11, NPIPB9,
RBMY1J, TCERG1, ZNF891, CCDC8, FAM111B, KBTBD12, NPL, RBP1, TCERG1L, ZNF90,
CCDC80, FAM114A1, KBTBD13, NPLOC4, RBP2, TCF12, ZNF91, CCDC81, FAM114A2,
KBTBD2, NPM1, RBP3, TCF15, ZNF92, CCDC82, FAM115A, KBTBD3, NPM2, RBP4, TCF19,
ZNF93, CCDC83, FAM115C, KBTBD4, NPM3, RBP5, TCF20, ZNF98, CCDC84, FAM117A,
KBTBD6, NPNT, RBP7, TCF21, ZNF99, CCDC85A, FAM117B, KBTBD7, NPPA, RBPJ, TCF23,
ZNFX1, CCDC85B, FAM118A, KBTBD8, NPPB, RBPJL, TCF24, ZNHIT1, CCDC85C,
FAM118B, KCMF1, NPPC, RBPMS, TCF25, ZNHIT2, CCDC86, FAM120A, KCNA1, NPR1,
RBPMS2, TCF3, ZNHIT3, CCDC87, FAM120AOS, KCNA10, NPR2, RBX1, TCF4, ZNHIT6,
CCDC88A, FAM120B, KCNA2, NPR3, RC3H1, TCF7, ZNRD1, CCDC88B, FAM120C, KCNA3,
NPRL2, RC3H2, TCF7L1, ZNRF1, CCDC88C, FAM122A, KCNA4, NPRL3, RCAN1, TCF7L2,
ZNRF2, CCDC89, FAM122B, KCNA5, NPS, RCAN2, TCFL5, ZNRF3, CCDC9, FAM122C,
KCNA6, NPSR1, RCAN3, TCHH, ZNRF4, CCDC90B, FAM124A, KCNA7, NPTN, RCBTB1,
TCHHL1, ZP1, CCDC91, FAM124B, KCNAB1, NPTX1, RCBTB2, TCHP, ZP2, CCDC92,
FAM126A, KCNAB2, NPTX2, RCC1, TCIRG1, ZP3, CCDC93, FAM126B, KCNAB3, NPTXR,
RCC2, TCL1A, ZP4, CCDC94, FAM127A, KCNB1, NPVF, RCCD1, TCL1B, ZPBP, CCDC96,
FAM127B, KCNB2, NPW, RCE1, TCN1, ZPBP2, CCDC97, FAM127C, KCNC1, NPY, RCHY1,
TCN2, ZPLD1, CCER1, FAM129A, KCNC2, NPY1R, RCL1, TCOF1, ZRANB1, CCHCR1,
FAM129B, KCNC3, NPY2R, RCN1, TCP1, ZRANB2, CCIN, FAM129C, KCNC4, NPY4R, RCN2,
TCP10, ZRANB3, CCK, FAM131A, KCND1, NPY5R, RCN3, TCP10L, ZRSR2, CCKAR,
FAM131B, KCND2, NQO1, RCOR1, TCP10L2, ZSCAN1, CCKBR, FAM131C, KCND3, NQO2,
RCOR2, TCP11, ZSCAN10, CCL1, FAM132A, KCNE1, NR0B1, RCOR3, TCP11L1, ZSCAN12,
CCL11, FAM132B, KCNE1L, NR0B2, RCSD1, TCP11L2, ZSCAN16, CCL13, FAM133A,
KCNE2, NR1D1, RCVRN, TCP11X1, ZSCAN18, CCL14, FAM133B, KCNE3, NR1D2, RD3,
TCP11X2, ZSCAN2, CCL15, FAM134A, KCNE4, NR1H2, RD3L, TCTA, ZSCAN20, CCL16,
FAM134B, KCNF1, NR1H3, RDH10, TCTE1, ZSCAN21, CCL17, FAM134C, KCNG1, NR1H4,
RDH11, TCTE3, ZSCAN22, CCL18, FAM135A, KCNG2, NR1I2, RDH12, TCTEX1D1,
ZSCAN23, CCL19, FAM135B, KCNG3, NR1I3, RDH13, TCTEX1D2, ZSCAN25, CCL2,
FAM136A, KCNG4, NR2C1, RDH14, TCTEX1D4, ZSCAN26, CCL20, FAM13A, KCNH1,
NR2C2, RDH16, TCTN1, ZSCAN29, CCL21, FAM13B, KCNH2, NR2C2AP, RDH5, TCTN2,
ZSCAN30, CCL22, FAM13C, KCNH3, NR2E1, RDH8, TCTN3, ZSCAN31, CCL23, FAM149A,
KCNH4, NR2E3, RDM1, TDG, ZSCAN32, CCL24, FAM149B1, KCNH5, NR2F1, RDX, TDGF1,
ZSCAN4, CCL25, FAM150A, KCNH6, NR2F2, REC8, TDO2, ZSCAN5A, CCL26, FAM150B,
KCNH7, NR2F6, RECK, TDP1, ZSCAN5B, CCL27, FAM151A, KCNH8, NR3C1, RECQL, TDP2,
ZSCAN9, CCL28, FAM151B, KCNIP1, NR3C2, RECQL4, TDRD1, ZSWIM1, CCL3, FAM153A,
KCNIP2, NR4A1, RECQL5, TDRD10, ZSWIM2, CCL3L1, FAM153B, KCNIP3, NR4A2, REEP1,
TDRD12, ZSWIM3, CCL3L3, FAM154A, KCNIP4, NR4A3, REEP2, TDRD3, ZSWIM4, CCL4,
FAM154B, KCNJ1, NR5A1, REEP3, TDRD5, ZSWIM5, CCL4L1, FAM155A, KCNJ10, NR5A2,
REEP4, TDRD6, ZSWIM6, CCL4L2, FAM155B, KCNJ11, NR6A1, REEP5, TDRD7, ZSWIM7,
CCL5, FAM156A, KCNJ12, NRAP, REEP6, TDRD9, ZSWIM8, CCL7, FAM156B, KCNJ13,
NRARP, REG1A, TDRKH, ZUFSP, CCL8, FAM157A, KCNJ14, NRAS, REG1B, TDRP, ZW10,
CCM2, FAM157B, KCNJ15, NRBF2, REG3A, TEAD1, ZWILCH, CCM2L, FAM159A, KCNJ16,
NRBP1, REG3G, TEAD2, ZWINT, CCNA1, FAM159B, KCNJ18, NRBP2, REG4, TEAD3,
ZXDA, CCNA2, FAM160A1, KCNJ2, NRCAM, REL, TEAD4, ZXDB, CCNB1, FAM160A2,
KCNJ3, NRD1, RELA, TEC, ZXDC, CCNB1IP1, FAM160B1, KCNJ4, NRDE2, RELB, TECPR1,
ZYG11A, CCNB2, FAM160B2, KCNJ5, NREP, RELL1, TECPR2, ZYG11B, CCNB3, FAM161A,
KCNJ6, NRF1, RELL2, TECR, ZYX, CCNC, FAM161B, KCNJ8, NRG1, RELN, TECRL, ZZEF1,
CCND1, FAM162A, KCNJ9, NRG2, RELT, TECTA, ZZZ3, CCND2, FAM162B, KCNK1, NRG,
RHODOPSIN, RdCVF, RdCVFL, GIRK, DUX4, and DBET (or DBET lncRNA).

TABLE 5
Non-limiting examples of a Cas protein or a derivative thereof.

| SEQ | |
|---|---|
| ID NO | Amino acid sequence of Cas protein or a derivative thereof |
| 51 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSDVCYTRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKIGEK |
| | SAWMLNLSIDVPKIDKGVDPSIIGGIDVGVKSPLVCAINNAFSRYSISDNDLFHFNKKMFA |
| | RRRILLKKNRHKRAGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENADYNAALNISNPKLKSTKEEP |
| 52 | MEVQKTVMKTLSLRILRPLYSQEIEKEIKEEKERRKQAGGTGELDGGFYKKLEKKHSEM |
| | FSFDRLNLLLNQLQREIAKVYNHAISELYIATIAQGNKSNKHYISSIVYNRAYGYFYNAYI |
| | ALGICSKVEANFRSNELLTQQSALPTAKSDNFPIVLHKQKGAEGEDGGFRISTEGSDLIFEI |
| | PIPFYEYNGENRKEPYKWVKKGGQKPVLKLILSTFRRQRNKGWAKDEGTDAEIRKVTE |
| | GKYQVSQIEINRGKKLGEHQKWFANFSIEQPIYERKPNRSIVGGLDVGIRSPLVCAINNSF |
| | SRYSVDSNDVFKFSKQVFAFRRRLLSKNSLKRKGHGAAHKLEPITEMTEKNDKFRKKIIE |
| | RWAKEVTNFFVKNQVGIVQIEDLSTMKDREDHFFNQYLRGFWPYYQMQTLIENKLKEY |
| | GIEVKRVQAKYTSQLCSNPNCRYWNNYFNFEYRKVNKFPKFKCEKCNLEISADYNAAR |
| | NLSTPDIEKFVAKATKGINLPEK |
| 53 | MIKVYRYEIVKPLDLDWKEFGTILRQLQQETRFALNKATQLAWEWMGFSSDYKDNHGE |
| | YPKSKDILGYTNVHGYAYHTIKTKAYRLNSGNLSQTIKRATDRFKAYQKEILRGDMSIPS |
| | YKRDIPLDLIKENISVNRMNHGDYIASLSLLSNPAKQEMNVKRKISVIIIVRGAGKTIMDRI |
| | LSGEYQVSASQIIHDDRKNKWYLNISYDFEPQTRVLDLNKIMGIDLGVAVAVYMAFQHT |
| | PARYKLEGGEIENFRRQVESRRISMLRQGKYAGGARGGHGRDKRIKPIEQLRDKIANFRD |
| | TTNHRYSRYIVDMAIKEGCGTIQMEDLTNIRDIGSRFLQNWTYYDLQQKIIYKAEEAGIK |
| | VIKIDPQYTSQRCSECGNIDSGNRIGQAIFKCRACGYEANADYNAARNIAIPNIDKIIAESI |
| | K |
| 54 | MEVEKIVADEKNNREKIALEKNKDKVKEACSKHLKVAAYCTTQVERNACLFCKARKLD |
| | DKFYQKLRGQFPDAVFWQEISEIFRQLQKQAAEIYNQSLIELYYEIFIKGKGIANASSVEH |
| | YLSRVCYRRAAELFKNAAIASGLRSKIKSNFRLKELKNMKSGLPTTKSDNFPIPLVKQKG |
| | GQYTGFEISNHNSDFIIKIPFGRWQVKKEIDKYRPWEKFDFEQVQKSPKPISLLLSTQRRK |
| | RNKGWSKDEGTEAEIKKVMNGDYQTSYIEVKRGSKICEKSAWMLNLSIDVPKIDKGVD |
| | PSIIGGIAVGVRSPLVCAINNAFSRYSISDNDLFHFNKKMFARRRILLKKNRHKRAGHGA |
| | KNKLKPITILTEKSERFRKKLIERWACEIADFFIKNKVGTVQMENLESMKRKEDSYFNIRL |
| | RGFWPYAEMQNKIEFKLKQYGIEIRKVAPNNTSKTCSKCGHLNNYFNFEYRKKNKFPHF |
| | KCEKCNFKENAAYNAALNISNPKLKSTKERP |
| 55 | MNNREKIALEKNKDKVKEACSKHLKVAAYCTTQVERNACLFCKARKLDDKFYQKLRG |
| | QFPDAVFWQEISEIFRQLQKQAAEIYNQSLIELYYEIFIKGKGIANASSVEHYLSRVCYRR |
| | AAELFKNAAIASGLRSKIKSNFRLKELKNMKSGLPTTKSDNFPIPLVKQKGGQYTGFEISN |
| | HNSDFIIKIPFGRWQVKKEIDKYRPWEKFDFEQVQKSPKPISLLLSTQRRKRNKGWSKDE |
| | GTEAEIKKVMNGDYQTSYIEVKRGSKICEKSAWMLNLSIDVPKIDKGVDPSIIGGIAVGV |
| | RSPLVCAINNAFSRYSISDNDLFHFNKKMFARRRILLKKNRHKRAGHGAKNKLKPITILT |
| | EKSERFRKKLIERWACEIADFFIKNKVGTVQMENLESMKRKEDSYFNIRLRGFWPYAEM |
| | QNKIEFKLKQYGIEIRKVAPNNTSKTCSKCGHLNNYFNFEYRKKNKFPHFKCEKCNFKE |
| | NAAYNAALNISNPKLKSTKERP |
| 56 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENA |
| 57 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFK |
| 58 | MAKNTITKTLKLRIVRPYNSAEVEKIVADEKNNREKIALEKNKDKVNACLFCKARKLDD |
| | KFYQKLRGQFPDAVFWQEISEIFRQLQKQAAEIYNQSLIELYYEIFIKGKGIANASSVEHY |
| | LSRVCYRRAAELFKNAAIASGLRSKIKSNFRLKELKNMKSGLPTTKSDNFPIPLVKQKGG |
| | QYTGFEISNHNSDFIIKIPFGRWQVKKEIDKYRPWEKFDFEQVQKSPKPISLLLSTQRRKR |
| | NKGWSKDEGTEAEIKKVMNGDYQTSYIEVKRGSKICEKSAWMLNLSIDVPKIDKGVDPS |
| | IIGGIAVGVRSPLVCAINNAFSRYSISDNDLFHFNKKMFARRRILLKKNRHKRAGHGAKN |
| | KLKPITILTEKSERFRKKLIERWACEIADFFIKNKVGTVQMENLESMKRKEDSYFNIRLRG |
| | FWPYAEMQNKIEFKLKQYGIEIRKVAPNNTSKTCSKCGHLNNYFNFEYRKKNKFPHFKC |
| | EKCNFKENAAYNAALNISNPKLKSTKERP |
| 59 | MAKNTITKTLKLRIVRPYNSAEVEKIVADEKNNREKIALECKARKLDDKFYQKLRGQFP |
| | DAVFWQEISEIFRQLQKQAAEIYNQSLIELYYEIFIKGKGIANASSVEHYLSRVCYRRAAE |
| | LFKNAAIASGLRSKIKSNFRLKELKNMKSGLPTTKSDNFPIPLVKQKGGQYTGFEISNHNS |
| | DFIIKIPFGRWQVKKEIDKYRPWEKFDFEQVQKSPKPISLLLSTQRRKRNKGWSKDEGTE |
| | AEIKKVMNGDYQTSYIEVKRGSKICEKSAWMLNLSIDVPKIDKGVDPSIIGGIAVGVRSPL |
| | VCAINNAFSRYSISDNDLFHFNKKMFARRRILLKKNRHKRAGHGAKNKLKPITILTEKSE |
| | RFRKKLIERWACEIADFFIKNKVGTVQMENLESMKRKEDSYFNIRLRGFWPYAEMQNKI |
| | EFKLKQYGIEIRKVAPNNTSKTCSKCGHLNNYFNFEYRKKNKFPHFKCEKCNFKENAAY |
| | NAALNISNPKLKSTKERP |
| 60 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 61 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 62 | MNMSKTTISVKLKIIDLSSEKKEFLDNYFNEYAKATTFCQLRIRRLLRNTHWLGKKEKSS |
| | KKWIFESGICDLCGENKELVNEDRNSGEPAKICKRCYNGRYGNQMIRKLFVSTKKREVQ |
| | ENMDIRRVAKLNNTHYHRIPEEAFDMIKAADTAEKRRKKNVEYDKKRQMEFIEMFNDE |
| | KKRAARPKKPNERETRYVHISKLESPSKGYTLNGIKRKIDGMGKKIERAEKGLSRKKIFG |
| | YQGNRIKLDSNWVRFDLAESEITIPSLFKEMKLRITGPTNVHSKSGQIYFAEWFERINKQP |
| | NNYCYLIRKTSSNGKYEYYLQYTYEAEVEANKEYAGCLGVDIGCSKLAAAVYYDSKNK |
| | KAQKPIEIFTNPIKKIKMRREKLIKLLSRVKVRHRRRKLMQLSKTEPIIDYTCHKTARKIVE |
| | MANTAKAFISMENLETGIKQKQQARETKKQKFYRNMFLFRKLSKLIEYKALLKGIKIVY |
| | VKPDYTSQTCSSCGADKEKTERPSQAIFRCLNPTCRYYQRDINADFNAAVNIAKKALNN |
| | TEVVTTLL |
| 63 | MPSETYITKTLSLKLIPSDEEKQALENYFITFQRAVNFAIDRIVDIRSSFRYLNKNEQFPAV |
| | CDCCGKKEKIMYVNISNKTFKFKPSRNQKDRYTKDIYTIKPNAHICKTCYSGVAGNMFIR |
| | KQMYPNDKEGWKVSRSYNIKVNAPGLTGTEYAMAIRKAISILRSFEKRRRNAERRIIEYE |
| | KSKKEYLELIDDVEKGKTNKIVVLEKEGHQRVKRYKHKNWPEKWQGISLNKAKSKVK |
| | DIEKRIKKLKEWKHPTLNRPYVELHKNNVRIVGYETVELKLGNKMYTIHFASISNLRKPF |
| | RKQKKKSIEYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMAKKLRGDKT |
| | KKIRLYHEIRKKFRHKVKYFRRNYLHNISKQIVEIAKENTPTVIVLEDLRYLRERTYRGK |
| | GRSKKAKKTNYKLNTFTYRMLIDMIKYKAEEAGVPVMIIDPRNTSRKCSKCGYVDENN |
| | RKQASFKCLKCGYSLNADLNAAVNIAKAFYECPTFRWEEKLHAYVCSEPDK |
| 64 | MKSFKLKLLPTDEQNVLLNEVFCKWASLCTRMASKGHDKERLAPPDSSGNYFNKTQLN |
| | QVNTDVTDHMGALEESASQKERAVEKVKRRLKLISDMLSEPNLRDVSQQKPTTFRPLE |
| | WVKEGLLKTKYHTVHYWQKECDKLTKQKERMEKTIEKIKKGKITFKPTKMSLHQNCFS |
| | LSFGKGTFSMRPFSDTKRGINLDMLTAPIQPAIGKNDGKSSLRSKEFIARNIENYIIFSIHSQ |
| | LFGLSRSEELLLNAKKEELVAKRDAMLKKKSDSLSKKIKELEKIVGRKITDSERSEIMSQ |
| | GGKLSSEKFSEDNSYLKTLKVLAKDIIGREELFRLKKYPIVIRKPLNERKKLKNLKPDEW |
| | EYYLQLSYDELEKKEFTPKTIMGIDRGLKHILAIAIYDPVQNKFVKNMLIPNPILGWKWK |
| | LRKIKRSIQHMERRIRAQQNAHVPENQLKKRLKSIENKIDYYYHNVSRQILNLAHDFKSA |
| | IVVEDLQNMKQHGRKKSKGLRGLNYALSNFDYGKIMGLVKYKAESENVPLLTVLPAGT |
| | SQNCAYCLLYGKEQGNYVRNNVNSKIGKCKLHGEIDADINAARTIAICYHKNINEPKPY |
| | GERKTFKRK |
| 65 | MKYTKVMRYQIIKPLNAEWDELGMVLRDIQKETRAALNKTIQLCWEYQGFSADYKQIH |
| | GQYPKPKDVLGYTSMHGYAYDRLKNEFSKIASSNLSQTIKRAVDKWNSDLKEILRGDRS |
| | IPNFRKDCPIDIVKQSTKIQKCNDGYVLSLGLINREYKNELGRKNGVFDVLIKANDKTQQ |
| | TILERIINGDYTYTASQIINHKNKWFINLTYQFETKETALDPNNVMGVDLGIVYPVYIAFN |
| | NSLHRYHIKGGEIERFRRQVEKRKRELLNQGKYCGDGRKGHGYATRTKSIESISDKIARF |
| | RDTCNHKYSRFIVDMALKHNCGIIQMEDLTGISKESTFLKNWTYYDLQQKIEYKAREAGI |
| | QVIKIEPQYTSQRCSKCGYIDKENRQEQATFKCIECGFKTNADYNAARNIAIPNIDKIIRKT |
| | LKMQ |
| 66 | MTLLVKVVKIHLISEQFDKAGNRIDYEEVNKILWELQKQTREAKNKTVQLLWEWNNFS |
| | SDYVKASGIYPKAKDIFGYSSVHGQANKELRTKLALNSSNLSTTTMDVCKNFNTYKKEV |
| | WKGKRSVPSYKSDQPLDLHKDSIKLIYENNEFYVRLALLKKAEFAKYGFKDGFRFKMQ |
| | VKDNSTKTILERCFDEVYKINASKLLYDQKKKKWKLNLSYSFDNKNISELDKEKILGVD |
| | VGVNCPLVASVFGDRDRFIIKGGEIEKFRKSVEARRRSMLEQTKYCGDGRIGHGRKKRT |
| | EPALNIGDKIARFRDTTNHKYSRALIEYAVKKGCGTIQMEKLTGITSKSDRFLKDWTYYD |
| | LQTKIENKAKEVGINVVYIAPKYTSQRCSKCGYIHKDNRPNQAKFRCLECDFESNADYN |
| | ASQNIGIKNIDKIIEKDLQKQESEVQVNENK |
| 67 | MGESVKAIKLKILDMFLDPECTKQDDNWRKDLSTMSRFCAEAGNMCLRDLYNYFSMP |
| | KEDRISSKDLYNAMYHKTKLLHPELPGKVANQIVNHAKDVWKRNAKLIYRNQISMPTY |
| | KITTAPIRLQNNIYKLIKNKNKYIIDVQLYSKEYSKDSGKGTHRYFLVAVRDSSTRMIFDR |
| | IMSKDHIDSSKSYTQGQLQIKKDHQGKWYCIIPYTFPTHETVLDPDKVMGVDLGVAKAV |
| | YWAFNSSYKRGCIDGGEIEHFRKMIRARRVSIQNQIKHSGDARKGHGRKRALKPIETLSE |
| | KEKNFRDTINHRYANRIVEAAIKQGCGTIQIENLEGIADTTGSKFLKNWPYYDLQTKIVN |
| | KAKEHGITVVAINPQYTSQRCSMCGYIEKTNRSSQAVFECKQCGYGSRTICINCRHVQVS |
| | GDVCEECGGIVKKENVNADYNAAKNISTPYIDQIIMEKCLELGIPYRSITCKECGHIQASG |
| | NTCEVCGSTNILKPKKIRKAK |
| 68 | MITVRKIKLTIMGDKDTRNSQYKWIRDEQYNQYRALNMGMTYLAVNDILYMNESGLEI |
| | RTIKDLKDCEKDIDKNKKEIEKLTARLEKEQNKKNSSSEKLDEIKYKISLVENKIEDYKLK |
| | IVELNKILEETQKERMDIQKEFKEKYVDDLYQVLDKIPFKHLDNKSLVTQRIKADIKSDK |
| | SNGLLKGERSIRNYKRNFPLMTRGRDLKFKYDDNDDIEIKWMEGIKFKVILGNRIKNSLE |
| | LRHTLHKVIEGKYKICDSSLQFDKNNNLILNLTLDIPIDIVNKKVSGRVVGVDLGLKIPAY |
| | CALNDVEYIKKSIGRIDDFLKVRTQMQSRRRRLQIAIQSAKGGKGRVNKLQALERFAEK |
| | EKNFAKTYNHFLSSNIVKFAVSNQAEQINMELLSLKETQNKSILRNWSYYQLQTMIEYK |
| | AQREGIKVKYIDPYHTSQTCSKCGNYEEGQRESQADFICKKCGYKVNADYNAARNIAM |
| | SNKYITKKEESKYYKIKESMV |
| 69 | MAKNTITKTLKLRIVRPYNSAEVEKIVADEKERRKQAGGTGELDGGFYKKLEKKHSEMF |
| | SFDRLNLLLNQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 70 | MEVQKTVMKTLSLRILRPLYSQEIEKEIKEEKERRKQAGGTGELDDKFYQKLRGQFPDA |
| | VFWQEISEIFRQLQKQAAEIYNQSLIELYYEIFIKGKGIANASSVEHYLSRVCYRRAAELF |
| | KNAAIASGLRSKIKSNFRLKELKNMKSGLPTTKSDNFPIPLVKQKGGQYTGFEISNHNSD |
| | FIIKIPFGRWQVKKEIDKYRPWEKFDFEQVQKSPKPISLLLSTQRRKRNKGWSKDEGTEA |
| | EIKKVMNGDYQTSYIEVKRGSKICEKSAWMLNLSIDVPKIDKGVDPSIIGGIAVGVRSPLV |
| | CAINNAFSRYSISDNDLFHFNKKMFARRRILLKKNRHKRAGHGAKNKLKPITILTEKSER |
| | FRKKLIERWACEIADFFIKNKVGTVQMENLESMKRKEDSYFNIRLRGFWPYAEMQNKIE |
| | FKLKQYGIEIRKVAPNNTSKTCSKCGHLNNYFNFEYRKKNKFPHFKCEKCNFKENAAYN |
| | AALNISNPKLKSTKERP |
| 71 | MAKNTITKTLKLRIVRPLYSQEIEKEIKEEKERRKQAGGTGELDDKFYQKLRGQFPDAVF |
| | WQEISEIFRQLQKQAAEIYNQSLIELYYEIFIKGKGIANASSVEHYLSRVCYRRAAELFKN |
| | AAIASGLRSKIKSNFRLKELKNMKSGLPTTKSDNFPIPLVKQKGGQYTGFEISNHNSDFIIK |
| | IPFGRWQVKKEIDKYRPWEKFDFEQVQKSPKPISLLLSTQRRKRNKGWSKDEGTEAEIK |
| | KVMNGDYQTSYIEVKRGSKICEKSAWMLNLSIDVPKIDKGVDPSIIGGIAVGVRSPLVCA |
| | INNAFSRYSISDNDLFHFNKKMFARRRILLKKNRHKRAGHGAKNKLKPITILTEKSERFR |
| | KKLIERWACEIADFFIKNKVGTVQMENLESMKRKEDSYFNIRLRGFWPYAEMQNKIEFK |
| | LKQYGIEIRKVAPNNTSKTCSKCGHLNNYFNFEYRKKNKFPHFKCEKCNFKENAAYNA |
| | ALNISNPKLKSTKERP |
| 72 | MIKVYRYEIVKPLDLDWQEISEIFRQLQKQAAEIYNQSLIELYYEIFIKGKGIANASSVEHY |
| | LSRVCYRRAAELFKNAAIASGLRSKIKSNFRLKELKNMKSGLPTTKSDNFPIPLVKQKGG |
| | QYTGFEISNHNSDFIIKIPFGRWQVKKEIDKYRPWEKFDFEQVQKSPKPISLLLSTQRRKR |
| | NKGWSKDEGTEAEIKKVMNGDYQTSYIEVKRGSKICEKSAWMLNLSIDVPKIDKGVDPS |
| | IIGGIAVGVRSPLVCAINNAFSRYSISDNDLFHFNKKMFARRRILLKKNRHKRAGHGAKN |
| | KLKPITILTEKSERFRKKLIERWACEIADFFIKNKVGTVQMENLESMKRKEDSYFNIRLRG |
| | FWPYAEMQNKIEFKLKQYGIEIRKVAPNNTSKTCSKCGHLNNYFNFEYRKKNKFPHFKC |
| | EKCNFKENAAYNAALNISNPKLKSTKERP |
| 73 | MITVRKIKLTIMGDKDTRNSQYKWIRDEQYNQYRALNMGMTYLAVNDAVFWQEISEIF |
| | RQLQKQAAEIYNQSLIELYYEIFIKGKGIANASSVEHYLSRVCYRRAAELFKNAAIASGLR |
| | SKIKSNFRLKELKNMKSGLPTTKSDNFPIPLVKQKGGQYTGFEISNHNSDFIIKIPFGRWQ |
| | VKKEIDKYRPWEKFDFEQVQKSPKPISLLLSTQRRKRNKGWSKDEGTEAEIKKVMNGD |
| | YQTSYIEVKRGSKICEKSAWMLNLSIDVPKIDKGVDPSIIGGIAVGVRSPLVCAINNAFSR |
| | YSISDNDLFHFNKKMFARRRILLKKNRHKRAGHGAKNKLKPITILTEKSERFRKKLIERW |
| | ACEIADFFIKNKVGTVQMENLESMKRKEDSYFNIRLRGFWPYAEMQNKIEFKLKQYGIEI |
| | RKVAPNNTSKTCSKCGHLNNYFNFEYRKKNKFPHFKCEKCNFKENAAYNAALNISNPK |
| | LKSTKERP |
| 74 | MGESVKAIKLKILDMFLDPECTKQDDNWQEISEIFRQLQKQAAEIYNQSLIELYYEIFIKG |
| | KGIANASSVEHYLSRVCYRRAAELFKNAAIASGLRSKIKSNFRLKELKNMKSGLPTTKSD |
| | NFPIPLVKQKGGQYTGFEISNHNSDFIIKIPFGRWQVKKEIDKYRPWEKFDFEQVQKSPKP |
| | ISLLLSTQRRKRNKGWSKDEGTEAEIKKVMNGDYQTSYIEVKRGSKICEKSAWMLNLSI |
| | DVPKIDKGVDPSIIGGIAVGVRSPLVCAINNAFSRYSISDNDLFHFNKKMFARRRILLKKN |
| | RHKRAGHGAKNKLKPITILTEKSERFRKKLIERWACEIADFFIKNKVGTVQMENLESMK |
| | RKEDSYFNIRLRGFWPYAEMQNKIEFKLKQYGIEIRKVAPNNTSKTCSKCGHLNNYFNFE |
| | YRKKNKFPHFKCEKCNFKENAAYNAALNISNPKLKSTKERP |
| 75 | MKYTKVMRYQIIKPLNAEWQEISEIFRQLQKQAAEIYNQSLIELYYEIFIKGKGIANASSV |
| | EHYLSRVCYRRAAELFKNAAIASGLRSKIKSNFRLKELKNMKSGLPTTKSDNFPIPLVKQ |
| | KGGQYTGFEISNHNSDFIIKIPFGRWQVKKEIDKYRPWEKFDFEQVQKSPKPISLLLSTQR |
| | RKRNKGWSKDEGTEAEIKKVMNGDYQTSYIEVKRGSKICEKSAWMLNLSIDVPKIDKG |
| | VDPSIIGGIAVGVRSPLVCAINNAFSRYSISDNDLFHFNKKMFARRRILLKKNRHKRAGH |
| | GAKNKLKPITILTEKSERFRKKLIERWACEIADFFIKNKVGTVQMENLESMKRKEDSYFN |
| | IRLRGFWPYAEMQNKIEFKLKQYGIEIRKVAPNNTSKTCSKCGHLNNYFNFEYRKKNKF |
| | PHFKCEKCNFKENAAYNAALNISNPKLKSTKERP |
| 76 | MTLLVKVVKIHLISEQFDKAGNRIDYEEISEIFRQLQKQAAEIYNQSLIELYYEIFIKGKGIA |
| | NASSVEHYLSRVCYRRAAELFKNAAIASGLRSKIKSNFRLKELKNMKSGLPTTKSDNFPI |
| | PLVKQKGGQYTGFEISNHNSDFIIKIPFGRWQVKKEIDKYRPWEKFDFEQVQKSPKPISLL |
| | LSTQRRKRNKGWSKDEGTEAEIKKVMNGDYQTSYIEVKRGSKICEKSAWMLNLSIDVP |
| | KIDKGVDPSIIGGIAVGVRSPLVCAINNAFSRYSISDNDLFHFNKKMFARRRILLKKNRHK |
| | RAGHGAKNKLKPITILTEKSERFRKKLIERWACEIADFFIKNKVGTVQMENLESMKRKED |
| | SYFNIRLRGFWPYAEMQNKIEFKLKQYGIEIRKVAPNNTSKTCSKCGHLNNYFNFEYRK |
| | KNKFPHFKCEKCNFKENAAYNAALNISNPKLKSTKERP |
| 77 | MAKNTITKTLKLRIVRPYYSQEIEKIVAEEKNRREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 78 | MAKNTITKTLKLRIVRPYYSAEVEKIVAEEKNNREKIALDKNKDKVKEACSKHLKVAAY |
| | CTTQVERNACLFCKARKLDDKFYKKLRGQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 79 | MAKNTITKTLKLRIVRPYYSAEIEKIVADEKNRREKIALEKNKDKVKEACSKHLKVAAY |
| | CTTQVERNACLFCKARKLDDKFYQKLRKQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 80 | MAKNTITKTLKLRIVRPYNSQEVEKIVAEEKNRREKIALDKNKDKVKEACSKHLKVAAY |
| | CTTQVERNACLFCKARKLDDKFYQKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 81 | MAKNTITKTLKLRIVRPYNSQEVEKIVAEEKNNREKIALDKNKDKVKEACSKHLKVAAY |
| | CTTQVERNACLFCKARKLDDKFYKKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 82 | MAKNTITKTLKLRIVRPYNSQEVEKIVAEEKNNREKIALDKNKDKVKEACSKHLKVAAY |
| | CTTQVERNACLFCKARKLDDKFYKKLRGQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 83 | MAKNTITKTLKLRIVRPYYSAEVEKIVAEEKNNREKIALDKNKDKVKEACSKHLKVAAY |
| | CTTQVERNACLFCKARKLDDKFYKKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 84 | MAKNTITKTLKLRIVRPYNSAEIEKIVADEKNRREKIALDKNKDKVKEACSKHLKVAAY |
| | CTTQVERNACLFCKARKLDDKFYKKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 85 | MAKNTITKTLKLRIVRPYNSAEIEKIVADEKNRREKIALDKNKDKVKEACSKHLKVAAY |
| | CTTQVERNACLFCKARKLDDKFYKKLRGQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 86 | MAKNTITKTLKLRIVRPYNSQEIEKIVAEEKNRREKIALDKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 87 | MAKNTITKTLKLRIVRPYNSAEIEKIVAEEKNRREKIALDKNKDKVKEACSKHLKVAAY |
| | CTTQVERNACLFCKARKLDDKFYKKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 88 | MAKNTITKTLKLRIVRPYNSAEVEKIVAEEKNRREKIALDKNKDKVKEACSKHLKVAAY |
| | CTTQVERNACLFCKARKLDDKFYKKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 89 | MAKNTITKTLKLRIVRPYNSAEVEKIVADEKNRREKIALDKNKDKVKEACSKHLKVAAY |
| | CTTQVERNACLFCKARKLDDKFYKKLRKQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 90 | MAKNTITKTLKLRIVRPYYSAEIEKIVADEKNRREKIALDKNKDKVKEACSKHLKVAAY |
| | CTTQVERNACLFCKARKLDDKFYQKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 91 | MAKNTITKTLKLRIVRPYYSAEIEKIVAEEKNRREKIALEKNKDKVKEACSKHLKVAAY |
| | CTTQVERNACLFCKARKLDDKFYQKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 92 | MAKNTITKTLKLRIVRPYYSAEIEKIVAEEKNNREKIALDKNKDKVKEACSKHLKVAAY |
| | CTTQVERNACLFCKARKLDDKFYQKLRGQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 93 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAARLFKNAAIASGLRSKIKSNFRLKELKNMK |
| | SGLPTTKSDNFPIPLVKQKGGQYTGFKISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 94 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFKISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 95 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAGLFKNAAIASGLRSKIKSNFRLKELKNMK |
| | SGLPTTKSDNFPIPLVKQKGGQYTGFKISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 96 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAARLFKNAAIASGLRSKIKSNFRLKELKNMK |
| | SGLPTTKSDNFPIPLVKQKGGQYTGFRISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 97 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFRISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 98 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAGLFKNAAIASGLRSKIKSNFRLKELKNMK |
| | SGLPTTKSDNFPIPLVKQKGGQYTGFRISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 99 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAARLFKNAAIASGLRSKIKSNFRLKELKNMK |
| | SGLPTTKSDNFPIPLVKQKGGQYTGFSISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 100 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFSISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 101 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAGLFKNAAIASGLRSKIKSNFRLKELKNMK |
| | SGLPTTKSDNFPIPLVKQKGGQYTGFSISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 102 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLENFNKKMFA |
| | RRRILLKKNRHKRGGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 103 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIEGGDLFHFNKKMFA |
| | RRRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 104 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIEGGDLENFNKKMFA |
| | RRRILLKKNRHKRGGHGRDKKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 105 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIEGGDLENFNKKMFA |
| | RRRILLKKNRHKRAGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 106 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGRDKKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 107 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLENFNKKMFA |
| | RRRILLKKNRHKRAGHGRDKKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 108 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLEHFNKKMFA |
| | RRRILLKKNRHKRKGHGAKNKLKPIETLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 109 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDGGDLEHFNKKMFA |
| | RRRILLKKNRHKRKGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 110 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDGGDLFHFNKKMFA |
| | RRRILLKKNRHKRAGHGAKNKLKPIETLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 111 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDGGDLEHFNKKMFA |
| | RRRILLKKNRHKRKGHGAKNKLKPIETLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 112 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSIDSNDLFKFNKKMFAR |
| | RRILLKKNRHKRKGHGAKNKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 113 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSIDSNDLFHFNKKMFAR |
| | RRILLKKNRHKRAGHGAAHKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 114 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSIDSNDLFKFNKKMFAR |
| | RRILLKKNRHKRAGHGAAHKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 115 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDSNDLFKFNKKMFAR |
| | RRILLKKNRHKRAGHGAAHKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 116 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSIDSNDLFKFNKKMFAR |
| | RRILLKKNRHKRAGHGAKNKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 117 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSISDNDLFKFNKKMFAR |
| | RRILLKKNRHKRKGHGAKNKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 118 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDSNDLFKFNKKMFAR |
| | RRILLKKNRHKRKGHGAKNKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 119 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFKFNKKMFAR |
| | RRILLKKNRHKRKGHGAAHKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 120 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLERFNKKMFA |
| | RRRILLKKNRHKRKGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 121 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLERFNKKMFA |
| | RRRILLKKNRHKRAGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 122 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLEKFNKKMFA |
| | RRRILLKKNRHKRAGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 123 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLFHFNKKMFA |
| | RRRILLKKNRHKRAGHGRKKKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 124 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLEKFNKKMFA |
| | RRRILLKKNRHKRAGHGRKKKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 125 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLEKFNKKMFA |
| | RRRILLKKNRHKRAGHGRKKKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 126 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWAKEIADFFIKNKVGTVQM |
| | EDLSTMKRKEDSYFNIRLRGFWPYYEMQNKIEFKLKQYGIEIRKVAPNNTSQLCSKCGH |
| | LNNYFNFEYRKKNKFPKFKCEKCNFKENAAYNAARNISTPDIKSTKERP |
| 127 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLESMKRKEDSYFNIRLRGFWPYAEMQNKIEFKLKQYGIEIRKVAPNNTSQLCSKCGH |
| | LNNYFNFEYRKKNKFPHFKCEKCNFKENAAYNAALNISNPDIKSTKERP |
| 128 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWAKEIADFFIKNKVGTVQM |
| | EDLSTMKRKEDSYFNIRLRGFWPYYEMQNKIEFKLKQYGIEIRKVAPNNTSKTCSKCGH |
| | LNNYFNFEYRKKNKFPHFKCEKCNFKENAAYNAALNISNPKLKSTKERP |
| 129 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSQLCSKCGH |
| | LNNYFNFEYRKKNKFPKFKCEKCNFKENAAYNAALNISNPDIKSTKERP |
| 130 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSQLCSKCGH |
| | LNNYFNFEYRKKNKFPHFKCEKCNFKENAAYNAALNISTPDIKSTKERP |
| 131 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLSTMKRKEDSYFNIRLRGFWPYYEMQNKIEFKLKQYGIEIRKVAPNNTSKTCSKCGH |
| | LNNYFNFEYRKKNKFPKFKCEKCNFKENAAYNAALNISNPKLKSTKERP |
| 132 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLESMKRKEDSYFNIRLRGFWPYAEMQNKIEFKLKQYGIEIRKVAPNNTSQLCSKCGH |
| | LNNYFNFEYRKKNKFPKFKCEKCNFKENAAYNAALNISTPDIKSTKERP |
| 133 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPKFKCEKCNFKENAAYNAARNISTPDIKSTKERP |
| 134 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWSRYIADFFIKNKVGTVQM |
| | EDLESMKRKEDSYFNIRLRGFWPYYEMQNKIEFKLKQYGIKIRKVAPNNTSQRCSKCGH |
| | LNNYFNFEYRKKNKFPHFKCEKCNFKANAAYNAARNISNPNIKSTKERP |
| 135 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACYIADFFIKNKVGTVQM |
| | EDLESMKRKEDSYFNIRLRGFWPYAEMQNKIEFKLKQYGIEIRKVAPNNTSQRCSKCGH |
| | LNNYFNFEYRKKNKFPHFKCEKCNFKENAAYNAARNISNPNIKSTKERP |
| 136 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACYIADFFIKNKVGTVQM |
| | ENLESMKRKEDSYFNIRLRGFWPYAEMQNKIEFKLKQYGIEIRKVAPNNTSQRCSKCGH |
| | LNNYFNFEYRKKNKFPHFKCEKCNFKRNAAYNAARNISNPKLKSTKERP |
| 137 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACYIADFFIKNKVGTVQM |
| | EDLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAARNISNPNIKSTKERP |
| 138 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWARYIADFFIKNKVGTVQM |
| | EDLESMKRKEDSYFNIRLRGFWPYYEMQNKIEFKLKQYGIKIRKVAPNNTSKTCSKCGH |
| | LNNYFNFEYRKKNKFPHFKCEKCNFKENAAYNAALNISNPKLKSTKERP |
| 139 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSQRCSKCGH |
| | LNNYFNFEYRKKNKFPHFKCEKCNFKRNAAYNAARNISNPNIKSTKERP |
| 140 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLESMKRKEDSYFNIRLRGFWPYAEMQNKIEFKLKQYGIEIRKVAPNNTSQRCSKCGH |
| | LNNYFNFEYRKKNKFPHFKCEKCNFKENAAYNAARNISNPNIKSTKERP |
| 141 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKRNAAYNAARNISNPNIKSTKERP |
| 142 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWANRIADFFIKNKVGTVQM |
| | ENLESMKRKEDSYFNIRLRGFWPYAEMQNKIEFKLKQYGIKIRKVAPNNTSQRCSKCGH |
| | LNNYFNFEYRKKNKFPHFKCEKCNFKRNAAYNAAKNISNPKLKSTKERP |
| 143 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSQRCSKCGH |
| | LNNYFNFEYRKKNKFPHFKCEKCNFKRNAAYNAAKNISNPKLKSTKERP |
| 144 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIKIRKVAPNNTSQRCSKCGH |
| | LNNYFNFEYRKKNKFPHFKCEKCNFKRNAAYNAAKNISNPKLKSTKERP |
| 145 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWANRIADFFIKNKVGTVQM |
| | ENLESMKRKEDSYFNIRLRGFWPYAEMQNKIEFKLKQYGIKIRKVAPNNTSQRCSKCGH |
| | LNNYFNFEYRKKNKFPHFKCEKCNFKENAAYNAALNISNPKLKSTKERP |
| 146 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWANRIADFFIKNKVGTVQM |
| | ENLESMKRKEDSYFNIRLRGFWPYAEMQNKIEFKLKQYGIEIRKVAPNNTSQRCSKCGH |
| | LNNYFNFEYRKKNKFPHFKCEKCNFKRNAAYNAALNISNPKLKSTKERP |
| 147 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWSRFIADFFIKNKVGTVQM |
| | EDLESMKRKEDSYFNIRLRGFWPYYEMQNKIEFKLKQYGIEIRKVAPNNTSQRCSKCGH |
| | LNNYFNFEYRKKNKFPHFKCEKCNFKENAAYNAARNISNPNIKSTKERP |
| 148 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIDVQLYSKEYSKDSGKGTHRYFLLS |
| | TQRRKRNKGWSKDEGTEAEIKKVMNGDYQTSYIEVKRGSKICEKSAWMLNLSIDVPKID |
| | KGVDPSIIGGIAVGVRSPLVCAINNAFSRYSISDNDLFHFNKKMFARRRILLKKNRHKRA |
| | GHGAKNKLKPITILTEKSERFRKKLIERWACEIADFFIKNKVGTVQMENLESMKRKEDSY |
| | FNIRLRGFWPYAEMQNKIEFKLKQYGIEIRKVAPNNTSKTCSKCGHLNNYFNFEYRKKN |
| | KFPHFKCEKCNFKENAAYNAALNISNPKLKSTKERP |
| 149 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIASLSLLSNPAKQEMNVKRKISLLLS |
| | TQRRKRNKGWSKDEGTEAEIKKVMNGDYQTSYIEVKRGSKICEKSAWMLNLSIDVPKID |
| | KGVDPSIIGGIAVGVRSPLVCAINNAFSRYSISDNDLFHFNKKMFARRRILLKKNRHKRA |
| | GHGAKNKLKPITILTEKSERFRKKLIERWACEIADFFIKNKVGTVQMENLESMKRKEDSY |
| | FNIRLRGFWPYAEMQNKIEFKLKQYGIEIRKVAPNNTSKTCSKCGHLNNYFNFEYRKKN |
| | KFPHFKCEKCNFKENAAYNAALNISNPKLKSTKERP |
| 150 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPIYERKPNRSIVGGLAVGIRSPLVCAINNSFSRYSVDSNDVFKFSKQVFAF |
| | RRRLLSKNSLKRKGHGAAHKLEPITEMTEKNDKFRKKIIERWAKEVTNFFVKNQVGIVQ |
| | IEDLSTMKDREDHFFNQYLRGFWPYYQMQTLIENKLKEYGIEVKRVQAKYTSQLCSNPN |
| | CRYWNNYFNFEYRKVNKFPKFKCEKCNLEISAAYNAARNLSTPDIEKFVAKATKGINLP |
| | EK |
| 151 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPTHETVLDPDKVMGVALGVAKAVYWAFNSSYKRGCIDGGEIEHFRKMI |
| | RARRVSIQNQIKHSGDARKGHGRKRALKPIETLSEKEKNFRDTINHRYANRIVEAAIKQG |
| | CGTIQIENLEGIADTTGSKFLKNWPYYDLQTKIVNKAKEHGITVVAINPQYTSQRCSMCG |
| | YIEKTNRSSQAVFECKQCGYGSRTICINCRHVQVSGDVCEECGGIVKKENVNAAYNAAK |
| | NISTPYIDQIIMEKCLELGIPYRSITCKECGHIQASGNTCEVCGSTNILKPKK |
| 152 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPQTRVLDLNKIMGIALGVAVAVYMAFQHTPARYKLEGGEIENFRRQVE |
| | SRRISMLRQGKYAGGARGGHGRDKRIKPIEQLRDKIANFRDTTNHRYSRYIVDMAIKEG |
| | CGTIQMEDLTNIRDIGSRFLQNWTYYDLQQKIIYKAEEAGIKVIKIDPQYTSQRCSECGNI |
| | DSGNRIGQAIFKCRACGYEANAAYNAARNIAIPNIDKIIAESIK |
| 153 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPIDIVNKKVSGRVVGVALGLKIPAYCALNDVEYIKKSIGRIDDFLKVRTQ |
| | MQSRRRRLQIAIQSAKGGKGRVNKLQALERFAEKEKNFAKTYNHFLSSNIVKFAVSNQA |
| | EQINMELLSLKETQNKSILRNWSYYQLQTMIEYKAQREGIKVKYIDPYHTSQTCSKCGN |
| | YEEGQRESQADFICKKCGYKVNAAYNAARNIAMSNKYITKKEESKYYKIKESMV |
| 154 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVETKETALDPNNVMGVALGIVYPVYIAFNNSLHRYHIKGGEIERFRRQVE |
| | KRKRELLNQGKYCGDGRKGHGYATRTKSIESISDKIARFRDTCNHKYSRFIVDMALKHN |
| | CGIIQMEDLTGISKESTFLKNWTYYDLQQKIEYKAREAGIQVIKIEPQYTSQRCSKCGYID |
| | KENRQEQATFKCIECGFKTNAAYNAARNIAIPNIDKIIRKTLKMQ |
| 155 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVNRSIVGGLAVGIRSPLVCAINNSFSRYSVDSNDVFKFSKQVFA |
| | FRRRLLSKNSLKRKGHGAAHKLEPITEMTEKNDKFRKKIIERWAKEVTNFFVKNQVGIV |
| | QIEDLSTMKDREDHFFNQYLRGFWPYYQMQTLIENKLKEYGIEVKRVQAKYTSQLCSNP |
| | NCRYWNNYFNFEYRKVNKFPKFKCEKCNLEISAAYNAARNLSTPDIEKFVAKATKGINL |
| | PEK |
| 156 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPDKVMGVALGVAKAVYWAFNSSYKRGCIDGGEIEHFRKMI |
| | RARRVSIQNQIKHSGDARKGHGRKRALKPIETLSEKEKNFRDTINHRYANRIVEAAIKQG |
| | CGTIQIENLEGIADTTGSKFLKNWPYYDLQTKIVNKAKEHGITVVAINPQYTSQRCSMCG |
| | YIEKTNRSSQAVFECKQCGYGSRTICINCRHVQVSGDVCEECGGIVKKENVNAAYNAAK |
| | NISTPYIDQIIMEKCLELGIPYRSITCKECGHIQASGNTCEVCGSTNILKPKK |
| 157 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDLNKIMGIALGVAVAVYMAFQHTPARYKLEGGEIENFRRQV |
| | ESRRISMLRQGKYAGGARGGHGRDKRIKPIEQLRDKIANFRDTTNHRYSRYIVDMAIKE |
| | GCGTIQMEDLTNIRDIGSRFLQNWTYYDLQQKIIYKAEEAGIKVIKIDPQYTSQRCSECGN |
| | IDSGNRIGQAIFKCRACGYEANAAYNAARNIAIPNIDKIIAESIK |
| 158 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPNNVMGVALGIVYPVYIAFNNSLHRYHIKGGEIERFRRQVE |
| | KRKRELLNQGKYCGDGRKGHGYATRTKSIESISDKIARFRDTCNHKYSRFIVDMALKHN |
| | CGIIQMEDLTGISKESTFLKNWTYYDLQQKIEYKAREAGIQVIKIEPQYTSQRCSKCGYID |
| | KENRQEQATFKCIECGFKTNAAYNAARNIAIPNIDKIIRKTLKMQ |
| 159 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDKEKILGVAVGVNCPLVASVFGDRDRFIIKGGEIEKFRKSVEA |
| | RRRSMLEQTKYCGDGRIGHGRKKRTEPALNIGDKIARFRDTTNHKYSRALIEYAVKKGC |
| | GTIQMEKLTGITSKSDRFLKDWTYYDLQTKIENKAKEVGINVVYIAPKYTSQRCSKCGYI |
| | HKDNRPNQAKFRCLECDFESNAAYNASQNIGIKNIDKIIEKDLQKQESEVQVNENK |
| 160 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNI |
| 161 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 162 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 163 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 164 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 165 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 166 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 167 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 168 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 169 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 170 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 171 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 172 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 173 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 174 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 175 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 176 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 177 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 178 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 179 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 180 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 181 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 182 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 183 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 184 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 185 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 186 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 187 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 188 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 189 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 190 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 191 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 192 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 193 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 194 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 195 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 196 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 197 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 198 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 199 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 200 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 201 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 202 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 203 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 204 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 205 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 206 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 207 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 208 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 209 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 210 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 211 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 212 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 213 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 214 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 215 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 216 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 217 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 218 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 219 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 220 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 221 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 222 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 223 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 224 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 225 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 226 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 227 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 228 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 229 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 230 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 231 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 232 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 233 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 234 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 235 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 236 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 237 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 238 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 239 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 240 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 241 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSISDNDLFKFNKKMFAR |
| | RRILLKKNRHKRKGHGAKNKLKPITELTEKSERFRKKLIERWACEIADFFIKNKVGTVQM |
| | EDLESMKRKEDSYFNIRLRGFWPYAEMQNKIEFKLKQYGIEIRKVAPNNTSQLCSKCGH |
| | LNNYFNFEYRKKNKFPHFKCEKCNFKENAAYNAALNISNPDIKSTKERP |
| 242 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLESMKRKEDSYFNIRLRGFWPYAEMQNKIEFKLKQYGIEIRKVAPNNTSQLCSKCGH |
| | LNNYFNFEYRKKNKFPHFKCEKCNFKENAAYNAALNISNPDIKSTKERP |
| 243 | MAKNTITKTLKLRIVRPYNSAEIEKIVADEKERRKQAGGTGELDDKFYKKLRKQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 244 | MAKNTITKTLKLRIVRPYNSAEIEKIVADEKERRKQAGGTGELDDKFYKKLRKQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 245 | MAKNTITKTLKLRIVRPYNSAEIEKIVADEKERRKQAGGTGELDDKFYKKLRKQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 246 | MAKNTITKTLKLRIVRPYNSAEIEKIVADEKERRKQAGGTGELDDKFYKKLRKQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 247 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 248 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPKLKSTKERP |
| 249 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPDIKSTKERP |
| 250 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |

TABLE 6
Non-limiting examples of a guide nucleic acid
scaffold sequence or a derivative thereof.

| SEQ | Guide nucleic acid (NA) scaffold sequence |
|---|---|
| ID NO | (without spacer) |
| 251 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAUUCAUUUGAAUGAAGGAAUGCAAC |
| 252 | GAACCGCUUCACCAAAAGCUGUCCCUUAGGGGAUUAGAACUUGAGUGAAGGUGG |
| | GCUGCUUGCAUCAGCCUAAUGUCGAGAAGUGCUUUCUUCGGAAAGUAACCCUCGA |
| | AACAAAUUCAUUUGAAUGAAGGAAUGCAAC |
| 253 | AACCGCUUCACCAAAAGCUGUCCCUUAGGGGAUUAGAACUUGAGUGAAGGUGGGC |
| | UGCUUGCAUCAGCCUAAUGUCGAGAAGUGCUUUCUUCGGAAAGUAACCCUCGAAA |
| | CAAAUUCAUUUGAAUGAAGGAAUGCAAC |
| 254 | ACCGCUUCACCAAAAGCUGUCCCUUAGGGGAUUAGAACUUGAGUGAAGGUGGGCU |
| | GCUUGCAUCAGCCUAAUGUCGAGAAGUGCUUUCUUCGGAAAGUAACCCUCGAAAC |
| | AAAUUCAUUUGAAUGAAGGAAUGCAAC |
| 255 | CCGCUUCACCAAAAGCUGUCCCUUAGGGGAUUAGAACUUGAGUGAAGGUGGGCUG |
| | CUUGCAUCAGCCUAAUGUCGAGAAGUGCUUUCUUCGGAAAGUAACCCUCGAAACA |
| | AAUUCAUUUGAAUGAAGGAAUGCAAC |
| 256 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAGCAAUAAGGAAUGCAAC |
| 257 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAGAAAGGAAUGCAAC |
| 258 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAUCUUCGGAUUAAGGAAUGCAAC |
| 259 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAUUGCAAAAGGAAUGCAAC |
| 260 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAUUCGUUAAGGAAUGCAAC |
| 261 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAUGCAAAGGAAUGCAAC |
| 262 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAGCAAUUAAGGAAUGCAAC |
| 263 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAGAAAGGAAUGCAAC |
| 264 | GGCUUCACUGAUAAAGUGGAGAACCGCUUCACUUAGAGUGAAGGUGGGCUGCUU |
| | GCAUCAGCCUAAUGUCGAGAAGUGCUUUCUUCGGAAAGUAACCCUCGAAACAAAU |
| | UCAUUUGAAUGAAGGAAUGCAAC |
| 265 | GGCUUCACUGAUAAAGUGGAGAACCGCUUCACUUCGAGUGAAGGUGGGCUGCUU |
| | GCAUCAGCCUAAUGUCGAGAAGUGCUUUCUUCGGAAAGUAACCCUCGAAACAAAU |
| | UCAUUUGAAUGAAGGAAUGCAAC |
| 266 | GGCUUCACUGAUAAAGUGGAGAACCGCUUCACUUCGGUGAAGGUGGGCUGCUUGC |
| | AUCAGCCUAAUGUCGAGAAGUGCUUUCUUCGGAAAGUAACCCUCGAAACAAAUUC |
| | AUUUGAAUGAAGGAAUGCAAC |
| 267 | GGCUUCACUGAUAAAGUGGAGAACCGCUUCACCUUAGGAGUGAAGGUGGGCUGC |
| | UUGCAUCAGCCUAAUGUCGAGAAGUGCUUUCUUCGGAAAGUAACCCUCGAAACAA |
| | AUUCAUUUGAAUGAAGGAAUGCAAC |
| 268 | GGCUUCACUGAUAAAGUGGAGAACCGCUUCACCUUCGGAGUGAAGGUGGGCUGCU |
| | UGCAUCAGCCUAAUGUCGAGAAGUGCUUUCUUCGGAAAGUAACCCUCGAAACAAA |
| | UUCAUUUGAAUGAAGGAAUGCAAC |
| 269 | GGCUUCACUGAUAAAGUGGAGAACCGCUUCACGCUUCGGCAGUGAAGGUGGGCUG |
| | CUUGCAUCAGCCUAAUGUCGAGAAGUGCUUUCUUCGGAAAGUAACCCUCGAAACA |
| | AAUUCAUUUGAAUGAAGGAAUGCAAC |
| 270 | ACCGCUUCACCAAAAGCUGUCCUUAGGGAUUAGAACUUGAGUGAAGGUGGGCUGC |
| | UUGCAUCAGCCUAAUGUCGAGAAGUGCUUUCUUCGGAAAGUAACCCUCGAAACAA |
| | AUUCAUUUGAAUGAAGGAAUGCAAC |
| 271 | ACCGCUUCACCAAAAGCUGUCUUAGGAUUAGAACUUGAGUGAAGGUGGGCUGCU |
| | UGCAUCAGCCUAAUGUCGAGAAGUGCUUUCUUCGGAAAGUAACCCUCGAAACAAA |
| | UUCAUUUGAAUGAAGGAAUGCAAC |
| 272 | ACCGCUUCACCAAAAGCUGUUUAGAUUAGAACUUGAGUGAAGGUGGGCUGCUUG |
| | CAUCAGCCUAAUGUCGAGAAGUGCUUUCUUCGGAAAGUAACCCUCGAAACAAAUU |
| | CAUUUGAAUGAAGGAAUGCAAC |
| 273 | ACCGCUUCACCAAAAGCUGUUAGUUAGAACUUGAGUGAAGGUGGGCUGCUUGCA |
| | UCAGCCUAAUGUCGAGAAGUGCUUUCUUCGGAAAGUAACCCUCGAAACAAAUUCA |
| | UUUGAAUGAAGGAAUGCAAC |
| 274 | ACCGCUUCACCAAAAGCUUUAGAGAACUUGAGUGAAGGUGGGCUGCUUGCAUCAG |
| | CCUAAUGUCGAGAAGUGCUUUCUUCGGAAAGUAACCCUCGAAACAAAUUCAUUUG |
| | AAUGAAGGAAUGCAAC |
| 275 | ACCGCUUCACCAAAAGCUUCGGCACUUGAGUGAAGGUGGGCUGCUUGCAUCAGCC |
| | UAAUGUCGAGAAGUGCUUUCUUCGGAAAGUAACCCUCGAAACAAAUUCAUUUGA |
| | AUGAAGGAAUGCAAC |
| 276 | ACCGCUUCACCAAAAGUUCGCACUUGAGUGAAGGUGGGCUGCUUGCAUCAGCCUA |
| | AUGUCGAGAAGUGCUUUCUUCGGAAAGUAACCCUCGAAACAAAUUCAUUUGAAU |
| | GAAGGAAUGCAAC |
| 277 | ACCGCUUCACCAAAAUUCGUCUUGAGUGAAGGUGGGCUGCUUGCAUCAGCCUAAU |
| | GUCGAGAAGUGCUUUCUUCGGAAAGUAACCCUCGAAACAAAUUCAUUUGAAUGA |
| | AGGAAUGCAAC |
| 278 | ACCGCUUCACCAAGUUCGCUUGAGUGAAGGUGGGCUGCUUGCAUCAGCCUAAUGU |
| | CGAGAAGUGCUUUCUUCGGAAAGUAACCCUCGAAACAAAUUCAUUUGAAUGAAG |
| | GAAUGCAAC |
| 279 | ACCGCUUCACCAAUUCGUUGAGUGAAGGUGGGCUGCUUGCAUCAGCCUAAUGUCG |
| | AGAAGUGCUUUCUUCGGAAAGUAACCCUCGAAACAAAUUCAUUUGAAUGAAGGA |
| | AUGCAAC |
| 280 | ACCGCUUCACCAUUCGUGAGUGAAGGUGGGCUGCUUGCAUCAGCCUAAUGUCGAG |
| | AAGUGCUUUCUUCGGAAAGUAACCCUCGAAACAAAUUCAUUUGAAUGAAGGAAU |
| | GCAAC |
| 281 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAUUCAUUUGAAUGAAGGAAUGCAAC |
| — | UU |
| 283 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAUUCAUUUGAAUGAAGGAAUGCAAC |
| — | UUUUA |
| 285 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAUUCAUUUGAAUGAAGGAAUGCAAC |
| — | UUUUG |
| 287 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAUUCAUUUGAAUGAAGGAAUGCAAC |
| 288 | UUUUAUUUUU |
| 289 | GGCUUCACUGAUAAAGUGGAGAACCGCUUCACCGAGUGAAGGUGGGCUGCUUGCA |
| | UCAGCCUAAUGUCGAGAAGUGCUUUCUUCGGAAAGUAACCCUCGAAACAAAUUCA |
| | UUUGAAUGAAGGAAUGCAAC |
| 290 | UUUUAUUUUU |
| 291 | GGCUUCACUGAUAAAGUGGAGAACCGCUUCACCAAAAGCUGUCCCUUAGGGGAUU |
| | AGAACUUGAGUGAAGGUGGGCUGCUUGCAUCAGCCUAAUGUCGAGAAGUGCUUU |
| | CUUCGGAAAGUAACCCUCGAAACAAAGAAAGGAAUGCAAC |
| 292 | GGCUUCACUGAUAAAGUGGAGAACCGCUUCACUUAGAGUGAAGGUGGGCUGCUU |
| | GCAUCAGCCUAAUGUCGAGAAGUGCUUUCUUCGGAAAGUAACCCUCGAAACAAAU |
| | UCAUUUGAAUGAAGGAAUGCAAC |
| 293 | UUUUAUUUUU |
| 294 | GGCUUCACUGAUAAAGUGGAGAACCGCUUCACCGAGUGAAGGUGGGCUGCUUGCA |
| | UCAGCCUAAUGUCGAGAAGUGCUUUCUUCGGAAAGUAACCCUCGAAACAAAUUCA |
| | UUUGAAUGAAGGAAUGCAAC |
| 295 | UUUUAUUUUU |
| 296 | GGCUUCACUGAUAAAGUGGAGAACCGCUUCACUUAGAGUGAAGGUGGGCUGCUU |
| | GCAUCAGCCUAAUGUCGAGAAGUGCUUUCUUCGGAAAGUAACCCUCGAAACAAAG |
| | AAAGGAAUGCAAC |
| 297 | GGCUUCACUGAUAAAGUGGAGAACCGCUUCACCGAGUGAAGGUGGGCUGCUUGCA |
| | UCAGCCUAAUGUCGAGAAGUGCUUUCUUCGGAAAGUAACCCUCGAAACAAAGAAA |
| | GGAAUGCAAC |
| 298 | ACCGCUUCACUUAGAGUGAAGGUGGGCUGCUUGCAUCAGCCUAAUGUCGAGAAGU |
| | GCUUUCUUCGGAAAGUAACCCUCGAAACAAAGAAAGGAAUGCAAC |
| 299 | ACCGCUUCACCGAGUGAAGGUGGGCUGCUUGCAUCAGCCUAAUGUCGAGAAGUGC |
| | UUUCUUCGGAAAGUAACCCUCGAAACAAAGAAAGGAAUGCAAC |
| 300 | ACCGCUUCACUUAGAGUGAAGGUGGGCUGCUUGCAUCAGCCUAAUGUCGAGAAGU |
| | GCUUUCUUCGGAAAGUAACCCUCGAAACAAAGAAAGGAAUGCAAC |
| 301 | UUUUAUUUUU |
| 302 | ACCGCUUCACCGAGUGAAGGUGGGCUGCUUGCAUCAGCCUAAUGUCGAGAAGUGC |
| | UUUCUUCGGAAAGUAACCCUCGAAACAAAGAAAGGAAUGCAAC |
| 303 | UUUUAUUUUU |
| 304 | ACCGCUUCACGCUUCGGCAGUGAAGGUGGGCUGCUUGCAUCAGCCUAAUGUCGAG |
| | AAGUGCUUUCUUCGGAAAGUAACCCUCGAAACAAAGAAAGGAAUGCAAC |
| 305 | ACCGCUUCACGCUUCGGCAGUGAAGGUGGGCUGCUUGCAUCAGCCUAAUGUCGAG |
| | AAGUGCUUUCUUCGGAAAGUAACCCUCGAAACAAAGAAAGGAAUGCAAC |
| | UUUUA |
| 307 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGAAAGGAAUGCAAC |
| 308 | CCGCUUCACGCUUAGGCAGUGAAGGUGGGCUGCUUGCAUCAGCCUAAUGUCGAGA |
| | AGUGCUUUCUUCGGAAAGUAACCCUCGAAACAAAGAAAGGAAUGCAAC |
| 309 | CCGCUUCACUCUUAGGAAGUGAAGGUGGGCUGCUUGCAUCAGCCUAAUGUCGAGA |
| | AGUGCUUUCUUCGGAAAGUAACCCUCGAAACAAAGAAAGGAAUGCAAC |
| 310 | CCGCUUCACGUUUAGACAGUGAAGGUGGGCUGCUUGCAUCAGCCUAAUGUCGAGA |
| | AGUGCUUUCUUCGGAAAGUAACCCUCGAAACAAAGAAAGGAAUGCAAC |
| 311 | GCCGCUUCACGCUUCGGCAGUGAAGGUGGGCUGCUUGCAUCAGCCUAAUGUCGAG |
| | AAGUGCUUUCUUCGGAAAGUAACCCUCGAAACAAAGAAAGGAAUGCAAC |
| 312 | GGCCGCUUCACGCUUCGGCAGUGAAGGUGGGCUGCUUGCAUCAGCCUAAUGUCGA |
| | GAAGUGCUUUCUUCGGAAAGUAACCCUCGAAACAAAGAAAGGAAUGCAAC |
| 313 | CGCUUCACGCUUCGGCAGUGAAGGUGGGCUGCUUGCAUCAGCCUAAUGUCGAGAA |
| | GUGCUUUCUUCGGAAAGUAACCCUCGAAACAAAGAAAGGAAUGCAAC |
| 314 | GCUUCACGCUUCGGCAGUGAAGGUGGGCUGCUUGCAUCAGCCUAAUGUCGAGAAG |
| | UGCUUUCUUCGGAAAGUAACCCUCGAAACAAAGAAAGGAAUGCAAC |
| 315 | GGCUUCACGCUUCGGCAGUGAAGGUGGGCUGCUUGCAUCAGCCUAAUGUCGAGAA |
| | GUGCUUUCUUCGGAAAGUAACCCUCGAAACAAAGAAAGGAAUGCAAC |
| 316 | GCUUCACGCUUCGGCAGUGAAGGUGGGCUGCUUGCAUCAGCCUAAUGUCGAGAAG |
| | UGCUUUCUUCGGAAAGUAACCCUCGAAACAAAGAAAGGAAUGCAAC |
| 317 | UUUUAUUUUU |
| 318 | GGCUUCACGCUUCGGCAGUGAAGGUGGGCUGCUUGCAUCAGCCUAAUGUCGAGAA |
| | GUGCUUUCUUCGGAAAGUAACCCUCGAAACAAAGAAAGGAAUGCAAC |
| 319 | UUUUAUUUUU |
| 320 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGAAGGAAUGCAAC |
| 321 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGAGGAAUGCAAC |
| 322 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGGGAAUGCAAC |
| 323 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGGAAUGCAAC |
| 324 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAGGAAUGCAAC |
| 325 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAGGAAUGCAAC |
| 326 | CCGCUUCACGCUUCGGCAGUGAAGGUGGGCUGCUUGCAUCAGCCUAAUGUCGAGA |
| | AGUGCUUUCUUCGGAAAGUAACCCUCGAAACGGAAUGCAAC |
| 327 | CCGCUUCACGCUUCGGCAGUGAAGGUGGGCUGCUUGCAUCAGCCUAAUGUCGAGA |
| | AGUGCUUUCUUCGGAAAGUAACCCUCGAAAGGAAUGCAAC |
| 328 | CCGCUUCACGCUUCGGCAGUGAAGGUGGGCUGCUUGCAUCAGCCUAAUGUCGAGA |
| | AGUGCUUUCUUCGGAAAGUAACCCUCGAAGGAAUGCAAC |
| 329 | CCGCUUCACGCUUCGGCAGUGAAGGUGGGCUGCUUGCAUCAGCCUAAUGUCGAGA |
| | AGUGCUUUCUUCGGAAAGUAACCCUCGAGGAAUGCAAC |
| 330 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAGAAUGCAAC |
| 331 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGGAAUGCAAC |
| 332 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGAAUGCAAC |
| 333 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGAAAGAAUGCAAC |
| 334 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGAGAAUGCAAC |
| 335 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAGAAAUGCAAC |
| 336 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAGAAUGCAAC |
| 337 | CCGCUUCACGCUUCGGCAGUGAAGGUGGGCUGCUUGCAUCAGCCUAAUGUCGAGA |
| | AGUGCUUUCUUCGGAAAGUAACCCUCGAAACAAAUGCAAC |
| 338 | CCGCUUCACGCUUCGGCAGUGAAGGUGGGCUGAUUGCAUCAGCCUAAUGUCGAGA |
| | AGUGCUUUCUUCGGAAAGUAACCCUCGAAACAAAGAAAGGAAUGCAAC |
| 339 | CCGCUUCACGCUUCGGCAGUGAAGGUAGGCUGCUUGCAUCAGCCUAAUGUCGAGA |
| | AGUGCUUUCUUCGGAAAGUAACCCUCGAAACAAAGAAAGGAAUGCAAC |
| 340 | CCGCUUCACGCUUCGGCAGUGAAGGUGGGCUGCUUGCAUCAGCCCAAUGUCGAGA |
| | AGUGCUUUCUUCGGAAAGUAACCCUCGAAACAAAGAAAGGAAUGCAAC |
| 341 | GGCUUCACGCUUCGGCAGUGAAGGUAGGCUGCUUGCAUCAGCCUAAUGUCGAGAA |
| | GUGCUUUCUUCGGAAAGUAACCCUCGAAACAAGGAAUGCAAC |
| 342 | GGCUUCACGCUUCGGCAGUGAAGGUGGGCUGCUUGCAUCAGCCCAAUGUCGAGAA |
| | GUGCUUUCUUCGGAAAGUAACCCUCGAAACAAGGAAUGCAAC |
| 343 | CCGCUUCACUCUUAGGAAGUGAAGGUGGGCUGAUUGCAUCAGCCUAAUGUCGAGA |
| | AGUGCUUUCUUCGGAAAGUAACCCUCGAAACAAAGAAAGGAAUGCAAC |
| 344 | CCGCUUCACGCUUCGGCAGUGAAGGUGGGCUGAUUGCAUCAGCCUAAUGUCGAGA |
| | AGUGCUUUCUUCGGAAAGUAACCCUCGAAACAAAGGGAAUGCAAC |
| 345 | CCGCUUCACGCUUCGGCAGUGAAGGUGGGCUGAUUGCAUCAGCCUAAUGUCGAGA |
| | AGUGCUUUCUUCGGAAAGUAACCCUCGAAACAAAGGAAUGCAAC |
| 346 | CCGCUUCACGCUUCGGCAGUGAAGGUGGGCUGAUUGCAUCAGCCUAAUGUCGAGA |
| | AGUGCUUUCUUCGGAAAGUAACCCUCGAAACAAGGAAUGCAAC |
| 347 | CCGCUUCACGCUUCGGCAGUGAAGGUGGGCUGAUUGCAUCAGCCUAAUGUCGAGA |
| | AGUGCUUUCUUCGGAAAGUAACCCUCGAAACAAGAAUGCAAC |
| 348 | CCGCUUCACGCUUCGGCAGUGAAGGUGGGCUGAUUGCAUCAGCCUAAUGUCGAGA |
| | AGUGCUUUCUUCGGAAAGUAACCCUCGAAACAAAGGAAUGCAAC |
| 349 | CCGCUUCACGCUUCGGCAGUGAAGGUGGGCUGAUUGCAUCAGCCUAAUGUCGAGA |
| | AGUGCUUUCUUCGGAAAGUAACCCUCGAAACAAAGAAUGCAAC |
| 350 | CCGCUUCACGCUUCGGCAGUGAAGGUGGGCUGAUUGCAUCAGCCUAAUGUCGAGA |
| | AGUGCUUUCUUCGGAAAGUAACCCUCGAAACAGAAUGCAAC |

### EXAMPLES

### Example 1: Polypeptide Molecule as Gene Suppressor

### A. Suppression of a Heterologous Target Gene

The polypeptide molecule (comprising at least a portion of an endonuclease and one or more gene modulators coupled to polypeptide chains) as described herein can be utilized (e.g., when in complex with a guide nucleic acid sequence) to modulate (e.g., edit, activate, suppress) a target gene (e.g., heterologous gene, endogenous gene) in a cell. For example, the polypeptide molecule described herein can suppress a target gene.

The suppression of the heterologous target gene by the polypeptide molecules (as shown in Table 3 and Table 4) was tested using an engineered fluorescent reporter system with sgRNA landing pads under EF1a promoter driving constitutive GFP expression in a target cell. Specifically, seven tandem copies of a synthetic sgRNA binding site (ESR) were installed upstream of human EF1a promoter-GFP to test a gene suppression by the polypeptide molecules. Each sgRNA site contains dual PAM sequences for modulator recruitment via either dCas9 or dCasMINI. EF1a-GFP reporter cells stably expressing an ESR-targeting sgRNA were chemically transfected with polypeptide molecules-expressing plasmids, then samples were collected at different time points (e.g., 8 days, 15 days, 22 days, 30 days, and 44 days post transfection) for maximal transcriptional effect via FACS-based separation of GFP-ON and GFP-OFF cells.

As shown in Table 3 and Table 4, the polypeptide molecules were, in an N>C terminal direction, (1) dCasMini-Linker (A or B)-Modulator X-Linker (A or B)-Modulator Y, or (2) dCasMini-Linker (C)-Modulator X-Linker (A or B)-Modulator Y-Linker (A or B)-Modulator Z. The “Linker A” corresponds to the polypeptide sequence of SEQ ID NO: 37. The “Linker B” corresponds to the polypeptide sequence of SEQ ID NO: 1. The “Linker C” corresponds to the polypeptide sequence of SEQ ID NO: 43.

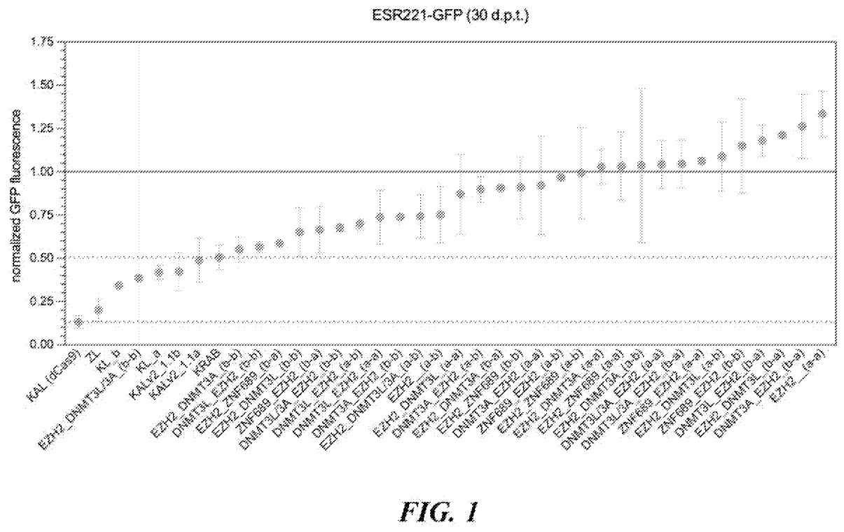

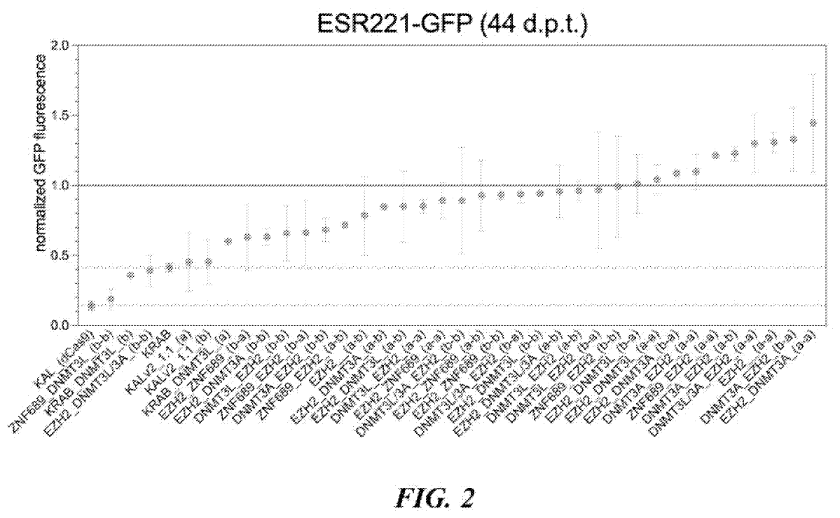

Referring to FIG. 1 and FIG. 2, plotted data displays relative GFP expression for dCas9- and dCasMini-canonical repressor controls (e.g., KRAB fusions) and the polypeptide molecules (as shown in Table 3 and Table 4) comprising (i) at least a portion of an endonuclease and a gene modulator coupled to one another via the polypeptide chain (e.g., “Linker B”, comprising the polypeptide sequence of SEQ ID NO: 1) and/or (ii) a gene modulator and an additional gene modulator (e.g., in conjunction with the at least a portion of an endonuclease) coupled to one another via the polypeptide chain. Some polypeptide molecules (e.g., KL_b, EZH2_DNMT3L/3A_(b-b), KL_a, KAL_(b), KAL_(a)) showed high potency (e.g., suppression) and high durability (e.g., persistency) even after 30 days (FIG. 1) and 44 days (FIG. 2) post transfection.

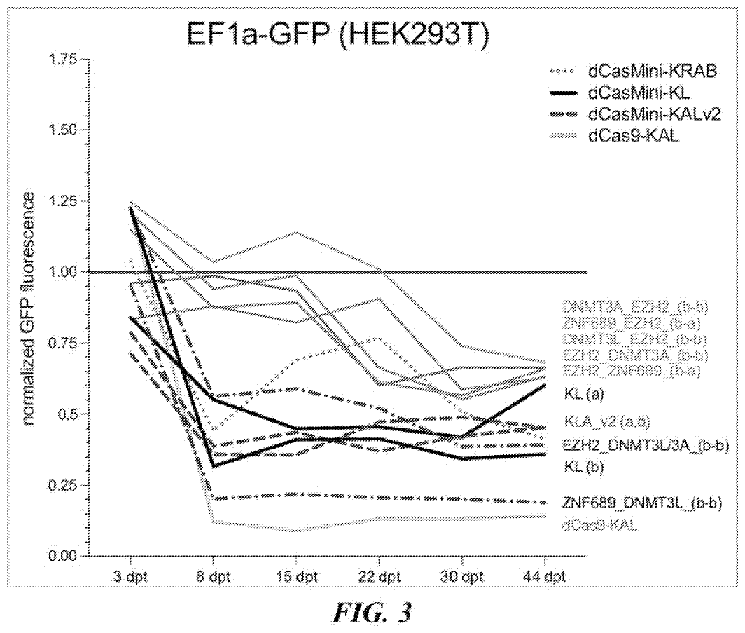

Referring to FIG. 3, plotted data displays relative GFP expression of the cells transfected with the polypeptide molecules (as shown in Table 3 and Table 4). Some of the heterologous polypeptide molecules comprising the polypeptide chain showed greater potency (e.g., suppression) and greater durability (e.g., persistency) when compared to a control (e.g., a polypeptide molecule in absence of the polypeptide chain). Specifically, some polypeptide molecules (e.g., KRAB_DNMT3L_(b) and KAL_(b), ZNF689_DNMT3L_(b-b), and EZH2_DNMT3L/3A_(b_b)) showed greater suppression that persisted up to 44 days post-transfection when compared to dCasMini-canonical repressor control (e.g., dCasMini-KRAB).

### B. Suppression of an Endogenous Target Gene

Cells were co-transfected with polypeptide molecule—(as shown in Table 3 and Table 4) and sgRNA (sgCXCR4)-expressing plasmids. 28 days post transfection, cells were collected and stained with CXCR4-APC conjugated antibody (BioLegend) for flow cytometry (Cytoflex LX) analysis to monitor CXCR4 protein expression.

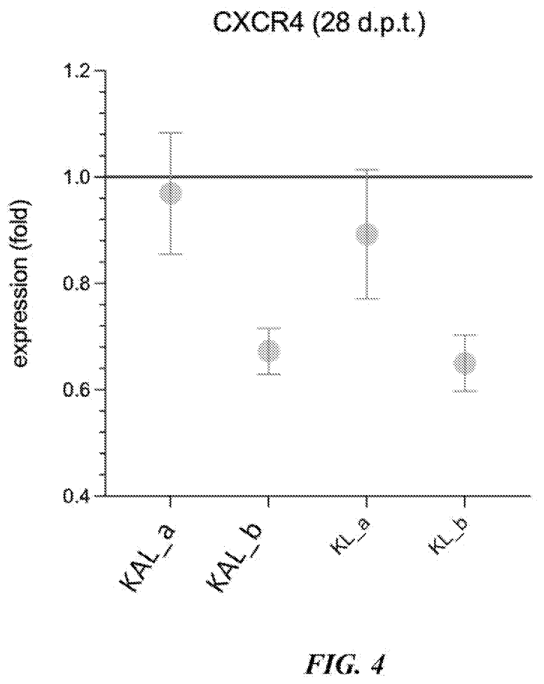

Referring to FIG. 4, plotted data displays relative CXCR4 expression for the polypeptide molecule comprising dCasMINI and one or more gene modulators in absences of the polypeptide chain for comparison against the polypeptide molecule comprising dCasMINI and one or more gene modulators coupled to the polypeptide chain. The polypeptide molecules comprising the polypeptide chain (e.g., KAL_a and KL_a) showed greater potency (e.g., suppression) and greater durability (e.g., persistency) compared to the polypeptide molecules that do not comprise the polypeptide chain (e.g., KAL_a and KL_a).

TABLE 3
Non-limiting examples of gene modulator and linker combination.

| Design | Modulator X | Modulator Y | Annotation |
|---|---|---|---|
| dCasMINI-(Linker A)-X-(Linker A)-Y | EZH2 | ZNF689 | EZH2_ZNF689_(a-a) |
| dCasMINI-(Linker A)-X-(Linker A)-Y | ZNF689 | EZH2 | ZNF689_EZH2_(a-a) |
| dCasMINI-(Linker A)-X-(Linker A)-Y | EZH2 | DNMT3A | EZH2_DNMT3A_(a-a) |
| dCasMINI-(Linker A)-X-(Linker A)-Y | DNMT3A | EZH2 | DNMT3A_EZH2_(a-a) |
| dCasMINI-(Linker A)-X-(Linker A)-Y | EZH2 | DNMT3L | EZH2_DNMT3L_(a-a) |
| dCasMINI-(Linker A)-X-(Linker A)-Y | DNMT3L | EZH2 | DNMT3L_EZH2_(a-a) |
| dCasMINI-(Linker A)-X-(Linker A)-Y | DNMT3L/3A | EZH2 | DNMT3L/3A_EZH2_(a-a) |
| dCasMINI-(Linker A)-X-(Linker A)-Y | EZH2 | | EZH2_(a-a) |
| dCasMINI-(Linker A)-X-(Linker B)-Y | EZH2 | ZNF689 | EZH2_ZNF689_(a-b) |
| dCasMINI-(Linker A)-X-(Linker B)-Y | ZNF689 | EZH2 | ZNF689_EZH2_(a-b) |
| dCasMINI-(Linker A)-X-(Linker B)-Y | EZH2 | DNMT3A | EZH2_DNMT3A _(a-b) |
| dCasMINI-(Linker A)-X-(Linker B)-Y | DNMT3A | EZH2 | DNMT3A_EZH2_(a-b) |
| dCasMINI-(Linker A)-X-(Linker B)-Y | EZH2 | DNMT3L | EZH2_DNMT3L _(a-b) |
| dCasMINI-(Linker A)-X-(Linker B)-Y | DNMT3L | EZH2 | DNMT3L_EZH2_(a-b) |
| dCasMINI-(Linker A)-X-(Linker B)-Y | EZH2 | DNMT3L/3A | EZH2_DNMT3L/3A_(a-b) |
| dCasMINI-(Linker A)-X-(Linker B)-Y | EZH2 | | EZH2_(b-a) |
| dCasMINI-(Linker B)-X-(Linker A)-Y | EZH2 | ZNF689 | EZH2_ZNF689_(b-a) |
| dCasMINI-(Linker B)-X-(Linker A)-Y | ZNF689 | EZH2 | ZNF689_EZH2_(b-a) |
| dCasMINI-(Linker B)-X-(Linker A)-Y | EZH2 | DNMT3A | EZH2_DNMT3A _(b-a) |
| dCasMINI-(Linker B)-X-(Linker A)-Y | DNMT3A | EZH2 | DNMT3A_EZH2_(b-a) |
| dCasMINI-(Linker B)-X-(Linker A)-Y | EZH2 | DNMT3L | EZH2_DNMT3L _(b-a) |
| dCasMINI-(Linker B)-X-(Linker A)-Y | DNMT3L | EZH2 | DNMT3L_EZH2_(b-a) |
| dCasMINI-(Linker B)-X-(Linker A)-Y | DNMT3L/3A | EZH2 | DNMT3L/3A_EZH2_(b-a) |
| dCasMINI-(Linker B)-X-(Linker B)-Y | EZH2 | ZNF689 | EZH2_ZNF689_(b-b) |
| dCasMINI-(Linker B)-X-(Linker B)-Y | ZNF689 | EZH2 | ZNF689_EZH2_(b-b) |
| dCasMINI-(Linker B)-X-(Linker B)-Y | EZH2 | DNMT3A | EZH2_DNMT3A _(b-b) |
| dCasMINI-(Linker B)-X-(Linker B)-Y | DNMT3A | EZH2 | DNMT3A_EZH2_(b-b) |
| dCasMINI-(Linker B)-X-(Linker B)-Y | EZH2 | DNMT3L | EZH2_DNMT3L _(b-b) |
| dCasMINI-(Linker B)-X-(Linker B)-Y | DNMT3L | EZH2 | DNMT3L_EZH2_(b-b) |
| dCasMINI-(Linker B)-X-(Linker B)-Y | EZH2 | DNMT3L/3A | DNMT3L/3A_EZH2_(b-b) |
| dCasMINI-(Linker B)-X-(Linker B)-Y | DNMT3L/3A | EZH2 | DNMT3L/3A_EZH2_(b-b) |
| dCasMINI-(Linker B)-X-(Linker B)-Y | ZNF689 | DNMT3L | ZNF689_DNMT3L_(b-b) |

TABLE 4
Non-limiting examples of gene modulator(s) and linker combination.

| Design | Modulator X | Modulator Y | Modulator Z | Annotation |
|---|---|---|---|---|
| Z-(Linker A)-dCas9- | DNMT3L | DNMT3A | KRAB | KAL_(dCas9) |
| (Linker C)-X-(Linker A)-Y | | | | |
| dCasMINI-(Linker C)-X- | KRAB | DNMT3A | DNMT3L | KAL(a) |
| (Linker A)-Y-(Linker A)-Z | | | | |
| dCasMINI-(Linker C)-X- | KRAB | DNMT3A | DNMT3L | KAL(b) |
| (Linker B)-Y-(Linker A)-Z | | | | |
| dCasMINI-(Linker C)-X- | KRAB | DNMT3L | | KRAB_DNMT3L_(a) |
| (Linker A)-Y | | | | or KL_a |
| dCasMINI-(Linker C)-X- | KRAB | DNMT3L | | KRAB_DNMT3L_(b) |
| (Linker B)-Y | | | | or KL_b |
| dCasMINI-(Linker C)-X | KRAB | | | KRAB |
| dCasMINI-(Linker C)-X- | ZNF689 | DNMT3L | | ZL |
| (Linker A)-Y | | | | |

It shall be understood that different aspects of the invention can be appreciated individually, collectively, or in combination with each other. Various aspects of the invention described herein may be applied to any of the particular applications disclosed herein. The compositions of matter disclosed herein in the composition section of the present disclosure may be utilized in the method section including methods of use and production disclosed herein, or vice versa.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A polypeptide molecule comprising a polypeptide chain exhibiting at least about 73% identity to the polypeptide sequence of SEQ ID NO: 1.

2.-141. (canceled)


SEQUENCE LISTING (350 sequences)

SEQ ID NO: 1 - protein, 25 aa, synthetic construct
GKESGSVGGSGGSSEQLAQFRSLDG

SEQ ID NO: 2 - protein, 18 aa, synthetic construct
KESGSVSSEQLAQFRSLD

SEQ ID NO: 3 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 4 - protein, 479 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNEKCNFKENAAYNAALNISNPKLKSTKERP

SEQ ID NO: 5 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 71 aa, synthetic construct
DAKSLTAWSRTLVTFKDVFVDFTREEWKLLDTAQQIVYRNVMLENYKNLVSLGYQLTKPDVILRLEKGEEP

SEQ ID NO: 7 - protein, 96 aa, synthetic construct
DAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 8 - protein, 322 aa, synthetic construct
TYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHADRPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 9 - protein, 380 aa, synthetic construct
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFEMEPVTIPDVHGGSLQNAVRVWSNIPAIRSRHWALVSELEELSLLAQNKQSSKLAAKWPTKLVKNCFLPLREYFKYFST

SEQ ID NO: 10 - protein, 746 aa, synthetic construct
MGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 11 - protein, 85 aa, synthetic construct
APPSAPLPAQGPGKARPSRKRGRRPRALKFVDVAVYFSPEEWGCLRPAQRALYRDVMRETYGHLGALGCAGPKPALISWLERNTD

SEQ ID NO: 12 - protein, 714 aa, synthetic construct
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFEMEPVTIPDVHGGSLQNAVRVWSNIPAIRSRHWALVSELEELSLLAQNKQSSKLAAKWPTKLVKNCFLPLREYFKYFSTGSGGSGGSGGSGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHADRPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 13 - protein, 11 aa, synthetic construct
DALDDFDLDML

SEQ ID NO: 14 - protein, 50 aa, synthetic construct
DALDDFDLDMLGSDALDDFDLDMLGSDALDDFDLDMLGSDALDDFDLDML

SEQ ID NO: 15 - protein, 265 aa, synthetic construct
QYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 16 - protein, 190 aa, synthetic construct
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 17 - protein, 470 aa, synthetic construct
DALDDFDLDMLGSDALDDFDLDMLGSDALDDFDLDMLGSDALDDFDLDMLGSGGSGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTL

SEQ ID NO: 18 - protein, 7 aa, synthetic construct
PKKKRKV

SEQ ID NO: 19 - protein, 10 aa, synthetic construct
PKKKRKVEAS

SEQ ID NO: 20 - protein, 11 aa, synthetic construct
PKKKRKVGSGS

SEQ ID NO: 21 - protein, 9 aa, synthetic construct
PAAKRVKLD

SEQ ID NO: 22 - protein, 11 aa, synthetic construct
RQRRNELKRSP

SEQ ID NO: 23 - protein, 13 aa, synthetic construct
PAAKRVKLDGSGS

SEQ ID NO: 24 - protein, 16 aa, synthetic construct
KRPAATKKAGQAKKKK

SEQ ID NO: 25 - protein, 38 aa, synthetic construct
NQSSNFGPMKGGNFGGRSSGPYGGGGQYFAKPRNQGGY

SEQ ID NO: 26 - protein, 42 aa, synthetic construct
RMRIZFKNKGKDTAELRRRRVEVSVELRKAKKDEQILKRRNV

SEQ ID NO: 27 - protein, 8 aa, synthetic construct
VSRKRPRP

SEQ ID NO: 28 - protein, 8 aa, synthetic construct
PPKKARED

SEQ ID NO: 29 - protein, 8 aa, synthetic construct
PQPKKKPL

SEQ ID NO: 30 - protein, 12 aa, synthetic construct
SALIKKKKKMAP

SEQ ID NO: 31 - protein, 5 aa, synthetic construct
DRLRR

SEQ ID NO: 32 - protein, 7 aa, synthetic construct
PKQKKRK

SEQ ID NO: 33 - protein, 10 aa, synthetic construct
RKLKKKIKKL

SEQ ID NO: 34 - protein, 10 aa, synthetic construct
REKKKFLKRR

SEQ ID NO: 35 - protein, 20 aa, synthetic construct
KRKGDEVDGVDEVAKKKSKK

SEQ ID NO: 36 - protein, 17 aa, synthetic construct
RKCLQAGMNLEARKTKK

SEQ ID NO: 37 - protein, 12 aa, synthetic construct
GSGGSGGSGGSG

SEQ ID NO: 38 - protein, 5 aa, synthetic construct
GGGGS

SEQ ID NO: 39 - protein, 4 aa, synthetic construct
GGGS

SEQ ID NO: 40 - skipped

SEQ ID NO: 41 - protein, 14 aa, synthetic construct
EGKSSGSGSESKST

SEQ ID NO: 42 - protein, 12 aa, synthetic construct
GSAGSAAGSGEF

SEQ ID NO: 43 - protein, 52 aa, synthetic construct
AYPYDVPDYASLGSGSPKKKRKVEDPKKKRKVDGIGSGSSGSSGSGGGSGTR

SEQ ID NO: 44 - protein, 80 aa, synthetic construct
GGPSSGAPPPSGGSPAGSPTSTEEGTSESATPESGPGTSTEPSEGSAPGSPAGSPTSTEEGTSTEPSEGSAPGTSTEPSE

SEQ ID NO: 45 - protein, 54 aa, synthetic construct
GGGGSKESGSVSSEQLAQFRSLDEGKSSGSGSESKSTGSAGSAAGSGEFGGGGS

SEQ ID NO: 46 - protein, 51 aa, synthetic construct
YPYDVPDYASLGSGSPKKKRKVEDPKKKRKVDGIGSGSNGSSGSGGGSGTR

SEQ ID NO: 47 - protein, 5 aa, synthetic construct
EAAAK

SEQ ID NO: 48 - protein, 5 aa, synthetic construct
EAAAR

SEQ ID NO: 49 - protein, 5 aa, synthetic construct
PAPAP

SEQ ID NO: 50 - protein, 12 aa, synthetic construct
AEAAAKEAAAKA

SEQ ID NO: 51 - protein, 529 aa, synthetic construct; identical to SEQ ID NO: 3

SEQ ID NO: 52 - protein, 500 aa, synthetic construct
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 53 - protein, 422 aa, synthetic construct
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 54 - protein, 509 aa, synthetic construct
MEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 55 - protein, 499 aa, synthetic construct
MNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 56 - protein, 509 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENA

SEQ ID NO: 57 - protein, 499 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFK

SEQ ID NO: 58 - protein, 509 aa, synthetic construct
MAKNTITKTLKLRIVRPYNSAEVEKIVADEKNNREKIALEKNKDKVNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 59 - protein, 498 aa, synthetic construct
MAKNTITKTLKLRIVRPYNSAEVEKIVADEKNNREKIALECKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 60 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 61 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 62 - protein, 544 aa, synthetic construct
MNMSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 63 - protein, 586 aa, synthetic construct
MPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 64 - protein, 603 aa, synthetic construct
MKSFKLKLLPTDEQNVLLNEVFCKWASLCTRMASKGHDKERLAPPDSSGNYFNKTQLNQVNTDVTDHMGALEESASQKERAVEKVKRRLKLISDMLSEPNLRDVSQQKPTTFRPLEWVKEGLLKTKYHTVHYWQKECDKLTKQKERMEKTIEKIKKGKITFKPTKMSLHQNCFSLSFGKGTFSMRPFSDTKRGINLDMLTAPIQPAIGKNDGKSSLRSKEFIARNIENYIIFSIHSQLFGLSRSEELLLNAKKEELVAKRDAMLKKKSDSLSKKIKELEKIVGRKITDSERSEIMSQGGKLSSEKFSEDNSYLKTLKVLAKDIIGREELFRLKKYPIVIRKPLNERKKLKNLKPDEWEYYLQLSYDELEKKEFTPKTIMGIDRGLKHILAIAIYDPVQNKFVKNMLIPNPILGWKWKLRKIKRSIQHMERRIRAQQNAHVPENQLKKRLKSIENKIDYYYHNVSRQILNLAHDFKSAIVVEDLQNMKQHGRKKSKGLRGLNYALSNFDYGKIMGLVKYKAESENVPLLTVLPAGTSQNCAYCLLYGKEQGNYVRNNVNSKIGKCKLHGEIDADINAARTIAICYHKNINEPKPYGERKTFKRK

SEQ ID NO: 65 - protein, 424 aa, synthetic construct
MKYTKVMRYQIIKPLNAEWDELGMVLRDIQKETRAALNKTIQLCWEYQGFSADYKQIHGQYPKPKDVLGYTSMHGYAYDRLKNEFSKIASSNLSQTIKRAVDKWNSDLKEILRGDRSIPNFRKDCPIDIVKQSTKIQKCNDGYVLSLGLINREYKNELGRKNGVFDVLIKANDKTQQTILERIINGDYTYTASQIINHKNKWFINLTYQFETKETALDPNNVMGVDLGIVYPVYIAFNNSLHRYHIKGGEIERFRRQVEKRKRELLNQGKYCGDGRKGHGYATRTKSIESISDKIARFRDTCNHKYSRFIVDMALKHNCGIIQMEDLTGISKESTFLKNWTYYDLQQKIEYKAREAGIQVIKIEPQYTSQRCSKCGYIDKENRQEQATFKCIECGFKTNADYNAARNIAIPNIDKIIRKTLKMQ

SEQ ID NO: 66 - protein, 443 aa, synthetic construct
MTLLVKVVKIHLISEQFDKAGNRIDYEEVNKILWELQKQTREAKNKTVQLLWEWNNFSSDYVKASGIYPKAKDIFGYSSVHGQANKELRTKLALNSSNLSTTTMDVCKNFNTYKKEVWKGKRSVPSYKSDQPLDLHKDSIKLIYENNEFYVRLALLKKAEFAKYGFKDGFRFKMQVKDNSTKTILERCFDEVYKINASKLLYDQKKKKWKLNLSYSFDNKNISELDKEKILGVDVGVNCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKSDRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 67 - protein, 497 aa, synthetic construct
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 68 - protein, 497 aa, synthetic construct
MITVRKIKLTIMGDKDTRNSQYKWIRDEQYNQYRALNMGMTYLAVNDILYMNESGLEIRTIKDLKDCEKDIDKNKKEIEKLTARLEKEQNKKNSSSEKLDEIKYKISLVENKIEDYKLKIVELNKILEETQKERMDIQKEFKEKYVDDLYQVLDKIPFKHLDNKSLVTQRIKADIKSDKSNGLLKGERSIRNYKRNFPLMTRGRDLKFKYDDNDDIEIKWMEGIKFKVILGNRIKNSLELRHTLHKVIEGKYKICDSSLQFDKNNNLILNLTLDIPIDIVNKKVSGRVVGVDLGLKIPAYCALNDVEYIKKSIGRIDDFLKVRTQMQSRRRRLQIAIQSAKGGKGRVNKLQALERFAEKEKNFAKTYNHFLSSNIVKFAVSNQAEQINMELLSLKETQNKSILRNWSYYQLQTMIEYKAQREGIKVKYIDPYHTSQTCSKCGNYEEGQRESQADFICKKCGYKVNADYNAARNIAMSNKYITKKEESKYYKIKESMV

SEQ ID NO: 69 - protein, 495 aa, synthetic construct
MAKNTITKTLKLRIVRPYNSAEVEKIVADEKERRKQAGGTGELDGGFYKKLEKKHSEMFSFDRLNLLLNQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 70 - protein, 496 aa, synthetic construct
MEVQKTVMKTLSLRILRPLYSQEIEKEIKEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 71 - protein, 495 aa, synthetic construct
MAKNTITKTLKLRIVRPLYSQEIEKEIKEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 72 - protein, 451 aa, synthetic construct
MIKVYRYEIVKPLDLDWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 73 - protein, 485 aa, synthetic construct
MITVRKIKLTIMGDKDTRNSQYKWIRDEQYNQYRALNMGMTYLAVNDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 74 - protein, 462 aa, synthetic construct
MGESVKAIKLKILDMFLDPECTKQDDNWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 75 - protein, 453 aa, synthetic construct
MKYTKVMRYQIIKPLNAEWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 76 - protein, 460 aa, synthetic construct
MTLLVKVVKIHLISEQFDKAGNRIDYEEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 77 - protein, 529 aa, synthetic construct
MAKNTITKTLKLRIVRPYYSQEIEKIVAEEKNRREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 78 - protein, 529 aa, synthetic construct
MAKNTITKTLKLRIVRPYYSAEVEKIVAEEKNNREKIALDKNKDKVKEACSKHLKVAAYCTTQVERNACLFCKARKLDDKFYKKLRGQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 79 - protein, 529 aa, synthetic construct
MAKNTITKTLKLRIVRPYYSAEIEKIVADEKNRREKIALEKNKDKVKEACSKHLKVAAYCTTQVERNACLFCKARKLDDKFYQKLRKQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 80 - protein, 529 aa, synthetic construct
MAKNTITKTLKLRIVRPYNSQEVEKIVAEEKNRREKIALDKNKDKVKEACSKHLKVAAYCTTQVERNACLFCKARKLDDKFYQKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 81 - protein, 529 aa, synthetic construct
MAKNTITKTLKLRIVRPYNSQEVEKIVAEEKNNREKIALDKNKDKVKEACSKHLKVAAYCTTQVERNACLFCKARKLDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 82 - protein, 529 aa, synthetic construct
MAKNTITKTLKLRIVRPYNSQEVEKIVAEEKNNREKIALDKNKDKVKEACSKHLKVAAYCTTQVERNACLFCKARKLDDKFYKKLRGQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 83 - protein, 529 aa, synthetic construct
MAKNTITKTLKLRIVRPYYSAEVEKIVAEEKNNREKIALDKNKDKVKEACSKHLKVAAYCTTQVERNACLFCKARKLDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 84 - protein, 529 aa, synthetic construct
MAKNTITKTLKLRIVRPYNSAEIEKIVADEKNRREKIALDKNKDKVKEACSKHLKVAAYCTTQVERNACLFCKARKLDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 85 - protein, 529 aa, synthetic construct
MAKNTITKTLKLRIVRPYNSAEIEKIVADEKNRREKIALDKNKDKVKEACSKHLKVAAYCTTQVERNACLFCKARKLDDKFYKKLRGQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 86 - protein, 529 aa, synthetic construct
MAKNTITKTLKLRIVRPYNSQEIEKIVAEEKNRREKIALDKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 87 - protein, 529 aa, synthetic construct
MAKNTITKTLKLRIVRPYNSAEIEKIVAEEKNRREKIALDKNKDKVKEACSKHLKVAAYCTTQVERNACLFCKARKLDDKFYKKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 88 - protein, 529 aa, synthetic construct
MAKNTITKTLKLRIVRPYNSAEVEKIVAEEKNRREKIALDKNKDKVKEACSKHLKVAAYCTTQVERNACLFCKARKLDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 89 - protein, 529 aa, synthetic construct
MAKNTITKTLKLRIVRPYNSAEVEKIVADEKNRREKIALDKNKDKVKEACSKHLKVAAYCTTQVERNACLFCKARKLDDKFYKKLRKQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 90 - protein, 529 aa, synthetic construct
MAKNTITKTLKLRIVRPYYSAEIEKIVADEKNRREKIALDKNKDKVKEACSKHLKVAAYCTTQVERNACLFCKARKLDDKFYQKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 91 - protein, 529 aa, synthetic construct
MAKNTITKTLKLRIVRPYYSAEIEKIVAEEKNRREKIALEKNKDKVKEACSKHLKVAAYCTTQVERNACLFCKARKLDDKFYQKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 92 - protein, 529 aa, synthetic construct
MAKNTITKTLKLRIVRPYYSAEIEKIVAEEKNNREKIALDKNKDKVKEACSKHLKVAAYCTTQVERNACLFCKARKLDDKFYQKLRGQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 93 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAARLFKNAAIASGLRSKIKSNFRLKELKNMKSGLPTTKSDNFPIPLVKQKGGQYTGFKISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 94 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFKISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 95 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAGLFKNAAIASGLRSKIKSNFRLKELKNMKSGLPTTKSDNFPIPLVKQKGGQYTGFKISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 96 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAARLFKNAAIASGLRSKIKSNFRLKELKNMKSGLPTTKSDNFPIPLVKQKGGQYTGFRISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 97 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFRISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 98 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAGLFKNAAIASGLRSKIKSNFRLKELKNMKSGLPTTKSDNFPIPLVKQKGGQYTGFRISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 99 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAARLFKNAAIASGLRSKIKSNFRLKELKNMKSGLPTTKSDNFPIPLVKQKGGQYTGFSISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 100 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFSISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 101 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAGLFKNAAIASGLRSKIKSNFRLKELKNMKSGLPTTKSDNFPIPLVKQKGGQYTGFSISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 102 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLENFNKKMFARRRILLKKNRHKRGGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 103 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIEGGDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 104 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIEGGDLENFNKKMFARRRILLKKNRHKRGGHGRDKKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 105 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIEGGDLENFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 106 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGRDKKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 107 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLENFNKKMFARRRILLKKNRHKRAGHGRDKKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 108 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLEHFNKKMFARRRILLKKNRHKRKGHGAKNKLKPIETLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 109 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDGGDLEHFNKKMFARRRILLKKNRHKRKGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 110 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDGGDLFHFNKKMFARRRILLKKNRHKRAGHGAKNKLKPIETLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 111 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDGGDLEHFNKKMFARRRILLKKNRHKRKGHGAKNKLKPIETLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 112 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSIDSNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 113 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSIDSNDLFHFNKKMFARRRILLKKNRHKRAGHGAAHKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 114 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSIDSNDLFKFNKKMFARRRILLKKNRHKRAGHGAAHKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 115 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDSNDLFKFNKKMFARRRILLKKNRHKRAGHGAAHKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 116 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSIDSNDLFKFNKKMFARRRILLKKNRHKRAGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 117 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 118 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDSNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 119 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFKFNKKMFARRRILLKKNRHKRKGHGAAHKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 120 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLERFNKKMFARRRILLKKNRHKRKGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 121 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLERFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 122 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLEKFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 123 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLFHFNKKMFARRRILLKKNRHKRAGHGRKKKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 124 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLEKFNKKMFARRRILLKKNRHKRAGHGRKKKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 125 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLEKFNKKMFARRRILLKKNRHKRAGHGRKKKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 126 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWAKEIADFFIKNKVGTVQMEDLSTMKRKEDSYFNIRLRGFWPYYEMQNKIEFKLKQYGIEIRKVAPNNTSQLCSKCGHLNNYFNFEYRKKNKFPKFKCEKCNFKENAAYNAARNISTPDIKSTKERP

SEQ ID NO: 127 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 128 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWAKEIADFFIKNKVGTVQMEDLSTMKRKEDSYFNIRLRGFWPYYEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 129 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 130 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISTPDIKSTKERP

SEQ ID NO: 131 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLSTMKRKEDSYFNIRLRGFWPYYEMQNKIEFKLKQYGIEIRKVAPNNTSKTCSKCGHLNNYFNFEYRKKNKFPKFKCEKCNFKENAAYNAALNISNPKLKSTKERP

SEQ ID NO: 132 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 133 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPKFKCEKCNFKENAAYNAARNISTPDIKSTKERP

SEQ ID NO: 134 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWSRYIADFFIKNKVGTVQMEDLESMKRKEDSYFNIRLRGFWPYYEMQNKIEFKLKQYGIKIRKVAPNNTSQRCSKCGHLNNYFNFEYRKKNKFPHFKCEKCNFKANAAYNAARNISNPNIKSTKERP

SEQ ID NO: 135 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACYIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 136 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACYIADFFIKNKVGTVQMENLESMKRKEDSYFNIRLRGFWPYAEMQNKIEFKLKQYGIEIRKVAPNNTSQRCSKCGHLNNYFNFEYRKKNKFPHFKCEKCNFKRNAAYNAARNISNPKLKSTKERP

SEQ ID NO: 137 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACYIADFFIKNKVGTVQMEDLESMKRKEDSYFNIRLRGFWPYAEMQNKIEFKLKQYGIEIRKVAPNNTSKTCSKCGHLNNYFNFEYRKKNKFPHFKCEKCNFKENAAYNAARNISNPNIKSTKERP

SEQ ID NO: 138 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWARYIADFFIKNKVGTVQMEDLESMKRKEDSYFNIRLRGFWPYYEMQNKIEFKLKQYGIKIRKVAPNNTSKTCSKCGHLNNYFNFEYRKKNKFPHFKCEKCNFKENAAYNAALNISNPKLKSTKERP

SEQ ID NO: 139 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQRCSKCGHLNNYFNFEYRKKNKFPHFKCEKCNFKRNAAYNAARNISNPNIKSTKERP

SEQ ID NO: 140 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 141 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKRNAAYNAARNISNPNIKSTKERP

SEQ ID NO: 142 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWANRIADFFIKNKVGTVQMENLESMKRKEDSYFNIRLRGFWPYAEMQNKIEFKLKQYGIKIRKVAPNNTSQRCSKCGHLNNYFNFEYRKKNKFPHFKCEKCNFKRNAAYNAAKNISNPKLKSTKERP

SEQ ID NO: 143 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQRCSKCGHLNNYFNFEYRKKNKFPHFKCEKCNFKRNAAYNAAKNISNPKLKSTKERP

SEQ ID NO: 144 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIKIRKVAPNNTSQRCSKCGHLNNYFNFEYRKKNKFPHFKCEKCNFKRNAAYNAAKNISNPKLKSTKERP

SEQ ID NO: 145 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWANRIADFFIKNKVGTVQMENLESMKRKEDSYFNIRLRGFWPYAEMQNKIEFKLKQYGIKIRKVAPNNTSQRCSKCGHLNNYFNFEYRKKNKFPHFKCEKCNFKENAAYNAALNISNPKLKSTKERP

SEQ ID NO: 146 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWANRIADFFIKNKVGTVQMENLESMKRKEDSYFNIRLRGFWPYAEMQNKIEFKLKQYGIEIRKVAPNNTSQRCSKCGHLNNYFNFEYRKKNKFPHFKCEKCNFKRNAAYNAALNISNPKLKSTKERP

SEQ ID NO: 147 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWSRFIADFFIKNKVGTVQMEDLESMKRKEDSYFNIRLRGFWPYYEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 148 - protein, 514 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIDVQLYSKEYSKDSGKGTHRYFLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 149 - protein, 514 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIASLSLLSNPAKQEMNVKRKISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 150 - protein, 540 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPIYERKPNRSIVGGLAVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISAAYNAARNLSTPDIEKFVAKATKGINLPEK

SEQ ID NO: 151 - protein, 590 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPTHETVLDPDKVMGVALGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNAAYNAAKNISTPYIDQIIMEKCLELGIPYRSITCKECGHIQASGNTCEVCGSTNILKPKK

SEQ ID NO: 152 - protein, 522 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPQTRVLDLNKIMGIALGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANAAYNAARNIAIPNIDKIIAESIK

SEQ ID NO: 153 - protein, 532 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPIDIVNKKVSGRVVGVALGLKIPAYCALNDVEYIKKSIGRIDDFLKVRTQMQSRRRRLQIAIQSAKGGKGRVNKLQALERFAEKEKNFAKTYNHFLSSNIVKFAVSNQAEQINMELLSLKETQNKSILRNWSYYQLQTMIEYKAQREGIKVKYIDPYHTSQTCSKCGNYEEGQRESQADFICKKCGYKVNAAYNAARNIAMSNKYITKKEESKYYKIKESMV

SEQ ID NO: 154 - protein, 524 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVETKETALDPNNVMGVALGIVYPVYIAFNNSLHRYHIKGGEIERFRRQVEKRKRELLNQGKYCGDGRKGHGYATRTKSIESISDKIARFRDTCNHKYSRFIVDMALKHNCGIIQMEDLTGISKESTFLKNWTYYDLQQKIEYKAREAGIQVIKIEPQYTSQRCSKCGYIDKENRQEQATFKCIECGFKTNAAYNAARNIAIPNIDKIIRKTLKMQ

SEQ ID NO: 155 - protein, 540 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVNRSIVGGLAVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISAAYNAARNLSTPDIEKFVAKATKGINLPEK

SEQ ID NO: 156 - protein, 590 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPDKVMGVALGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNAAYNAAKNISTPYIDQIIMEKCLELGIPYRSITCKECGHIQASGNTCEVCGSTNILKPKK

SEQ ID NO: 157 - protein, 523 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDLNKIMGIALGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANAAYNAARNIAIPNIDKIIAESIK

SEQ ID NO: 158 - protein, 524 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPNNVMGVALGIVYPVYIAFNNSLHRYHIKGGEIERFRRQVEKRKRELLNQGKYCGDGRKGHGYATRTKSIESISDKIARFRDTCNHKYSRFIVDMALKHNCGIIQMEDLTGISKESTFLKNWTYYDLQQKIEYKAREAGIQVIKIEPQYTSQRCSKCGYIDKENRQEQATFKCIECGFKTNAAYNAARNIAIPNIDKIIRKTLKMQ

SEQ ID NO: 159 - protein, 535 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDKEKILGVAVGVNCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKSDRFLKDWTYYDLQTKIENKAKEVGINVVYIAPKYTSQRCSKCGYIHKDNRPNQAKFRCLECDFESNAAYNASQNIGIKNIDKIIEKDLQKQESEVQVNENK

SEQ ID NO: 160 - protein, 517 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 161 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 162 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 163 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 164 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 165 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 166 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 167 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 168 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 169 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 170 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 171 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 172 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 173 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 174 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 175 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 176 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 177 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 178 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 179 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 180 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 181 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 182 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 183 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 184 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 185 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 186 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 187 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 188 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 189 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 190 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 191 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 192 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 193 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 194 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 195 - protein, 495 aa, synthetic construct; identical to SEQ ID NO: 61

SEQ ID NO: 196 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 197 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 198 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 199 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 200 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 201 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 202 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 203 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 204 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 205 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 206 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 207 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 208 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 209 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 210 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 211 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 212 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 213 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 214 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 215 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 216 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 217 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 218 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 219 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 220 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 221 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 222 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 223 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 224 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 225 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 226 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 227 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 228 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 229 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 230 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 231 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 232 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 233 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 234 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 235 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 236 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 237 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 238 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 239 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 240 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 241 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 242 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 243 - protein, 495 aa, synthetic construct
MAKNTITKTLKLRIVRPYNSAEIEKIVADEKERRKQAGGTGELDDKFYKKLRKQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 244 - protein, 495 aa, synthetic construct
MAKNTITKTLKLRIVRPYNSAEIEKIVADEKERRKQAGGTGELDDKFYKKLRKQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 245 - protein, 495 aa, synthetic construct
MAKNTITKTLKLRIVRPYNSAEIEKIVADEKERRKQAGGTGELDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 246 - protein, 495 aa, synthetic construct
MAKNTITKTLKLRIVRPYNSAEIEKIVADEKERRKQAGGTGELDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 247 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 248 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPKLKSTKERP

SEQ ID NO: 249 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPDIKSTKERP

SEQ ID NO: 250 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 251 - RNA, 159 nt, synthetic construct
  source 1..159: mol_type = other RNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 252 - RNA, 139 nt, synthetic construct
  source 1..139: mol_type = other RNA
gaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 253 - RNA, 138 nt, synthetic construct
  source 1..138: mol_type = other RNA
aaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 254 - RNA, 137 nt, synthetic construct
  source 1..137: mol_type = other RNA
accgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 255 - RNA, 136 nt, synthetic construct
  source 1..136: mol_type = other RNA
ccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 256 - RNA, 151 nt, synthetic construct
  source 1..151: mol_type = other RNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaagcaataaggaatgcaac

SEQ ID NO: 257 - RNA, 148 nt, synthetic construct
  source 1..148: mol_type = other RNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaagaaaggaatgcaac

SEQ ID NO: 258 - RNA, 157 nt, synthetic construct
  source 1..157: mol_type = other RNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaatcttcggattaaggaatgcaac

SEQ ID NO: 259 - RNA, 153 nt, synthetic construct
  source 1..153: mol_type = other RNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattgcaaaaggaatgcaac

SEQ ID NO: 260 - RNA, 153 nt, synthetic construct
  source 1..153: mol_type = other RNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcgttaaggaatgcaac

SEQ ID NO: 261 - RNA, 151 nt, synthetic construct
  source 1..151: mol_type = other RNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaatgcaaaggaatgcaac

SEQ ID NO: 262 - RNA, 153 nt, synthetic construct
  source 1..153: mol_type = other RNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaagcaattaaggaatgcaac

SEQ ID NO: 263 - RNA, 149 nt, synthetic construct
  source 1..149: mol_type = other RNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaagaaaggaatgcaac

SEQ ID NO: 264 - RNA, 132 nt, synthetic construct
  source 1..132: mol_type = other RNA
ggcttcactgataaagtggagaaccgcttcacttagagtgaaggtgggctgcttgcatcagcctaatgtcgagaagtgctttcttcggaaagtaaccctcgaaacaaattcatttgaatgaaggaatgcaac

SEQ ID NO: 265 - RNA, 132 nt, synthetic construct
  source 1..132: mol_type = other RNA
ggcttcactgataaagtggagaaccgcttcacttcgagtgaaggtgggctgcttgcatcagcctaatgtcgagaagtgctttcttcggaaagtaaccctcgaaacaaattcatttgaatgaaggaatgcaac

SEQ ID NO: 266 - RNA, 131 nt, synthetic construct
  source 1..131: mol_type = other RNA
ggcttcactgataaagtggagaaccgcttcacttcggtgaaggtgggctgcttgcatcagcctaatgtcgagaagtgctttcttcggaaagtaaccctcgaaacaaattcatttgaatgaaggaatgcaac

SEQ ID NO: 267 - RNA, 134 nt, synthetic construct
  source 1..134: mol_type = other RNA
ggcttcactgataaagtggagaaccgcttcaccttaggagtgaaggtgggctgcttgcatcagcctaatgtcgagaagtgctttcttcggaaagtaaccctcgaaacaaattcatttgaatgaaggaatgcaac

SEQ ID NO: 268 - RNA, 134 nt, synthetic construct
  source 1..134: mol_type = other RNA
ggcttcactgataaagtggagaaccgcttcaccttcggagtgaaggtgggctgcttgcatcagcctaatgtcgagaagtgctttcttcggaaagtaaccctcgaaacaaattcatttgaatgaaggaatgcaac

SEQ ID NO: 269 - RNA, 136 nt, synthetic construct
  source 1..136: mol_type = other RNA
ggcttcactgataaagtggagaaccgcttcacgcttcggcagtgaaggtgggctgcttgcatcagcctaatgtcgagaagtgctttcttcggaaagtaaccctcgaaacaaattcatttgaatgaaggaatgcaac

SEQ ID NO: 270 - RNA, 135 nt, synthetic construct
  source 1..135: mol_type = other RNA
accgcttcaccaaaagctgtccttagggattagaacttgagtgaaggtgggctgcttgcatcagcctaatgtcgagaagtgctttcttcggaaagtaaccctcgaaacaaattcatttgaatgaaggaatgcaac

SEQ ID NO: 271 - RNA, 133 nt, synthetic construct
  source 1..133: mol_type = other RNA
accgcttcaccaaaagctgtcttaggattagaacttgagtgaaggtgggctgcttgcatcagcctaatgtcgagaagtgctttcttcggaaagtaaccctcgaaacaaattcatttgaatgaaggaatgcaac

SEQ ID NO: 272 - RNA, 131 nt, synthetic construct
  source 1..131: mol_type = other RNA
accgcttcaccaaaagctgtttagattagaacttgagtgaaggtgggctgcttgcatcagcctaatgtcgagaagtgctttcttcggaaagtaaccctcgaaacaaattcatttgaatgaaggaatgcaac

SEQ ID NO: 273 - RNA, 129 nt, synthetic construct
  source 1..129: mol_type = other RNA
accgcttcaccaaaagctgttagttagaacttgagtgaaggtgggctgcttgcatcagcctaatgtcgagaagtgctttcttcggaaagtaaccctcgaaacaaattcatttgaatgaaggaatgcaac

SEQ ID NO: 274 - RNA, 126 nt, synthetic construct
  source 1..126: mol_type = other RNA
accgcttcaccaaaagctttagagaacttgagtgaaggtgggctgcttgcatcagcctaatgtcgagaagtgctttcttcggaaagtaaccctcgaaacaaattcatttgaatgaaggaatgcaac

SEQ ID NO: 275 - RNA, 124 nt, synthetic construct
  source 1..124: mol_type = other RNA
accgcttcaccaaaagcttcggcacttgagtgaaggtgggctgcttgcatcagcctaatgtcgagaagtgctttcttcggaaagtaaccctcgaaacaaattcatttgaatgaaggaatgcaac

SEQ ID NO: 276 - RNA, 122 nt, synthetic construct
  source 1..122: mol_type = other RNA
accgcttcaccaaaagttcgcacttgagtgaaggtgggctgcttgcatcagcctaatgtcgagaagtgctttcttcggaaagtaaccctcgaaacaaattcatttgaatgaaggaatgcaac

SEQ ID NO: 277 - RNA, 120 nt, synthetic construct
  source 1..120: mol_type = other RNA
accgcttcaccaaaattcgtcttgagtgaaggtgggctgcttgcatcagcctaatgtcgagaagtgctttcttcggaaagtaaccctcgaaacaaattcatttgaatgaaggaatgcaac

SEQ ID NO: 278 - RNA, 118 nt, synthetic construct
  source 1..118: mol_type = other RNA
accgcttcaccaagttcgcttgagtgaaggtgggctgcttgcatcagcctaatgtcgagaagtgctttcttcggaaagtaaccctcgaaacaaattcatttgaatgaaggaatgcaac

SEQ ID NO: 279 - RNA, 116 nt, synthetic construct
  source 1..116: mol_type = other RNA
accgcttcaccaattcgttgagtgaaggtgggctgcttgcatcagcctaatgtcgagaagtgctttcttcggaaagtaaccctcgaaacaaattcatttgaatgaaggaatgcaac

SEQ ID NO: 280 - RNA, 114 nt, synthetic construct
  source 1..114: mol_type = other RNA
accgcttcaccattcgtgagtgaaggtgggctgcttgcatcagcctaatgtcgagaagtgctttcttcggaaagtaaccctcgaaacaaattcatttgaatgaaggaatgcaac

SEQ ID NO: 281 - RNA, 159 nt, synthetic construct; identical to SEQ ID NO: 251
  source 1..159: mol_type = other RNA

SEQ ID NO: 282 - skipped

SEQ ID NO: 283 - RNA, 159 nt, synthetic construct; identical to SEQ ID NO: 251
  source 1..159: mol_type = other RNA

SEQ ID NO: 284 - skipped

SEQ ID NO: 285 - RNA, 159 nt, synthetic construct; identical to SEQ ID NO: 251
  source 1..159: mol_type = other RNA

SEQ ID NO: 286 - skipped

SEQ ID NO: 287 - RNA, 159 nt, synthetic construct; identical to SEQ ID NO: 251
  source 1..159: mol_type = other RNA

SEQ ID NO: 288 - RNA, 10 nt, synthetic construct
  source 1..10: mol_type = other RNA
ttttattttt

SEQ ID NO: 289 - RNA, 130 nt, synthetic construct
  source 1..130: mol_type = other RNA
ggcttcactgataaagtggagaaccgcttcaccgagtgaaggtgggctgcttgcatcagcctaatgtcgagaagtgctttcttcggaaagtaaccctcgaaacaaattcatttgaatgaaggaatgcaac

SEQ ID NO: 290 - RNA, 10 nt, synthetic construct
  source 1..10: mol_type = other RNA
ttttattttt

SEQ ID NO: 291 - RNA, 149 nt, synthetic construct; identical to SEQ ID NO: 263
  source 1..149: mol_type = other RNA

SEQ ID NO: 292 - RNA, 132 nt, synthetic construct; identical to SEQ ID NO: 264
  source 1..132: mol_type = other RNA

SEQ ID NO: 293 - RNA, 10 nt, synthetic construct
  source 1..10: mol_type = other RNA
ttttattttt

SEQ ID NO: 294 - RNA, 130 nt, synthetic construct; identical to SEQ ID NO: 289
  source 1..130: mol_type = other RNA

SEQ ID NO: 295 - RNA, 10 nt, synthetic construct
  source 1..10: mol_type = other RNA
ttttattttt

SEQ ID NO: 296 - RNA, 122 nt, synthetic construct
  source 1..122: mol_type = other RNA
ggcttcactgataaagtggagaaccgcttcacttagagtgaaggtgggctgcttgcatcagcctaatgtcgagaagtgctttcttcggaaagtaaccctcgaaacaaagaaaggaatgcaac

SEQ ID NO: 297 - RNA, 120 nt, synthetic construct
  source 1..120: mol_type = other RNA
ggcttcactgataaagtggagaaccgcttcaccgagtgaaggtgggctgcttgcatcagcctaatgtcgagaagtgctttcttcggaaagtaaccctcgaaacaaagaaaggaatgcaac

SEQ ID NO: 298 - RNA, 100 nt, synthetic construct
  source 1..100: mol_type = other RNA
accgcttcacttagagtgaaggtgggctgcttgcatcagcctaatgtcgagaagtgctttcttcggaaagtaaccctcgaaacaaagaaaggaatgcaac

SEQ ID NO: 299 - RNA, 98 nt, synthetic construct
  source 1..98: mol_type = other RNA
accgcttcaccgagtgaaggtgggctgcttgcatcagcctaatgtcgagaagtgctttcttcggaaagtaaccctcgaaacaaagaaaggaatgcaac

SEQ ID NO: 300 - RNA, 100 nt, synthetic construct; identical to SEQ ID NO: 298
  source 1..100: mol_type = other RNA

SEQ ID NO: 301 - RNA, 10 nt, synthetic construct
  source 1..10: mol_type = other RNA
ttttattttt

SEQ ID NO: 302 - RNA, 98 nt, synthetic construct; identical to SEQ ID NO: 299
  source 1..98: mol_type = other RNA

SEQ ID NO: 303 - RNA, 10 nt, synthetic construct
  source 1..10: mol_type = other RNA
ttttattttt

SEQ ID NO: 304 - RNA, 104 nt, synthetic construct
  source 1..104: mol_type = other RNA
accgcttcacgcttcggcagtgaaggtgggctgcttgcatcagcctaatgtcgagaagtgctttcttcggaaagtaaccctcgaaacaaagaaaggaatgcaac

SEQ ID NO: 305 - RNA, 104 nt, synthetic construct; identical to SEQ ID NO: 304
  source 1..104: mol_type = other RNA

SEQ ID NO: 306 - skipped

SEQ ID NO: 307 - RNA, 103 nt, synthetic construct
  source 1..103: mol_type = other RNA
ccgcttcacgcttcggcagtgaaggtgggctgcttgcatcagcctaatgtcgagaagtgctttcttcggaaagtaaccctcgaaacaaagaaaggaatgcaac

SEQ ID NO: 308 - RNA, 103 nt, synthetic construct
  source 1..103: mol_type = other RNA
ccgcttcacgcttaggcagtgaaggtgggctgcttgcatcagcctaatgtcgagaagtgctttcttcggaaagtaaccctcgaaacaaagaaaggaatgcaac

SEQ ID NO: 309 - RNA, 103 nt, synthetic construct
  source 1..103: mol_type = other RNA
ccgcttcactcttaggaagtgaaggtgggctgcttgcatcagcctaatgtcgagaagtgctttcttcggaaagtaaccctcgaaacaaagaaaggaatgcaac

SEQ ID NO: 310 - RNA, 103 nt, synthetic construct
  source 1..103: mol_type = other RNA
ccgcttcacgtttagacagtgaaggtgggctgcttgcatcagcctaatgtcgagaagtgctttcttcggaaagtaaccctcgaaacaaagaaaggaatgcaac

SEQ ID NO: 311 - RNA, 104 nt, synthetic construct
  source 1..104: mol_type = other RNA
gccgcttcacgcttcggcagtgaaggtgggctgcttgcatcagcctaatgtcgagaagtgctttcttcggaaagtaaccctcgaaacaaagaaaggaatgcaac

SEQ ID NO: 312 - RNA, 105 nt, synthetic construct
  source 1..105: mol_type = other RNA
ggccgcttcacgcttcggcagtgaaggtgggctgcttgcatcagcctaatgtcgagaagtgctttcttcggaaagtaaccctcgaaacaaagaaaggaatgcaac

SEQ ID NO: 313 - RNA, 102 nt, synthetic construct
  source 1..102: mol_type = other RNA
cgcttcacgcttcggcagtgaaggtgggctgcttgcatcagcctaatgtcgagaagtgctttcttcggaaagtaaccctcgaaacaaagaaaggaatgcaac

SEQ ID NO: 314 - RNA, 101 nt, synthetic construct
  source 1..101: mol_type = other RNA
gcttcacgcttcggcagtgaaggtgggctgcttgcatcagcctaatgtcgagaagtgctttcttcggaaagtaaccctcgaaacaaagaaaggaatgcaac

SEQ ID NO: 315 - RNA, 102 nt, synthetic construct
  source 1..102: mol_type = other RNA
ggcttcacgcttcggcagtgaaggtgggctgcttgcatcagcctaatgtcgagaagtgctttcttcggaaagtaaccctcgaaacaaagaaaggaatgcaac

SEQ ID NO: 316 - RNA, 101 nt, synthetic construct; identical to SEQ ID NO: 314
  source 1..101: mol_type = other RNA

SEQ ID NO: 317 - RNA, 10 nt, synthetic construct
  source 1..10: mol_type = other RNA
ttttattttt

SEQ ID NO: 318 - RNA, 102 nt, synthetic construct; identical to SEQ ID NO: 315
  source 1..102: mol_type = other RNA

SEQ ID NO: 319 - RNA, 10 nt, synthetic construct
  source 1..10: mol_type = other RNA
ttttattttt

SEQ ID NO: 320 - RNA, 102 nt, synthetic construct
  source 1..102: mol_type = other RNA
ccgcttcacgcttcggcagtgaaggtgggctgcttgcatcagcctaatgtcgagaagtgctttcttcggaaagtaaccctcgaaacaaagaaggaatgcaac

SEQ ID NO: 321 - RNA, 101 nt, synthetic construct
  source 1..101: mol_type = other RNA
ccgcttcacgcttcggcagtgaaggtgggctgcttgcatcagcctaatgtcgagaagtgctttcttcggaaagtaaccctcgaaacaaagaggaatgcaac

SEQ ID NO: 322 - RNA, 100 nt, synthetic construct
  source 1..100: mol_type = other RNA
ccgcttcacgcttcggcagtgaaggtgggctgcttgcatcagcctaatgtcgagaagtgctttcttcggaaagtaaccctcgaaacaaagggaatgcaac

SEQ ID NO: 323 - RNA, 99 nt, synthetic construct
  source 1..99: mol_type = other RNA
ccgcttcacgcttcggcagtgaaggtgggctgcttgcatcagcctaatgtcgagaagtgctttcttcggaaagtaaccctcgaaacaaaggaatgcaac

SEQ ID NO: 324 - RNA, 98 nt, synthetic construct
  source 1..98: mol_type = other RNA
ccgcttcacgcttcggcagtgaaggtgggctgcttgcatcagcctaatgtcgagaagtgctttcttcggaaagtaaccctcgaaacaaggaatgcaac

SEQ ID NO: 325 - RNA, 97 nt, synthetic construct
  source 1..97: mol_type = other RNA
ccgcttcacgcttcggcagtgaaggtgggctgcttgcatcagcctaatgtcgagaagtgctttcttcggaaagtaaccctcgaaacaggaatgcaac

SEQ ID NO: 326 - RNA, 96 nt, synthetic construct
  source 1..96: mol_type = other RNA
ccgcttcacgcttcggcagtgaaggtgggctgcttgcatcagcctaatgtcgagaagtgctttcttcggaaagtaaccctcgaaacggaatgcaac

SEQ ID NO: 327 - RNA, 95 nt, synthetic construct
  source 1..95: mol_type = other RNA
ccgcttcacgcttcggcagtgaaggtgggctgcttgcatcagcctaatgtcgagaagtgctttcttcggaaagtaaccctcgaaaggaatgcaac

SEQ ID NO: 328 - RNA, 94 nt, synthetic construct
  source 1..94: mol_type = other RNA
ccgcttcacgcttcggcagtgaaggtgggctgcttgcatcagcctaatgtcgagaagtgctttcttcggaaagtaaccctcgaaggaatgcaac

SEQ ID NO: 329 - RNA, 93 nt, synthetic construct
  source 1..93: mol_type = other RNA
ccgcttcacgcttcggcagtgaaggtgggctgcttgcatcagcctaatgtcgagaagtgctttcttcggaaagtaaccctcgaggaatgcaac

SEQ ID NO: 330 - RNA, 97 nt, synthetic construct
  source 1..97: mol_type = other RNA
ccgcttcacgcttcggcagtgaaggtgggctgcttgcatcagcctaatgtcgagaagtgctttcttcggaaagtaaccctcgaaacaagaatgcaac

SEQ ID NO: 331 - RNA, 99 nt, synthetic construct; identical to SEQ ID NO: 323
  source 1..99: mol_type = other RNA

SEQ ID NO: 332 - RNA, 98 nt, synthetic construct
  source 1..98: mol_type = other RNA
ccgcttcacgcttcggcagtgaaggtgggctgcttgcatcagcctaatgtcgagaagtgctttcttcggaaagtaaccctcgaaacaaagaatgcaac

SEQ ID NO: 333 - RNA, 102 nt, synthetic construct
  source 1..102: mol_type = other RNA
ccgcttcacgcttcggcagtgaaggtgggctgcttgcatcagcctaatgtcgagaagtgctttcttcggaaagtaaccctcgaaacaaagaaagaatgcaac

SEQ ID NO: 334 - RNA, 100 nt, synthetic construct
  source 1..100: mol_type = other RNA
ccgcttcacgcttcggcagtgaaggtgggctgcttgcatcagcctaatgtcgagaagtgctttcttcggaaagtaaccctcgaaacaaagagaatgcaac

SEQ ID NO: 335 - RNA, 99 nt, synthetic construct
  source 1..99: mol_type = other RNA
ccgcttcacgcttcggcagtgaaggtgggctgcttgcatcagcctaatgtcgagaagtgctttcttcggaaagtaaccctcgaaacaaagaaatgcaac

SEQ ID NO: 336 - RNA, 96 nt, synthetic construct
  source 1..96: mol_type = other RNA
ccgcttcacgcttcggcagtgaaggtgggctgcttgcatcagcctaatgtcgagaagtgctttcttcggaaagtaaccctcgaaacagaatgcaac

SEQ ID NO: 337 - RNA, 95 nt, synthetic construct
  source 1..95: mol_type = other RNA
ccgcttcacgcttcggcagtgaaggtgggctgcttgcatcagcctaatgtcgagaagtgctttcttcggaaagtaaccctcgaaacaaatgcaac

SEQ ID NO: 338 - RNA, 103 nt, synthetic construct
  source 1..103: mol_type = other RNA
ccgcttcacgcttcggcagtgaaggtgggctgattgcatcagcctaatgtcgagaagtgctttcttcggaaagtaaccctcgaaacaaagaaaggaatgcaac

SEQ ID NO: 339 - RNA, 103 nt, synthetic construct
  source 1..103: mol_type = other RNA
ccgcttcacgcttcggcagtgaaggtaggctgcttgcatcagcctaatgtcgagaagtgctttcttcggaaagtaaccctcgaaacaaagaaaggaatgcaac

SEQ ID NO: 340 - RNA, 103 nt, synthetic construct
  source 1..103: mol_type = other RNA
ccgcttcacgcttcggcagtgaaggtgggctgcttgcatcagcccaatgtcgagaagtgctttcttcggaaagtaaccctcgaaacaaagaaaggaatgcaac

SEQ ID NO: 341 - RNA, 97 nt, synthetic construct
  source 1..97: mol_type = other RNA
ggcttcacgcttcggcagtgaaggtaggctgcttgcatcagcctaatgtcgagaagtgctttcttcggaaagtaaccctcgaaacaaggaatgcaac

SEQ ID NO: 342 - RNA, 97 nt, synthetic construct
  source 1..97: mol_type = other RNA
ggcttcacgcttcggcagtgaaggtgggctgcttgcatcagcccaatgtcgagaagtgctttcttcggaaagtaaccctcgaaacaaggaatgcaac

SEQ ID NO: 343 - RNA, 103 nt, synthetic construct
  source 1..103: mol_type = other RNA
ccgcttcactcttaggaagtgaaggtgggctgattgcatcagcctaatgtcgagaagtgctttcttcggaaagtaaccctcgaaacaaagaaaggaatgcaac

SEQ ID NO: 344 - RNA, 100 nt, synthetic construct
  source 1..100: mol_type = other RNA
ccgcttcacgcttcggcagtgaaggtgggctgattgcatcagcctaatgtcgagaagtgctttcttcggaaagtaaccctcgaaacaaagggaatgcaac

SEQ ID NO: 345 - RNA, 99 nt, synthetic construct
  source 1..99: mol_type = other RNA
ccgcttcacgcttcggcagtgaaggtgggctgattgcatcagcctaatgtcgagaagtgctttcttcggaaagtaaccctcgaaacaaaggaatgcaac

SEQ ID NO: 346 - RNA, 98 nt, synthetic construct
  source 1..98: mol_type = other RNA
ccgcttcacgcttcggcagtgaaggtgggctgattgcatcagcctaatgtcgagaagtgctttcttcggaaagtaaccctcgaaacaaggaatgcaac

SEQ ID NO: 347 - RNA, 97 nt, synthetic construct
  source 1..97: mol_type = other RNA
ccgcttcacgcttcggcagtgaaggtgggctgattgcatcagcctaatgtcgagaagtgctttcttcggaaagtaaccctcgaaacaagaatgcaac

SEQ ID NO: 348 - RNA, 99 nt, synthetic construct; identical to SEQ ID NO: 345
  source 1..99: mol_type = other RNA

SEQ ID NO: 349 - RNA, 98 nt, synthetic construct
  source 1..98: mol_type = other RNA
ccgcttcacgcttcggcagtgaaggtgggctgattgcatcagcctaatgtcgagaagtgctttcttcggaaagtaaccctcgaaacaaagaatgcaac

SEQ ID NO: 350 - RNA, 96 nt, synthetic construct
  source 1..96: mol_type = other RNA
ccgcttcacgcttcggcagtgaaggtgggctgattgcatcagcctaatgtcgagaagtgctttcttcggaaagtaaccctcgaaacagaatgcaac